(19) United States
(12) Patent Application Publication
(10) Pub. No.: US 2008/0038363 A1
(43) Pub. Date: Feb. 14, 2008

(54) AEROSOL DELIVERY SYSTEM AND USES THEREOF

(76) Inventors: Alejandro C. Zaffaroni, Atherton, CA (US); Joshua D. Rabinowitz, Princeton, NJ (US)

Correspondence Address: WILSON SONSINI GOODRICH & ROSATI, 650 PAGE MILL ROAD, PALO ALTO, CA, 94304-1050, US

(21) Appl. No.: 11/687,466

(22) Filed: Mar. 16, 2007

Related U.S. Application Data
(63) Continuation of application No. 11/460,530, filed on Jul. 27, 2006, now abandoned.
Application No. 11/460,530 is a continuation of application No. 11/283,414, filed on Nov. 17, 2005, now abandoned.
(63) Continuation-in-part of application No. 10/633,876, filed on Aug. 4, 2003.
(63) Continuation-in-part of application No. 10/057,197, filed on Oct. 26, 2001.
(63) Continuation-in-part of application No. 10/057,198, filed on Oct. 26, 2001.
(63) Continuation-in-part of application No. 10/146,080, filed on May 13, 2002.
Application No. 10/146,080 is a continuation-in-part of application No. 10/057,198, filed on Oct. 26, 2001.
(63) Continuation-in-part of application No. 10/057,197, filed on Oct. 26, 2001.
(63) Continuation-in-part of application No. 10/146,086, filed on May 13, 2002.
(63) Continuation-in-part of application No. 10/146,088, filed on May 13, 2002.
Application No. 10/146,088 is a continuation-in-part of application No. 10/057,198, filed on Oct. 26, 2001.
(63) Continuation-in-part of application No. 10/057,197, filed on Oct. 26, 2001.
(63) Continuation-in-part of application No. 10/146,515, filed on May 13, 2002, now Pat. No. 6,682,716.
Application No. 10/146,515 is a continuation-in-part of application No. 10/057,198, filed on Oct. 26, 2001.
(63) Continuation-in-part of application No. 10/577,197, filed on Apr. 27, 2006.
(63) Continuation-in-part of application No. 10/146,516, filed on May 13, 2002, now Pat. No. 6,737,042.
(63) Continuation-in-part of application No. 10/150,056, filed on May 15, 2002, now Pat. No. 6,805,853.
(63) Continuation-in-part of application No. 10/150,267, filed on May 15, 2002, now Pat. No. 6,797,259.
(63) Continuation-in-part of application No. 10/150,268, filed on May 15, 2002, now Pat. No. 6,780,399.
(63) Continuation-in-part of application No. 10/150,591, filed on May 17, 2002, now Pat. No. 6,780,400.
(63) Continuation-in-part of application No. 10/150,857, filed on May 17, 2002, now Pat. No. 6,716,415.
(63) Continuation-in-part of application No. 10/151,596, filed on May 16, 2002, now Pat. No. 6,855,310.
(63) Continuation-in-part of application No. 10/151,626, filed on May 16, 2002, now Pat. No. 6,783,753.
(63) Continuation-in-part of application No. 10/152,639, filed on May 20, 2002, now Pat. No. 6,716,416.
(63) Continuation-in-part of application No. 10/152,640, filed on May 20, 2002, now Pat. No. 6,743,415.
(63) Continuation-in-part of application No. 10/152,652, filed on May 20, 2002, now Pat. No. 6,740,307.
(63) Continuation-in-part of application No. 10/153,139, filed on May 20, 2002, now Pat. No. 6,814,954.
(63) Continuation-in-part of application No. 10/153,311, filed on May 21, 2002, now Pat. No. 6,884,408.
(63) Continuation-in-part of application No. 10/153,313, filed on May 21, 2002, now abandoned.
(63) Continuation-in-part of application No. 10/153,831, filed on May 21, 2002, now Pat. No. 6,740,308.
(63) Continuation-in-part of application No. 10/153,839, filed on May 21, 2002, now Pat. No. 6,776,978.
(63) Continuation-in-part of application No. 10/154,594, filed on May 23, 2002, now Pat. No. 6,740,309.
(63) Continuation-in-part of application No. 10/154,765, filed on May 23, 2002, now Pat. No. 6,814,955.
(63) Continuation-in-part of application No. 10/155,097, filed on May 23, 2002, now Pat. No. 6,716,417.
(63) Continuation-in-part of application No. 10/155,373, filed on May 22, 2002, now Pat. No. 6,737,043.
(63) Continuation-in-part of application No. 10/155,621, filed on May 22, 2002, now Pat. No. 6,759,029.
(63) Continuation-in-part of application No. 10/155,703, filed on May 22, 2002, now Pat. No. 6,803,031.
(63) Continuation-in-part of application No. 10/155,705, filed on May 22, 2002, now Pat. No. 6,805,854.
(63) Continuation-in-part of application No. 10/280,315, filed on Oct. 25, 2002, now abandoned.
(63) Continuation-in-part of application No. 10/302,010, filed on Nov. 21, 2002, now Pat. No. 7,078,016.
(63) Continuation-in-part of application No. 10/302,614, filed on Nov. 21, 2002.
(63) Continuation-in-part of application No. 10/322,227, filed on Dec. 17, 2002, now abandoned.
(60) Provisional application No. 60/296,225, filed on Jun. 5, 2001.
(60) Provisional application No. 60/296,225, filed on Jun. 5, 2001.
(60) Provisional application No. 60/296,225, filed on Jun. 5, 2001.
(60) Provisional application No. 60/296,225, filed on Jun. 5, 2001.
(60) Provisional application No. 60/296,225, filed on Jun. 5, 2001.
(60) Provisional application No. 60/296,225, filed on Jun. 5, 2001.
(60) Provisional application No. 60/296,225, filed on Jun. 5, 2001.
(60) Provisional application No. 60/294,203, filed on May 24, 2001.
(60) Provisional application No. 60/317,479, filed on Sep. 5, 2001.
(60) Provisional application No. 60/345,882, filed on Nov. 9, 2001.
(60) Provisional application No. 60/294,203, filed on May 24, 2001.
(60) Provisional application No. 60/317,479, filed on Sep. 5, 2001.
(60) Provisional application No. 60/294,203, filed on May 24, 2001.
(60) Provisional application No. 60/317,479, filed on Sep. 5, 2001.
(60) Provisional application No. 60/294,203, filed on May 24, 2001.
(60) Provisional application No. 60/317,479, filed on Sep. 5, 2001.
(60) Provisional application No. 60/294,203, filed on May 24, 2001.
(60) Provisional application No. 60/317,479, filed on Sep. 5, 2001.
(60) Provisional application No. 60/294,203, filed on May 24, 2001.
(60) Provisional application No. 60/317,479, filed on Sep. 5, 2001.
(60) Provisional application No. 60/294,203, filed on May 24, 2001.
(60) Provisional application No. 60/317,479, filed on Sep. 5, 2001.
(60) Provisional application No. 60/294,203, filed on May 24, 2001.
(60) Provisional application No. 60/317,479, filed on Sep. 5, 2001.
(60) Provisional application No. 60/294,203, filed on May 24, 2001.
(60) Provisional application No. 60/317,479, filed on Sep. 5, 2001.
(60) Provisional application No. 60/294,203, filed on May 24, 2001.
(60) Provisional application No. 60/317,479, filed on Sep. 5, 2001.
(60) Provisional application No. 60/294,203, filed on May 24, 2001.
(60) Provisional application No. 60/317,479, filed on Sep. 5, 2001.
(60) Provisional application No. 60/294,203, filed on May 24, 2001.
(60) Provisional application No. 60/317,479, filed on Sep. 5, 2001.
(60) Provisional application No. 60/294,203, filed on May 24, 2001.
(60) Provisional application No. 60/317,479, filed on Sep. 5, 2001.
(60) Provisional application No. 60/345,145, filed on Nov. 9, 2001.
(60) Provisional application No. 60/294,203, filed on May 24, 2001.
(60) Provisional application No. 60/317,479, filed on Sep. 5, 2001.
(60) Provisional application No. 60/294,203, filed on May 24, 2001.
(60) Provisional application No. 60/317,479, filed on Sep. 5, 2001.
(60) Provisional application No. 60/294,203, filed on May 24, 2001.
(60) Provisional application No. 60/317,479, filed on Sep. 5, 2001.
(60) Provisional application No. 60/294,203, filed on May 24, 2001.
(60) Provisional application No. 60/317,479, filed on Sep. 5, 2001.
(60) Provisional application No. 60/294,203, filed on May 24, 2001.
(60) Provisional application No. 60/317,479, filed on Sep. 5, 2001.
(60) Provisional application No. 60/294,203, filed on May 24, 2001.
(60) Provisional application No. 60/317,479, filed on Sep. 5, 2001.
(60) Provisional application No. 60/345,876, filed on Nov. 9, 2001.
(60) Provisional application No. 60/294,203, filed on May 24, 2001.
(60) Provisional application No. 60/317,479, filed on Sep. 5, 2001.
(60) Provisional application No. 60/332,280, filed on Nov. 21, 2001.
(60) Provisional application No. 60/336,218, filed on Oct. 30, 2001.
(60) Provisional application No. 60/294,203, filed on May 24, 2001.
(60) Provisional application No. 60/317,479, filed on Sep. 5, 2001.
(60) Provisional application No. 60/294,203, filed on May 24, 2001.
(60) Provisional application No. 60/317,479, filed on Sep. 5, 2001.
(60) Provisional application No. 60/335,049, filed on Oct. 30, 2001.
(60) Provisional application No. 60/371,457, filed on Apr. 9, 2002.
(60) Provisional application No. 60/332,279, filed on Nov. 21, 2001.
(60) Provisional application No. 60/332,165, filed on Nov. 21, 2001.
(60) Provisional application No. 60/342,066, filed on Dec. 18, 2001.
(60) Provisional application No. 60/412,068, filed on Sep. 18, 2002.

(51) Int. Cl. A61K 9/14; A61K 31/5513; A61P 25/00; A61K 31/5517

(52) U.S. Cl. 424/502; 424/489; 514/220; 514/221

(57) ABSTRACT

A device, method, and system for producing a condensation aerosol are disclosed. The device includes a chamber having an upstream opening and a downstream opening which allow gas to flow through the chamber, and a heat-conductive substrate located at a position between the upstream and downstream openings. Formed on the substrate is a drug composition film containing a therapeutically effective dose of a drug when the drug is administered in aerosol form. A heat source in the device is operable to supply heat to the substrate to produce a substrate temperature greater than 300 oC, and to substantially volatilize the drug composition film from the substrate in a period of 2 seconds or less. The device produces an aerosol containing less than about 10% by weight drug composition degradation products and at least 50% of the drug composition of said film.

### CROSS-REFERENCE

The present application is a continuation-in-part of application Ser. No. 10/633,876, filed Aug. 4, 2003.

The present application is a continuation-in-part of application Ser. No. 10/057,197, filed Oct. 26, 2001, which claims benefit of Provisional Application No. 60/296,225, filed Jun. 5, 2001.

This application is also a continuation-in-part of application Ser. No. 10/057,198, filed Oct. 26, 2001, which claims benefit of Provisional Application No. 60/296,225, filed Jun. 5, 2001.

This application is also a continuation-in-part of application Ser. No. 10/146,080, filed May 13, 2002, which is a continuation-in-part of application Ser. No. 10/057,198, filed Oct. 26, 2001, which claims the benefit of Provisional Application No. 60/296,225, filed Jun. 5, 2001. This application is also a continuation-in-part of application Ser. No. 10/057,197, filed Oct. 26, 2001, which claims the benefit of Provisional Application No. 60/296,225, filed Jun. 5, 2001.

This application is also a continuation-in-part of application Ser. No. 10/146,086, filed May 13, 2002.

This application is also a continuation-in-part of application Ser. No. 10/146,088, filed May 13, 2002, which is a continuation-in-part of patent application Ser. No. 10/057,198, filed Oct. 26, 2001, which claims the benefit of Provisional Application No. 60/296,225, filed Jun. 5, 2001. This application is also a continuation-in-part of patent application Ser. No. 10/057,197, filed Oct. 26, 2001, which claims the benefit of Provisional Application No. 60/296,225, filed Jun. 5, 2001.

This application is also a continuation-in-part of application Ser. No. 10/146,515, filed May 13, 2002, which is a continuation-in-part of patent application Ser. No. 10/057,198, filed Oct. 26, 2001, which claims the benefit of Provisional Application No. 60/296,225, filed Jun. 5, 2001. This application is also a continuation-in-part of patent application Ser. No. 10/057,197, filed Oct. 26, 2001, which claims the benefit of Provisional Application No. 60/296,225, filed Jun. 5, 2001.

This application is also a continuation-in-part of application Ser. No. 10/146,516, filed May 13, 2002, which claims the benefit of Provisional Application No. 60/294,203, filed May 24, 2001, and also claims the benefit of Provisional Application No. 60/317,479, filed Sep. 5, 2001.

This application is also a continuation-in-part of application Ser. No. 10/150,056, filed May 15, 2002, which claims the benefit of Provisional Application No. 60/345,882, filed Nov. 9, 2001.

This application is also a continuation-in-part of application Ser. No. 10/150,267, filed May 15, 2002, which claims the benefit of Provisional Application No. 60/294,203, filed May 24, 2001, and of Provisional Application No. 60/317,479, filed Sep. 5, 2001.

This application is also a continuation-in-part of application Ser. No. 10/150,268, filed May 15, 2002, which claims the benefit of Provisional Application No. 60/294,203, filed May 24, 2001, and of Provisional Application No. 60/317,479, filed Sep. 5, 2001.

This application is also a continuation-in-part of application Ser. No. 10/150,591, filed May 17, 2002, which claims the benefit of Provisional Application No. 60/294,203, filed May 24, 2001, and of Provisional Application No. 60/317,479, filed Sep. 5, 2001.

This application is also a continuation-in-part of application Ser. No. 10/150,857, filed May 17, 2002, which claims the benefit of Provisional Application No. 60/294,203, filed May 24, 2001, and of Provisional Application No. 60/317,479, filed Sep. 5, 2001.

This application is also a continuation-in-part of application Ser. No. 10/151,596, filed May 16, 2002, which claims the benefit of Provisional Application No. 60/294,203, filed May 24, 2001, and of Provisional Application No. 60/317,479, filed Sep. 5, 2001.

This application is also a continuation-in-part of application Ser. No. 10/151,626, filed May 16, 2002, which claims the benefit of Provisional Application No. 60/294,203, filed May 24, 2001, and of Provisional Application No. 60/317,479, filed Sep. 5, 2001.

This application is also a continuation-in-part of application Ser. No. 10/152,639, filed May 20, 2002, which claims the benefit of Provisional Application No. 60/294,203, filed May 24, 2001, and of Provisional Application No. 60/317,479, filed Sep. 5, 2001.

This application is also a continuation-in-part of application Ser. No. 10/152,640, filed May 20, 2002, which claims the benefit of Provisional Application No. 60/294,203, filed May 24, 2001, and of Provisional Application No. 60/317,479, filed Sep. 5, 2001.

This application is also a continuation-in-part of application Ser. No. 10/152,652, filed May 20, 2002, which claims the benefit of Provisional Application No. 60/294,203, filed May 24, 2001, and of Provisional Application No. 60/317,479, filed Sep. 5, 2001.

This application is also a continuation-in-part of application Ser. No. 10/153,139, filed May 20, 2002, which claims the benefit of Provisional Application No. 60/294,203, filed May 24, 2001, and of Provisional Application No. 60/317,479, filed Sep. 5, 2001.

This application is also a continuation-in-part of application Ser. No. 10/153,311, filed May 21, 2002, which claims the benefit of Provisional Application No. 60/294,203, filed May 24, 2001, and of Provisional Application No. 60/317,479, filed Sep. 5, 2001.

This application is also a continuation-in-part of application Ser. No. 10/153,313, filed May 20, 2002, which claims the benefit of Provisional Application No. 60/294,203, filed May 24, 2001, and of Provisional Application No. 60/317,479, filed Sep. 5, 2001, and of Provisional Application No. 60/345,145, filed Nov. 9, 2001.

This application is also a continuation-in-part of application Ser. No. 10/153,831, filed May 21, 2002, which claims the benefit of Provisional Application No. 60/294,203, filed May 24, 2001, and of Provisional Application No. 60/317,479, filed Sep. 5, 2001.

This application is also a continuation-in-part of application Ser. No. 10/153,839, filed May 21, 2002, which claims the benefit of Provisional Application No. 60/294,203, filed May 24, 2001, and of Provisional Application No. 60/317,479, filed Sep. 5, 2001.

This application is also a continuation-in-part of application Ser. No. 10/154,594, filed May 23, 2002, which claims the benefit of Provisional Application No. 60/294,203, filed May 24, 2001, and of Provisional Application No. 60/317,479, filed Sep. 5, 2001.

This application is also a continuation-in-part of application Ser. No. 10/154,765, filed May 23, 2002, which claims the benefit of Provisional Application No. 60/294,203, filed May 24, 2001, and of Provisional Application No. 60/317,479, filed Sep. 5, 2001.

This application is also a continuation-in-part of application Ser. No. 10/155,097, filed May 23, 2002, which claims the benefit of Provisional Application No. 60/294,203, filed May 24, 2001, and of Provisional Application No. 60/317,479, filed Sep. 5, 2001.

This application is also a continuation-in-part of application Ser. No. 10/155,373, filed May 22, 2002, which claims the benefit of Provisional Application No. 60/294,203, filed May 24, 2001, and of Provisional Application No. 60/317,479, filed Sep. 5, 2001, and of Provisional Application No. 60/345,876, filed Nov. 9, 2001.

This application is also a continuation-in-part of application Ser. No. 10/155,621, filed May 22, 2002, which claims the benefit of Provisional Application No. 60/294,203, filed May 24, 2001, and of Provisional Application No. 60/317,479, filed Sep. 5, 2001, and of Provisional Application No. 60/332,280, filed Nov. 21, 2001, and of Provisional Application No. 60/336,218, filed Oct. 30, 2001.

This application is also a continuation-in-part of application Ser. No. 10/155,703, filed May 22, 2002, which claims the benefit of Provisional Application No. 60/294,203, filed May 24, 2001, and of Provisional Application No. 60/317,479, filed Sep. 5, 2001.

This application is also a continuation-in-part of application Ser. No. 10/155,705, filed May 22, 2002, which claims the benefit of Provisional Application No. 60/294,203, filed May 24, 2001, and of Provisional Application No. 60/317,479, filed Sep. 5, 2001.

This application is also a continuation-in-part of application Ser. No. 10/280,315, filed Nov. 25, 2002, which claims the benefit of Provisional Application No. 60/335,049, filed Oct. 30, 2001, and of Provisional Application No. 60/371,457, filed Apr. 9, 2002.

This application is also a continuation-in-part of application Ser. No. 10/302,010, filed Nov. 21, 2002, which claims the benefit of Provisional Application No. 60/332,279, filed Nov. 21, 2001.

This application is also a continuation-in-part of application Ser. No. 10/302,614, filed Nov. 21, 2002, which claims the benefit of Provisional Application No. 60/332,165, filed Nov. 21, 2001.

This application is also a continuation-in-part of application Ser. No. 10/322,227, filed Dec. 17, 2002, which claims the benefit of Provisional Application No. 60/342,066, filed Dec. 18, 2001, and of Provisional Application No. 60/412,068, filed Sep. 18, 2002.

All of the applications cited above are incorporated by reference in their entirety.

### FIELD OF THE INVENTION

The present invention relates generally to the field of thermal vapors or aerosols of drugs, devices and methods for administration of such compositions.

### BACKGROUND OF THE INVENTION

There are many factors to consider when evaluating the benefits of a particular type of drug therapy. Some of these factors include bioavailability of the drug delivered, rate of onset of drug action, severity of side effects, and convenience of patient use. A patient controlled analgesic delivery system is available that produces rapid onset of drug action and minimizes drug side effects (Bennett et al., Annals of Surgery 195(6): 700-705 (1982); Graves et al., Annals of Internal Medicine 99(3): 360-366 (1983)). However, this system administers the drug by intravenous bolus, which often requires the inconvenience of hospitalization.

Traditionally, inhalation therapy has played a relatively minor role in the administration of therapeutic agents when compared to more traditional drug administration routes of oral delivery and delivery via injection. Due to drawbacks associated with traditional routes of administration, including slow onset, poor patient compliance, inconvenience, and/or discomfort, alternative administration routes have been sought. Pulmonary delivery is one such alternative administration route which can offer several advantages over the more traditional routes. These advantages include rapid onset, the convenience of patient self-administration, the potential for reduced drug side-effects, ease of delivery by inhalation, the elimination of needles, and the like. Many preclinical and clinical studies with inhaled compounds have demonstrated that efficacy can be achieved both within the lungs and systemically. Inhalation therapy is capable of providing a drug delivery system that is easy to use in an inpatient or outpatient setting, results in very rapid onset of drug action, and produces minimal side effects. Inhalation drug therapy in clinical use currently focuses on the delivery of respiratory drugs via metered dose inhalers (MDIs). MDIs generally involve suspending small solid drug particles in a volatile liquid under pressure. Opening of a valve releases the suspension at relatively high velocity. The liquid then volatilizes, leaving behind a fast-moving aerosol of drug particles. Although MDIs have revolutionized the treatment of asthma, they are reliable for drug delivery only to mid-sized airways for the treatment of respiratory ailments

By manipulation of particle size and/or density, delivery of drugs into the alveoli may be facilitated. Alveoli have a large surface area for drug absorption and are surrounded by an extensive capillary network which facilitates rapid passage of drugs into the pulmonary circulation. Furthermore, because blood returning from the lungs is pumped directly to the systemic arterial circulation, drugs inhaled into the alveoli have the potential to reach target organs very rapidly. Of particular importance is that drugs delivered in this manner reach their target site without being exposed to potentially degrading conditions in the gastrointestinal tract and without undergoing modification by first pass metabolism in the liver. With these advantages in mind, dry powder formulations and new liquid aerosol devices are actively being developed for the systemic delivery of drugs after inhalation.

Dry powder inhalation involves generating very fine solid particles, mixing the particles with air, and inhaling the particles. Dry powder formulations for inhalation therapy are described in U.S. Pat. No. 5,993,805 to Sutton et al.; WO 0000176 to Robinson et al.; WO 9916419 to Tarara et al.; WO 0000215 to Bot et al; U.S. Pat. No. 5,855,913 to Hanes et al.; and U.S. Pat. Nos. 6,136,295 and 5,874,064 to Edwards et al.

For example, U.S. Pat. No. 5,993,805 to Sutton et al. describes spray-dried microparticles of a water-soluble material, which are smooth and spherical, and at least 90% of which have a mass median particle size of 1 to 10 microns, and which carry a therapeutic or diagnostic agent can successfully be used in dry powder inhalers to deliver the agent. See Abstract of U.S. Pat. No. 5,993,805. There is an optimal size of particle which will access the lowest regions of the pulmonary airways, i.e. an aerodynamic diameter of <5 μm. Particles above this size will be caught by impaction in the upper airways. Sutton et al. teaches the suitable size for respiratory drug delivery, i.e. 1-5 μm (col. 1, lines 36-39 and col. 2, 23-24). The Sutton patent specification further describes that preferably the wall-forming material is proteinaceous. For example, it may be collagen, gelatin or (serum) albumin, in each case preferably of human origin (i.e. derived from humans or corresponding in structure to the human protein). Most preferably, it is human serum albumin (HA) derived from blood donations or, ideally, from the fermentation of microorganisms (including cell lines) which have been transformed or transfected to express HA. See column 7, lines 1 to 8. The preparation to be sprayed may contain substances other than the wall-forming material and solvent or carrier liquid. The aqueous phase may contain 1-20% by weight of water-soluble hydrophilic compounds like sugars and polymers as stabilisers, e.g. polyvinyl alcohol (PVA) polyvinyl pyrrolidone (PVP), polyethylene glycol (PEG), gelatin, polyglutamic acid and polysaccharides such as starch, dextran, agar, xanthan and the like. Similar aqueous phases can be used as the carrier liquid in which the final microsphere product is suspended before use. Emulsifiers may be used (0.1-5% by weight) including most physiologically acceptable emulsifiers, for instance egg lecithin or soya bean lecithin, or synthetic lecithins such as saturated synthetic lecithins, for example, dimyristoyl lo phosphatidyl choline, dipalmitoyl phosphatidyl choline or distearoyl phosphatidyl choline or unsaturated synthetic lecithins, such as dioleyl phosphatidyl choline or dilinoleyl phosphatidyl choline. Emulsifiers also include surfactants such as free fatty acids, esters of fatty acids with polyoxyalkylene compounds like polyoxypropylene glycol and polyoxyethylene glycol; ethers of fatty alcohols with polyoxyalkylene glycols; esters of fatty acids with polyoxyalkylated sorbitan; soaps; glycerol-polyalkylene stearate; glycerol-polyoxyethylene ricinoleate; homo- and copolymers of polyalkylene glycols; polyethoxylated soya-oil and castor oil as well as hydrogenated derivatives; ethers and esters of sucrose or other carbohydrates with fatty acids, fatty alcohols, these being optionally polyoxyalkylated; mono-, di- and triglycerides of saturated or unsaturated fatty acids, glycerides or soya-oil and sucrose. See column 7, lines 40 to col. 8, line 2.

In another example, U.S. Pat. No. 5,874,064 to Edwards et al. describes improved aerodynamically light particles for drug delivery to the pulmonary system, and methods for their synthesis and administration. In a preferred embodiment, the particles are made of a biodegradable material, have a tap density less than 0.4 g/cm³ and a mean diameter between 5 μm and 30 μm. In one embodiment, for example, at least 90% of the particles have a mean diameter between 5 μm and 30 μm. The particles may be formed of biodegradable materials such as biodegradable polymers, proteins, or other water-soluble materials. See column 3, lines 13 to 22. For example, the particles may be formed of polymers including polyamides, polycarbonates, polyalkylenes such as polyethylene, polypropylene, poly(ethylene glycol), poly(ethylene oxide), poly(ethylene terephthalate), poly vinyl compounds such as polyvinyl alcohols, polyvinyl ethers, and polyvinyl esters, polymers of acrylic and methacrylic acids, celluloses and other polysaccharides, and peptides or proteins, or copolymers or blends thereof which are capable of forming aerodynamically light particles with a tap density less than about 0.4 g/cm³. Polymers may be selected with or modified to have the appropriate stability and degradation rates in vivo for different controlled drug delivery applications. See column 6, lines 58 to col. 7, line 2. In addition, the particles may be formed of a functionalized polyester graft copolymer consisting of a linear .alpha.-hydroxy-acid polyester backbone having at least one amino acid residue incorporated per molecule therein and at least one poly(amino acid) side chain extending from an amino acid group in the polyester backbone. See column 3, lines 22 to 28. Other examples include particles formed of water-soluble excipients, such as trehalose or lactose, or proteins, such as lysozyme or insulin. The aerodynamically light particles can be used for enhanced delivery of a therapeutic agent to the airways or the alveolar region of the lung. The particles incorporating a therapeutic agent may be effectively aerosolized for administration to the respiratory tract to permit systemic or local delivery of a wide variety of therapeutic agents. They optionally may be co-delivered with larger carrier particles, not carrying a therapeutic agent, which have for example a mean diameter ranging between about 50 μm and 100 μm. See column 3, lines 28 to 40.

As described in Edwards' specification, the mass mean diameter of the particles can be measured using a Coulter Counter. The aerodynamically light particles are preferably at least about 5 microns in diameter. The diameter of particles in a sample will range depending upon depending on factors such as particle composition and methods of synthesis. The distribution of size of particles in a sample can be selected to permit optimal deposition within targeted sites within the respiratory tract. See column 4, lines 11 to 8.

The Edwards' specification further illustrates that the aerodynamically light particles may be fabricated or separated, for example by filtration, to provide a particle sample with a preselected size distribution. For example, greater than 30%, 50%, 70%, or 80% of the particles in a sample can have a diameter within a selected range of at least 5 μm. The selected range within which a certain percentage of the particles must fall may be for example, between about 5 and 30 μm, or optionally between 5 and 15 μm. In one preferred embodiment, at least a portion of the particles have a diameter between about 9 and 11 μm. Optionally, the particle sample also can be fabricated wherein at least 90%, or optionally 95% or 99%, have a diameter within the selected range. The presence of the higher proportion of the aerodynamically light, larger diameter (at least about 5 .mu.m) particles in the particle sample enhances the delivery of therapeutic or diagnostic agents incorporated therein to the deep lung. See column 4, lines 19 to 35.

In one embodiment as described in Edwards et al., the interquartile particle range may be 2 μm, with a mean diameter for example of 7.5, 8.0, 8.5, 9.0, 9.5, 10.0, 10.5, 11.0, 11.5, 12.0, 12.5, 13.0 or 13.5 μm. Thus, for example, at least 30%, 40%, 50% or 60% of the particles may have diameters within the selected range 5.5-7.5 μm, 6.0-8.0 μm, 6.5-8.5 μm, 7.0-9.0 μm, 7.5-9.5 μm, 8.0-10.0 μm, 8.5-10.5 μm, 9.0-11.0 μm, 9.5-11.5 μm, 10.0-12.0 μm, 10.5-12.5 μm, 11.0-13.0 μm, 11.5-13.5 μm, 12.0-14.0 μm, 12.5-14.5 μm or 13.0-15.0 .μm. Preferably the said percentages of particles have diameters within a 1 μm range, for example, 6.0-7.0 μm, 10.0-11.0 μm or 13.0-14.0 μm. See column 4, lines 36 to 47.

The Edwards specification also illustrates that administration of the low density particles to the lung by aerosolization permits deep lung delivery of relatively large diameter therapeutic aerosols, for example, greater than 5 μm in mean diameter. See column 3, lines 65 to col. 4, line 1. Moreover, column 12, lines 50 to 64, describes that variables which may be manipulated to alter the size distribution of the particles include: polymer concentration, polymer molecular weight, surfactant type (e.g., PVA, PEG, etc.), surfactant concentration, and mixing intensity. Variables which may be manipulated to alter the surface shape and porosity of the particles include: polymer concentration, polymer molecular weight, rate of methylene chloride extraction by isopropyl alcohol (or another miscible solvent), volume of isopropyl alcohol added, inclusion of an inner water phase, volume of inner water phase, inclusion of salts or other highly water-soluble molecules in the inner water phase which leak out of the hardening sphere by osmotic pressure, causing the formation of channels, or pores, in proportion to their concentration, and surfactant type and concentration.

Two particle size ranges are known to have particular value when creating aerosols for inhalation delivery of drugs. Particles in the 10 to 100 nanometer (nm) size range are called ultra fine aerosols and particles in the 1-3 micron size range are called fine aerosols. These two size ranges are desirable for inhalation administration for lung physiology reasons, because both size ranges result in a high level of deposition of dmg particles in desirable regions within the lung for optimal drug absorption through the lung membranes into the blood stream.

Each of these two particle size ranges achieves its own optimal deposition by a different mechanism. In the 10-100 nm or ultra fine range, the mechanism of deposition is through diffusion. Because the particles in this size range are small compared to the mean free path (MFP), deposition in the lung is a result of collisions of the particles with the wall of the lung due to random movement of the aerosol from thermal energy. On the other hand, particles in the 1-3 micron range deposit in the pulmonary section of the lung though gravitational settling.

Drug delivery to the lung is used as a route to treat both diseases of the lung such as asthma and cystic fibrosis as well as a portal for delivering drugs to the systemic blood circulation system. Therefore, to deliver drugs to the systemic circulation system efficiently, the aerosol must deposit in the gas exchange region of the lung that is composed primarily of alveoli. A plot of deposition efficiency versus particle size for this region of the lung shows a bimodal distribution. This is due to the two different deposition mechanisms. Large particles greater than about 3 micron are filtered out before they can get into this region by inertial impaction. The 1-3 micron particles are deposited in the lung mostly by gravitational sedimentation, while particles less than about 0.1 μm are deposited by diffusion to the wall. The middle range, in which the lung deposition is inefficient, is from about 0.1 pm to about 1 μm where the particles settle too slowly to be deposited efficiently by sedimentation and are too large for diffusion to cause efficient deposition. The best common example of an aerosol of this size is cigarette smoke, where the smoke is in the range of about 0.2-0.5 μm. The smoke is small enough to get into the deep lung where some of it will deposit but the deposition is inefficient and most of it is exhaled; see Gonda, I., “Particle Deposition in the Human Respiratory Tract,” The Lung: Scientific Foundations, 2nd ed., Crystal, West, et al. editors, Lippincott-Raven Publishers, 1997.

There is a great deal of study regarding particle deposition in the lung in the fields of public health, environmental toxicology and radiation safety. Most of this modeling and in vivo data concerns the exposure of people to aerosols homogeneously distributed in the air that they breathe, where the subject does nothing actively to minimize or maximize deposition. The International Commission On Radiological Protection (ICRP) models are an example of this, and there are a great number of in vivo studies where the subject is doing what is known as tidal breathing. In the field of aerosol drug delivery, the patient is instructed to breathe in such a way that the deposition of the drug in the lung is maximized, and this usually involves a full exhalation, followed by a deep inhalation sometimes at a prescribed inhalation flow rate range, followed by a breath hold of several seconds. Ideally, the aerosol is not uniformly distributed in the air being inhaled, but is loaded into the early part of the breath as a bolus of aerosol, followed by a volume of clean air so that the aerosol is drawn into the alveoli and flushed out of the conductive airways, bronchi and trachea by the volume of clean air. A typical deep adult human breath has a volume of about 2 to 5 liters. In order to help insure consistent delivery in the whole population of adult patients, the delivery of the drug bolus should be completed in the first 1-1% liters or so of inhaled air.

As the inhalation flow rate increases, the rate of inertial impaction of the larger sizes increases. “The greater a particle's mass and velocity, the longer it persists flying in the original direction and, therefore, increases its chances of hitting the obstacle placed in front of it.” (See Gonda, I., “Particle Deposition in the Human Respiratory Tract,” referred to above.) Thus, given a velocity, generated by the inhalation flow rate, the effect of inertial impaction is greater on larger rather than smaller particles. Too high an inhalation flow rate will cause a loss of efficiency for the fine aerosols due to inertial impaction in the conductive airways.

One advantage of an ultra fine aerosol is that approximately 50,000 times as many particles exist within a volume of ultra fine aerosols as exist in the same mass of fine aerosols. Since each particle deposits on the membrane of the lung, a correspondingly greater number of deposition sites are created in the lungs and at each site less material has to be dissolved and transported into the blood stream. This may be important for improving the rate of absorption permeability and thus the bioavailabilty of compounds that are not rapidly absorbed by the lung, e.g., lipophilic compounds, large molecules such as proteins, peptides and DNA. It is suspected that a portion of some drugs that have a slow absorption rate from the alveoli are assimilated by macrophages before they can be absorbed, leading to a low bioavailability despite efficient deposition in the alveoli. There is a need for a method and device for generating fine and ultra fine aerosols that can be effectively administered to a patient or other user.

To date, the clinical application of dry powders has primarily focused on the delivery of macromolecules, such as insulin. Clinical application of dry powder inhalation delivery is limited by difficulties in generating dry powders of appropriate particle size and particle density, in keeping the powder stored in a dry state, and in developing a convenient, hand-held device that effectively disperses the particles to be inhaled in air. In addition, the particle size of dry powders for inhalation delivery is inherently limited by the fact that smaller particles are harder to disperse in air.

Liquid aerosol delivery is one of the oldest forms of pulmonary drug delivery. Typically, liquid aerosols are created by a nebulizer, which releases compressed air from a small orifice at high velocity, resulting in low pressure at the exit region due to the Bernoulli effect, as described in U.S. Pat. No. 5,511,726 to Greenspan et al. The low pressure is used to draw the fluid to be aerosolized out of a second tube. This fluid breaks into small droplets as it accelerates in the air stream. Disadvantages of this standard nebulizer design include relatively large particle size, lack of particle size uniformity, and low densities of small particles in the inhaled air.

Newer liquid aerosol technologies involve generating smaller and more uniform liquid particles by passing the liquid to be aerosolized through micron-sized holes. U.S. Pat. No. 6,131,570 to Schuster et al.; U.S. Pat. No. 5,724,957 to Rubsamen et al.; and U.S. Pat. No. 6,098,620 to Lloyd et al. describe the use of pressure generated by a piston to push fluid through a membrane with laser drilled holes. U.S. Pat. Nos. 5,586,550; 5,758,637; and 6,085,740 to Ivri et al.; and U.S. Pat. No. 5,938,117 to Ivri describe the use of vibration to move fluid through apertures in a shell that are larger on the fluid-coated side.

The role of inhalation therapy in the health care field has remained limited mainly to treatment of asthma, in part due to a set of problems unique to the development of inhalable drug formulations, especially formulations for systemic delivery by inhalation. Dry powder formulations, while offering advantages over cumbersome liquid dosage forms and propellant-driven formulations, are prone to aggregation and low flowability phenomena which considerably diminish the efficiency of dry powder-based inhalation therapies.

A further limitation that is shared by each of the above methods is that the aerosols produced typically include substantial quantities of inert carriers, solvents, emulsifiers, propellants, and other non-drug material. In general, the large quantities of non-drug material are required for effective formation of particles small enough for alveolar delivery (e.g. less than 5 microns and preferably less than 3 microns). However, these amounts of non-drug material also serve to reduce the purity and amount of active drug substance that can be delivered. Thus, these methods remain substantially incapable of introducing large drug dosages accurately to a patient for systemic delivery.

Vaporizing drugs may provide a method of maximizing alveolar delivery and rapidly delivering drugs to target organs. Scented candles and oil lamps are known to volatilize various fragrances and herbal remedies when the wax or oil is heated. For example, U.S. Pat. No. 5,840,246 to Hammons et al. describes an oil lamp that volatilizes insect repellent compositions, deodorizing compositions, medicinal compounds, herbal compositions, and disinfectant compositions. U.S. Pat. No. 5,456,247 to Schilling et al. describes the administration of vaporized sulfamethazine, sulfamethoxazole, sulfamethoxine, and gentamicin by inhalation of the vapor in a treatment chamber. Portable vaporizers and humidifiers that volatilize various compounds are also known. U.S. Pat. Nos. 4,734,560 and 4,853,517 to Bowen describe a vaporizing unit for medications, room deodorizers, room scenting compounds, and room insecticides. U.S. Pat. No. 4,566,451 to Badewien relates to a device that vaporizes medicated liquid. U.S. Pat. Nos. 4,906,417 to Gentry and 3,982,095 to Robinson describe humidifiers that vaporize medication. In the preceding examples, the vaporization of compounds occurs freely into air.

International application WO 94/09842 to Rosen describes a device with an electric heating element that vaporizes a predetermined amount of some agents. U.S. Pat. Nos. 4,917,119 to Potter et al.; 4,941,483 to Ridings et al.; 5,099,861 to Clearman et al.; 4,922,901 to Brooks et al.; and 4,303,083 to Buruss, Jr. also describe hand-held devices that vaporize various medications.

However, the heat required to vaporize a drug often also generates degradation products, which may decrease the efficacy of the thermal vapor and are undesirable to be delivered to the patient. Thus, a method that enhances drug volatilization without the formation of a substantial amount of degradation products is needed.

There also remains a need to enhance the formation of small particle size aerosols are needed. In addition, methods that produce aerosols comprising greater quantities of drug and lesser quantities of non-drug material are needed. Further, a method for producing small particle size aerosols comprising substantially pure drug is needed. Finally, a method that allows a patient to administer a unit dosage rapidly with a single, small volume breath is needed.

### SUMMARY OF THE INVENTION

In one aspect, the invention provides novel composition for delivery of a drug comprising a condensation aerosol formed by volatilizing a heat stable drug composition under conditions effective to produce a heated vapor of said drug composition and condensing the heated vapor of the drug composition to form condensation aerosol particles, wherein said condensation aerosol particles are characterized by less than 10% drug degradation products, and wherein the aerosol MMAD is less than 3 microns.

In some variations, the aerosol comprises at least 50% by weight of drug condensation particles. In other variations the aerosol comprises at least 90% or 95% by weight of the drug condensation particles. Similarly, in some variations, the aerosol is substantially free of thermal degradation products, and in some variations, the condensation aerosol has a MMAD in the range of 1-3 μm. Also, in some variations the molecular weight of the compound is typically between 200 and 700. Typically, the aerosol comprises a therapeutically effective amount of drug and in some variations may comprise pharmaceutically acceptable excipients. In some variations, the carrier gas is air. In some variations, other gases or a combination of various gases may be used.

In another aspect of the invention, the invention provides compositions for inhalation therapy, comprising an aerosol of vaporized drug condensed into particles, characterized by less than 5% drug degradation products, and wherein said aerosol has a mass median aerodynamic diameter between 1-3 microns.

In some variations of the aerosol compositions, the carrier gas is a non-propellant, non-organic solvent carrier gas. In other variations, the aerosol is substantially free of organic solvents and propellants.

In yet other embodiments, aerosols of a therapeutic drug are provided that contain less than 5% drug degradation products, and a mixture of a carrier gas and condensation particles, formed by condensation of a vapor of the drug in said carrier gas; where the MMAD of the aerosol increases over time, within the size range of 0.01 to 3 microns as said vapor cools by contact with the carrier gas.

In some variations, the aerosol comprises at least 50% by weight of drug condensation particles. In other variations the aerosol comprises at least 90% or 95% by weight of the drug condensation particles. In some variations, the MMAD of the aerosol is less than 1 micron and increases over time. Also, in some variations the molecular weight of the compound is typically between 200 and 700. In other variations, the compound has a molecular weight of greater than 350 and is heat stable. Typically, the aerosol comprises a therapeutically effective amount of drug and in some variations may comprise pharmaceutically acceptable excipients. In some variations, the carrier gas is air. In some variations, other gases or a combination of various gases may be used.

The condensation aerosols of the various embodiments are typically formed by preparing a film containing a drug composition of a desired thickness on a heat-conductive and impermeable substrate and heating said substrate to vaporize said film, and cooling said vapor thereby producing aerosol particles containing said drug composition. Rapid heating in combination with the gas flow helps reduce the amount of decomposition. Thus, a heat source is used that typically heats the substrate to a temperature of greater than 200° C., preferably at least 250° C., more preferably at least 300° C. or 350° C. and produces substantially complete volatilization of the drug composition from the substrate within a period of 2 seconds, preferably, within 1 second, and more preferably, within 0.5 seconds.

Typically, the gas flow rate over the vaporizing compound is between about 4 and 50 L/minute.

The film thickness is such that an aerosol formed by vaporizing the compound by heating the substrate and condensing the vaporized compound contains 10% by weight or less drug-degradation product. The use of thin films allows a more rapid rate of vaporization and hence, generally, less thermal drug degradation. Typically, the film has a thickness between 0.05 and 20 microns. In some variations, the film has a thickness between 0.5 and 5 microns. The selected area of the substrate surface expanse is such as to yield an effective human therapeutic dose of the drug aerosol.

In still another aspect, the invention provides a drug with desirable properties for thermal vapor delivery. Such improvement may involve providing a modified drug with enhanced volatility, including for example, thermal vapors of the ester, free base, and free acid forms of drugs. The ester, free base or free acid form of drug includes antibiotics, anticonvulsants, antidepressants, antihistamines, antiparkinsonian drugs, drugs for migraine headache, drugs for the treatment of alcoholism, muscle relaxants, anxiolytics (e.g., benzodiazepines), nonsteroidal anti-inflammatory drugs, other analgesics, and steroids. In one embodiment, a pharmaceutically acceptable drug is delivered to a patient by providing a drug ester, heating the drug ester to a temperature to form a thermal vapor that includes the drug ester, and then delivering the thermal vapor of the drug ester to the patient.

In still another aspect, the invention provides a thermal vapor for inhalation therapy that does not contain a significant amount of thermal degradation products. Yet another aspect of the invention is to provide a form of inhalation therapy where patients can titrate their intake of a drug.

The thermal vapors contain unit dose amounts of drug ester, drug free base, or drug free acid and less than 1% degradation products. The thermal vapors are delivered using a thermal vapor delivery device that contains a pharmaceutically acceptable drug ester, drug free base, or drug free acid, a heating element, and a passageway that links the site of volatilization with the site of inhalation.

The dose of that drug in thermal vapor form is generally less than the standard oral dose. Preferably it will be less than 80%, more preferably less than 40%, and most preferably less than 20% of the standard oral dose.

A further embodiment of the invention is a device for delivery of an aerosol of a drug, comprising an aerosolizer, a site of inhalation, and a passageway that links the site of aerosolization with the site of inhalation. In a further embodiment, the device also comprises a heater for heating the drug. In various embodiments, the aerosolizer may be a jet nebulizer or an ultrasonic nebulizer. The aerosolizer may apply a static electric charge to the drug. The aerosolizer may pass the drug through holes in a perforated membrane, wherein the holes have a mean diameter of between about 0.2 microns and about 10 microns. The aerosolizer may vaporize the drug and allow it to cool to form a condensation aerosol. The device may deliver an aerosol comprising a unit dose amount of the drug.

The thermal vapor delivery device may also include a monitor that controls the timing of drug volatilization relative to inhalation, a feature that gives feedback to patients on the rate or volume of inhalation or both the rate and volume of inhalation, a feature that prevents excessive use of the device, a feature that prevents use by unauthorized individuals, and a feature that records dosing histories.

A kit for delivery of the thermal vapors may also be supplied that includes a pharmaceutically acceptable drug ester, drug free base, or drug free acid, and a device that vaporizes those drugs. In the kit, the device delivers a unit dose amount of the drug ester, drug free base, or drug free acid in the thermal vapor.

In yet another aspect of the invention kits are provided for delivering a drug aerosol comprising a thin film of a drug composition and a device for dispensing said film as a condensation aerosol. Typically, the film thickness is between 0.5 and 20 microns. The film can comprise pharmaceutically acceptable excipients and is typically heated at a rate so as to substantially volatilize the film in 500 milliseconds or less.

The invention includes, in one aspect, a device for producing a condensation aerosol. The device includes a chamber having an upstream opening and a downstream opening which allow gas to flow through the chamber, and a heat-conductive substrate located at a position between the upstream and downstream openings. Formed on the substrate is a drug composition film containing a therapeutically effective dose of a drug when the drug is administered in aerosol form. A heat source in the device is operable to supply heat to the substrate to produce a substrate temperature greater than 300° C., and to substantially volatilize the drug composition film from the substrate in a period of 2 seconds or less. The device produces an aerosol containing less than about 10% by weight drug composition degradation products and at least 50% of the drug composition of said film. The device may include a mechanism for initiating said heat source.

The substrate may have an impermeable surface and/or a contiguous surface area of greater than 1 mm² and a material density of greater than 0.5 g/cc.

The thickness of the film may be selected to allow the drug composition to volatilize from the substrate with less than about 5% by weight drug composition degradation products.

The drug composition may be one that when vaporized from a film on an impermeable surface of a heat conductive substrate, the aerosol exhibits an increasing level of drug composition degradation products with increasing film thicknesses. Examples includes the following drugs, and associated ranges of film thicknesses:

alprazolam, film thickness between 0.1 and 10 μm;

amoxapine, film thickness between 2 and 20 μm;

atropine, film thickness between 0.1 and 10 μm;

bumetanide film thickness between 0.1 and 5 μm;

buprenorphine, film thickness between 0.05 and 10 μm;

butorphanol, film thickness between 0.1 and 10 μm;

clomipramine, film thickness between 1 and 8 μm;

donepezil, film thickness between 1 and 10 μm;

hydromorphone, film thickness between 0.05 and 10 μm;

loxapine, film thickness between 1 and 20 μm;

midazolam, film thickness between 0.05 and 20 μm;

morphine, film thickness between 0.2 and 10 μm;

nalbuphine, film thickness between 0.2 and 5 μm;

naratriptan, film thickness between 0.2 and 5 μm;

olanzapine, film thickness between 1 and 20 μm;

paroxetine, film thickness between 1 and 20 μm;

prochlorperazine, film thickness between 0.1 and 20 μm;

quetiapine, film thickness between 1 and 20 μm;

sertraline, film thickness between 1 and 20 μm;

sibutramine, film thickness between 0.5 and 2 μm;

sildenafil, film thickness between 0.2 and 3 μm;

sumatriptan, film thickness between 0.2 and 6 μm;

tadalafil, film thickness between 0.2 and 5 μm;

vardenafil, film thickness between 0.1 and 2 μm;

venlafaxine, film thickness between 2 and 20 μm;

zolpidem, film thickness between 0.1 and 10 μm;

apomorphine HCl, film thickness between 0.1 and 5 μm;

celecoxib, film thickness between 2 and 20 μm;

ciclesonide, film thickness between 0.05 and 5 μm;

eletriptan, film thickness between 0.2 and 20 μm;

parecoxib, film thickness between 0.5 and 2 μm;

valdecoxib, film thickness between 0.5 and 10 μm; and

fentanyl, film thickness between 0.05 and 5 μm.

The heat source may substantially volatilize the drug composition film from the substrate within a period of less than 0.5 seconds, and may produce a substrate temperature greater than 350° C. The heat source may comprise an ignitable solid chemical fuel disposed adjacent an interior surface of the substrate, such that the ignition of the fuel is effective to vaporize the drug composition film.

In a related aspect, the invention includes a method for producing a condensation aerosol. The method includes heating to a temperature greater than 300° C., a heat-conductive substrate having a drug composition film on the surface, the film comprising a therapeutically effective dose of a drug when the drug is administered in aerosol form. The heating is effective to substantially volatilize the drug composition film from the substrate in a period of 2 seconds or less. Air is flowed through the volatilized drug composition, under conditions to effective produce an aerosol containing less than 10% by weight drug composition degradation products and at least 50% of the drug composition in said film.

Various embodiments of the device noted above may form part of the method.

In still another aspect, the invention includes an assembly for use in a condensation aerosol device. The assembly includes a heat-conductive substrate having an interior surface and an exterior surface; a drug composition film on the substrate exterior surface, the film comprising a therapeutically effective dose of a drug when the drug is administered in aerosol form, and a heat source for supplying heat to said substrate to produce a substrate temperature greater than 300° C. and to substantially volatilize the drug composition film from the substrate in a period of 2 seconds or less.

Various embodiments of the device noted above may form part of the assembly.

The present invention also relates to the delivery of alprazolam, estazolam, midazolam or triazolam through an inhalation route. Specifically, it relates to aerosols containing alprazolam, estazolam, midazolam or triazolam that are used in inhalation therapy.

In a composition aspect of the present invention, the aerosol comprises particles comprising at least 5 percent by weight of alprazolam, estazolam, midazolam or triazolam. Preferably, the particles comprise at least 10 percent by weight of alprazolam, estazolam, midazolam or triazolam. More preferably, the particles comprise at least 20 percent, 30 percent, 40 percent, 50 percent, 60 percent, 70 percent, 80 percent, 90 percent, 95 percent, 97 percent, 99 percent, 99.5 percent or 99.97 percent by weight of alprazolam, estazolam, midazolam or triazolam.

Typically, the aerosol has a mass of at least 1 μg. Preferably, the aerosol has a mass of at least 10 μg. More preferably, the aerosol has a mass of at least 20 μg.

Typically, the aerosol particles comprise less than 10 percent by weight of alprazolam, estazolam, midazolam or triazolam degradation products. Preferably, the particles comprise less than 5 percent by weight of alprazolam, estazolam, midazolam or triazolam degradation products. More preferably, the particles comprise less than 2.5, 1, 0.5, 0.1 or 0.03 percent by weight of alprazolam, estazolam, midazolam or triazolam degradation products.

Typically, the aerosol particles comprise less than 90 percent by weight of water. Preferably, the particles comprise less than 80 percent by weight of water. More preferably, the particles comprise less than 70 percent, 60 percent, 50 percent, 40 percent, 30 percent, 20 percent, 10 percent, or 5 percent by weight of water.

Typically, at least 50 percent by weight of the aerosol is amorphous in form, wherein crystalline forms make up less than 50 percent by weight of the total aerosol weight, regardless of the nature of individual particles. Preferably, at least 75 percent by weight of the aerosol is amorphous in form. More preferably, at least 90 percent by weight of the aerosol is amorphous in form.

Typically, the aerosol has an inhalable aerosol drug mass density of between 0.02 mg/L and 10 mg/L. Preferably, the aerosol has an inhalable aerosol drug mass density of between 0.05 mg/L and 5 mg/L. More preferably, the aerosol has an inhalable aerosol drug mass density of between 0.1 mg/L and 2 mg/L.

Typically, the aerosol has an inhalable aerosol particle density greater than 10⁶ particles/mL. Preferably, the aerosol has an inhalable aerosol particle density greater than 10⁷ particles/mL. More preferably, the aerosol has an inhalable aerosol particle density greater than 10⁸ particles/mL.

Typically, the aerosol particles have a mass median aerodynamic diameter of less than 5 microns. Preferably, the particles have a mass median aerodynamic diameter of less than 3 microns. More preferably, the particles have a mass median aerodynamic diameter of less than 2 or 1 micron(s).

Typically, the geometric standard deviation around the mass median aerodynamic diameter of the aerosol particles is less than 3.0. Preferably, the geometric standard deviation is less than 2.5. More preferably, the geometric standard deviation is less than 2.1.

Typically, the aerosol is formed by heating a composition containing alprazolam, estazolam, midazolam or triazolam to form a vapor and subsequently allowing the vapor to condense into an aerosol.

In a method aspect of the present invention, either alprazolam, estazolam, midazolam or triazolam is delivered to a mammal through an inhalation route. The method comprises: a) heating a composition, wherein the composition comprises at least 5 percent by weight of alprazolam, estazolam, midazolam or triazolam; and, b) allowing the vapor to cool, thereby forming a condensation aerosol comprising particles, which is inhaled by the mammal. Preferably, the composition that is heated comprises at least 10 percent by weight of alprazolam, estazolam, midazolam or triazolam. More preferably, the composition comprises 20 percent, 30 percent, 40 percent, 50 percent, 60 percent, 70 percent, 80 percent, 90 percent, 95 percent, 97 percent, 99 percent, 99.5 percent, 99.9 percent or 99.97 percent by weight of alprazolam, estazolam, midazolam or triazolam.

Typically, the delivered aerosol particles comprise at least 5 percent by weight of alprazolam, estazolam, midazolam or triazolam. Preferably, the particles comprise at least 10 percent by weight of alprazolam, estazolam, midazolam or triazolam. More preferably, the particles comprise at least 20 percent, 30 percent, 40 percent, 50 percent, 60 percent, 70 percent, 80 percent, 90 percent, 95 percent, 97 percent, 99 percent, 99.5 percent, 99.9 percent or 99.97 percent by weight of alprazolam, estazolam, midazolam or triazolam.

Typically, the aerosol has a mass of at least 1 μg. Preferably, the aerosol has a mass of at least 10 μg. More preferably, the aerosol has a mass of at least 20 μg.

Typically, the delivered aerosol particles comprise less than 10 percent by weight of alprazolam, estazolam, midazolam or triazolam degradation products. Preferably, the particles comprise less than 5 percent by weight of alprazolam, estazolam, midazolam or triazolam degradation products. More preferably, the particles comprise less than 2.5, 1, 0.5, 0.1 or 0.03 percent by weight of alprazolam, estazolam, midazolam or triazolam degradation products.

Typically, the particles of the delivered condensation aerosol have a mass median aerodynamic diameter of less than 5 microns. Preferably, the particles have a mass median aerodynamic diameter of less than 3 microns. More preferably, the particles have a mass median aerodynamic diameter of less than 2 or 1 micron(s).

Typically, the delivered aerosol has an inhalable aerosol drug mass density of between 0.02 mg/L and 10 mg/L. Preferably, the aerosol has an inhalable aerosol drug mass density of between 0.05 mg/L and 5 mg/L. More preferably, the aerosol has an inhalable aerosol drug mass density of between 0.1 mg/L and 2 mg/L.

Typically, the delivered aerosol has an inhalable aerosol particle density greater than 10⁶ particles/mL. Preferably, the aerosol has an inhalable aerosol particle density greater than 10⁷ particles/mL. More preferably, the aerosol has an inhalable aerosol particle density greater than 10⁸ particles/mL.

Typically, the rate of inhalable aerosol particle formation of the delivered condensation aerosol is greater than 10⁸ particles per second. Preferably, the aerosol is formed at a rate greater than 10⁹ inhalable particles per second. More preferably, the aerosol is formed at a rate greater than 10¹⁰ inhalable particles per second.

Typically, the delivered aerosol is formed at a rate greater than 0.1 mg/second. Preferably, the aerosol is formed at a rate greater than 0.25 mg/second. More preferably, the aerosol is formed at a rate greater than 0.5, 1 or 2 mg/second.

Typically, where the condensation aerosol comprises alprazolam, between 0.05 mg and 4 mg of alprazolam are delivered to the mammal in a single inspiration. Preferably, between 0.1 mg and 2 mg of alprazolam are delivered to the mammal in a single inspiration. More preferably, between 0.2 mg and 1 mg of alprazolam are delivered to the mammal in a single inspiration.

Typically, where the condensation aerosol comprises estazolam, between 0.05 mg and 4 mg of estazolam are delivered to the mammal in a single inspiration. Preferably, between 0.1 mg and 2 mg of estazolam are delivered to the mammal in a single inspiration. More preferably, between 0.2 mg and 1 mg of estazolam are delivered to the mammal in a single inspiration.

Typically, where the condensation aerosol comprises midazolam, between 0.05 mg and 4 mg of midazolam are delivered to the mammal in a single inspiration. Preferably, between 0.1 mg and 2 mg of midazolam are delivered to the mammal in a single inspiration. More preferably, between 0.2 mg and 1 mg of midazolam are delivered in a single inspiration.

Typically, where the condensation aerosol comprises triazolam, between 0.006 mg and 0.5 mg of triazolam are delivered to the mammal in a single inspiration. Preferably, between 0.0125 mg and 0.25 mg of triazolam are delivered to the mammal in a single inspiration. More preferably, between 0.025 mg and 0.125 mg of triazolam are delivered to the mammal in a single inspiration.

Typically, the delivered condensation aerosol results in a peak plasma concentration of alprazolam, estazolam, midazolam or triazolam in the mammal in less than 1 h. Preferably, the peak plasma concentration is reached in less than 0.5 h. More preferably, the peak plasma concentration is reached in less than 0.2, 0.1, 0.05, 0.02, 0.01, or 0.005 h (arterial measurement).

In a kit aspect of the present invention, a kit for delivering alprazolam, estazolam, midazolam or triazolam through an inhalation route to a mammal is provided which comprises: a) a composition comprising at least 5 percent by weight of alprazolam, estazolam, midazolam or triazolam; and, b) a device that forms an alprazolam, estazolam, midazolam or triazolam containing aerosol from the composition, for inhalation by the mammal. Preferably, the composition comprises at least 10 percent by weight of alprazolam, estazolam, midazolam or triazolam. More preferably, the composition comprises at least 20 percent, 30 percent, 40 percent, 50 percent, 60 percent, 70 percent, 80 percent, 90 percent, 95 percent, 97 percent, 99 percent, 99.5 percent, 99.9 percent or 99.97 percent by weight of alprazolam, estazolam, midazolam or triazolam.

Typically, the device contained in the kit comprises: a) an element for heating the alprazolam, estazolam, midazolam or triazolam composition to form a vapor; b) an element allowing the vapor to cool to form an aerosol; and, c) an element permitting the mammal to inhale the aerosol.

These and other objects and features of the invention will be more fully appreciated when the following detailed description of the invention is read in conjunction with the accompanying drawings. All publications, patents, and patent applications referred to herein are incorporated herein by reference in their entirety.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

FIGS. 1A-1B are cross-sectional views of general embodiments of a drug-supply article in accordance with the invention;

FIG. 2A is a perspective view of a drug-delivery device that incorporates a drug-supply article;

FIG. 2B shows another drug-delivery device that incorporates a drug-supply article, where the device components are shown in unassembled form;

FIGS. 3A-3E are high speed photographs showing the generation of aerosol particles from a drug-supply unit;

FIGS. 4A-4B are plots of substrate temperature increase, measured in still air with a thin thermocouple (Omega, Model CO2-K), as a function of time. The substrate in FIG. 4A was heated resistively by connection to a capacitor charged to 13.5 Volts (lower line), 15 Volts (middle line), and 16 Volts (upper line); the substrate in FIG. 4B was heated resistively by discharge of a capacitor at 16 Volts;

FIGS. 5A-5B are plots of substrate temperature, in ° C., as a function of time, in seconds, for a hollow stainless steel cylindrical substrate heated resistively by connection to a capacitor charged to 21 Volts, where FIG. 5A shows the temperature profile over a 4 second time period and FIG. 5B is a detail showing the temperature profile over the first second of heating;

FIG. 6 is plot showing purity of thermal vapor as a function of drug film thickness, in micrometers, for the drug atropine free base;

FIG. 7 is plot showing purity of thermal vapor as a function of drug film thickness, in micrometers, for donepezil free base;

FIG. 8 is plot showing purity of thermal vapor as a function of drug film thickness, in micrometers, for hydromorphone free base;

FIG. 9 is plot showing purity of thermal vapor as a function of drug film thickness, in micrometers, for buprenorphine free base;

FIG. 10 is plot showing purity of thermal vapor as a function of drug film thickness, in micrometers, for clomipramine free base;

FIG. 11 is plot showing purity of thermal vapor as a function of drug film thickness, in micrometers, for ciclesonide;

FIG. 12 is plot showing purity of thermal vapor as a function of drug film thickness, in micrometers, for midazolam free base;

FIG. 13 is plot showing purity of thermal vapor as a function of drug film thickness, in micrometers, for nalbuphine free base;

FIG. 14 is plot showing purity of thermal vapor as a function of drug film thickness, in micrometers, for naratriptan free base;

FIG. 15 is plot showing purity of thermal vapor as a function of drug film thickness, in micrometers, for olanzapine free base;

FIG. 16 is plot showing purity of thermal vapor as a function of drug film thickness, in micrometers, for quetiapine free base;

FIG. 17 is plot showing purity of thermal vapor as a function of drug film thickness, in micrometers, for tadalafil free base;

FIG. 18 is plot showing purity of thermal vapor as a function of drug film thickness, in micrometers, for prochlorperazine free base;

FIG. 19 is plot showing purity of thermal vapor as a function of drug film thickness, in micrometers, for zolpidem free base;

FIG. 20 is plot showing purity of thermal vapor as a function of drug film thickness, in micrometers, for fentanyl free base;

FIG. 21 is plot showing purity of thermal vapor as a function of drug film thickness, in micrometers, for alprazolam free base;

FIG. 22 is plot showing purity of thermal vapor as a function of drug film thickness, in micrometers, for sildenafil free base;

FIG. 23 is plot showing purity of thermal vapor as a function of drug film thickness, in micrometers, for albuterol free base;

FIGS. 24A-24D are high speed photographs showing the generation of a thermal vapor of phenyloin from a film of drug coated on a substrate drug-supply unit, where the photographs are taken prior to substrate heating (t=0 ms, FIG. 24A) and during substrate heating at times of 50 milliseconds (FIG. 24B), 100 milliseconds (FIG. 24C), and 200 milliseconds (FIG. 24D);

FIGS. 25A-25D are high speed photographs showing the generation of a thermal vapor of disopyramide from a film of drug coated on a substrate drug-supply unit, where the photographs are taken at prior to substrate heating (t=0 ms, FIG. 25A) and during substrate heating at times of 50 milliseconds (FIG. 25B), 100 milliseconds (FIG. 25C), and 200 milliseconds (FIG. 25D).

FIGS. 26A-26E are high speed photographs showing the generation of a thermal vapor of buprenorphine from a film of drug coated on a substrate drug-supply unit, where the photographs are taken at prior to substrate heating (t=0 ms, FIG. 26A) and during substrate heating at times of 50 milliseconds (FIG. 26B), 100 milliseconds (FIG. 26C), 200 milliseconds (FIG. 26D), and 300 milliseconds (FIG. 26E).

FIG. 27 is an illustration of an exemplary device that may be used to form and administer the aerosols described herein.

FIG. 28 shows particle sizes in condensation aerosols of caffeine, cyclobenzaprine, and diazepam.

FIG. 29 shows particle sizes in condensation aerosols of ketoprofen ethyl ester.

FIG. 30 shows a device used to deliver alprazolam, estazolam, midazolam or triazolam containing aerosols to a mammal through an inhalation route.

FIG. 31 is a cross-sectional side view of a preferred embodiment of the present invention.

FIG. 32 is a top view of the preferred embodiment shown in FIG. 31.

FIG. 33 is the end view of the preferred embodiment shown in FIG. 31.

FIG. 34 is an isometric view of the slide and foil with the compound deposited on the foil in the preferred embodiment shown in FIG. 31.

FIG. 35 is an isometric show a heating zone and an inductive heater assembly made of a ferrite toroid;

FIG. 36 is a side view of the inductive heater assembly of the preferred embodiment shown in FIG. 1 showing the magnetic field lines and the foil substrate cutting the field lines;

FIG. 37 is a side view of the preferred embodiment shown in FIG. 31 showing the venturi and the area of increased air velocity.

FIG. 38 is a schematic view of the drive resonant circuit of the preferred embodiment shown in FIG. 31.

FIG. 39 is the schematic view of the drive circuit of a second preferred embodiment of the present invention that involves very rapid heating.

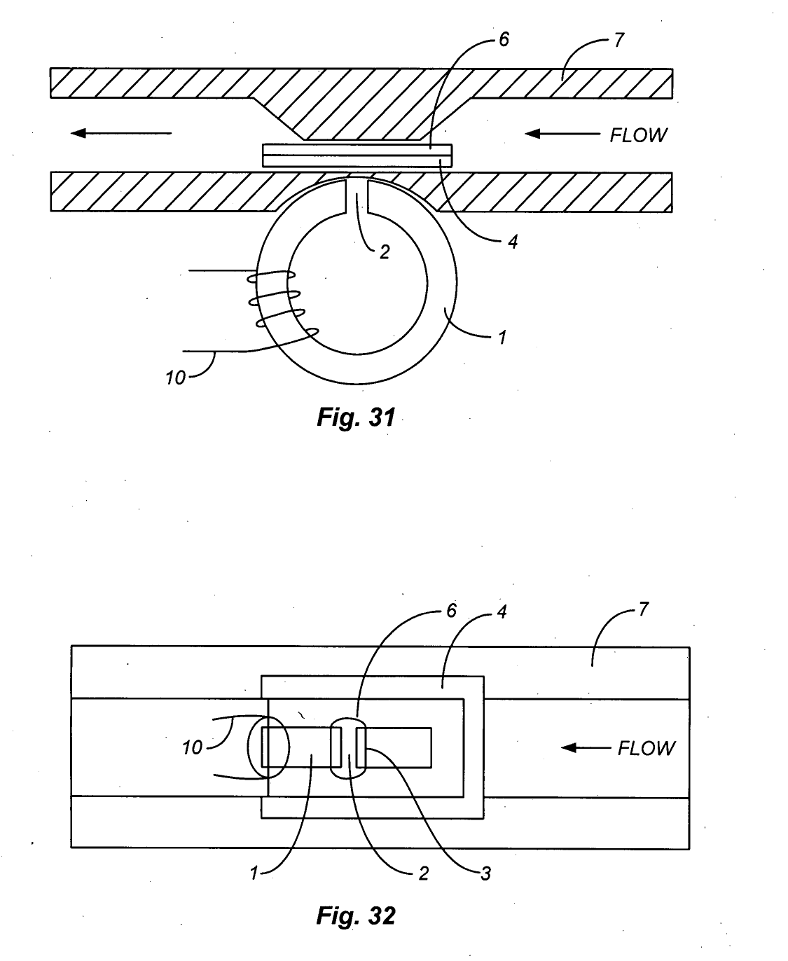

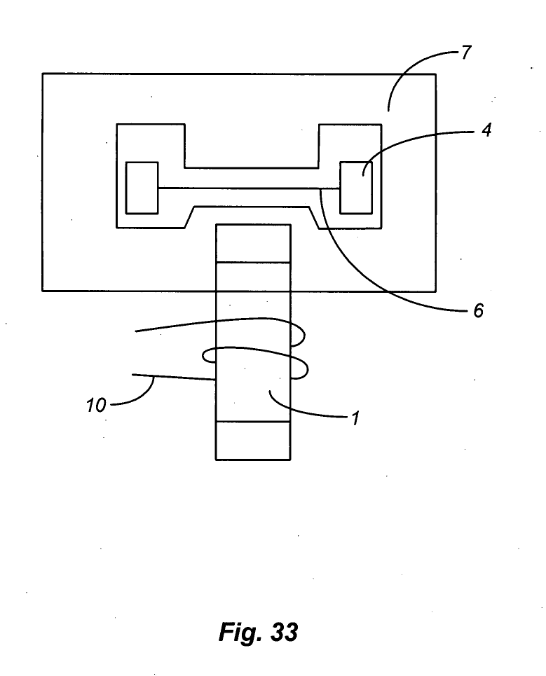

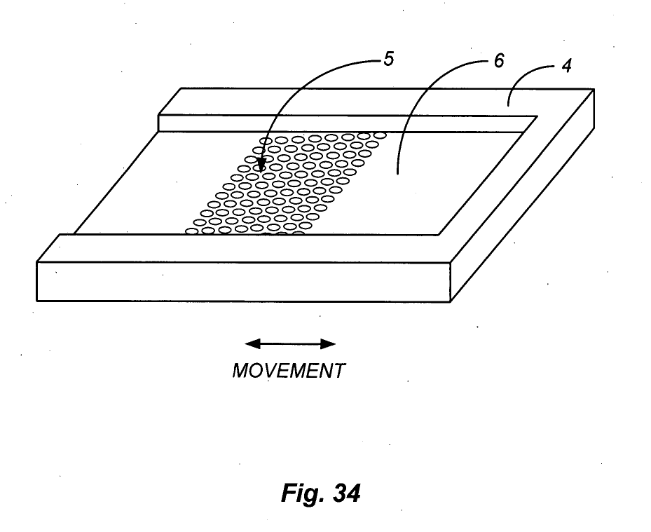

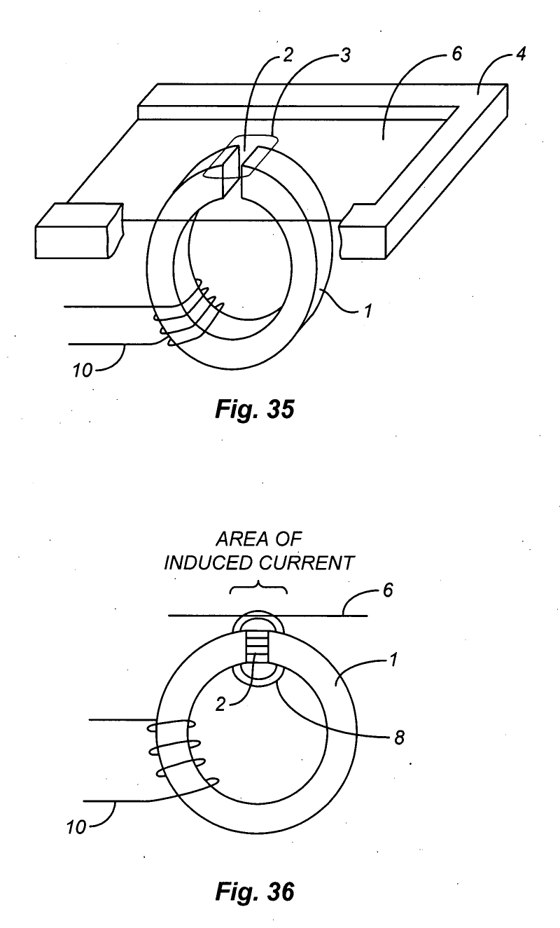

The following is a summary of the major elements of the invention shown in FIGS. 31-39: #1 is the ferrite toroid used to shape and contain the magnetic field in the inductive heater; #2 is the air gap in the ferrite where the magnet field is allowed to escape the toroid and enter the substrate; #3 is the heating zone of the inductive heater; #4 is the frame that holds the foil; #5 is the compound that has been deposited on the foil; #6 is the foil; #7 is the airway passage; #8 are the magnetic field lines in the inductive heater; #9 is the venturi area where the speed of the air in increased; #10 is the wire winding used to create a magnetic field; #11 is the foil used in the rapid heat up device of the second preferred embodiment of the present invention; #12 is the switch used to discharge the capacitor in the rapid heat up device of the second preferred embodiment.

### DETAILED DESCRIPTION OF THE INVENTION

Definitions

“Aerodynamic diameter” of a given particle refers to the diameter of a spherical droplet with a density of 1 g/mL (the density of water) that has the same settling velocity as the given particle.

“Aerosol” refers to a collection of solid or liquid particles suspended in a gas.

“Aerosol mass concentration” refers to the mass of particulate matter per unit volume of aerosol.

“Condensation aerosol” refers to an aerosol formed by vaporization of a substance followed by condensation of the substance into an aerosol.

“Decomposition index” refers to a number derived from an assay described in Example 238. The number is determined by subtracting the purity of the generated aerosol, expressed as a fraction, from 1. The term “drug” as used herein means any substance that is used in the prevention, diagnosis, alleviation, treatment or cure of a condition. The drug is preferably in a form suitable for thermal vapor delivery, such as an ester, free acid, or free base form. The drugs are preferably other than recreational drugs. More specifically, the drugs are preferably other than recreational drugs used for non-medicinal recreational purposes, e.g., habitual use to solely alter one's mood, affect, state of consciousness, or to affect a body function unnecessarily, for recreational purposes. In one aspect, Nicotine and cocaine are recreational drugs specifically excluded from the term “drug”. In another aspect, drugs encompass prodrug, i.e., a chemical compound that is inactive in the form administered to a patient, but is converted to an active substance for the altering affect, treatment, cure, prevention or diagnosis of a disease after it is administered. The terms “drug” and “medication” are herein used interchangeably.

In some instances, “compound” is herein used interchangeably.

“Drug supply article” or “drug supply unit” are used interchangeably and refers to a substrate with at least a portion of its surface coated with one or more drug compositions. Drug supply articles of the invention may also include additional elements such as, for example, but not limitation, a heating element.

“Heat stable drug” refers to a drug that has a TSR≧9 when vaporized from a film of some thickness between 0.05 μm and 20 μm. A determination of whether a drug classifies as a heat stable drug can be made as described in Example 237.

“Number concentration” refers to the number of particles per unit volume of aerosol.

“Purity” as used herein, with respect to the aerosol purity, means the fraction of drug composition in the aerosol/ the fraction of drug composition in the aerosol plus drug degradation products. Thus purity is relative with regard to the purity of the starting material. For example, when the starting drug or drug composition used for substrate coating contained detectable impurities, the reported purity of the aerosol does not include those impurities present in the starting material that were also found in the aerosol, e.g., in certain cases if the starting material contained a 1% impurity and the aerosol was found to contain the identical 1% impurity, the aerosol purity may nevertheless be reported as >99% pure, reflecting the fact that the detectable 1% purity was not produced during the vaporization-condensation aerosol generation process.

“Settling velocity” refers to the terminal velocity of an aerosol particle undergoing gravitational settling in air.

“Support” refers to a material on which the composition is adhered, typically as a coating or thin film. The term “support” and “substrate” are used herein interchangeably.

“Substantially free of” means that the material, compound, aerosol, etc., being described is at least 95% free of the other component from which it is substantially free.

“Typical patient tidal volume” refers to 1 L for an adult patient and 15 mL/kg for a pediatric patient.

“Thermal stability ratio” or “TSR” means the % purity/(100%−% purity) if the % purity is <99.9%, and 1000 if the % purity is ≧99.9%. For example, a respiratory drug vaporizing at 90% purity would have a TSR of 9. An example of how to determine whether a respiratory drug is heat stable is provided in Example 237.

“4 μm thermal stability ratio” or “4TSR” means the TSR of a drug determined by heating a drug-comprising film of about 4 microns in thickness under conditions sufficient to vaporize at least 50% of the drug in the film, collecting the resulting aerosol, determining the purity of the aerosol, and using the purity to compute the TSR. In such vaporization, generally the about 4-micron thick drug film is heated to around 350° C. but not less than 200° C. for around 1 second to vaporize at least 50% of the drug in the film.

“1.5 μm thermal stability ratio” or “1.5TSR” means the TSR of a drug determined by heating a drug-comprising film of about 1.5 microns in thickness under conditions sufficient to vaporize at least 50% of the drug in the film, collecting the resulting aerosol, determining the purity of the aerosol, and using the purity to compute the TSR. In such vaporization, generally the about 1.5-micron thick drug film is heated to around 350° C. but not less than 200° C. for around 1 second to vaporize at least 50% of the drug in the film.

“0.5 μm thermal stability ratio” or “0.5TSR” means the TSR of a drug determined by heating a drug-comprising film of about 0.5 microns in thickness under conditions sufficient to vaporize at least 50% of the drug in the film, collecting the resulting aerosol, determining the purity of the aerosol, and using the purity to compute the TSR. In such vaporization, generally the about 0.5-micron thick drug film is heated to around 350° C. but not less than 200° C. for around 1 second to vaporize at least 50% of the drug in the film.

As used herein, “treatment” is an approach for obtaining beneficial or desired clinical results. For purposes of this invention, beneficial or desired clinical results include, but are not limited to, one or more of the following: alleviation of symptoms, diminishment of extent of a disease, stabilization (i.e., not worsening) of a state of disease, preventing spread (i.e., metastasis) of disease, preventing occurrence or recurrence of disease, delay or slowing of disease progression, amelioration of the disease state, and remission (whether partial or total).

“Alprazolam” refers to 8-chloro-1-methyl-6-phenyl-4H-s-triazolo-[4,3-α][1,4]benzodiazepine, which has an empirical formula of C17H13ClN4.

“Alprazolam degradation product” refers to a compound resulting from a chemical modification of alprazolam. The modification, for example, can be the result of a thermally or photochemically induced reaction. Such reactions include, without limitation, oxidation (e.g., of the methyl or methylene unit) and hydrolysis (e.g., of the imine portion).

The aerosols may be formed in substantially pure form. The term “substantially pure aerosol of a drug” as used herein refers to an aerosol of a drug that is about 50% free, by weight, of additional compounds, or about 80% free, by weight, of additional compounds, or about 90% free, by weight, of additional compounds, or about 99% free, by weight, of additional compounds, or about 99.9% free, by weight, of additional compounds, or about 99.97% free, by weight, of additional compounds. Additional compounds include, but are not limited to, compounds such as carriers, solvents, emulsifiers, propellants, and drug degradation products. In addition, the aerosols preferably contain greater than 105 particles per mL, greater than 10⁶ particles per mL, or greater than 10⁸ particles per mL.

“Estazolam” refers to 8-chloro-6-phenyl-4H-s-triazolo[4,3-α][1,4]benzodiazepine, which has an empirical formula of C₁₆H₁₁ClN₄.

“Estazolam degradation product” refers to a compound resulting from a chemical modification of estazolam. The modification, for example, can be the result of a thermally or photochemically induced reaction. Such reactions include, without limitation, oxidation (e.g., of the methylene unit) and hydrolysis (e.g., of the imine portion).

“Mass median aerodynamic diameter” or “MMAD” of an aerosol refers to the aerodynamic diameter for which half the particulate mass of the aerosol is contributed by particles with an aerodynamic diameter larger than the MMAD and half by particles with an aerodynamic diameter smaller than the MMAD.

“Midazolam” refers to 8-chloro-6-(2-fluorophenyl)-1-methyl-4H-imidazo[1,5-a][1,4]benzodiazepine, which has an empirical formula of C₁₈H₁₃ClFN₃.

“Midazolam degradation product” refers to a compound resulting from a chemical modification of midazolam. The modification, for example, can be the result of a thermally or photochemically induced reaction. Such reactions include, without limitation, oxidation (e.g., of the methyl or methylene unit) and hydrolysis (e.g., of the imine portion).

“Triazolam” refers to 8-chloro-6-(-o-chlorophenyl)-1-methyl-4H-s-triazolo-[4,3-α][1,4]benzodiazepine, which has an empirical formula of C₁₇H₁₂Cl₂N₄.

“Triazolam degradation product” refers to a compound resulting from a chemical modification of triazolam. The modification, for example, can be the result of a thermally or photochemically induced reaction. Such reactions include, without limitation, oxidation (e.g., of the methyl or methylene unit) and hydrolysis (e.g., of the imine portion).

Drugs:

The compositions described herein typically comprise at least one drug compound. The drug compositions may comprise other compounds as well. For example, the composition may comprise a mixture of drug compounds, a mixture of a drug compound and a pharmaceutically acceptable excipient, or a mixture of a drug compound with other compounds having useful or desirable properties. The composition may comprise a pure drug compound as well.

The drugs of use in the invention typically have a molecular weight in the range of about 150-700, preferably in the range of about 200-650, more preferably in the range of 250-600, still more preferably in the range of about 250-500, and most preferably in the range of about 300-450.

In general, we have found that suitable drug have properties that make them acceptable candidates for use with the devices and methods herein described. For example, the drug compound is typically one that is, or can be made to be, vaporizable. Typically, the drug is a heat stable drug. Exemplary drugs include acebutolol, acetaminophen, alprazolam, amantadine, amitriptyline, apomorphine diacetate, apomorphine hydrochloride, atropine, azatadine, betahistine, brompheniramine, bumetanide, buprenorphine, bupropion hydrochloride, butalbital, butorphanol, carbinoxamine maleate, celecoxib, chlordiazepoxide, chlorpheniramine, chlorzoxazone, ciclesonide, citalopram, clomipramine, clonazepam, clozapine, codeine, cyclobenzaprine, cyproheptadine, dapsone, diazepam, diclofenac ethyl ester, diflunisal, disopyramide, doxepin, estradiol, ephedrine, estazolam, ethacrynic acid, fenfluramine, fenoprofen, flecainide, flunitrazepam, galanthamine, granisetron, haloperidol, hydromorphone, hydroxychloroquine, ibuprofen, imipramine, indomethacin ethyl ester, indomethacin methyl ester, isocarboxazid, ketamine, ketoprofen, ketoprofen ethyl ester, ketoprofen methyl ester, ketorolac ethyl ester, ketorolac methyl ester, ketotifen, lamotrigine, lidocaine, loperamide, loratadine, loxapine, maprotiline, memantine, meperidine, metaproterenol, methoxsalen, metoprolol, mexiletine HCl, midazolam, mirtazapine, morphine, nalbuphine, naloxone, naproxen, naratriptan, nortriptyline, olanzapine, orphenadrine, oxycodone, paroxetine, pergolide, phenyloin, pindolol, piribedil, pramipexole, procainamide, prochloperazine, propafenone, propranolol, pyrilamine, quetiapine, quinidine, rizatriptan, ropinirole, sertraline, selegiline, sildenafil, spironolactone, tacrine, tadalafil, terbutaline, testosterone, thalidomide, theophylline, tocainide, toremifene, trazodone, triazolam, trifluoperazine, valproic acid, venlafaxine, vitamin E, zaleplon, zotepine, amoxapine, atenolol, benztropine, caffeine, doxylamine, estradiol 17-acetate, flurazepam, flurbiprofen, hydroxyzine, ibutilide, indomethacin norcholine ester, ketorolac norcholine ester, melatonin, metoclopramide, nabumetone, perphenazine, protriptyline HCl, quinine, triamterene, trimipramine, zonisamide, bergapten, chlorpromazine, colchicine, diltiazem, donepezil, eletriptan, estradiol-3,17-diacetate, efavirenz, esmolol, fentanyl, flunisolide, fluoxetine, hyoscyamine, indomethacin, isotretinoin, linezolid, meclizine, paracoxib, pioglitazone, rofecoxib, sumatriptan, tolterodine, tramadol, tranylcypromine, trimipramine maleate, valdecoxib, vardenafil, verapamil, zolmitriptan, zolpidem, zopiclone, bromazepam, buspirone, cinnarizine, dipyridamole, naltrexone, sotalol, telmisartan, temazepam, albuterol, apomorphine hydrochloride diacetate, carbinoxamine, clonidine, diphenhydramine, thambutol, fluticasone proprionate, fluconazole, lovastatin, lorazepam N,O-diacetyl, methadone, nefazodone, oxybutynin, promazine, promethazine, sibutramine, tamoxifen, tolfenamic acid, aripiprazole, astemizole, benazepril, clemastine, estradiol 17-heptanoate, fluphenazine, protriptyline, ethambutal, frovatriptan, pyrilamine maleate, scopolamine, and triamcinolone acetonide and pharmaceutically acceptable analogs and equivalents thereof.

The drug may be one that when vaporized from a film on an impermeable surface of a heat conductive substrate, the aerosol exhibits an increasing level of drug composition degradation products with increasing film thickness. Examples include but are not limited to the following drugs, and associated ranges of film thicknesses:

alprazolam, film thickness between 0.1 and 10 μm;

amoxapine, film thickness between 2 and 20 μm;

atropine, film thickness between 0.1 and 10 μm;

bumetanide film thickness between 0.1 and 5 μm;

buprenorphine, film thickness between 0.05 and 10 μm;

butorphanol, film thickness between 0.1 and 10 μm;

clomipramine, film thickness between 1 and 8 μm;

donepezil, film thickness between 1 and 10 μm;

hydromorphone, film thickness between 0.05 and 10 μm;

loxapine, film thickness between 1 and 20 μm;

midazolam, film thickness between 0.05 and 20 μm;

morphine, film thickness between 0.2 and 10 μm;

nalbuphine, film thickness between 0.2 and 5 μm;

naratriptan, film thickness between 0.2 and 5 μm;

olanzapine, film thickness between 1 and 20 μm;

paroxetine, film thickness between 1 and 20 μm;

prochlorperazine, film thickness between 0.1 and 20 μm;

pramipexole, film thickness between 0.05 and 10 μm;

quetiapine, film thickness between 1 and 20 μm;

rizatriptan, film thickness between 0.2 and 20 μm;

sertraline, film thickness between 1 and 20 μm;

sibutramine, film thickness between 0.5 and 2 μm;

sildenafil, film thickness between 0.2 and 3 μm;

sumatriptan, film thickness between 0.2 and 6 μm;

tadalafil, film thickness between 0.2 and 5 μm;

vardenafil, film thickness between 0.1 and 2 μm;

venlafaxine, film thickness between 2 and 20 μm;

zolpidem, film thickness between 0.1 and 10 μm;

apomorphine HCl, film thickness between 0.1 and 5 μm;

celecoxib, film thickness between 2 and 20 μm;

ciclesonide, film thickness between 0.05 and 5 μm;

eletriptan, film thickness between 0.2 and 20 μm;

parecoxib, film thickness between 0.5 and 2 μm;

valdecoxib, film thickness between 0.5 and 10 μm;

fentanyl, film thickness between 0.05 and 5 μm;

citalopram, film thickness between 1 and 20 μm;

escitalopram, film thickness between 0.2 and 20 μm;

clonazepam, film thickness between 0.05 and 8 μm;

oxymorphone, film thickness between 0.1 and 10 μm;

albuterol, film thickness between 0.2 and 2 μm;

sufentanyl, film thickness between 0.05 and 5 μm; and

remifentanyl, film thickness between 0.05 and 5 μm.

Typically, the drugs of use in the invention have a molecular weight in the range of about 150-700, preferably in the range of about 200-700, more preferably in the range of 250-600, still more preferably in the range of about 250-500. In some variations, the drugs have a molecular weight in the range 350-600 and in others the drugs have a molecular weigh in the range of about 300-450. In other variations, where the drug is a heat stable drug, the drug can have a molecular weight of 350 or greater.

Typically, the compound is in its ester, free acid, or its free-base form. However, it is not without possibility that the compound will be vaporizable from its salt form. Indeed, a variety of pharmaceutically acceptable salts are suitable for aerosolization. Illustrative salts include, without limitation, the following: hydrochloric acid, hydrobromic acid, acetic acid, maleic acid, formic acid, and fumaric acid salts. Salt forms can be purchased commercially, or can be obtained from their corresponding free acid or free base forms using well known methods in the art.

Suitable pharmaceutically acceptable excipients may be volatile or nonvolatile. Volatile excipients, when heated, are concurrently volatilized, aerosolized and inhaled with the drug. Classes of such excipients are known in the art and include, without limitation, gaseous, supercritical fluid, liquid and solid solvents. The following is a list of exemplary carriers within these classes: water; terpenes, such as menthol; alcohols, such as ethanol, propylene glycol, glycerol and other similar alcohols; dimethylformamide; dimethylacetamide; wax; supercritical carbon dioxide; dry ice; and mixtures thereof.

Additionally, pharmaceutically acceptable carriers, surfactants, enhancers, and inorganic compounds may be included in the composition. Examples of such materials are known in the art.

In some variations, the aerosols are substantially free of organic solvents and propellants. Additionally, water is typically not added as a solvent for the drug, although water from the atmosphere may be incorporated in the aerosol during formation, in particular, while passing air over the film and during the cooling process. In other variations, the aerosols are completely devoid of organic solvents and propellants. In yet other variations, the aerosols are completely devoid of organic solvents, propellants, and any excipients. These aerosols comprise only pure drug, less than 10% drug degradation products, and a carrier gas, which is typically air.

Typically, the drug has a decomposition index less than 0.15. Preferably, the drug has a decomposition index less than 0.10. More preferably, the drug has a decomposition index less than 0.05. Most preferably, the drug has a decomposition index less than 0.025

In some variations, the condensation aerosol comprises at least 5% by weight of condensation drug aerosol particles. In other variations, the aerosol comprises at least 10%, 20%, 30%, 40%, 50%, 60%, or 75% by weight of condensation drug aerosol particles. In still other variations, the aerosol comprises at least 95%, 99%, or 99.5% by weight of condensation aerosol particles.

In some variations, the condensation aerosol particles comprise less than 10% by weight of a thermal degradation product. In other variations, the condensation drug aerosol particles comprise less than 5%, 1%, 0.5%, 0.1%, or 0.03% by weight of a thermal degradation product.

In certain embodiments of the invention, the drug aerosol has a purity of between 90% and 99.8%, or between 93% and 99.7%, or between 95% and 99.5%, or between 96.5% and 99.2%.

Typically, the aerosol has a number concentration greater than 10⁶ particles/mL. In other variations, the aerosol has a number concentration greater than 10⁷ particles/mL. In yet other variations, the aerosol has a number concentration greater than 10⁸ particles/mL, greater than 10⁹ particles/mL, greater than 10¹⁰ particles/mL, or greater than 10¹¹ particles/mL.

The gas of the aerosol typically is air. Other gases, however, can be used, in particular inert gases, such as argon, nitrogen, helium, and the like. The gas can also include vapor of the composition that has not yet condensed to form particles. Typically, the gas does not include propellants or vaporized organic solvents. In some variations, the condensation aerosol comprises at least 5% by weight of condensation drug aerosol particles. In other variations, the aerosol comprises at least 10%, 20%, 30%, 40%, 50%, 60%, or 75% by weight of condensation drug aerosol particles. In still other variations, the aerosol comprises at least 95%, 99%, or 99.5% by weight of condensation aerosol particles.

In some variations the condensation drug aerosol has a MMAD in the range of about 1-3 μm. In some variations the geometric standard deviation around the MMAD of the condensation drug aerosol particles is less than 3.0. In other variations, the geometric standard deviation around the MMAD of the condensation drug aerosol particles is less than 2.5, or less than 2.0.

In certain embodiments of the invention, the drug aerosol comprises one or more drugs having a 4TSR of at least 5 or 10, a 1.5TSR of at least 7 or 14, or a 0.5TSR of at least 9 or 18. In other embodiments of the invention, the drug aerosol comprises one or more drugs having a 4TSR of between 5 and 100 or between 10 and 50, a 1.5TSR of between 7 and 200 or between 14 and 100, or a 0.5TSR of between 9 and 900 or between 18 and 300.

Specific drugs that can be used include, for example but not limitation, drugs of one of the following classes: anesthetics, anticonvulsants, antidepressants, antidiabetic agents, antidotes, antiemetics, antihistamines, anti-infective agents, antineoplastics, antiparkisonian drugs, antirheumatic agents, antipsychotics, anxiolytics, appetite stimulants and suppressants, blood modifiers, cardiovascular agents, central nervous system stimulants, drugs for Alzheimer's disease management, drugs for cystic fibrosis management, diagnostics, dietary supplements, drugs for erectile dysfunction, gastrointestinal agents, hormones, drugs for the treatment of alcoholism, drugs for the treatment of addiction, immunosuppressives, mast cell stabilizers, migraine preparations, motion sickness products, drugs for multiple sclerosis management, muscle relaxants, nonsteroidal anti-inflammatories, opioids, other analgesics and stimulants, opthalmic preparations, osteoporosis preparations, prostaglandins, respiratory agents, sedatives and hypnotics, skin and mucous membrane agents, smoking cessation aids, Tourette's syndrome agents, urinary tract agents, and vertigo agents.

Typically, where the drug is an anesthetic, it is selected from one of the following compounds: ketamine and lidocaine.

Typically, where the drug is an anticonvulsant, it is selected from one of the following classes: GABA analogs, tiagabine, vigabatrin; barbiturates such as pentobarbital; benzodiazepines such as clonazepam; hydantoins such as phenyloin; phenyltriazines such as lamotrigine; miscellaneous anticonvulsants such as carbamazepine, topiramate, valproic acid, and zonisamide.

Typically, where the drug is an antidepressant, it is selected from one of the following compounds: amitriptyline, amoxapine, benmoxine, butriptyline, clomipramine, desipramine, dosulepin, doxepin, imipramine, kitanserin, lofepramine, medifoxamine, mianserin, maprotoline, mirtazapine, nortriptyline, protriptyline, trimipramine, venlafaxine, viloxazine, citalopram, cotinine, duloxetine, fluoxetine, fluvoxamine, milnacipran, nisoxetine, paroxetine, reboxetine, sertraline, tianeptine, acetaphenazine, binedaline, brofaromine, cericlamine, clovoxamine, iproniazid, isocarboxazid, moclobemide, phenyhydrazine, phenelzine, selegiline, sibutramine, tranylcypromine, ademetionine, adrafinil, amesergide, amisulpride, amperozide, benactyzine, bupropion, caroxazone, gepirone, idazoxan, metralindole, milnacipran, minaprine, nefazodone, nomifensine, ritanserin, roxindole, S-adenosylmethionine, tofenacin, trazodone, tryptophan, and zalospirone.

Typically, where the drug is an antidiabetic agent, it is selected from one of the following compounds: pioglitazone, rosiglitazone, and troglitazone.

Typically, where the drug is an antidote, it is selected from one of the following compounds: edrophonium chloride, flumazenil, deferoxamine, nalmefene, naloxone, and naltrexone.

Typically, where the drug is an antiemetic, it is selected from one of the following compounds: alizapride, azasetron, benzquinamide, bromopride, buclizine, chlorpromazine, cinnarizine, clebopride, cyclizine, diphenhydramine, diphenidol, dolasetron, droperidol, granisetron, hyoscine, lorazepam, dronabinol, metoclopramide, metopimazine, ondansetron, perphenazine, promethazine, prochlorperazine, scopolamine, triethylperazine, trifluoperazine, triflupromazine, trimethobenzamide, tropisetron, domperidone, and palonosetron.

Typically, where the drug is an antihistamine, it is selected from one of the following compounds: astemizole, azatadine, brompheniramine, carbinoxamine, cetrizine, chlorpheniramine, cinnarizine, clemastine, cyproheptadine, dexmedetomnidine, diphenhydramine, doxylamine, fexofenadine, hydroxyzine, loratidine, promethazine, pyrilamine and terfenidine.

Typically, where the drug is an anti-infective agent, it is selected from one of the following classes: antivirals such as efavirenz; AIDS adjunct agents such as dapsone; aminoglycosides such as tobramycin; antifungals such as fluconazole; antimalarial agents such as quinine; antituberculosis agents such as ethambutol; β-lactams such as cefmetazole, cefazolin, cephalexin, cefoperazone, cefoxitin, cephacetrile, cephaloglycin, cephaloridine; cephalosporins, such as cephalosporin C, cephalothin; cephamycins such as cephamycin A, cephamycin B, and cephamycin C, cephapirin, cephradine; leprostatics such as clofazimine; penicillins such as ampicillin, amoxicillin, hetacillin, carfecillin, carindacillin, carbenicillin, amylpenicillin, azidocillin, benzylpenicillin, clometocillin, cloxacillin, cyclacillin, methicillin, nafcillin, 2-pentenylpenicillin, penicillin N, penicillin O, penicillin S, penicillin V, dicloxacillin; diphenicillin; heptylpenicillin; and metampicillin; quinolones such as ciprofloxacin, clinafloxacin, difloxacin, grepafloxacin, norfloxacin, ofloxacine, temafloxacin; tetracyclines such as doxycycline and oxytetracycline; miscellaneous anti-infectives such as linezolide, trimethoprim and sulfamethoxazole.

Typically, where the drug is an anti-neoplastic agent, it is selected from one of the following compounds: droloxifene, tamoxifen, and toremifene.

Typically, where the drug is an antiparkisonian drug, it is selected from one of the following compounds: amantadine, baclofen, biperiden, benztropine, orphenadrine, procyclidine, trihexyphenidyl, levodopa, carbidopa, andropinirole, apomorphine, benserazide, bromocriptine, budipine, cabergoline, eliprodil, eptastigmine, ergoline, galanthamine, lazabemide, lisuride, mazindol, memantine, mofegiline, pergolide, piribedil, pramipexole, propentofylline, rasagiline, remacemide, ropinerole, selegiline, spheramine, terguride, entacapone, and tolcapone.

Typically, where the drug is an antirheumatic agent, it is selected from one of the following compounds: diclofenac, hydroxychloroquine and methotrexate.

Typically, where the drug is an antipsychotic, it is selected from one of the following compounds: acetophenazine, alizapride, amisulpride, amoxapine, amperozide, aripiprazole, benperidol, benzquinamide, bromperidol, buramate, butaclamol, butaperazine, carphenazine, carpipramine, chlorpromazine, chlorprothixene, clocapramine, clomacran, clopenthixol, clospirazine, clothiapine, clozapine, cyamemazine, droperidol, flupenthixol, fluphenazine, fluspirilene, haloperidol, loxapine, melperone, mesoridazine, metofenazate, molindrone, olanzapine, penfluridol, pericyazine, perphenazine, pimozide, pipamerone, piperacetazine, pipotiazine, prochlorperazine, promazine, quetiapine, remoxipride, risperidone, sertindole, spiperone, sulpiride, thioridazine, thiothixene, trifluperidol, triflupromazine, trifluoperazine, ziprasidone, zotepine, and zuclopenthixol.

Typically, where the drug is an anxiolytic, it is selected from one of the following compounds: alprazolam, bromazepam, oxazepam, buspirone, hydroxyzine, mecloqualone, medetomidine, metomidate, adinazolam, chlordiazepoxide, clobenzepam, flurazepam, lorazepam, loprazolam, midazolam, alpidem, alseroxlon, amphenidone, azacyclonol, bromisovalum, captodiamine, capuride, carbcloral, carbromal, chloral betaine, enciprazine, flesinoxan, ipsapiraone, lesopitron, loxapine, methaqualone, methprylon, propanolol, tandospirone, trazadone, zopiclone, and zolpidem.

Typically, where the drug is an appetite stimulant, it is dronabinol.

Typically, where the drug is an appetite suppressant, it is selected from one of the following compounds: fenfluramine, phentermine and sibutramine.

Typically, where the drug is a blood modifier, it is selected from one of the following compounds: cilostazol and dipyridamol.

Typically, where the drug is a cardiovascular agent, it is selected from one of the following compounds: benazepril, captopril, enalapril, quinapril, ramipril, doxazosin, prazosin, clonidine, labetolol, candesartan, irbesartan, losartan, telmisartan, valsartan, disopyramide, flecanide, mexiletine, procainamide, propafenone, quinidine, tocainide, amiodarone, dofetilide, ibutilide, adenosine, gemfibrozil, lovastatin, acebutalol, atenolol, bisoprolol, esmolol, metoprolol, nadolol, pindolol, propranolol, sotalol, diltiazem, nifedipine, verapamil, spironolactone, bumetanide, ethacrynic acid, furosemide, torsemide, amiloride, triamterene, and metolazone.

Typically, where the drug is a central nervous system stimulant, it is selected from one of the following compounds: amphetamine, brucine, caffeine, dexfenfluramine, dextroamphetamine, ephedrine, fenfluramine, mazindol, methyphenidate, pemoline, phentermine, sibutramine, and modafinil.

Typically, where the drug is a drug for Alzheimer's disease management, it is selected from one of the following compounds: donepezil, galanthamine and tacrin.

Typically, where the drug is a drug for cystic fibrosis management, it is selected from one of the following compounds: tobramycin and cefadroxil.

Typically, where the drug is a diagnostic agent, it is selected from one of the following compounds: adenosine and aminohippuric acid.

Typically, where the drug is a dietary supplement, it is selected from one of the following compounds: melatonin and vitamin-E.

Typically, where the drug is a drug for erectile dysfunction, it is selected from one of the following compounds: tadalafil, sildenafil, vardenafil, apomorphine, apomorphine diacetate, phentolamine, and yohimbine.

Typically, where the drug is a gastrointestinal agent, it is selected from one of the following compounds: loperamide, atropine, hyoscyamine, famotidine, lansoprazole, omeprazole, and rebeprazole.

Typically, where the drug is a hormone, it is selected from one of the following compounds: testosterone, estradiol, and cortisone.

Typically, where the drug is a drug for the treatment of alcoholism, it is selected from one of the following compounds: naloxone, naltrexone, and disulfiram.

Typically, where the drug is a drug for the treatment of addiction it is buprenorphine.

Typically, where the drug is an immunosuppressive, it is selected from one of the following compounds: mycophenolic acid, cyclosporin, azathioprine, tacrolimus, and rapamycin.

Typically, where the drug is a mast cell stabilizer, it is selected from one of the following compounds: cromolyn, pemirolast, and nedocromil.

Typically, where the drug is a drug for migraine headache, it is selected from one of the following compounds: almotriptan, alperopride, codeine, dihydroergotamine, ergotamine, eletriptan, frovatriptan, isometheptene, lidocaine, lisuride, metoclopramide, naratriptan, oxycodone, propoxyphene, rizatriptan, sumatriptan, tolfenamic acid, zolmitriptan, amitriptyline, atenolol, clonidine, cyproheptadine, diltiazem, doxepin, fluoxetine, lisinopril, methysergide, metoprolol, nadolol, nortriptyline, paroxetine, pizotifen, pizotyline, propanolol, protriptyline, sertraline, timolol, and verapamil.

Typically, where the drug is a motion sickness product, it is selected from one of the following compounds: diphenhydramine, promethazine, and scopolamine.

Typically, where the drug is a drug for multiple sclerosis management, it is selected from one of the following compounds: bencyclane, methylprednisolone, mitoxantrone, and prednisolone.

Typically, where the drug is a muscle relaxant, it is selected from one of the following compounds: baclofen, chlorzoxazone, cyclobenzaprine, methocarbamol, orphenadrine, quinine, and tizanidine.

Typically, where the drug is a nonsteroidal anti-inflammatory, it is selected from one of the following compounds: aceclofenac, acetaminophen, alminoprofen, amfenac, aminopropylon, amixetrine, aspirin, benoxaprofen, bromfenac, bufexamac, carprofen, celecoxib, choline, salicylate, cinchophen, cinmetacin, clopriac, clometacin, diclofenac, diflunisal, etodolac, fenoprofen, flurbiprofen, ibuprofen, indomethacin, indoprofen, ketoprofen, ketorolac, mazipredone, meclofenamate, nabumetone, naproxen, parecoxib, piroxicam, pirprofen, rofecoxib, sulindac, tolfenamate, tolmetin, and valdecoxib.

Typically, where the drug is an opioid, it is selected from one of the following compounds: alfentanil, allylprodine, alphaprodine, anileridine, benzylmorphine, bezitramide, buprenorphine, butorphanol, carbiphene, cipramadol, clonitazene, codeine, dextromoramide, dextropropoxyphene, diamorphine, dihydrocodeine, diphenoxylate, dipipanone, fentanyl, hydromorphone, L-alpha acetyl methadol, lofentanil, levorphanol, meperidine, methadone, meptazinol, metopon, morphine, nalbuphine, nalorphine, oxycodone, papaveretum, pethidine, pentazocine, phenazocine, remifentanil, sufentanil, and tramadol.

Typically, where the drug is an other analgesic it is selected from one of the following compounds: apazone, benzpiperylon, benzydramine, caffeine, clonixin, ethoheptazine, flupirtine, nefopam, orphenadrine, propacetamol, and propoxyphene.

Typically, where the drug is an opthalmic preparation, it is selected from one of the following compounds: ketotifen and betaxolol.

Typically, where the drug is an osteoporosis preparation, it is selected from one of the following compounds: alendronate, estradiol, estropitate, risedronate and raloxifene.

Typically, where the drug is a prostaglandin, it is selected from one of the following compounds: epoprostanol, dinoprostone, misoprostol, and alprostadil.

Typically, where the drug is a respiratory agent, it is selected from one of the following compounds: albuterol, ephedrine, epinephrine, fomoterol, metaproterenol, terbutaline, budesonide, ciclesonide, dexamethasone, flunisolide, fluticasone propionate, triamcinolone acetonide, ipratropium bromide, pseudoephedrine, theophylline, montelukast, and zafirlukast.

Typically, where the drug is a sedative and hypnotic, it is selected from one of the following compounds: butalbital, chlordiazepoxide, diazepam, estazolam, flunitrazepam, flurazepam, lorazepam, midazolam, temazepam, triazolam, zaleplon, zolpidem, and zopiclone.

Typically, where the drug is a skin and mucous membrane agent, it is selected from one of the following compounds: isotretinoin, bergapten and methoxsalen.

Typically, where the drug is a smoking cessation aid, it is selected from one of the following compounds: nicotine and varenicline.

Typically, where the drug is a Tourette's syndrome agent, it is pimozide.

Typically, where the drug is a urinary tract agent, it is selected from one of the following compounds: tolteridine, darifenicin, propantheline bromide, and oxybutynin.

Typically, where the drug is a vertigo agent, it is selected from one of the following compounds: betahistine and meclizine.

The term “drug composition” as used herein refers to a composition that comprises only pure drug, two or more drugs in combination, or one or more drugs in combination with additional components. Additional components can include, for example, pharmaceutically acceptable excipients, carriers, and surfactants.

The term “thermal vapor” as used herein refers to a vapor phase, aerosol, or mixture of aerosol-vapor phases, formed preferably by heating. The thermal vapor may comprise a drug and optionally a carrier, and may be formed by heating the drug and optionally a carrier. The term “vapor phase” refers to a gaseous phase. The term “aerosol phase” refers to solid and/or liquid particles suspended in a gaseous phase.

The term “drug degradation product” as used herein refers to a compound resulting from a chemical modification of the drug compound during the drug vaporization-condensation process. The modification, for example, can be the result of a thermally or photochemically induced reaction. Such reactions include, without limitation, oxidation and hydrolysis.

The term “fraction drug degradation product” as used herein refers to the quantity of drug degradation products present in the aerosol particles divided by the quantity of drug plus drug degradation product present in the aerosol, i.e. (sum of quantities of all drug degradation products present in the aerosol)/((quantity of drug composition present in the aerosol)+(sum of quantities of all drug degradation products present in the aerosol)). The term “percent drug degradation product” as used herein refers to the fraction drug degradation product multiplied by 100%, whereas purity of the aerosol refers to 100% minus the percent drug degradation products. To determine the percent or fraction drug degradation product, typically, the aerosol is collected in a trap, such as a filter, glass wool, an impinger, a solvent trap, or a cold trap, with collection in a filter particularly preferred. The trap is then typically extracted with a solvent, e.g. acetonitrile, and the extract subjected to analysis by any of a variety of analytical methods known in the art, with gas and liquid chromatography preferred methods, and high performance liquid chromatography particularly preferred. The gas or liquid chromatography method includes a detector system, such as a mass spectrometry detector or ultraviolet absorption detector. Ideally, the detector system allows determination of the quantity of the components of the drug composition and drug degradation product by weight. This is achieved in practice by measuring the signal obtained upon analysis of one or more known mass(es) of components of the drug composition or drug degradation product (standards) and comparing the signal obtained upon analysis of the aerosol to that obtained upon analysis of the standard(s), an approach well known in the art. In many cases, the structure of a drug degradation product may not be known or a standard of the drug degradation product may not be available. In such cases, it is acceptable to calculate the weight fraction of the drug degradation product by assuming that the drug degradation product has an identical response coefficient (e.g. for ultraviolet absorption detection, identical extinction coefficient) to the drug component or components in the drug composition. When conducting such analysis, for practicality drug degradation products present at less than a very small fraction of the drug compound, e.g. less than 0.2% or 0.1% or 0.03% of the drug compound, are generally excluded from analysis. Because of the frequent necessity to assume an identical response coefficient between drug and drug degradation product in calculating a weight percentage of drug degradation product, it is preferred to use an analytical approach in which such an assumption has a high probability of validity. In this respect, high performance liquid chromatography with detection by absorption of ultraviolet light at 225 nm is a preferred approach. UV absorption at other than 225 nm, most commonly 250 nm, was used for detection of compounds in limited cases where the compound absorbed substantially more strongly at 250 nm or for other reasons one skilled in the art would consider detection at 250 nm the most appropriate means of estimating purity by weight using HPLC analysis. In certain cases where analysis of the drug by UV was not viable, other analytical tools such as GC/MS or LC/MS were used to determine purity.

The term “effective human therapeutic dose” means the amount required to achieve the desired effect or efficacy, e.g., abatement of symptoms or cessation of the episode, in a human. The dose of a drug delivered in the thermal vapor refers to a unit dose amount that is generated by heating of the drug under defined delivery conditions.

Typically, the bioavailability of thermal vapors ranges from 20-100% and is preferably in the range of 50-100% relative to the bioavailability of drugs infused intravenously. The potency of the thermal vapor drug or drugs per unit plasma drug concentration is preferably equal to or greater than that of the drug or drugs delivered by other routes of administration. It may substantially exceed that of oral, intramuscular, or other routes of administration in cases where the clinical effect is related to the rate of rise in plasma drug concentration more strongly than the absolute plasma drug concentration. In some instances, thermal vapor delivery results in increased drug concentration in a target organ such as the brain, relative to the plasma drug concentration (Lichtman et al., The Journal of Pharmacology and Experimental Therapeutics 279:69-76 (1996)). Thus, for medications currently given orally, the human dose or effective therapeutic amount of that drug in thermal vapor form is generally less than the standard oral dose. Preferably it will be less than 80%, more preferably less than 40%, and most preferably less than 20% of the standard oral dose. For medications currently given intravenously, the drug dose in a thermal vapor will generally be similar to or less than the standard intravenous dose. Preferably it will be less than 200%, more preferably less than 100%, and most preferably less than 50% of the standard intravenous dose.

Determination of the appropriate dose of thermal vapor to be used to treat a particular condition can be performed via animal experiments and a dose-finding (Phase I/II) clinical trial. Preferred animal experiments involve measuring plasma drug concentrations after exposure of the test animal to the drug thermal vapor. These experiments may also be used to evaluate possible pulmonary toxicity of the thermal vapor. Because accurate extrapolation of these results to humans is facilitated if the test animal has a respiratory system similar to humans, mammals such as dogs or primates are a preferred group of test animals. Conducting such experiments in mammals also allows for monitoring of behavioral or physiological responses in mammals. Initial dose levels for testing in humans will generally be less than or equal to the least of the following: current standard intravenous dose, current standard oral dose, dose at which a physiological or behavioral response was obtained in the mammal experiments, and dose in the mammal model which resulted in plasma drug levels associated with a therapeutic effect of drug in humans. Dose escalation may then be performed in humans, until either an optimal therapeutic response is obtained or dose-limiting toxicity is encountered.

The actual effective amount of drug for a particular patient can vary according to the specific drug or combination thereof being utilized, the particular composition formulated, the mode of administration and the age, weight, and condition of the patient and severity of the episode being treated.

Drugs may be modified to produce desirable aerosolization properties. These include low melting point, low liquid viscosity, high vapor pressure, high thermal stability, high degree of purity, and high concentration of active drug compound. A low melting point is desirable because a variety of aerosolization techniques require the drug to be in a liquid state. A low viscosity is desirable because a variety of liquid aerosolization techniques are more effective for liquids of lower viscosity. A high vapor pressure is desirable because high density, small particle size aerosols are readily produced by condensation of drug vapors. A high thermal stability is desirable because application of heat melts solid drugs, decreases the viscosity of liquid drugs, and increases drug vapor pressure. Thus a high thermal stability allows heating of the drug formulation to improve its aerosolization properties without thermal degradation occurring. A high degree of purity is desirable to increase the delivery of active drug relative to other components, which are generally not beneficial and may in some cases be harmful. A high concentration of active drug compound is desirable to increase the amount of drug that can be delivered in a single unit dose. In addition, a high concentration of active drug compound allows a given unit dosage to be in a smaller net volume. Since the net volume is smaller, the patient does not need a large inspiratory volume and can more accurately introduce a measurable amount of drug in a single breath.

Esterification of Drugs to Enhance Drug Volatility:

The esterification of drugs tends to decrease the melting point, increase the vapor pressure, and increase the thermal stability of drugs containing carboxylic acid groups, and of some drugs containing hydroxyl groups. Drugs that were previously solids at room temperature may be esterified to form pure liquids at room temperature, which then may be aerosolized by a variety of methods known in the art. Further, drugs not suitable for volatilization due to low vapor pressures may be esterified to thereby preferably make them suitable. Drugs that have improved properties in forming thermal vapors can also have improved properties as aerosols, or as an aerosol-vapor mixture. Moreover, drugs that previously thermally degraded upon heating, can be esterified to have sufficient thermal stability to form a pure, low viscosity liquid upon heating, or to form a pure thermal vapor (for formation of condensation aerosols) upon heating. In this manner, significant amounts of degradation products are not delivered in the aerosol or the aerosol-vapor mixture. In one embodiment, drugs may be modified so that they volatilize at a temperature where they are more thermally stable. In this manner, significant amounts of degradation products are not delivered in the thermal vapor. Some drugs that are modified by esterification exhibit enhanced volatility due to their lower boiling point or higher vapor pressure, or increased thermal stability in comparison to the unesterified drug. Examples of changes in the melting point or boiling point of a drug based on changes in its form is presented in Table 2. The melting point and boiling point values in Table 2 were obtained from Budavari et al. eds. (1996). The Merck Index, Twelfth Edition. Merck & Co., Inc., New Jersey. The temperatures that are listed are melting points at standard pressure unless otherwise indicated. Examples for which boiling points (bp) are listed indicate that the substance is a liquid at room temperature and pressure. “Dec” means decomposes. Decreases in the melting point of a drug may generally correspond to a decrease in its boiling point.

U.S. Pat. Nos. 4,423,071 to Chignac et al.; 4,376,767 to Sloan et al; 4,654,370 to Marriott, III et al.; and International Applications WO 97/16181 to Hussain et al. and WO 85/00520 to Shashoua describe esterifying pharmaceuticals, not to enhance their volatility, but to increase their bioavailability as solid or liquid unit dose preparations.

As used herein, the terms “esterified drug” and “drug ester” are used interchangeably and refer to any drug that contains an ester group. Drug esters that may be used in the present invention may be synthesized by reacting an alcohol with a drug or one of its pharmaceutically acceptable salts that contain a carboxylic acid group, or by reacting an organic acid with a drug or one of its pharmaceutically acceptable salts that contain a hydroxyl group, as described in Streitwieser A., Jr. and Heathcock C. H. (1981). Introduction to Organic Chemistry, Macmillan Publishing Co., Inc., New York. As used herein, a “pharmaceutically acceptable salt” is a salt form of a drug that is suitable for administration to a patient. New drug esters and drug ester compositions as well as those that are publicly available may be used for thermal vapor delivery. As used herein, “pharmaceutically acceptable drug ester” is a drug ester that is in a form that is suitable for administration to a patient.

It is particularly desirable to modify drugs by esterification because enzymes that catalyze ester hydrolysis are present in a wide variety of human tissues (Kao et al., Pharmaceutical Research 17(8): 978-984 (2000)). Thus, esterified drugs are generally converted back into the parent drug compound rapidly after being delivered. Esters may be formed by reacting a drug containing an acid group with any organic alcohol such as a C₁-C₆ straight, branched chain, or cyclic alkanol, alkenol, alkynol, or aromatic alcohol such as methanol, ethanol, isopropanol, n-propanol, isobutanol, n-butanol, propylene glycol, glycerol, and phenol.

In addition, esters may be formed by reacting a drug containing a carboxylic acid group with an alcohol that has an organic functional group containing a heteroatom such as oxygen, nitrogen, sulfur, or one of the halides, as well as with an alcohol containing an aldehyde, amido, amino, ester, ether, keto, nitrile, sulfide, or sulfoxide group. The preferred esters for volatilization are simple esters of alcohols of molecular weight less than 120 g/mol, e.g., the esters of methanol and ethanol. In another embodiment, drug esters may be formed by reacting a drug containing an alcohol group with a carboxylic acid such as formic acid or acetic acid. This reaction eliminates a hydrogen bond donor that interacts with other molecules to stabilize the solid or liquid state of a drug, thereby enhancing its volatility. Steroid drug esters are preferably formed by this method.

Drug Esters Volatilized for Thermal Vapor Delivery:

The drug esters that may be volatilized for thermal vapor delivery include ester forms of antibiotics, anticonvulsants, antidepressants, antihistamines, antiparkinsonian drugs, drugs for migraine headache, drugs for the treatment of alcholism, muscle relaxants, anxiolytics, nonsteroidal anti-inflammatories, other analgesics, and steroids.

The antibiotic drug esters that may be volatilized for thermal vapor delivery include ester forms of cefmetazole, cefazolin, cephalexin, cefoxitin, cephacetrile, cephaloglycin, cephaloridine, cephalosporin c, cephalotin, cephamycin a, cephamycin b, cephamycin c, cepharin, cephradine, ampicillin, amoxicillin, hetacillin, carfecillin, carindacillin, carbenicillin, amylpenicillin, azidocillin, benzylpenicillin, clometocillin, cloxacillin, cyclacillin, methicillin, nafcillin, 2-pentenylpenicillin, penicillin n, penicillin o, penicillin s, penicillin v, chlorobutin penicillin, dicloxacillin, diphenicillin, heptylpenicillin, and metampicillin.

The anticonvulsant drug esters that may be volatilized for thermal vapor delivery include ester forms of 4-amino-3-hydroxybutyric acid, ethanedisulfonate, gabapentin, and vigabatrin.

The antidepressant drug esters that may be volatilized for thermal vapor delivery include ester forms of selective serotonin reuptake inhibitors and atypical antidepressants. The selective serotonin reuptake inhibitor drug esters that may be volatilized for thermal vapor delivery include ester forms of tianeptine. The atypical antidepressant drug esters that may be volatilized for thermal vapor delivery include ester forms of S-adenosylmethionine.

The antihistamine drug esters that may be volatilized for thermal vapor delivery include ester forms of fexofenadine.

The antiparkinsonian drug esters that may be volatilized for thermal vapor delivery include ester forms of baclofen, levodopa, carbidopa, and thioctate.

The anxiolytic drug esters that may be volatilized for thermal vapor delivery include ester forms of benzodiazepines and other anxiolytic/sedative-hypnotics. Benzodiazepine drug esters that may be volatilized for thermal vapor delivery include ester forms of chlorazepate. Other anxiolytic/sedative-hypnotic drug esters that may be volatilized for thermal vapor delivery include ester forms of calcium N-carboamoylaspartate and chloral betaine.

The drug esters for migraine headache that may be volatilized for thermal vapor delivery include ester forms of aspirin, diclofenac, naproxen, tolfenamic acid, and valproate.

The drug esters for the treatment of alcoholism that may be volatilized for thermal vapor delivery include ester forms of acamprosate.

The muscle relaxant drug esters that may be volatilized for thermal vapor delivery include ester forms of baclofen.

The nonsteroidal anti-inflammatory drug esters that may be volatilized for thermal vapor delivery include ester forms of aceclofenac, alclofenac, alminoprofen, amfenac, aspirin, benoxaprofen, bermoprofen, bromfenac, bufexamac, butibufen, bucloxate, carprofen, cinchophen, cinmetacin, clidanac, clopriac, clometacin, diclofenac, diflunisal, etodolac, fenclozate, fenoprofen, flutiazin, flurbiprofen, ibuprofen, ibufenac, indomethacin, indoprofen, ketoprofen, ketorolac, loxoprofen, meclofenamate, naproxen, oxaprozin, pirprofen, prodolic acid, salsalate, sulindac, tofenamate, and tolmetin.

The other analgesic drug esters that may be volatilized for thermal vapor delivery include ester forms of bumadizon, clometacin, and clonixin.

Steroid drug esters may be formed by esterifying an alcohol group of the steroid with a carboxylic acid. For example, a steroid, along with an appropriate protecting or activating group, if needed, may be esterified using a low molecular weight acid such as formic acid or acetic acid. The steroid drug esters that may be volatilized for thermal vapor delivery include ester forms of betamethasone, chloroprednisone, clocortolone, cortisone, desonide, dexamethasone, desoximetasone, difluprednate, estradiol, fludrocortisone, flumethasone, flunisolide, fluocortolone, fluprednisolone, hydrocortisone, meprednisone, methylprednisolone, paramethasone, prednisolone, prednisone, pregnan-3-alpha-ol-20-one, testosterone, and triamcinolone.

Thus, a variety of drug esters that can be synthesized in ester form or are publicly available in ester form may be delivered as thermal vapors. If synthesized, in one embodiment, the drug containing a carboxylic acid group is reacted with an alcohol to form an ester by the elimination of water. A drug containing an alcohol group conversely could be reacted with a carboxylic acid. See Streitwieser, supra. For example, as seen in Table 2, valeric acid, which contains a carboxylic acid group, has a boiling point of 186° C. By forming the ethyl ester of valeric acid, the boiling point of the drug decreases to 145° C. Volatilization of drugs at lower temperatures provides a way to avoid decomposing the drug upon heating and generating significant amounts of degradation products. By “significant amount” it is meant that the degradation products make up more than 0.1%, more than 1%, more than 10%, or more than 20% of the thermal vapor.

Formation of the Free Base to Enhance Thermal Vapor Delivery:

In another embodiment, drugs are used in a free base form to enhance their thermal vapor delivery. The free base in this variation lowers the boiling point or increases the vapor pressure or thermal stability of a drug. See Table 2. As used herein, the terms “free base drug”, “free-based drug”, and “drug free base” are used interchangeably and refer to any drug that is in free base form. Novel free base drugs and those known in the art may be used for thermal vapor delivery.

Free Base Drugs for Thermal Vapor Delivery:

The free base drugs that may be volatilized for thermal vapor delivery include the free bases of antibiotics, anticonvulsants, antidepressants, antiemetics, antihistamines, antiparkinsonian drugs, antipsychotics, anxiolytics, drugs for erectile dysfunction, drugs for migraine headache, drugs for the treatment of alcoholism, muscle relaxants, nonsteroidal anti-inflammatories, opioids, other analgesics, and stimulants.

The antibiotic free bases that may be volatilized for thermal vapor delivery include the free bases of cefmetazole, cefazolin, cephalexin, cefoxitin, cephacetrile, cephaloglycin, cephaloridine, cephalosporin C, cephalotin, cephamycin A, cephamycin B, cephamycin C, cepharin, cephradine, ampicillin, amoxicillin, hetacillin, carfecillin, carindacillin, carbenicillin, amylpenicillin, azidocillin, benzylpenicillin, clometocillin, cloxacillin, cyclacillin, methicillin, nafcillin, 2-pentenylpenicillin, penicillin N, penicillin O, penicillin S, penicillin V, chlorobutin penicillin, dicloxacillin, diphenicillin, heptylpenicillin, and metampicillin.

The anticonvulsant drug free bases that may be volatilized for thermal vapor delivery include the free bases of gabapentin, tiagabine, and vigabatrin.

The antidepressant drug free bases that may be volatilized for thermal vapor delivery include the free bases of tricyclic and tetracyclic antidepressants, selective serotonin reuptake inhibitors, monoamine oxidase inhibitors, and atypical antidepressants. The tricyclic and tetracyclic antidepressant drug free bases that may be volatilized for thermal vapor delivery include the free bases of amitriptyline, amoxapine, benmoxine, butriptyline, clomipramine, desipramine, dosulepin, doxepin, imipramine, kitanserin, lofepramine, medifoxamine, mianserin, maprotoline, mirtazapine, nortriptyline, protriptyline, trimipramine, and viloxazine. The serotonin reuptake inhibitor drug free bases that may be volatilized for thermal vapor delivery include the free bases of citalopram, cotinine, duloxetine, fluoxetine, fluvoxamine, milnacipran, nisoxetine, paroxetine, reboxetine, sertraline, and tianeptine. The monoamine oxidase inhibitor drug free bases that may be volatilized for thermal vapor delivery include the free bases of acetaphenazine, binedaline, brofaromine, cericlamine, clovoxamine, iproniazid, isocarboxazid, moclobemide, phenyhydrazine, phenelzine, selegiline, sibutramine, and tranylcypromine. The atypical anti-depressant drug free bases that may be volatilized for thermal vapor delivery include the free bases of ademetionine, adrafinil, amesergide, amisulpride, amperozide, benactyzine, bupropion, caroxazone, gepirone, idazoxan, metralindole, milnacipran, minaprine, nefazodone, nomifensine, ritanserin, roxindole, S-adenosylmethionine, tofenacin, trazodone, tryptophan, venlafaxine, and zalospirone.

The antiemetic drug free bases that may be volatilized for thermal vapor delivery include the free bases of alizapride, azasetron, benzquinamide, bromopride, buclizine, chlorpromazine, cinnarizine, clebopride, cyclizine, diphenhydramine, diphenidol, dolasetron methanesulfonate, droperidol, granisetron, hyoscine, lorazepam, metoclopramide, metopimazine, ondansetron, perphenazine, promethazine, prochlorperazine, scopolamine, triethylperazine, trifluoperazine, triflupromazine, trimethobenzamide, tropisetron, domeridone, and palonosetron.

The antihistamine drug free bases that may be volatilized for thermal vapor delivery include the free bases of azatadine, brompheniramine, chlorpheniramine, clemastine, cyproheptadine, dexmedetomidine, diphenhydramine, doxylamine, hydroxyzine, cetrizine, fexofenadine, loratidine, and promethazine.

The antiparkinsonian drug free bases that may be volatilized for thermal vapor delivery include the free bases of amantadine, baclofen, biperiden, benztropine, orphenadrine, procyclidine, trihexyphenidyl, levodopa, carbidopa, selegiline, deprenyl, andropinirole, apomorphine, benserazide, bromocriptine, budipine, cabergoline, dihydroergokryptine, eliprodil, eptastigmine, ergoline pramipexole, galanthamine, lazabemide, lisuride, mazindol, memantine, mofegiline, pergolike, pramipexole, propentofylline, rasagiline, remacemide, spheramine, terguride, entacapone, and tolcapone.

The anxiolytic drug free bases that may be volatilized for thermal vapor delivery include the free bases of barbituates, benzodiazepines, and other anxiolytic/sedative-hypnotics. The barbituate free bases that may be volatilized for thermal vapor delivery include the free bases of mecloqualone, medetomidine, and metomidate. The benzodiazepine free bases that may be volatilized for thermal vapor delivery include the free bases of adinazolam, chlordiazepoxide, clobenzepam, flurazepam, lorazepam, loprazolam, and midazolam. The other anxiolytic/sedative-hypnotic free bases that may be volatilized for thermal vapor delivery include the free bases of alpidem, alseroxlon, amphenidone, azacyclonol, bromisovalum, buspirone, calcium N-carboamoylaspartate, captodiamine, capuride, carbcloral, carbromal, chloral betaine, enciprazine, flesinoxan, ipsapiraone, lesopitron, loxapine, methaqualone, methprylon, propanolol, tandospirone, trazadone, zopiclone, and zolpidem.

The free base drugs for migraine headache that may be volatilized for thermal vapor delivery include the free bases of almotriptan, alperopride, codeine, dihydroergotamine, ergotamine, eletriptan, frovatriptan, isometheptene, lidocaine, lisuride, metoclopramide, naratriptan, oxycodone, propoxyphene, rizatriptan, sumatriptan, tolfenamic acid, zolmitriptan, amitriptyline, atenolol, clonidine, cyproheptadine, diltiazem, doxepin, fluoxetine, lisinopril, methysergide, metoprolol, nadolol, nortriptyline, paroxetine, pizotifen, pizotyline, propanolol, protriptyline, sertraline, timolol, and verapamil.

The free base drugs for the treatment of alcoholism that may be volatilized for thermal vapor delivery include the free bases of naloxone, naltrexone, and disulfiram.

The muscle relaxant drug free bases that may be volatilized for thermal vapor delivery include the free bases of baclofen, cyclobenzaprine, orphenadrine, quinine, and tizanidine.

The nonsteroidal anti-inflammatory drug free bases that may be volatilized for thermal vapor delivery include the free bases of aceclofenac, alminoprofen, amfenac, aminopropylon, amixetrine, benoxaprofen, bromfenac, bufexamac, carprofen, choline, salicylate, cinchophen, cinmetacin, clopriac, clometacin, diclofenac, etodolac, indoprofen, mazipredone, meclofenamate, piroxicam, pirprofen, and tolfenamate.

The opioid free bases that may be volatilized for thermal vapor delivery include the free bases of alfentanil, allylprodine, alphaprodine, anileridine, benzylmorphine, bezitramide, buprenorphine, butorphanol, carbiphene, cipramadol, clonitazene, codeine, dextromoramide, dextropropoxyphene, diamorphine, dihydrocodeine, diphenoxylate, dipipanone, fentanyl, hydromorphone, L-alpha acetyl methadol, lofentanil, levorphanol, meperidine, methadone, meptazinol, metopon, morphine, nalbuphine, nalorphine, oxycodone, papaveretum, pethidine, pentazocine, phenazocine, remifentanil, sufentanil, and tramadol.

Other analgesic free bases that may be volatilized for thermal vapor delivery include the free bases of apazone, benzpiperylon, benzydramine, clonixin, ethoheptazine, flupirtine, nefopam, orphenadrine, propacetamol, and propoxyphene.

The stimulant drug free bases that may be volatilized for thermal vapor delivery include the free bases of amphetamine, brucine, dexfenfluramine, dextroamphetamine, ephedrine, fenfluramine, mazindol, methyphenidate, pemoline, phentermine, and sibutramine.

Thus, a variety of drug free bases that can be synthesized in free base form or are publicly available in free base form may be delivered as thermal vapors. If synthesized, in one embodiment, the free base is obtained by methods known in the art. For example, the salt form of a drug containing an amino group may be dissolved in any solvent in which it is soluble, such as water. Base such as sodium hydroxide or sodium bicarbonate is then added in approximately equimolar amounts to that of the salt form added. Direct evaporation of the solvent yields the free base drug compound mixed with a biocompatible salt such as sodium chloride. Extraction of the free base drug-salt mixture with a solvent in which the free base drug is highly soluble and the salt is not soluble (e.g. ether), followed by evaporation of the solvent, yields the pure free base drug compound.

In the same manner as drug esters, drug free bases allow for volatilization at lower temperatures to avoid decomposing the drug upon heating and generating significant amounts of degradation products. For example, as seen in Table 2, naltrexone HCl, which contains an amino group, has a melting point of 274° C. By forming the free base of naltrexone, the melting point of the drug decreases to 168° C.

In another embodiment, it is desirable to synthesize the drug free base from the drug ester, e.g., when vaporization occurs at a lower boiling point or when the drug is more thermally stable as a free base. The free-based drug esters that can be volatilized for thermal vapor delivery include the free base of antibiotic, anticonvulsant, antihistamine, antiparkinsonian drug, anxiolytic, muscle relaxant, nonsteroidal anti-inflammatory, and other analgesic esters.

The free-based antibiotic esters that may be volatilized for thermal vapor delivery include the free base of cefmetazole, cefazolin, cephalexin, cefoxitin, cephacetrile, cephaloglycin, cephaloridine, cephalosporin C, cephalotin, cephamycin A, cephamycin B, cephamycin C, cepharin, cephradine, ampicillin, amoxicillin, hetacillin, carfecillin, carindacillin, carbenicillin, amylpenicillin, azidocillin, benzylpenicillin, clometocillin, cloxacillin, cyclacillin, methicillin, nafcillin, 2-pentenylpenicillin, penicillin N, penicillin O, penicillin S, penicillin V, chlorobutin penicillin, dicloxacillin, diphenicillin, heptylpenicillin, and metampicillin esters.

The free-based anticonvulsant drug esters that may be volatilized for thermal vapor delivery include the free bases of gabapentin, tiagabine, and vigabatrin esters.

The free-based antidepressant drug esters that may be volatilized for thermal vapor delivery include the free bases of tianeptine and S-adenosylmethionine esters.

The free-based antihistamine drug esters that may be volatilized for thermal vapor delivery include the free base of fexofenadine esters.

The free-based antiparkinsonian drug esters that may be volatilized for thermal vapor delivery include the free base of baclofen, levodopa, and carbidopa esters.

The free-based anxiolytic drug esters that may be volatilized for thermal vapor delivery include the free bases of calcium N-carboamoylaspartate and chloral betaine esters.

The free-based muscle relaxant drug esters that may be volatilized for thermal vapor delivery include the free base of baclofen esters.

The free-based nonsteroidal anti-inflammatory drug esters that may be volatilized for thermal vapor delivery include the free bases of aceclofenac, alminoprofen, amfenac, benoxaprofen, bromfenac, carprofen, cinchophen, cinmetacin, clopriac, clometacin, diclofenac, etodolac, indoprofen, meclofenamate, pirprofen, and tolfenamate esters.

Other free-based analgesic drug esters that may be volatilized for thermal vapor delivery include the free base of clonixin esters.

Formation of the free acid to enhance thermal vapor delivery:

In another embodiment, the free acid of a drug is formed to enhance its thermal vapor delivery. Forming the free acid in this variation lowers the boiling point or increases the vapor pressure or thermal stability of a drug. See Table 2. As used herein, the terms “free acid” and “drug free acid” are used interchangeably and refer to any drug that is in free acid form. Novel free acid drugs and free acid drugs known in the art may be used for thermal vapor delivery.

Free Acids for Thermal Vapor Delivery:

The drug free acids that may be volatilized for thermal vapor delivery include the free acids of antibiotics, anticonvulsants, antidepressants, antihistamines, antiparkinsonian drugs, anxiolytics, drugs for migraine headache, drugs for the treatment of alcoholism, muscle relaxants, nonsteroidal anti-inflammatories, and other analgesics. In one embodiment, the free acid is formed from a drug that includes a carboxylic acid group.

The antibiotic free acids that may be volatilized for thermal vapor delivery include the free acids of cefmetazole, cefazolin, cephalexin, cefoxitin, cephacetrile, cephaloglycin, cephaloridine, cephalosporin C, cephalotin, cephamycin A, cephamycin B, cephamycin C, cepharin, cephradine, ampicillin, amoxicillin, hetacillin, carfecillin, carindacillin, carbenicillin, amylpenicillin, azidocillin, benzylpenicillin, clometocillin, cloxacillin, cyclacillin, methicillin, nafcillin, 2-pentenylpenicillin, penicillin N, penicillin O, penicillin S, penicillin V, chlorobutin penicillin, dicloxacillin, diphenicillin, heptylpenicillin, and metampicillin.

The anticonvulsant drug free acids that may be volatilized for thermal vapor delivery include the free acids of 4-amino-3-hydroxybutyric acid, ethanedisulfonate, gabapentin, tiagabine, valproate, and vigabatrin.

The antidepressant drug free acids that may be volatilized for thermal vapor delivery include the free acids of selective serotonin reuptake inhibitors and atypical antidepressants. The selective serotonin reuptake inhibitor drug free acids that may be volatilized for thermal vapor delivery include the free acid of tianeptine. The atypical antidepressant drug free acids that may be volatilized for thermal vapor delivery include the free acid of S-adenosylmethionine.

The antihistamine drug free acids that may be volatilized for thermal vapor delivery include the free acid of fexofenadine.

The antiparkinsonian drug free acids that may be volatilized for thermal vapor delivery include the free acids of baclofen, levodopa, carbidopa, and thioctate.

The anxiolytic drug free acids that may be volatilized for thermal vapor delivery include the free acids of benzodiazepines and other anxiolytic/sedative-hypnotics. The benzodiazepine free acids that may be volatilized for thermal vapor delivery include the free acid of clorazepate. Other anxiolytic/sedative-hypnotic free acids that may be volatilized for thermal vapor delivery include the free acids of calcium N-carboamoylaspartate and chloral betaine.

The drug free acids for migraine headache that may be volatilized for thermal vapor delivery include the free acids of aspirin, diclofenac, naproxen, tolfenamic acid, and valproate.

The muscle relaxant drug free acids that may be volatilized for thermal vapor delivery include the free acid of baclofen.

The nonsteroidal anti-inflammatory drug free acids that may be volatilized for thermal vapor delivery include the free acids of aceclofenac, alclofenac, alminoprofen, afenac, aspirin, benoxaprofen, bermoprofen, bromfenac, bufexamac, butibufen, bucloxate, carprofen, cinchophen, cinmetacin, clindanac clopriac, clometacin, diclofenac, diflunisal, etodolac, fenclozate, fenoprofen, flutiazin, flurbiprofen, ibuprofen, ibufenac, indomethacin, indoprofen, ketoprofen, ketorolac, loxoprofen, meclofenamate, naproxen, oxaprozin, pirprofen, prodolic acid, salsalate, sulindac, tolfenamate, and tolmetin.

Other analgesic drug free acids that may be volatilized for thermal vapor delivery include the free acids of bumadizon, clometacin, and clonixin.

Thus, a variety of drug free acids that can be synthesized in free acid form or are publicly available in free acid form may be delivered as thermal vapors. If synthesized, in one embodiment, the drug free acid is formed by methods known in the art. For example, the salt form of a drug containing a carboxylic acid group can be dissolved in any solvent in which it is soluble, such as water. Acid such as hydrochloric acid is then added in approximately equimolar amounts to that of the salt form added. Direct evaporation of the solvent yields the free acid drug compound mixed with a biocompatible salt such as sodium chloride. Extraction of the free acid drug-salt mixture with a solvent in which the free acid drug is highly soluble and the salt is not soluble (e.g. ether), followed by evaporation of the solvent, yields the pure free acid drug compound.

In this particular embodiment, formation of the drug free acid allows for volatilization of drugs at a lower temperatures to avoid decomposing the drug upon heating and generating significant amounts of degradation products. For example, as seen in Table 2, naproxen sodium salt, which contains a carboxylic acid group, has a melting point of 244° C. By forming the free acid of naproxen, the melting point of the drug decreases to 152° C.

In another embodiment, the free acid is formed from drugs that contain an organic functional group other than a carboxylic acid group, such as a nitrous acid or sulfonic acid group. The drug may also be an acidic heterocycle that readily deprotonates, e.g., when dissolved in water. Certain anxiolytics and muscle relaxants contain an acidic heterocycle, and are termed heterocyclic acids. Examples of the free acid form of anxiolytic heterocyclic acids that may be volatilized for thermal vapor delivery include the free acid form of allylbarbiturate, amobarbital, aprobarbital, barbital, butabarbital, butallylonal, butobarbital, carbubarb, cyclobarbital, cyclopentobarbital, mephobarbital, and secobarbital. Examples of the free acid form of muscle relaxant heterocyclic acids that may be volatilized for thermal vapor delivery include the free acid form of dantrolene. Furthermore, the sulfonic acid group of acamprosate, a drug for the treatment of alcoholism, may be modified to the free acid form and then volatilized for thermal vapor delivery.

In another embodiment, drugs that are volatilized for thermal vapor delivery contain both a carboxylic acid group and an amino group or contain more than one functional group that can be modified to enhance drug volatility or thermal stability preferably without affecting drug activity. When more than one functional group is present that can be modified, such as a carboxylic acid group and an amino group, or two or more carboxylic acid groups, protecting groups may be added to the drug so that a specific functional group can be targeted for modification. Well known methods for the use of protecting groups and methods for deprotection are described for example in Greene and Wuts. (1991). Protective Groups in Organic Synthesis, Second Edition. John Wiley and Sons, New York.

As is known to one skilled in the art, certain medications may be used in a variety of settings. For example, naltrexone may be used for treatment of either alcoholism or opiate intoxication, and diazepam may be used for treatment of conditions including panic attacks, insomnia, epileptic seizures, and nausea. Similarly, the thermal vapor delivery of many of the above medications will be of use in a variety of settings beyond those directly implied by the categories in which the medications are listed above.

Examples of drugs that are particularly useful for delivery as thermal vapors are listed in Table 2.

Drug Carriers:

The volatilization of a drug may be facilitated by combining the drug with a pharmaceutically acceptable carrier. Pharmaceutically acceptable carriers are known in the art and are relatively inert substances that facilitate administration of a drug. Carriers include solid surfaces that are stable to heating, as well as gaseous, supercritical fluid, liquid, or solid solvents that may change state (e.g., melt or vaporize) as the drug is volatilized. Carrier solvents need not solvate a drug completely. Most preferred solvents will be chemically inert to heat, and will, when mixed with a drug ester, free base, or free acid, tend to decrease the attractive forces maintaining drug molecules in the solid or liquid phase, thereby increasing the drug's vapor pressure. Generally, the carrier liquid will decrease such attractive forces by replacing attractive interactions between like drug molecules with less attractive (or repulsive) interactions between drug molecules and carrier molecules. Such less attractive (or repulsive) interactions include hydrophobic-hydrophilic interactions, polar-nonpolar interactions, and repulsive electrostatic interactions between like charges. Because water is non-toxic, chemically inert, and tends to repel hydrophobic organic compounds, water is a highly preferred carrier solvent. Other carrier solvents include terpenes such as menthol, ethanol, propylene glycol, glycerol, and other similar alcohols, polyethylene glycol, dimethylformamide, dimethylacetamide, wax, supercritical carbon dioxide, dry ice, and mixtures thereof.

The solid surfaces that may be used as carriers provide a stationary phase from which the drug ester, drug free base, or drug free acid is volatilized. They are chemically inert to heat, and will, when coated with a drug ester, drug free base, or drug free acid tend to repel molecules of the drug, provide an increased surface area for contact between the gas phase and drug, or provide less attractive drug-carrier interactions than drug-drug interactions, thereby increasing the vapor pressure of the drug ester, drug free base, or drug free acid. Solids that provide such surfaces can be in virtually any shape, but most preferably a shape that has a large surface area to volume ratio, e.g. greater than 1000 per meter. Such shapes include beads or wire of less than 1.0 mm in diameter, or wafers of less than 1.0 mm in thickness. Inert solid carrier materials may be comprised of carbonaceous materials; inorganic materials such as silica (e.g., amphorous silica S-5631 (Sigma, St. Louis, Mo.)), glass (e.g., glass cover slips), or alumina; metals such as aluminum (e.g., aluminum foil), tungsten, or platinum; polymers such as polyethylene glycol or Teflon™; coated variants of polymers or inorganic materials such as various chromatography resins, (e.g., C18 beads for reverse phase liquid chromatography); colloids such as sol-gel (Brinker C. J. and Scherer G. W. (1990). Sol-Gel Science, Academic Press, San Diego); or mixtures thereof.

Solid carbonaceous carriers include porous grade carbons, graphite, activated and non-activated carbons, e.g., PC-25 and PG-60 (Union Carbide Corp., Danbury, Conn.) and SGL 8×30 (Calgon Carbon Corp., Pittsburgh, Pa.). Solid alumina carriers include various alumina for column chromatography (Aldrich, St. Louis, Mo.), alumina of defined surface area greater than 2 m2/g, e.g., BCR171 (Aldrich, St. Louis, Mo.), and alumina sintered at temperatures greater than 1000° C., e.g., SMR-14-1896 (Davison Chemical Div., W.R. Grace & Co., Baltimore, Md.). Importantly, because the above solid surfaces are themselves inert to heat and have a low vapor pressure, the aerosols formed by use of such carrier solid surfaces contain pure drug compound without any carrier, solvent, emulsifier, propellant, or other non-drug material.

Formation and Delivery of Thermal Vapor or Condensation Aerosols:

The aerosol particles for administration can typically be formed using any of the described methods at a rate of greater than 10⁸ inhalable particles per second. In some variations, the aerosol particles for administration are formed at a rate of greater than 10⁹ or 10¹⁰ inhalable particles per second. Similarly, with respect to aerosol formation (i.e., the mass of aerosolized particulate matter produced by a delivery device per unit time) the aerosol may be formed at a rate greater than 0.25 mg/second, greater than 0.5 mg/second, or greater than 1 or 2 mg/second. Further, with respect to aerosol formation, focusing on the drug aerosol formation rate (i.e., the rate of drug compound released in aerosol form by a delivery device per unit time), the drug may be aerosolized at a rate greater than 0.5 mg drug per second, greater than 0.1 mg drug per second, greater than 0.5 mg drug per second, or greater than 1 or 2 mg drug per second.

In some variations, the drug condensation aerosols are formed from compositions that provide at least 5% by weight of drug condensation aerosol particles. In other variations, the aerosols are formed from compositions that provide at least 10%, 20%, 30%, 40%, 50%, 60%, or 75% by weight of drug condensation aerosol particles. In still other variations, the aerosols are formed from compositions that provide at least 95%, 99%, or 99.5% by weight of drug condensation aerosol particles.

In some variations, the drug condensation aerosol particles when formed comprise less than 10% by weight of a thermal degradation product. In other variations, the drug condensation aerosol particles when formed comprise less than 5%, 1%, 0.5%, 0.1%, or 0.03% by weight of a thermal degradation product.

In some variations the drug condensation aerosols are produced in a gas stream at a rate such that the resultant aerosols have a MMAD in the range of about 1-3 μm. In some variations the geometric standard deviation around the MMAD of the drug condensation aerosol particles is less than 3.0. In other variations, the geometric standard deviation around the MMAD of the drug condensation aerosol particles is less than 2.5, or less than 2.0.

In one aspect, the drug, in a form such as an ester, free base, or free acid, may also be directly heated in a thermal vapor drug delivery device intended for use by the patient, with gas flow through the delivery device being controlled, e.g., by a flow-regulated vacuum source, to mimic flow rates during patient inhalation. This variation more closely approximates the exact events involved in thermal vapor drug delivery to a patient. In-line analysis of the composition of the thermal vapor may also be completed which avoids the necessity of trapping the vapor then analyzing the contents of the trap. Such in-line analysis is most conveniently performed by gas chromatography-mass spectrometry (GC-MS). Commercially available pyrolyzers such as the Curie Point Pyrolyzer (DyChrom, Santa Clara, Calif.) or the Frontier Double Shot Pyrolyzer (Frontier Lab, Fukushima, Japan) that are specifically designed to couple to GC-MS devices provide one convenient way to heat the starting composition for in-line analysis.

The application of heat may be coupled with a decrease in pressure that facilitates drug volatilization. Such a decrease in pressure may be achieved by patient inhalation, and enhanced by, e.g., drawing air through a narrow opening, which results in an increase in flow velocity and an accompanying decrease in pressure due to the Venturi principle. The drug is then delivered in a therapeutically effective amount to exert its effect in the lung or systemically on a target organ. By “therapeutically effective amount” it is meant an amount that is sufficient to treat the condition of a patient.

In another aspect, a drug can be heated followed by cooling to form condensation aerosol. Condensation aerosols have several favorable properties for inhalation drug delivery. They may be substantially pure aerosols, because, due to the high vapor pressure and thermal stability of the pure drug ester, drug free base, or drug free acid, the drug volatilizes at temperatures substantially below those at which thermal degradation occurs. Thus the condensation aerosol may contain pure drug without carriers or thermal degradation products.

In one embodiment, they include particles preferably of a size less than five microns in mass median aerodynamic diameter, e.g., 0.2 to 3 microns in mass median aerodynamic diameter. Additionally, the condensation aerosols may possess high particle concentrations, e.g., greater than 10⁶ particles/mL, greater than 10⁸ particles/mL, or greater than 10⁹ particles/mL. Also, large numbers of particles may be generated per unit time, e.g. greater than 10⁸ particles/s, greater than 10⁹ particles/s, or greater than 10¹¹ particles/s. Importantly, because of the high vapor pressure and thermal stability of certain drugs, drug esters, drug free bases, and drug free acids, they volatilize preferably without the use of carriers or without the generation of thermal degradation products to form a substantially pure aerosol of drug.

The purity of a thermal vapor can be determined by a variety of methods, examples of which are described in Sekine et al., Journal of Forensic Science 32:1271-80 (1987), and Martin et al., Journal of Analytic Toxicology 13: 158-162 (1989). One simple approach involves heating of the starting composition in an experimental apparatus, such as a furnace, to the same temperature as that used for thermal vapor delivery to a patient, for an analogous duration of time. A gas such as air is flowed through the heating device at rates generally between 0.4-40 L/min either continuously or after the above period of time, which draws the thermal vapor out of the heating chamber and into one or more traps that collect the vapor. One convenient trap can be made by packing glass wool into glass tubing, such that about 1.0 gram of glass wool is used per 10 cm stretch of 2.0 cm inner diameter tubing. Another useful and also well known trap is a C18 filter which is a solid phase bed consisting of small particles coated with a straight chain hydrocarbon containing 18 carbons. Other convenient traps include solvent traps, such as ethanol, methanol, acetone, or dichloromethane traps, which may be at various pH values and may be conveniently cooled using dry ice. The contents of the traps are then analyzed, generally by gas or liquid chromatography coupled to any of various detection systems well known in the art, e.g., flame ionization detection or photon absorption detection systems. In the case of solid traps like the glass wool or C18 traps, it is generally convenient to extract the trap with a solvent such as ethanol, methanol, acetone, and/or dichloromethane, and to analyze the extract rather than the trap itself. While a variety of detection systems are practical, a preferred detection system is mass spectrometry because of its sensitivity and ability to identify directly the chemical components present in the thermal vapor. Such identification is particularly valuable for thermal degradation products, as knowledge of the chemical structure of the degradation products allows prediction of their potential toxicities and direct testing of the toxicities of the degradation products in animal models.

In the examples, the following drugs were vaporized and condensed to generate condensation aerosol having a purity of 90% or greater: acebutolol, acetaminophen, alprazolam, amantadine, amitriptyline, apomorphine diacetate, apomorphine hydrochloride, atropine, azatadine, betahistine, brompheniramine, bumetanide, buprenorphine, bupropion hydrochloride, butalbital, butorphanol, carbinoxamine maleate, celecoxib, chlordiazepoxide, chlorpheniramine, chlorzoxazone, ciclesonide, citalopram, clomipramine, clonazepam, clozapine, codeine, cyclobenzaprine, cyproheptadine, dapsone, diazepam, diclofenac ethyl ester, diflunisal, disopyramide, doxepin, estradiol, ephedrine, estazolam, ethacrynic acid, fenfluramine, fenoprofen, flecainide, flunitrazepam, galanthamine, granisetron, haloperidol, hydromorphone, hydroxychloroquine, ibuprofen, imipramine, indomethacin ethyl ester, indomethacin methyl ester, isocarboxazid, ketamine, ketoprofen, ketoprofen ethyl ester, ketoprofen methyl ester, ketorolac ethyl ester, ketorolac methyl ester, ketotifen, lamotrigine, lidocaine, loperamide, loratadine, loxapine, maprotiline, memantine, meperidine, metaproterenol, methoxsalen, metoprolol, mexiletine HCl, midazolam, mirtazapine, morphine, nalbuphine, naloxone, naproxen, naratriptan, nortriptyline, olanzapine, orphenadrine, oxycodone, paroxetine, pergolide, phenyloin, pindolol, piribedil, pramipexole, procainamide, prochloperazine, propafenone, propranolol, pyrilamine, quetiapine, quinidine, rizatriptan, ropinirole, sertraline, selegiline, sildenafil, spironolactone, tacrine, tadalafil, terbutaline, testosterone, thalidomide, theophylline, tocainide, toremifene, trazodone, triazolam, trifluoperazine, valproic acid, venlafaxine, vitamin E, zaleplon, zotepine, amoxapine, atenolol, benztropine, caffeine, doxylamine, estradiol 17-acetate, flurazepam, flurbiprofen, hydroxyzine, ibutilide, indomethacin norcholine ester, ketorolac norcholine ester, melatonin, metoclopramide, nabumetone, perphenazine, protriptyline HCl, quinine, triamterene, trimipramine, zonisamide, bergapten, chlorpromazine, colchicine, diltiazem, donepezil, eletriptan, estradiol-3,17-diacetate, efavirenz, esmolol, fentanyl, flunisolide, fluoxetine, hyoscyamine, indomethacin, isotretinoin, linezolid, meclizine, paracoxib, pioglitazone, rofecoxib, sumatriptan, tolterodine, tramadol, tranylcypromine, trimipramine maleate, valdecoxib, vardenafil, verapamil, zolmitriptan, zolpidem, zopiclone, bromazepam, buspirone, cinnarizine, dipyridamole, naltrexone, sotalol, telmisartan, temazepam, albuterol, apomorphine hydrochloride diacetate, carbinoxamine, clonidine, diphenhydramine, thambutol, fluticasone proprionate, fluconazole, lovastatin, lorazepam N,O-diacetyl, methadone, nefazodone, oxybutynin, promazine, promethazine, sibutramine, tamoxifen, tolfenamic acid, aripiprazole, astemizole, benazepril, clemastine, estradiol 17-heptanoate, fluphenazine, protriptyline, ethambutal, frovatriptan, pyrilamine maleate, scopolamine, and triamcinolone acetonide.

Of these compounds, the following drugs were vaporized from thin films and formed condensation aerosols having greater than 95% purity: acebutolol, acetaminophen, alprazolam, amantadine, amitriptyline, apomorphine diacetate, apomorphine hydrochloride, atropine, azatadine, betahistine, brompheniramine, bumetanide, buprenorphine, bupropion hydrochloride, butalbital, butorphanol, carbinoxamine maleate, celecoxib, chlordiazepoxide, chlorpheniramine, chlorzoxazone, ciclesonide, citalopram, clomipramine, clonazepam, clozapine, codeine, cyclobenzaprine, cyproheptadine, dapsone, diazepam, diclofenac ethyl ester, diflunisal, disopyramide, doxepin, estradiol, ephedrine, estazolam, ethacrynic acid, fenfluramine, fenoprofen, flecainide, flunitrazepam, galanthamine, granisetron, haloperidol, hydromorphone, hydroxychloroquine, ibuprofen, imipramine, indomethacin ethyl ester, indomethacin methyl ester, isocarboxazid, ketamine, ketoprofen, ketoprofen ethyl ester, ketoprofen methyl ester, ketorolac ethyl ester, ketorolac methyl ester, ketotifen, lamotrigine, lidocaine, loperamide, loratadine, loxapine, maprotiline, memantine, meperidine, metaproterenol, methoxsalen, metoprolol, mexiletine HCl, midazolam, mirtazapine, morphine, nalbuphine, naloxone, naproxen, naratriptan, nortriptyline, olanzapine, orphenadrine, oxycodone, paroxetine, pergolide, phenyloin, pindolol, piribedil, pramipexole, procainamide, prochloperazine, propafenone, propranolol, pyrilamine, quetiapine, quinidine, rizatriptan, ropinirole, sertraline, selegiline, sildenafil, spironolactone, tacrine, tadalafil, terbutaline, testosterone, thalidomide, theophylline, tocainide, toremifene, trazodone, triazolam, trifluoperazine, valproic acid, venlafaxine, vitamin E, zaleplon, zotepine, amoxapine, atenolol, benztropine, caffeine, doxylamine, estradiol 17-acetate, flurazepam, flurbiprofen, hydroxyzine, ibutilide, indomethacin norcholine ester, ketorolac norcholine ester, melatonin, metoclopramide, nabumetone, perphenazine, protriptyline HCl, quinine, triamterene, trimipramine, zonisamide, bergapten, chlorpromazine, colchicine, diltiazem, donepezil, eletriptan, estradiol-3,17-diacetate, efavirenz, esmolol, fentanyl, flunisolide, fluoxetine, hyoscyamine, indomethacin, isotretinoin, linezolid, meclizine, paracoxib, pioglitazone, rofecoxib, sumatriptan, tolterodine, tramadol, tranylcypromine, trimipramine maleate, valdecoxib, vardenafil, verapamil, zolmitriptan, zolpidem, zopiclone, bromazepam, buspirone, cinnarizine, dipyridamole, naltrexone, sotalol, telmisartan, and temazepam.

Drugs, exemplified in the Examples below, which formed condensation aerosols from a thin film having a purity of 98% or greater were the following: acebutolol, acetaminophen, alprazolam, amantadine, amitriptyline, apomorphine diacetate, apomorphine hydrochloride, atropine, azatadine, betahistine, brompheniramine, bumetanide, buprenorphine, bupropion hydrochloride, butalbital, butorphanol, carbinoxamine maleate, celecoxib, chlordiazepoxide, chlorpheniramine, chlorzoxazone, ciclesonide, citalopram, clomipramine, clonazepam, clozapine, codeine, cyclobenzaprine, cyproheptadine, dapsone, diazepam, diclofenac ethyl ester, diflunisal, disopyramide, doxepin, estradiol, ephedrine, estazolam, ethacrynic acid, fenfluramine, fenoprofen, flecainide, flunitrazepam, galanthamine, granisetron, haloperidol, hydromorphone, hydroxychloroquine, ibuprofen, imipramine, indomethacin ethyl ester, indomethacin methyl ester, isocarboxazid, ketamine, ketoprofen, ketoprofen ethyl ester, ketoprofen methyl ester, ketorolac ethyl ester, ketorolac methyl ester, ketotifen, lamotrigine, lidocaine, loperamide, loratadine, loxapine, maprotiline, memantine, meperidine, metaproterenol, methoxsalen, metoprolol, mexiletine HCl, midazolam, mirtazapine, morphine, nalbuphine, naloxone, naproxen, naratriptan, nortriptyline, olanzapine, orphenadrine, oxycodone, paroxetine, pergolide, phenyloin, pindolol, piribedil, pramipexole, procainamide, prochloperazine, propafenone, propranolol, pyrilamine, quetiapine, quinidine, rizatriptan, ropinirole, sertraline, selegiline, sildenafil, spironolactone, tacrine, tadalafil, terbutaline, testosterone, thalidomide, theophylline, tocainide, toremifene, trazodone, triazolam, trifluoperazine, valproic acid, venlafaxine, vitamin E, zaleplon, zotepine, amoxapine, atenolol, benztropine, caffeine, doxylamine, estradiol 17-acetate, flurazepam, flurbiprofen, hydroxyzine, ibutilide, indomethacin norcholine ester, ketorolac norcholine ester, melatonin, metoclopramide, nabumetone, perphenazine, protriptyline HCl, quinine, triamterene, trimipramine, and zonisamide.

To obtain higher purity aerosols one can coat a lesser amount of drug, yielding a thinner film to heat, or alternatively use the same amount of drug but a larger surface area. Generally, except for, as discussed above, extremely thin thickness of drug film, a linear decrease in film thickness is associated with a linear decrease in impurities.

Thus for the drug composition where the aerosol exhibits an increasing level of drug degradation products with increasing film thicknesses, particularly at a thickness of greater than 0.05-20 microns, the film thickness on the substrate will typically be between 0.05 and 20 microns, e.g., the maximum or near-maximum thickness within this range that allows formation of a particle aerosol with drug degradation less than 5%. Other drugs may show less than 5-10% degradation even at film thicknesses greater than 20 microns. For these compounds, a film thickness greater than 20 microns, e.g., 20-50 microns, may be selected, particularly where a relatively large drug dose is desired.

In addition, to adjusting film thickness other modifications can be made to improve the purity or yield of the aerosol generated. One such method involves the use of an altered form of the drug, such as, for example but not limitation, use of a prodrug, or a free base, free acid or salt form of the drug. As demonstrated in various Examples below, modifying the form of the drug can impact the purity and or yield of the aerosol obtained. Although not always the case, the free base or free acid form of the drug as opposed to the salt, generally results in either a higher purity or yield of the resultant aerosol. Thus, in a preferred embodiment of the invention, the free base and free acid forms of the drugs are used.

Another approach contemplates generation of drug-aerosol particles having a desired level of drug composition purity by forming the thermal vapor under a controlled atmosphere of an inert gas, such as argon, nitrogen, helium, and the like. Various Examples below show that a change in purity can be observed upon changing the gas under which vaporization occurs.

Examples 166-233 correspond to studies conducted on drugs that when deposited as a thin film on a substrate produced a thermal vapor having a drug purity of less than about 90% but greater than about 60% or where the percent yield was less than about 50%. Purity of the thermal vapor of many of these drugs would be improved by using one or more of the approaches discussed above.

Once a desired purity and yield have been achieved or can be estimated from a graph of aerosol purity versus film thickness and the corresponding film thickness determined, the area of substrate required to provide a therapeutically effective dose is determined

Formation of Carrier-Free Aerosols by Liquid Aerosolization

As described above, drug esters, drug free bases, and drug free acids have favorable properties for liquid aerosolization, including a low melting point and a low liquid viscosity. Thus, drug esters, drug free bases, and drug free acids may be aerosolized using a variety of liquid aerosolization techniques known in the art, without the need for adding carriers, solvents, emulsifiers, propellants, or other non-drug material that are required in the prior art. Methods of aerosolizing drug esters, drug free bases, and drug free acids include jet nebulization, ultrasonic nebulization, passage of the liquid to be aerosolized through micron-sized holes, and electrohydrodynamic nebulization (aerosolization via application of an electric field). Such methods are known in the art and are described, e.g., in Aerosol Technology, by William C. Hinds, John Wiley and Sons, NY, 1999.

Jet nebulizers release compressed air from a small orifice at high velocity, resulting in low pressure at the exit region due to the Bernoulli effect, as described in U.S. Pat. No. 5,511,726 to Greenspan et al. The low pressure is used to draw the fluid to be aerosolized out of a second tube. This fluid breaks into small droplets as it accelerates in the air stream. The pressure of compressed gas determines the flow rate through the nebulizer, and can be modulated to alter the particle size distribution and rate of aerosolization of the drug ester, drug free base, or drug free acid. As used in the prior art, jet nebulizers require added carriers, solvents, emulsifiers, and other non-drug material. The invention herein, however, allows jet nebulization of pure or substantially pure compounds.

Ultrasonic nebulizers convert electrical energy into ultrasound frequency vibrations of a piezoelectric crystal. The vibrations are transmitted to the liquid to be nebulized. The nebulizer forms a fountain that breaks up into an assembly of polydisperse droplets, and the patient's breathing or externally supplied gas flow acts as the carrier medium for the droplets. Ultrasonic nebulizers can deliver substantially more liquid (1 to 2 g per minute) to a patient than jet nebulizers (100 to 200 mg per minute).

Devices in which liquid is passed through micron-sized holes can also be used for aerosolization of drug ester, drug free base, or drug free acid. In one embodiment, these devices use pressure to force liquid through micron-sized pores in a membrane (see e.g. U.S. Pat. No. 6,131,570 to Schuster et al.; U.S. Pat. No. 5,724,957 to Rubsamen et al.; and U.S. Pat. No. 6,098,620 to Lloyd et al.). Preferred pore size for systemic delivery is in a range about 0.5 microns to about 3 microns. Preferred liquid viscosity is in the range of 0.25 to 10 times the viscosity of water. In another embodiment, these devices use vibration to drive liquid through micron-sized holes in a plate or shell (see e.g. U.S. Pat. Nos. 5,586,550; 5,758,637; and 6,085,740 to Ivri et al.; and U.S. Pat. No. 5,938,117 to Ivri). Both types of devices are generally limited in that they can deliver only 50 mg or less of liquid per inhalation. However, they both benefit from producing smaller-sized particles and a more uniform particle size distribution than jet or ultrasonic nebulizer devices.

Electrohydrodynamic nebulizers use a voltage source to apply a large electric field across a droplet of fluid or a capillary tube containing fluid (see U.S. Pat. No. 5,511,726 to Greenspan et al.). Application of the large electric field results in breaking of a droplet of fluid into an aerosol, or release of a fan of aerosol from a fluid-filled capillary tube. Aerosols produced by this method can have a very fine particle size, in the range of 0.2 to 5 microns. This method is generally effective for liquids with conductivities close to the conductivity of water, and ineffective for liquids of very low conductivity (e.g. benzene) or very high conductivity (e.g. concentrated hydrochloric acid). Generally, drug esters, drug free bases, and drug free acids have appropriate conductivities for electrohydrodynamic nebulization. An advantage of this approach is small particle size, uniformity of particle size distribution, and high aerosol particle density (e.g. up to 108 particles/mL). A disadvantage is limited quantity of material that can be aerosolized (e.g. 5-50 microliters) and the requirement for a large voltage (e.g. 5 to 50 kV) to achieve the high electric field.

In certain cases, the above aerosolization methods may result in production of both small particles that deliver drug to the systemic circulation effectively, and also larger particles that are inappropriate for systemic drug delivery. To eliminate such larger particles, baffles capable of filtering out excessively large droplets may also be incorporated in an aerosol apparatus. In addition, in certain instances aerosol properties such as particle size may be strongly dependent on ambient conditions such as humidity or temperature. In such cases, a heating apparatus between the aerosol source and aerosol introduction to a patient as disclosed in U.S. Pat. No. 5,743,251 to Cox et al. and U.S. Pat. No. 5,743,251 to Howell may improve the aerosol's properties or improve the reproducibility of those properties.

The esterified, free base, or free acid drug aerosols have properties that allow for improved aerosolization. The aerosols may be formed in pure or substantially pure form. Unit doses of the modified drugs may be delivered. In addition, the aerosols contain greater than 10⁵ particles per mL, preferably greater than 10⁶ particles per mL, more preferably greater than 10⁷ particles per mL.

Use of Heat in the Formation of Carrier-Free Aerosols by Liquid Aerosolization

Beyond their low melting point and low liquid viscosity, drug esters, drug free bases, and drug free acids may have high thermal stabilities. Thus, drug esters, drug free bases, and drug free acids may be heated to temperatures preferably in the range of 50° C. to 350° C. without thermal degradation. Such heating, e.g., converts solid drug forms to liquid forms and decreases the viscosity of liquid drug forms. Thus in another embodiment of the invention, drug formulations are heated prior to aerosolization to facilitate aerosolization of the drug formulation using a method for aerosolization of liquids as described above. Because of the high thermal stability of drug esters, drug free bases, and drug free acids, such heating facilitates aerosolization of the carrier-free drug formulation without decreasing the purity of the aerosol, and, without using a carrier, a substantially pure aerosol may be formed. Such an approach can generate solid aerosols as well as liquid aerosols. In particular, a drug formulation that is a solid at room temperature may be heated to form a liquid, aerosolized by a liquid aerosolization method, and freeze during cooling following aerosolization to yield a solid particle aerosol.

Drug-Supply Article

The subject methods of delivering a drug aerosol composition may be accomplished through any of a variety of drug delivery devices which provide for heating of a selected drug and allow simultaneous or sequential inhalation of the evolved thermal vapor. The device may comprise any ergonomically designed, inert passageway that links the site of volatilization of the drug to the mouth of the inhaling patient. The drug is preferably delivered in a therapeutically effective amount to exert its effect in the lung or systemically on a target organ.

It may also be desirable to include in the device any monitor known in the art that controls the timing of drug volatilization relative to inhalation, gives feedback to patients on the rate and/or volume of inhalation, prevents excessive use (i.e. provides a “lock-out” feature), prevents use by unauthorized individuals, and records dosing histories.

The heat used to vaporize drugs may be generated by such means as passage of current through an electrical resistance element, by absorption of electromagnetic radiation (e.g. microwaves or laser light), by non-covalent chemical reactions (e.g. hydration of pyrophosphorous material), and by covalent chemical reactions (e.g. burning). Thus, an example of a very simple heating device that can readily be used to volatilize drugs of the present invention involves a tungsten or platinum wire that is coated with a drug ester, drug free base, or drug free acid by dipping the wire into a concentrated solution containing one of those drugs and allowing the solvent to evaporate. Passage of current through the wire then results in heating of the wire and volatilization of the drug. Temperatures achieved during heating of the drug are controlled so as to avoid substantial thermal degradation of the drug.

Another example of a simple heating device that can be used to volatilize drugs includes an inert, heat conducting inhalation passageway onto which a drug such as a drug ester, drug free base, or drug free acid, is coated on the inside. The passageway is surrounded by valves such as those present on a typical cigarette lighter. When the valves are opened, they release a combustible fuel such as ethanol or butane which is ignited by an electrical spark. Combustion of the fuel results in heating of the inert passageway and volatilization of the drug. Combustion by-products are physically segregated from the drug by the inert passageway and thus are not inhaled by the patient.

Yet another example of a simple heating device that can be used to volatilize drugs for formation of condensation aerosols or thermal vapors includes a sealed chamber containing a chemical fuel that generates heat upon burning (e.g. butane or magnesium), surrounded by a high surface area material (e.g. “fins” made of aluminum) that is coated with a thin layer of drug. Ignition of the fuel (e.g. by an electrical spark) results in heating of the chamber, which is in heat-transfer contact with high surface area material from which the drug is rapidly volatilized.

The condensation aerosols disclosed herein are beneficial in that the ester, free base, or free acid forms of the drug may be delivered in pure or substantially pure form. The esterified, free base, or free acid drugs may also be delivered with less than 10%, preferably less than 1%, or more preferably less than 0.1% or less than 0.03% degradation products. Furthermore, unit doses of the drug may be delivered. Condensation aerosols also have very small particle sizes, generally having a mass median aerodynamic diameter less than 5 microns, and preferably less than 1-2 microns. The condensation aerosols disclosed herein also have a high particle density, typically greater than 10⁵ particles per mL, preferably greater than 10⁶ particles per mL, more preferably greater than 10⁸ particles per mL or greater than 10⁹ particles per mL. In addition, large numbers of particles may be generated per unit time, e.g. typically greater than 10⁹ particles per second, preferably greater than 10⁹ particles per second, and more preferably greater than 10¹⁰ particles per second. In addition, condensation aerosols have a low velocity relative to the patient after formation, i.e. the aerosol is not ejected towards the patient at a high velocity, thus avoiding a major problem with current inhalation technologies such as MDIs, in which failure to time inhalation precisely to generation of the aerosol results in collision of the aerosol with the posterior oropharynx and a failure to achieve the desired clinical effect

The dose of drug delivered in the thermal vapor is controlled by the physical quantity of drug provided prior to heating, the temperature to which that drug is heated, any carriers that may be present, and the degree of loss of drug on surfaces of the delivery device. The bioavailability of the delivered drug depends on the distribution of the thermal vapor drug between gas phase and aerosol phase, particle size of the drug in the aerosol phase, and the characteristics of patient inhalation. Techniques for measurement of particle size, and relationships between particle size, pulmonary deposition, and bioavailability are described in Heyder et al., Journal of Aerosol Science 17:811-825 (1986), and Clark et al., Z. Erkrank. Atmungsorgane 166:13-24 (1986). Other techniques are known. A device such as an Eight-Stage Non-Viable Cascade Impactor (Anderson Instruments, Inc., Symerna, Ga.) may be employed to measure particle size using these techniques. Concerning the relationship between patient inhalation characteristics and bioavailability, bioavailability of the drug is increased if the patient exhales fully prior to inhalation (Davies et al., Journal of Applied Physiology 32: 591-600 (1972)), inhales the drug at a moderate flow rate (Brand et al., Journal of Pharmaceutical Science 89:724-731 (2000)), inhales a bolus of drug followed by additional inhalation of normal air (Darquenne et al., Journal of Applied Physiology 83:966 (1997)), and holds his or her breath at the point of maximal inhalation.

In one embodiment, the thermal vapor is self-administered and the drug is rapidly delivered to a target site by pulmonary inhalation and absorption into the arterial circulation resulting in a rapid clinical response, e.g. within 10 minutes after inhalation, preferably within 120 seconds after inhalation, and most preferably within 30 seconds after inhalation. Because of this rapid response, thermal vapor administration provides a patient-controlled drug delivery system that allows patients to titrate their intake of drug and minimize their chance of experiencing drug side effects.

Thermal vapors may be used to treat various conditions, but are particularly effective in the treatment of neurologic and psychiatric disorders. For example, thermal vapors may be administered to prevent or treat pain, tension headache, migraine headache, cluster headache, anxiety, panic attacks, insomnia, appetite disorders, compulsive behavior, drug or cigarette craving, nausea, erectile dysfunction, epileptic seizures preceded by auras, and Parkinsonism. There are one or more key symptoms in these conditions that alert patients to the need for medication. Thus, they are able to modulate their drug intake to the minimum amount required to treat those symptoms. Such drug dose modulation may be achieved for thermal vapor delivery in several ways. For example, the starting amount of drug upon repeated volatilization and inhalation, may deliver a maximum safe unit dose amount of an ester, free base, or free acid form of drug in the thermal vapor to be taken within a given time period, e.g. 4 hours. The patient may then be free to take any number of inhalations, stopping as soon as his symptoms are ameliorated (and potentially resuming if the symptoms recur). Alternatively, the starting composition of drug ester, drug free base, or drug free acid may be completely volatilized to deliver less than a maximum safe unit dose amount of thermal vapor medication in one inhalation within a given time period. A patient may then volatilize multiple such dosage units, until either his symptoms are ameliorated, or a maximum safe number of dosage units are reached.

The drug delivery system that volatilizes the drugs may be supplied as a kit that comprises an inhalation device and one or more of these drugs. Depending on the drug to be vaporized or symptom to be treated, the kits may be tailored to provide devices with certain features or particular unit dose starting compositions. For example, narcotic medications used for the treatment of postoperative pain or various headaches may be supplied as a kit that comprises an inhalation device with a lockout and patient identification feature as well as a starting composition that provides less than a maximum safe unit dose amount of drug in the thermal vapor. However, for the treatment of a disorder such as epileptic seizures preceded by auras, the lockout feature may be unnecessary and the thermal vapor should contain a maximum safe unit dose amount of medication that is delivered with one inhalation.

In one aspect, the invention provides a drug-supply article for production of drug-aerosol particles. The article is particularly suited for use in a device for inhalation therapy for delivery of a therapeutic agent to the lungs of a patient, for local or systemic treatment. The article is also suited for use in a device that generates an air stream, for application of drug-aerosol particles to a target site. For example, a stream of air carrying drug-aerosol particles can be applied to treat an acute or chronic skin condition, can be applied during surgery at the incision site, or can be applied to an open wound. In Section A below, the drug-supply article and use of the drug-supply article in an inhalation device are described. In Section B, the relationship between drug-film thickness, substrate area, and purity of drug-aerosol particles are discussed.

A. Thin-Film Coated Substrate

A drug-supply article according to one embodiment of the invention is shown in cross-sectional view in FIG. 1A. Drug-supply article 10 is comprised of a heat-conductive substrate 12. Heat-conductive materials for use in forming the substrate are well known, and typically include metals, such as aluminum, iron, copper, stainless steel, and the like, alloys, ceramics, and filled polymers. The substrate can be of virtually any geometry, the square or rectangular configuration shown in FIG. 1A merely exemplary. Heat-conductive substrate 12 has an upper surface 14 and a lower surface 16.

Preferred substrates are those substrates that have surfaces with relatively few or substantially no surface irregularities so that a molecule of a compound vaporized from a film of the compound on the surface is unlikely to acquire sufficient energy through contact with either other hot vapor molecules, hot gases surrounding the area, or the substrate surface to result in cleavage of chemical bonds and hence compound decomposition. To avoid such decomposition, the vaporized compound should transition rapidly from the heated surface or surrounding heated gas to a cooler environment. While a vaporized compound from a surface may transition through Brownian motion or diffusion, the temporal duration of this transition may be impacted by the extent of the region of elevated temperature at the surface which is established by the velocity gradient of gases over the surface and the physical shape of surface. A high velocity gradient (a rapid increase in velocity gradient near the surface) results in minimization of the hot gas region above the heated surface and decreases the time of transition of the vaporized compound to a cooler environment. Likewise, a smoother surface facilitates this transition, as the hot gases and compound vapor are not precluded from rapid transition by being trapped in, for example, depressions, pockets or pores. Although a variety of substrates can be used, specifically preferred substrates are those that have impermeable surfaces or have an impermeable surface coating, such as, for example, metal foils, smooth metal surfaces, non-porous ceramics, etc. For the reasons stated above, non-preferred substrates for producing a therapeutic amount of a compound with less than 10% compound degradation via vaporization are those that have a substrate density of less than 0.5 g/cc, such as, for example, yarn, felts and foams, or those that have a surface area of less than 1 mm²/particle such as, for example small alumina particles, and other inorganic particles.

With continuing reference to FIG. 1A, deposited on all or a portion of the upper surface 14 of the substrate is a film 18 of drug. Preferably the film has a thickness of between about 0.05 μm and 20 μm. Film deposition is achieved by a variety of methods, depending in part on the physical properties of the drug and on the desired drug film thickness. Exemplary methods include, but are not limited to, preparing a solution of drug in solvent, applying the solution to the exterior surface and removing the solvent to leave a film of drug. The drug solution can be applied by dipping the substrate into the solution, spraying, brushing or otherwise applying the solution to the substrate. Alternatively, a melt of the drug can be prepared and applied to the substrate. For drugs that are liquids at room temperature, thickening agents can be admixed with the drug to permit application of a solid drug film. Examples of drug film deposition on a variety of substrates are given below.

FIG. 1B is a perspective, cut-away view of an alternative geometry of the drug-supply article. Article 20 is comprised of a cylindrically-shaped substrate 22 formed from a heat-conductive material. Substrate 22 has an exterior surface 24 that is preferably impermeable by virtue of material selection, surface treatment, or the like. Deposited on the exterior surface of the substrate is a film 26 of the drug composition. As will be described in more detail below, in use the substrate of the drug-supply article is heated to vaporize all or a portion of the drug film. Control of air flow across the substrate surface during vaporization produces the desired size of drug-aerosol particles. In FIG. 1B, the drug film and substrate surface is partially cut-away in the figure to expose a heating element 28 disposed in the substrate. The substrate can be hollow with a heating element inserted into the hollow space or solid with a heating element incorporated into the substrate. The heating element in the embodiment shown takes the form of an electrical resistive wire that produces heat when a current flows through the wire. Other heating elements are suitable, including but not limited to a solid chemical fuel, chemical components that undergo an exothermic reaction, inductive heat, etc. Heating of the substrate by conductive heating is also suitable. One exemplary heating source is described in U.S. patent application for SELF-CONTAINED HEATING UNIT AND DRUG-SUPPLY UNIT EMPLOYING SAME, U.S. Ser. No. 60/472,697 filed May 21, 2003 which is incorporated herein by reference.

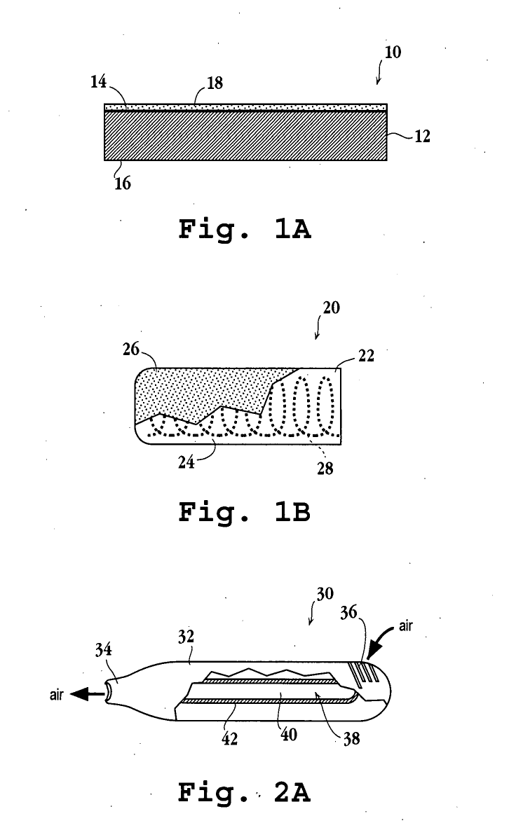

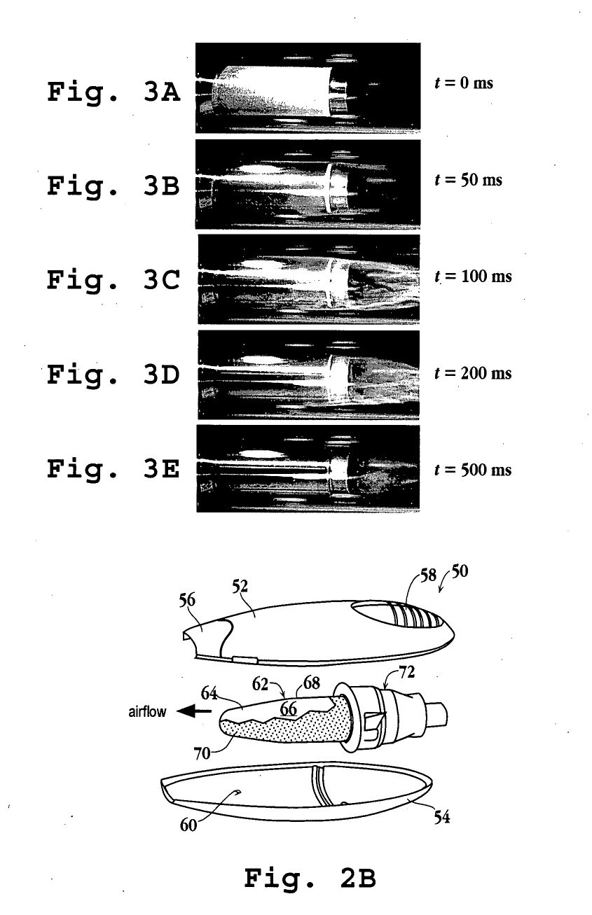

FIG. 2A is a perspective view of a drug-delivery device that incorporates a drug-supply article similar to that shown in FIG. 1B. Device 30 includes a housing 32 with a tapered end 34 for insertion into the mouth of a user. On the end opposite tapered end 34, the housing has one or more openings, such as slot 36, for air intake when a user places the device in the mouth and inhales a breath. Disposed within housing 32 is a drug-supply article 38, visible in the cut-away portion of the figure. Drug-supply article includes a substrate 40 coated on its external surface with a film 42 of a therapeutic drug to be delivered to the user. The drug-supply article can be rapidly heated to a temperature sufficient to vaporize all or a portion of the film of drug to form a drug vapor that becomes entrained in the stream of air during inhalation, thus forming the drug-aerosol particles. Heating of the drug-supply article is accomplished by, for example, an electrically-resistive wire embedded or inserted into the substrate and connected to a battery disposed in the housing. Substrate heating can be actuated by a user-activated button on the housing or via breath actuation, as is known in the art.

FIG. 2B shows another drug-delivery device that incorporates a drug-supply article, where the device components are shown in unassembled form. Inhalation device 50 is comprised of an upper external housing member 52 and a lower external housing member 54 that fit together. The downstream end of each housing member is gently tapered for insertion into a user's mouth, best seen on upper housing member 52 at downstream end 56. The upstream end of the upper and lower housing members are slotted, as seen best in the figure in the upper housing member at 58, to provide for air intake when a user inhales. The upper and lower housing members when fitted together define a chamber 60. Positioned within chamber 60 is a drug-supply unit 62, shown in a partial cut-away view. The drug supply unit has a tapered, substantially cylindrical substrate 64 coated with a film 66 of drug on its external, smooth, impermeable surface 68. Visible in the cut-away portion of the drug-supply unit is an interior region 70 of the substrate containing a substance suitable to generate heat. The substance can be a solid chemical fuel, chemical reagents that mix exothermically, electrically resistive wire, etc. A power supply source, if needed for heating, and any necessary valving for the inhalation device are contained in end piece 72.

In a typical embodiment, the device includes a gas-flow control valve disposed upstream of the drug-supply unit for limiting gas-flow rate through the condensation region to the selected gas-flow rate, for example, for limiting air flow through the chamber as air is drawn by the user's mouth into and through the chamber. In a specific embodiment, the gas-flow valve includes an inlet port communicating with the chamber, and a deformable flap adapted to divert or restrict air flow away from the port increasingly, with increasing pressure drop across the valve. In another embodiment, the gas-flow valve includes the actuation switch, with valve movement in response to an air pressure differential across the valve acting to close the switch. In still another embodiment, the gas-flow valve includes an orifice designed to limit airflow rate into the chamber.

The device may also include a bypass valve communicating with the chamber downstream of the unit for offsetting the decrease in airflow produced by the gas-flow control valve, as the user draws air into the chamber. The bypass valve cooperates with the gas-control valve to control the flow through the condensation region of the chamber as well as the total amount of air being drawn through the device. Thus the total volumetric airflow through the device, is the sum of the volumetric airflow rate through the gas-control valve, and the volumetric airflow rate through the bypass valve. The gas control valve acts to limit air drawn into the device to a preselected level, e.g., 15 L/minute, corresponding to the selected air-flow rate for producing aerosol particles of a selected size. Once this selected airflow level is reached, additional air drawn into the device creates a pressure drop across the bypass valve which then accommodates airflow through the bypass valve into the downstream end of the device adjacent the user's mouth. Thus, the user senses a full breath being drawn in, with the two valves distributing the total airflow between desired airflow rate and bypass airflow rate.

These valves may be used to control the gas velocity through the condensation region of the chamber and hence to control the particle size of the aerosol particles produced by vapor condensation. More rapid airflow dilutes the vapor such that it condenses into smaller particles. In other words, the particle size distribution of the aerosol is determined by the concentration of the compound vapor during condensation. This vapor concentration is, in turn, determined by the extent to which airflow over the surface of the heating substrate dilutes the evolved vapor. Thus, to achieve smaller or larger particles, the gas velocity through the condensation region of the chamber may be altered by modifying the gas-flow control valve to increase or decrease the volumetric airflow rate. For example, to produce condensation particles in the size range 1-3.5 μm MMAD, the chamber may have substantially smooth-surfaced walls, and the selected gas-flow rate may be in the range of 4-50 L/minute.

Additionally, as will be appreciated by one of skill in the art, particle size may be also altered by modifying the cross-section of the chamber condensation region to increase or decrease linear gas velocity for a given volumetric flow rate, and/or the presence or absence of structures that produce turbulence within the chamber. Thus, for example to produce condensation particles in the size range 20-100 nm MMAD, the chamber may provide gas-flow barriers for creating air turbulence within the condensation chamber. These barriers are typically placed within a few thousands of an inch from the substrate surface.

The heat source in one general embodiment is effective to supply heat to the substrate at a rate that achieves a substrate temperature of at least 200° C., preferably at least 250° C., or more preferably at least 300° C. or 350° C., and produces substantially complete volatilization of the drug composition from the substrate within a period of 2 seconds, preferably, within 1 second, or more preferably within 0.5 seconds. Suitable heat sources include resistive heating devices which are supplied current at a rate sufficient to achieve rapid heating, e.g., to a substrate temperature of at least 200-C, 250-C, 3000C, or 350° C. preferably within 50-500 ms, more preferably in the range of 50-200 ms. Heat sources or devices that contain a chemically reactive material which undergoes an exothermic reaction upon actuation, e.g., by a spark or heat element, such as flashbulb type heaters of the type described in several examples, and the heating source described in the above-cited U.S. patent application for SELF-CONTAINED HEATING UNIT AND DRUG-SUPPLY UNIT EMPLOYING SAME, are also suitable. In particular, heat sources that generate heat by exothermic reaction, where the chemical “load” of the source is consumed in a period of between 50-500 msec or less are generally suitable, assuming good thermal coupling between the heat source and substrate.

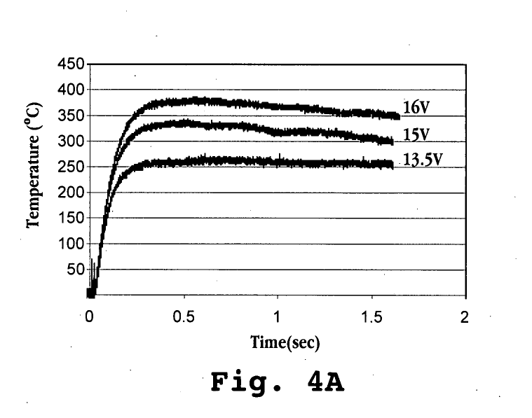

FIGS. 3A-3E are high speed photographs showing the generation of aerosol particles from a drug-supply unit. FIG. 3A shows a heat-conductive substrate about 2 cm in length coated with a film of drug. The drug-coated substrate was placed in a chamber through which a stream of air was flowing in an upstream-to-downstream direction (indicated by the arrow in FIG. 3A) at rate of about 15 L/min. The substrate was electrically heated and the progression of drug vaporization monitored by real-time photography. FIGS. 3B-3E show the sequence of drug vaporization and aerosol generation at time intervals of 50 milliseconds (msec), 100 msec, 200 msec, and 500 msec, respectively. The white cloud of drug-aerosol particles formed from the drug vapor entrained in the flowing air is visible in the photographs. Complete vaporization of the drug film was achieved by 500 msec.

The drug-supply unit generates a drug vapor that can readily be mixed with gas to produce an aerosol for inhalation or for delivery, typically by a spray nozzle, to a topical site for a variety of treatment regimens, including acute or chronic treatment of a skin condition, administration of a drug to an incision site during surgery or to an open wound. Rapid vaporization of the drug film occurs with minimal thermal decomposition of the drug, as will be further demonstrated in Section B.

B. Selection of Drug Film Thickness and Substrate Area

As discussed above, the drug supply article includes a film of drug formed on a substrate. In a preferred embodiment, the drug composition consists of two or more drugs. In a more preferred embodiment, the drug composition comprises pure drug. The drug film in one general embodiment of the invention has a thickness of between about 0.05-20 μm, and preferably between 0.1-15 μm, more preferably between 0.2-10 μm and still more preferably 0.5-10 μm, and most preferably 1-10 μm. The film thickness for a given drug composition is such that drug-aerosol particles, formed by vaporizing the drug composition by heating the substrate and entraining the vapor in a gas stream, have (i) 10% by weight or less drug-degradation product, more preferably 5% by weight or less, most preferably 2.5% by weight or less and (ii) at least 50% of the total amount of drug composition contained in the film. The area of the substrate on which the drug composition film is formed is selected to achieve an effective human therapeutic dose of the drug aerosol. Each of these features of the drug article is described below.

1. Aerosol Particle Purity and Yield

In studies conducted in support of the invention, a variety of drugs were deposited on a heat-conductive, impermeable substrate and the substrate was heated to a temperature sufficient to generate a thermal vapor. Purity of drug-aerosol particles in the thermal vapor was determined by a suitable analytical method. Three different substrate materials were used in the studies: stainless steel foil, aluminum foil, and a stainless steel cylinder. Methods B-G below detail the procedures for forming a drug film on each substrate and the method of heating each substrate.

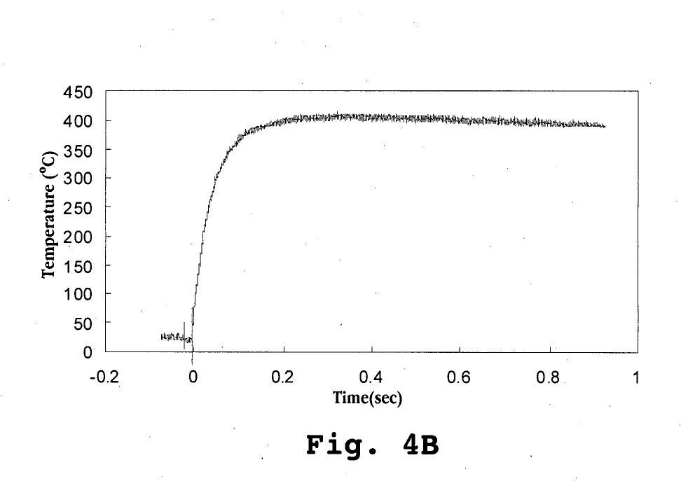

The stainless steel foil substrate employed for drugs tested according to Method B was resistively heated by placing the substrate between a pair of electrodes connected to a capacitor. The capacitor was charged to between 14-17 Volts to resistively heat the substrate. FIG. 4A is of substrate temperature increase, measured in still air with a thin thermocouple (Omega, Model CO₂-K), as a function of time, in seconds, for a stainless steel foil substrate resistively heated by charging the capacitor to 13.5 V (lower line), 15 V (middle line), and 16 V (upper line). When charged with 13.5 V, the substrate temperature increase was about 250° C. within about 200-300 milliseconds. As the capacitor voltage increased, the peak temperature of the substrate also increased. Charging the capacitor to 16V heated the foil substrate temperature about 375° C. in 200-300 milliseconds (to a maximum temperature of about 400° C.).

FIG. 4B shows the time-temperature relationship for a stainless steel foil substrate having a thickness of 0.005 inches. The foil substrate was heated by charging a capacitor, connected to the substrate through electrodes, to 16 V. The substrate reached its peak temperature of 400° C. in about 200 milliseconds, and maintained that temperature for the 1 second testing period.

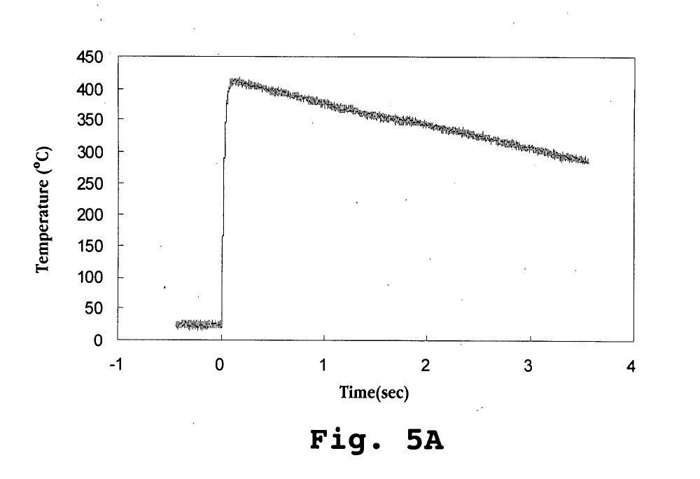

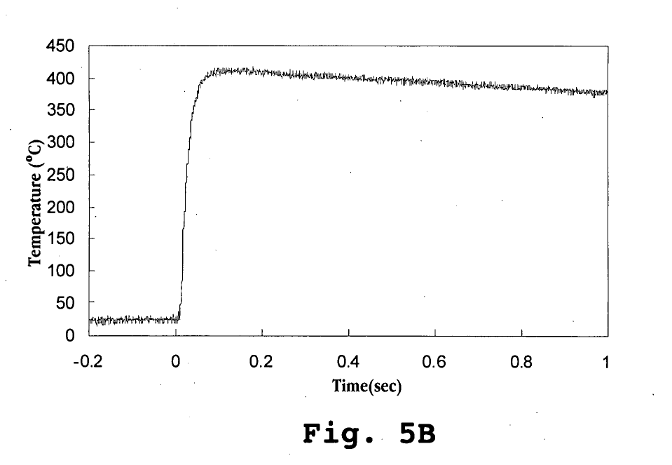

In Methods D and E, a hollow, stainless steel tube was used as the drug-film substrate. The cylindrical tube in Method D had a diameter of 13 mm and a length of 34 mm. The cylindrical tube in Method E had a diameter of 7.6 mm and a length of 51 mm. In Method D, the substrate was connected to two 1 Farad capacitors wired in parallel, whereas in Method E, the substrate was connected to two capacitors (a 1 Farad and a 0.5 Farad) wired in parallel. FIGS. 5A-5B show substrate temperature as a function of time, for the cylindrical substrate of Method D. FIG. 5B shows a detail of the first 1 second of heating.

Aluminum foil was used as a substrate for testing other compounds, as described in Methods C, F, and G. The drug-coated substrate was heated either by wrapping it around a halogen tube and applying 60 V or 90 V alternating current through the bulb or by placing the substrate in a furnace.

For each substrate type, a drug film was formed by applying a solution containing the drug onto the substrate. As described in Method A, a solution of the drug in a solvent was prepared. A variety of solvents can be used and selection is based, in part, on the solubility properties of the drug and the desired solution concentration. Common solvent choices included methanol, chloroform, acetone, dichloromethane, other volatile organic solvents, dimethylformamide, water, and solvent mixtures. The drug solution was applied to the substrate by dip coating, yet other methods such as spray coating are contemplated as well. Alternatively, a melt of the drug can be applied to the substrate.

In Examples 1-236 below a substrate containing a drug film of a certain thickness was prepared. To determine the thickness of the drug film, one method that can be used is to determine the area of the substrate and calculate drug film thickness using the following relationship:
film thickness (cm)=drug mass (g)/[drug density (g/cm³)×substrate area (cm²)]

The drug mass can be determined by weighing the substrate before and after formation of the drug film or by extracting the drug and measuring the amount analytically. Drug density can be experimentally determined by a variety of techniques, known by those of skill in the art or found in the literature or in reference texts, such as in the CRC. An assumption of unit density is acceptable if an actual drug density is not known.

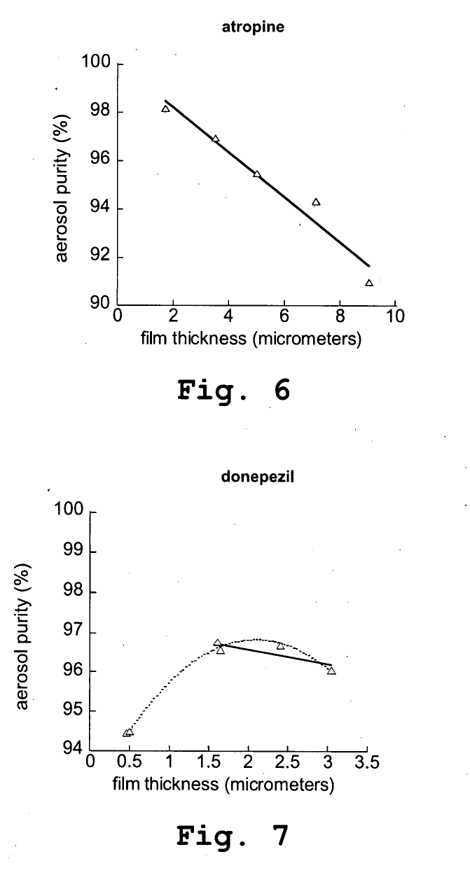

In the studies reported in the Examples, the substrate having a drug film of known thickness was heated to a temperature sufficient to generate a thermal vapor. All or a portion of the thermal vapor was recovered and analyzed for presence of drug-degradation products, to determine purity of the aerosol particles in the thermal vapor. Several drugs are discussed here as merely exemplary of the studies reported in Examples 1-236. Example 10 describes preparation of a drug-supply article containing atropine, a muscarinic antagonist. Substrates containing films of atropine ranging in thickness from between about 1.7 μm to about 9.0 μm were prepared. The stainless steel substrates were heated and the purity of the drug-aerosol particles in the thermal vapor generated from each substrate was determined. FIG. 6 shows the results, where drug aerosol purity as a function of drug film thickness is plotted. There is a clear relationship between film thickness and aerosol particle purity, where as the film thickness decreases, the purity increases. An atropine film having a thickness of 9.0 μm produced a thermal vapor having a purity of 91%; an atropine film having a thickness of 1.7 μm produced a thermal vapor having a purity of 98%.

Hydromorphone, an analgesic, was also tested, as describe in Example 66. Substrates having a drug film thickness of between about 0.7 μm to about 2.7 μm were prepared and heated to generate a thermal vapor. Purity of the aerosol particles improved as the thickness of the drug film on the substrate decreased.

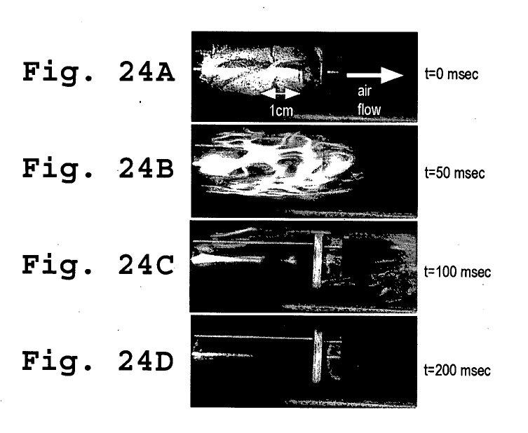

FIG. 7 shows the relationship between drug film thickness and aerosol-purity for donepezil. As described in Example 44, donepezil was coated onto foil substrates to film thicknesses ranging from about 0.5 μm to about 3.2 μm. Purity of the aerosol particles from each of the films on the substrates was analyzed. At drug film thicknesses of 1.5 μm to 3.2 μm, purity of the aerosol particles improved as thickness of the drug film on the substrate decreased, similar to the trend found for atropine and hydromorphone. In contrast, at less than 1.5 μm thickness, purity of the aerosol particles worsened as thickness of the drug film on the substrate decreased. A similar pattern was also observed for albuterol, as described in Example 3, with aerosol particles purity peaking for films of approximately 3 μm, and decreasing for both thinner and thicker films as shown in FIG. 23.

FIGS. 9-23 present data for aerosol purity as a function of film thickness for the following compounds: buprenorphine (Example 16), clomipramine (Example 28), ciclesonide (Example 26), midazolam (Example 100), nalbuphine (Example 103), naratriptan (Example 106), olanzapine (Example 109), quetiapine (Example 127), tadalafil (Example 140), prochlorperazine (Example 122), zolpidem (Example 163), fentanyl (Example 57), alprazolam (Example 4), sildenafil (Example 134), and albuterol (Example 3).

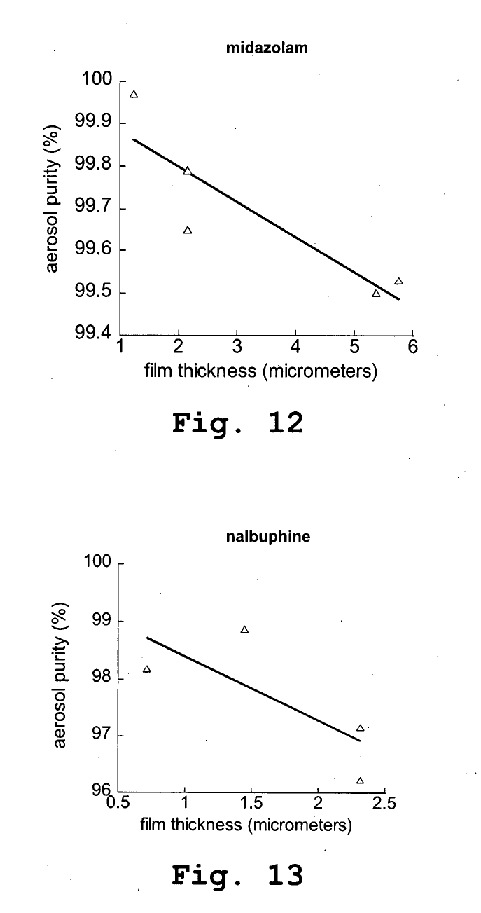

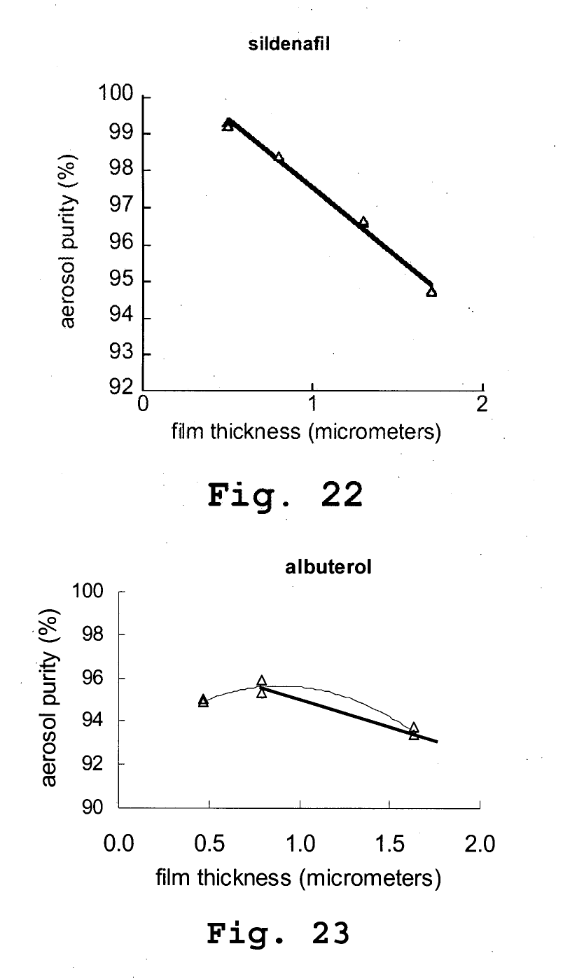

In FIGS. 6-23, the general relationship between increasing aerosol purity with decreasing film thickness is apparent; however the extent to which aerosol purity varies with a change in film thickness varies for each drug composition. For example, aerosol purity of sildenafil (FIG. 22) exhibited a strong dependence on film thickness, where films about 0.5 μm in thickness had a purity of greater than 99% and films of about 1.6 μm in thickness had a purity of between 94-95%. In contrast, for midazolam (FIG. 12), increasing the film thickness from approximately 1.2 μm to approximately 5.8 μm resulted in a decrease in aerosol particle purity from greater than 99.9% to approximately 99.5%, a smaller change in particle purity despite a larger increase in film thickness compared with the sildenafil example. Moreover, as was discussed above, the inverse relationship between film thickness and purity of aerosolized drug observed for many compounds in the thickness range less than about 20 μm does not necessarily apply at the thinnest film thicknesses that were tested. Some compounds, such as illustrated by donepezil (FIG. 7) show a rather pronounced decrease in purity at film thicknesses both below and above an optimal film thickness, in this case, above and below about 2 μm film thicknesses.

One way to express the dependence of aerosol purity on film thickness is by the slope of the line from a plot of aerosol purity against film thickness. For compounds such as donepezil (FIG. 7), the slope of the line is taken from the maximum point in the curve towards the higher film thickness. Table 1, discussed below, shows the slope of the line for the curves shown in FIGS. 6-23. Particularly preferred compounds for delivery by the various embodiments of the present invention are compounds with a substantial (i.e., highly negative) slope of the line on the aerosol purity versus thickness plot, e.g., a slope more negative than −0.1% purity per micron and more preferably −0.5% purity per micron.

In addition to selection of a drug film thickness that provides aerosol particles containing 10% or less drug-degradation product (i.e., an aerosol particle purity of 90% or more), the film thickness is selected such that at least about 50% of the total amount of drug composition contained in the film is vaporized when the substrate is heated to a temperature sufficient to vaporize the film. In the studies described herein, the percentage of drug film vaporized was determined by quantifying (primarily by HPLC or weight) the mass of drug composition collected upon vaporization or alternatively by the amount of substrate mass decrease. The mass of drug composition collected after vaporization and condensation was compared with the starting mass of the drug composition film that was determined prior to vaporization to determine a percent yield, also referred to herein as a percent emitted. This value is indicated in many of the Examples set forth below. For example, in Example 1 a film having a thickness of 1.1 μm was formed from the drug acebutolol, a beta adrenergic blocking agent. The mass coated on the substrate was 0.89 mg and the mass of drug collected in the thermal vapor was 0.53 mg, to give a 59.6 percent yield. After vaporization, the substrate and the testing chamber were washed to recover any remaining drug. The total drug recovered from the test apparatus, including the emitted thermal vapor, was 0.8 mg, to give a 91% total recovery. In another example, midazolam was coated onto a impermeable substrate, as described in Example 100. A drug film having a thickness of 9 μm was formed. Heating of the substrate generated a thermal vapor containing drug aerosol particles having a purity of 99.5%. The fraction of drug film collected on the filter, i.e., the percent yield, was 57.9%. After vaporization, the substrate and the testing chamber were washed to recover any remaining drug. The total drug recovered from the test apparatus and the filter was 5.06 mg, to give a 94.2% total recovery.

2. Substrate Area

Another feature of the drug-supply article is that the selected substrate surface area is sufficient to yield a therapeutic dose of the drug aerosol when used by a subject. The amount of drug to provide a therapeutic dose is generally known in the art or can be determined as discussed above. The required dosage and selected film thickness, discussed above, dictate the minimum required substrate area in accord with the following relationship:
film thickness (cm)×drug density (g/cm³)×substrate area (cm²)=dose (g)

As noted above, drug density can be determined experimentally or from the literature, or if unknown, can be assumed to be 1 g/cc. To prepare a drug supply article comprised of a drug film on a heat-conductive substrate that is capable of administering an effective human therapeutic dose, the minimum substrate surface area is determined using the relationships described above to determine a substrate area for a selected film thickness that will yield a therapeutic dose of drug aerosol. Table 1 shows a calculated substrate surface area for a variety of drugs on which an aerosol purity—film thickness profile was constructed.

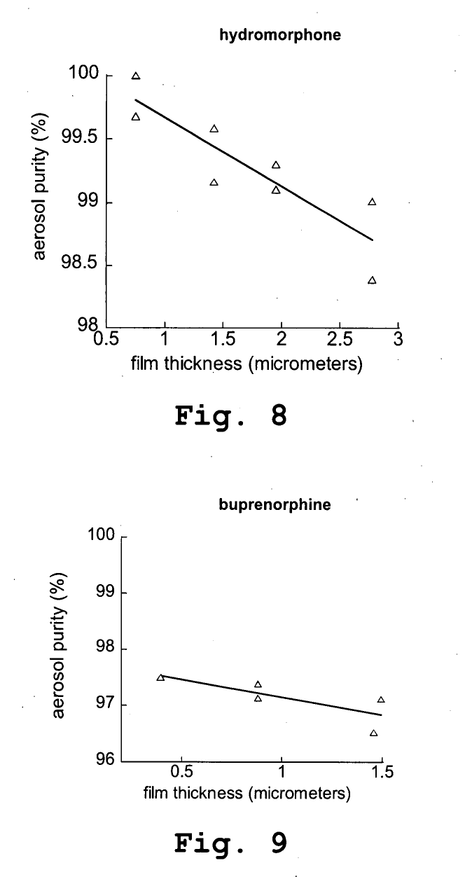

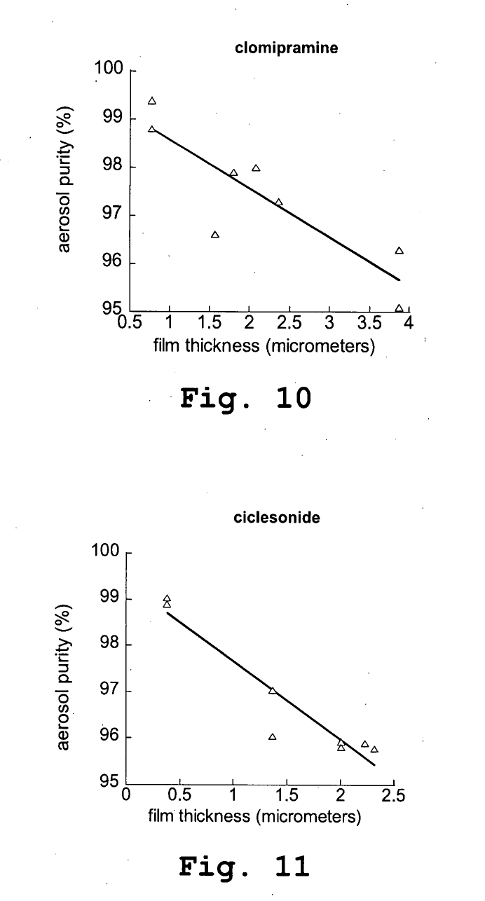

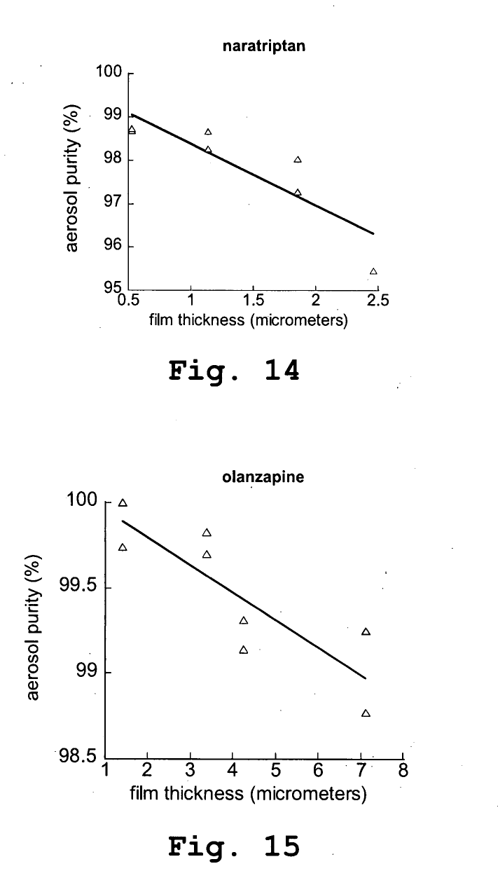

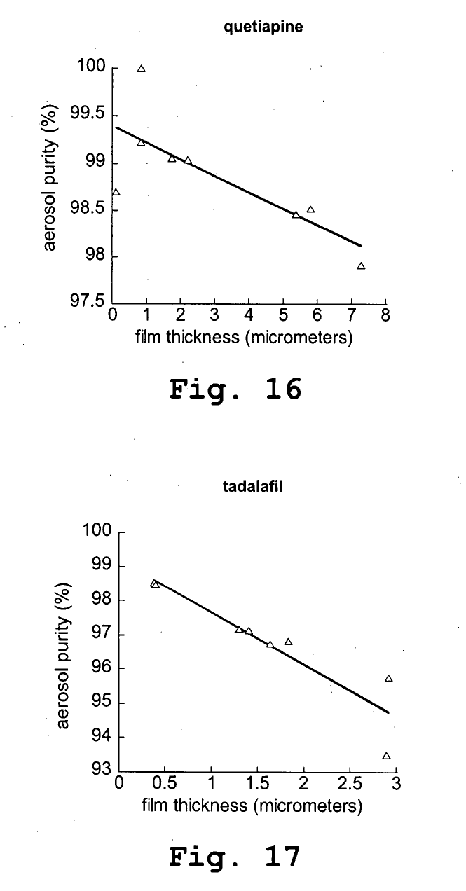

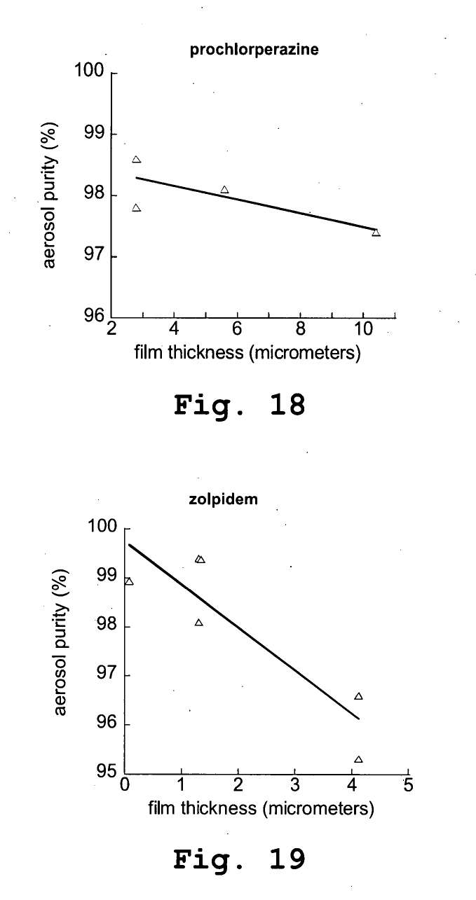

TABLE 1

| | | | | Slope of Line on | |
|---|---|---|---|---|---|
| | Typical | | | aerosol purity vs. | |
| | Dose | Preferred Film | Calculated Substrate | thickness plot (% | |
| Drug | (mg) | Thickness (μm) | Surface Area (cm²) | purity/micron) | |
| Albuterol | 0.2 | 0.1-10 | 0.2-20 | −0.64 | (FIG. 23) |
| Alprazolam | 0.25 | 0.1-10 | 0.25-25 | −0.44 | (FIG. 21) |
| Amoxapine | 25 | 2-20 | 12.5-125 | | |
| Atropine | 0.4 | 0.1-10 | 0.4-40 | −0.93 | (FIG. 6) |
| Bumetanide | 0.5 | 0.1-5 | 1-50 | | |
| Buprenorphine | 0.3 | 0.05-10 | 0.3-60 | −0.63 | (FIG. 9) |
| Butorphanol | 1 | 0.1-10 | 1-100 | | |
| Clomipramine | 50 | 1-8 | 62-500 | −1.0 | (FIG. 10) |
| Donepezil | 5 | 1-10 | 5-50 | −0.38 | (FIG. 7) |
| Hydromorphone | 2 | 0.05-10 | 2-400 | −0.55 | (FIG. 8) |
| Loxapine | 10 | 1-20 | 5-100 | | |
| Midazolam | 1 | 0.05-20 | 0.5-200 | −0.083 | (FIG. 12) |
| Morphine | 5 | 0.2-10 | 5-250 | | |
| Nalbuphine | 5 | 0.2-5 | 10-250 | −1.12 | (FIG. 13) |
| Naratriptan | 1 | 0.2-5 | 2-50 | −1.42 | (FIG. 14) |
| Olanzapine | 10 | 1-20 | 5-100 | −0.16 | (FIG. 15) |
| Paroxetine | 20 | 1-20 | 10-200 | | |
| Prochlorperazine | 5 | 0.1-20 | 2.5-500 | −0.11 | (FIG. 18) |
| Quetiapine | 50 | 1-20 | 25-500 | −0.18 | (FIG. 16) |
| Rizatriptan | 3 | 0.2-20 | 1.5-150 | | |
| Sertraline | 25 | 1-20 | 12.5-250 | | |
| Sibutramine | 10 | 0.5-2 | 50-200 | | |
| Sildenafil | 6 | 0.2-3 | 20-300 | −3.76 | (FIG. 22) |
| Sumatriptan | 3 | 0.2-6 | 5-150 | | |
| Tadalafil | 3 | 0.2-5 | 6-150 | −1.52 | (FIG. 17) |
| Testosterone | 3 | 0.2-20 | 1.5-150 | | |
| Vardenafil | 3 | 0.1-2 | 15-300 | | |
| Venlafaxine | 50 | 2-20 | 25-250 | | |
| Zolpidem | 5 | 0.1-10 | 5-500 | −0.88 | (FIG. 19) |
| Apomorphine HCl | 2 | 0.1-5 | 4-200 | | |
| Celecoxib | 50 | 2-20 | 25-250 | | |
| Ciclesonide | 0.2 | 0.05-5 | 0.4-40 | −1.70 | (FIG. 11) |
| Fentanyl | 0.5 | 0.05-5 | 0.1-10 | | |
| Eletriptan | 3 | 0.2-20 | 1.5-150 | | |
| Parecoxib | 10 | 0.5-2 | 50-200 | | |
| Valdecoxib | 10 | 0.5-10 | 10-200 | | |

The actual dose of drug delivered, i.e., the percent yield or percent emitted, from the drug-supply article will depend on, along with other factors, the percent of drug film that is vaporized upon heating the substrate. Thus, for drug films that yield upon heating 100% of the drug film and aerosol particles that have a 100% drug purity, the relationship between dose, thickness, and area given above correlates directly to the dose provided to the user. As the percent yield and/or particle purity decrease, adjustments in the substrate area can be made as needed to provide the desired dose. Also, as one of skill in the art will recognize, larger substrate areas other than the minimum calculated area for a particular film thickness can be used to deliver a therapeutically effective dose of the drug. Moreover as can be appreciated by one of skill in art, the film need not coat the complete surface area if a selected surface area exceeds the minimum required for delivering a therapeutic dose from a selected film thickness.

3. Characteristics of the Drug-Supply Article

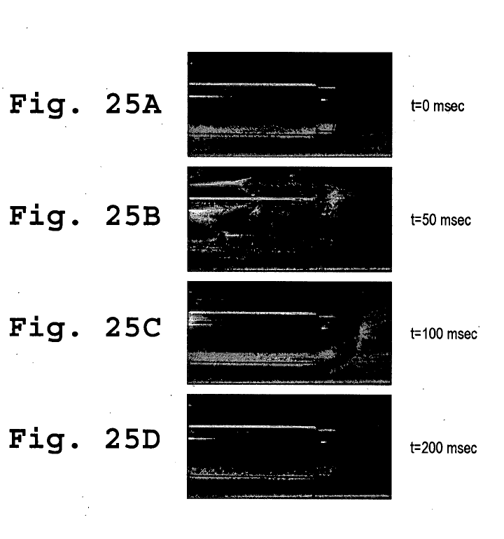

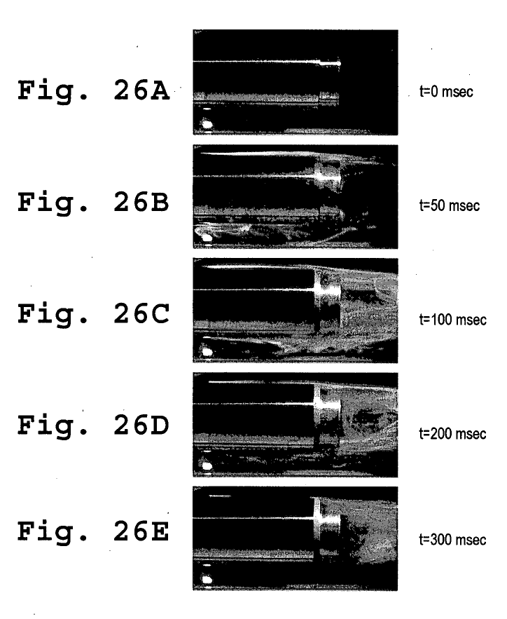

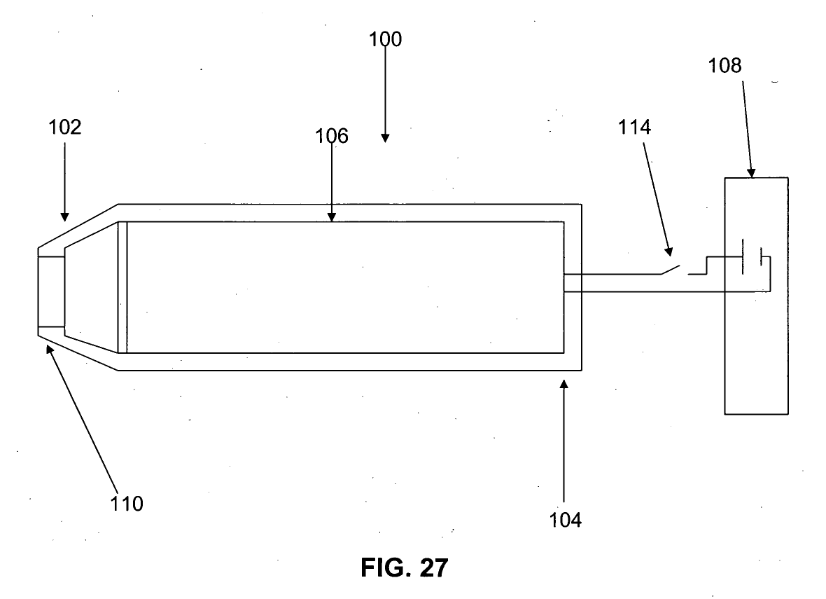

The drug-supply article of the invention is heated to generate a thermal vapor containing drug aerosol particles for therapeutic administration to a patient. In studies performed in support of the invention, high speed photography was used to monitor visually production of the thermal vapor. FIGS. 24A-24D are high speed photographs showing the generation of a thermal vapor of phenyloin from a film coated on a substrate, prepared as described in Example 116. FIG. 24A is a photograph showing the drug-coated substrate prior to heating (t=0 milliseconds (ms)). The photographs in FIGS. 24B-24D show formation of a thermal vapor as a function of time after initiation of substrate heating. The photograph in FIG. 24B, taken 50 milliseconds after initiation of substrate beating, shows formation of a thermal vapor over the substrate surface. The subsequent photographs show that the majority of the thermal vapor is formed prior to 100 milliseconds after initiation of substrate heating (FIG. 24C), with formation substantially completed by about 200 milliseconds after initiation of substrate heating (FIG. 24D).

FIGS. 25A-25D are high speed photographs showing the generation of a thermal vapor of disopyramide from a film of drug coated on a substrate, prepared as described in Example 42. FIG. 25A shows the drug-coated substrate prior to heating (t=0 milliseconds (ms)). The photographs in FIGS. 25B-25D show formation of a thermal vapor as a function of time after initiation of substrate heating. As seen, 50 milliseconds after initiation of substrate heating (FIG. 25B), a thermal vapor is present over the substrate surface. The subsequent photographs show that the majority of the thermal vapor is formed prior to 100 milliseconds after initiation of substrate heating (FIG. 25C), with formation substantially completed by about 200 milliseconds after initiation of substrate heating (FIG. 25D).

Similar photographs are shown for buprenorphine in FIGS. 26A-26E. Upon heating of a buprenorphine substrate, prepared as described in Example 16, presence of a thermal vapor is evident in the photograph taken 50 milliseconds after heating was initiated (FIG. 26B). At 100 milliseconds (FIG. 26C) and 200 milliseconds (FIG. 26D) after initiation of substrate heating the thermal vapor was still observed in the photographs. Generation of the thermal vapor was complete by 300 milliseconds (FIG. 26E).

4. Modifications to Optimize Aerosol Purity and/or Yield

As discussed above, purity of aerosol particles for many drugs correlates directly with film thickness, where thinner films typically produce aerosol particles with greater purity. Thus, one method to optimize purity disclosed in this invention is the use of thinner films. Likewise, the aerosol yield may also be optimized in this manner. The invention, however, further contemplates strategies in addition to, or in combination with, adjusting film thickness to increase either aerosol purity or yield or both. These strategies include modifying the structure or form of the drug, and/or producing the thermal vapor in an inert atmosphere.

Thus, in one embodiment, the invention contemplates generation of and/or use of an altered form of the drug, such as, for example but not limitation, use of a pro-drug, or a free base, free acid or salt form of the drug. As demonstrated in various Examples below, modifying the form of the drug can impact the purity and or yield of the aerosol obtained. Although not always the case, the free base or free acid form of the drug as opposed to the salt, generally results in either a higher purity or yield of the resultant aerosol. Thus, in a preferred embodiment of the invention, the free base and free acid forms of the drugs are used.

Another approach contemplates generation of drug-aerosol particles having a desired level of drug composition purity by forming the thermal vapor under a controlled atmosphere of an inert gas, such as argon, nitrogen, helium, and the like. Various Examples below show that a change in purity can be observed upon changing the gas under which vaporization occurs.

More generally, and in another aspect, the invention contemplates a method of forming an article for use in an aerosol device, for producing aerosol particles of a drug composition that have the desired purity and a film that provides a desired percent yield. In the method, a drug film with a known film thickness is prepared on a heat-conductive, impermeable substrate. The substrate is heated to vaporize the film, thereby producing aerosol particles containing the drug compound. The drug composition purity of the aerosol particles in the thermal vapor is determined, as well as the percent yield, i.e., the fraction of drug composition film vaporized and delivered by the method. If the drug composition purity of the particles is less than about 90%, but greater than about 60%, more preferably greater than about 70%, or if the percent yield is less than about 50%, the thickness of the drug film is adjusted to a thickness different from the initial film thickness for testing. That is, a substrate having an adjusted film thickness is heated and the percent purity and percent yield are determined. The film thickness is continually adjusted until the desired drug composition aerosol purity and yield are achieved. For example, the initial film thickness can be between about 1-20 μm. A second, different film thickness would be between about 0.05-10 μm. This method is particularly suited for drug compositions that exhibit a percent yield of greater than about 30% and a drug composition aerosol purity of between about 60%-90%, more preferably between about 70%-90%.

Examples 166-233 correspond to studies conducted on drugs that when deposited as a thin film on a substrate produced a thermal vapor having a drug purity of less than about 90% but greater than about 60% or where the percent yield was less than about 50%. Purity of the thermal vapor of many of these drugs would be improved by using one or more of the approaches discussed above. More specifically, for some drugs a simple adjustment in film thickness, typically to a thinner film, improves purity of the aerosol particles. For other drugs, heating the substrate in an inert atmosphere, such as an argon or nitrogen atmosphere, alone or in combination with an adjustment in film thickness, achieves aerosol particles with the requisite purity of 90% or more and volatilization of a fraction of the drug film that is greater than about 50%.

Based on the studies conducted, the following drugs are particularly suited to the method and approaches to optimizing purity or yield: adenosine, amoxapine, apomorphine, aripiprazole, aspirin, astemizole, atenolol, benazepril, benztropine, bromazepam, budesonide, buspirone, caffeine, captopril, carbamazepine, cinnarizine, clemastine, clemastine fumarate, clofazimine, desipramine, dipyridamole, dolasetron, doxylamine, droperidol, enlapril maleate, fluphenazine, flurazepam, flurbiprofen, fluvoxamine, frovatriptan, hydrozyzine, ibutilide, indomethacine norcholine ester, ketorolac, ketorolac norcholine ester, levodopa, melatonin, methotrexate, methysergide, metoclopramide, nabumetone, naltrexone, nalmefene, perphenazine, pimozide, piroxicam, pregnanolone, prochlorperazine 2HCl, protriptyline HCl, protriptyline, pyrilamine, pyrilamine maleate, quinine, ramipril, risperidone, scopolamine, sotalol, sulindac, terfenadine, triamcinolone acetonide, trihexyphenidyl, thiothixene, telmisartan, temazepam, triamterene, trimipramine, ziprasidone, and zonisamide.

Examples 234-235 correspond to studies conducted on combinations of drugs that when deposited as a thin film of produced a thermal vapor (aerosol) having a drug purity of greater than 90% and a recovered yield of each drug in the aerosol of greater than 50%.

Example 235 corresponds to studies conducted on drugs that when deposited as a thin film on a substrate produce a thermal vapor having a drug purity of less than about 60%.

It will be appreciated that to provide a therapeutic dose the substrate surface area is adjusted according to the film thickness that yields the desired particle purity and percent yield, as discussed above.

Utility: Thin-Film Article, Device, and Methods

As can be appreciated from the above examples showing generation of a pure drug thermal vapor, from thin films (i.e. 0.02-20 μm) of the drug, the invention finds use in the medical field in compositions and articles for delivery of a therapeutic of a drug. Thus, the invention includes, in one aspect, a drug-supply article for production of a thermal vapor that contains drug-aerosol particles. The drug-supply article includes a substrate coated with a film of a drug composition to be delivered to a subject, preferably a human subject. The thickness of the drug composition film is selected such that upon vaporizing the film by heating the substrate to a temperature sufficient to vaporize at least 50% of the drug composition film, typically to a temperature of at least about 200° C., preferably at least about 250° C., more preferably at least about 300° C. or 350° C., a thermal vapor is generated that has 10% or less drug-degradation product. The area of the substrate is selected to provide a therapeutic dose, and is readily determined based on the equations discussed above.

In another aspect the invention relates to a method of forming a drug-supply article comprised of a substrate and a film of a drug composition. The method includes identifying a thickness of drug composition film that yields after vaporization of the film the drug composition in a substantially non-pyrolyzed form, as evidenced, for example, by the purity of the vapor. This may be done by an iterative process where one first prepares on a heat-conductive substrate, a drug composition having a given film thickness, e.g., 1-10 microns. The substrate is then heated, e.g., to a selected temperature between 200° C.-600° C., preferably 250° C. to 550° C., more preferably, 300° C.-500° C., or 350° C. to 500° C., to produce an aerosol of particles containing the compound. As seen in the examples below, the aerosol may be collected in particle form or simply collected on the walls of a surrounding container. The purity of the drug composition is then determined, e.g., expressed as a weight percent or analytical percent degradation product. If the percent degradation product is above a selected threshold, e.g., 1, 2.5, 5, or 10 percent, the steps above are repeated with different compound thicknesses, typically with successively lower thicknesses, until the aerosolized compound is within the desired limit of degradation, e.g., 1, 2.5, 5, or 10%. Similarly, if the initial volatilization study shows very low levels of degradation, e.g., less than 0.1, 1, 2, or 5%, it may be desirable in subsequent tests to increase film thickness, to obtain a greatest film thickness at which an acceptable level of drug degradation is observed.

After identification of the film thickness that generates a highly pure thermal drug composition vapor (e.g., drug composition purity greater than about 90%), the area of substrate required to accommodate a therapeutic dose, when inhaled by a human, is determined. For example, the required oral dose for atropine is 0.4 mg (Example 10). Using the data shown in FIG. 6, a thermal vapor comprised of substantially non-pyrolyzed drug, e.g., a vapor having greater than about 90% drug purity, is produced from film thicknesses of less than about 10 μm. Assuming unit density for atropine, a substrate area of about 0.8 cm2 coated with a 5 μm thick drug film is required to accommodate the oral dose of 0.4 mg if a drug of 95% purity is desired. Selection of an atropine film thickness of about 1.7 μm generated a thermal vapor having drug-aerosol particles with less than 2% pyrolysis (i.e., greater than 98% drug purity). Selection of a film having a thickness of 1.7 μm requires a substrate area of at least about 2.4 cm2 to accommodate a dose of 0.4 mg.

The drug-delivery article comprised of a substrate coated with a thin drug film is particularly suited, in another aspect of the invention, for forming a therapeutic inhalation dose of drug-aerosol particles. The inhalation route of drug administration offers several advantages for many drugs, including rapid uptake into the bloodstream, and avoidance of the first pass effect allowing for an inhalation dose of a drug that can be substantially less, e.g., one half, that required for oral dosing. Efficient aerosol delivery to the lungs requires that the particles have certain penetration and settling or diffusional characteristics. For larger particles, deposition in the deep lungs occurs by gravitational settling and requires particles to have an effective settling size, defined as mass median aerodynamic diameter (MMAD), of between 1-3.5 μm. For smaller particles, deposition to the deep lung occurs by a diffusional process that requires having a particle size in the 10-100 nm, typically 20-100 nm range. Particle sizes that fall in the range between 100 nm and 1 μm tend to have poor deposition and those above 3.5 μm tend to have poor penetration. Therefore, an inhalation drug-delivery device for deep lung delivery should produce an aerosol having particles in one of these two size ranges, preferably between about 1-3 μm MMAD.

Accordingly, a drug-supply article comprised of a substrate and having a drug composition film thickness selected to generate a thermal vapor having drug composition-aerosol particles with less than about 10% drug degradation product is provided, more preferably less than about 5% drug degradation product, and most preferably less than about 2.5% drug degradation product. A gas, air or an inert fluid, is passed over the substrate at a flow rate effective to produce the particles having a desired MMAD. The more rapid the airflow, the more diluted the vapor and hence the smaller the particles that are formed. In other words the particle size distribution of the aerosol is determined by the concentration of the compound vapor during condensation. This vapor concentration is, in turn, determined by the extent to which airflow over the surface of the heating substrate dilutes the evolved vapor. Thus, to achieve smaller or larger particles, the gas velocity through the condensation region of the chamber may be altered by modifying the gas-flow control valve to increase or decrease the volumetric airflow rate. For example, to produce condensation particles in the size range 1-3.5 μm MMAD, the chamber may have substantially smooth-surfaced walls, and the selected gas-flow rate may be in the range of 4-50 L/minute.

Additionally, as will be appreciated by one of skill in the art, particle size may be also altered by modifying the cross-section of the chamber condensation region to increase or decrease linear gas velocity for a given volumetric flow rate, and/or the presence or absence of structures that produce turbulence within the chamber. Thus, for example to produce condensation particles in the size range 20-100 nm MMAD, the chamber may provide gas-flow barriers for creating air turbulence within the condensation chamber. These barriers are typically placed within a few thousands of an inch from the substrate surface.

Typically, the flow rate of gas over the substrate ranges from about 4-50 L/min, preferably from about 5-30 L/min.

Prior to, simultaneous with, or subsequent to passing a gas over the substrate, heat is applied to the substrate to vaporize the drug composition film. It will be appreciated that the temperature to which the substrate is heated will vary according to the drug's vaporization properties, but is typically heated to a temperature of at least about 200° C., preferably of at least about 250° C., more preferably at least about 300° C. or 350° C. Heating the substrate produces a drug composition vapor that in the presence of the flowing gas generates aerosol particles in the desired size range. In one embodiment, the substrate is heated for a period of less than about 1 second, and more preferably for less than about 500 milliseconds, still more preferably for less than about 200 milliseconds. The drug-aerosol particles are inhaled by a subject for delivery to the lung.

Utility: Rapid-Heating Device and Method

In another general embodiment, there is provided a device for producing an aerosol of compound condensation particles, e.g., for use in inhalation therapy. The device has the elements described above with respect to FIGS. 2A and 2B, where the heat source is designed to supply heat to the substrate in the device at a rate effective to produce a substrate temperature greater than 200° C. or in other embodiments greater than 250° C., 300° C. or 350° C., and to substantially volatilize the drug composition film from the substrate in a period of 2 seconds or less. The thickness of the film of drug composition on the substrate is such that the device produces an aerosol containing less than 10% by weight drug degradation and at least 50% of the drug composition on the film.

The device includes a drug composition delivery assembly composed of the substrate, a film of the selected drug composition on the substrate surface, and a heat source for supplying heat to the substrate at a rate effective to heat the substrate to a temperature greater than 200° C. or in other embodiments to a temperature greater than 250° C., 300° C. or 350° C., and to produce substantially complete volatilization of the drug composition within a period of 2 seconds or less.

The drug composition in the assembly and device may be one that, when vaporized from a film on an impermeable surface of a heat conductive substrate, the aerosol exhibits an increasing level of drug degradation products with increasing film thicknesses, particularly at a thickness of greater than 0.05-20 microns. For this general group of drug compositions, the film thickness on the substrate will typically be between 0.05 and 20 microns, e.g., the maximum or near-maximum thickness within this range that allows formation of a particle aerosol with drug degradation less than 5%.

Alternatively, the drug may show less than 5-10% degradation even at film thicknesses greater than 20 microns. For these compounds, a film thickness greater than 20 microns, e.g., 20-50 microns, may be selected, particularly where a relatively large drug dose is desired.

The device is useful in a method for producing a condensation aerosol by the steps of heating the device substrate at a rate that heats the substrate to a temperature greater than 200° C., or in other embodiments to a temperature greater than 250° C., 300° C., or 350° C., and produces substantially complete volatilization of the compounds within a period of 2 seconds or less.

Alternative Drug-Supply Devices:

One embodiment of the present invention is a method for generating an aerosol comprising heating the physiologically active compound to vaporize the compound or at least a portion thereof, mixing the resulting vapor with a predetermined volume of a gas to form a desired particle size after a stable concentration of particles in the gas is reached, and then administering the resulting aerosol to the patient.

The following is a summary of various alternatives that can be taken to achieve the desired aerosol for administration to the patient in accordance with this embodiment of the present invention:

1. Simultaneous vaporization of the compound and mixing with air or other gas followed by condensation and aggregation to the desired particle size.

2. Vaporization of the compound to form a pure compound gas then followed by mixing with air or other gas, then condensation and aggregation to the desired particle size.

3. Simultaneous vaporization of the compound and mixing with a portion of the final volume of air or other gas, followed by additional mixing with the balance of the air, then by condensation and aggregation to desired particle size.

4. Vaporization of the compound followed by mixing with a small portion of air or other gas, then condensation, then aggregation to a desired particle size and then additional mixing the aerosol with the balance of the air. (1-3 micron method)

5. Simultaneous vaporization and mixing with a small portion of air or other gas followed by condensation and aggregation to a desired particle size and then additional mixing with the balance of the air. (1-3 micron method).

To create an ultra fine particle, as defined in the Background of Invention section, in an aerosol utilizing compounds with molecular weights between 100 and 300, 0.1 to 2 mg of each compound (depending on the compound) in its vapor-state are mixed into approximately one liter of air. This resulted in the desired concentration and once this concentration was achieved, aggregation slowed considerably, such that a “stable” particle size was achieved for the duration of time a patient would draw a breath to carry the particles into the lung. One liter of air is typically the amount of air that one would want to use, to deliver a compound to the lung.

One embodiment of creating ultra fine particles in an aerosol is to allow air to sweep over a thin film of the compound during the heating process. This allows the compound to become vaporize at a lower temperature due to the lowering of the partial pressure of the compound near the surface of the film.

Another embodiment is to introduce the compound into the air as a pure gas. This involved vaporizing the compound in a container and then injecting the vapor into a gas stream through a variety of mixing nozzles.

Yet another embodiment overcomes the problem that certain compounds that react rapidly with oxygen at elevated temperatures. To solve this problem, the compound is heated in a small container housing a small amount, e.g., about 1 to about 10 ml, of an inert gas. Once the compound is vaporized and is mixed with the inert gas while the gaseous mixture is maintained at a temperature sufficient to keep the compound in its gaseous state, the gaseous mixture is then injected into the air stream. The volume of inert gas can also be circulated over the surface of the heated compound to aid in its vaporization.

To create fine particles in the 1-3 micron range in an aerosol, the volume of air (or other gas) is reduced within which the compound is allowed to aggregate. This is done so the compound can condense and aggregate to the desired particle size at a point when the concentration is such that the particle size becomes stable. In producing fine particles, it is necessary to reduce the volume of the initial mixing gas. This leads to an increase in the concentration of the compound, which in turn results in a greater growth in particle size before the desired concentration is reached and aggregation slowed. When a stable particle size is reached in the smaller volume, the mixture is injected into the balance of the air. As in the above embodiments, this initial mixing stage can be, if needed, accomplished in the presence of an inert gas to reduce decomposition resulting from oxygenation.

Decomposition of the compound occurs by a variety of mechanisms, depending on the chemical nature of the compound to be volatilized. Thermal decomposition, the breaking and rearranging of chemical bonds as the compound absorbs increasing heat energy, is a major concern with the devices of the present invention. The present invention minimizes the temperature and time that the compound is exposed to elevated temperatures. Vitamin E, for example, decomposes by more than 90% when heated at 425° C. or higher for 5 minutes, but only 20% when the temperature is lowered to 350° C. This decomposition is lowered further to about 12% if the time is decreased to 30 seconds, and less than 2% if the time is decreased to 10-50 milliseconds. Similarly, fentanyl when heated to 200° C. for 30 seconds decomposed entirely, but when heated to 280° C. for 0.01 second only 15-30% of the compound is decomposed. Therefore, the device of the present invention can vaporize a drug such as vitamin E for administration directly to organs such as the lung or eye.

It is also advantageous that the temperature of vaporization be kept to a minimum. In order for the compound to be vaporized in 1 second and for the temperature to be kept to a minimum, rapid air movement across the surface of the compound is used.

In one aspect, the following parameters are imposed on a preferred device of the present invention, due to human lung physiology, the physics of aerosol growth, or the physical chemistry of desirable compounds: (1) The compound needs to be vaporized over approximately 1 second. (2) The compound needs to be raised to the vaporization temperature as rapidly as possible. (3) The compound, once vaporized, needs to be cooled as quickly as possible. (4) The compound needs to be raised to the maximum temperature for a maximum duration of time to minimize decomposition. (5) The air or other gas needs to be moved rapidly across the surface of the compound to achieve the required rate of vaporization. (6) The cross sectional area of the heating/vaporization zone decreases as the air speed increases across the compound being volatilized. (7) The heating of the air increases as the cross sectional area of the heating/vaporization decreases. (8) The air temperature should be kept to a minimum, i.e., an increase of no greater than about 15° C. (9) The compound needs to be mixed into the air at a consistent rate to have a consistent and repeatable particle size.

The parameters of the design for this preferred embodiment are the result of meeting and balancing the competing requirements listed above. One especially important requirement is that the compound, while needing to be vaporized over a 1 second period, also needs to have each segment of the compound exposed to as brief a heat up period as possible. In the preferred embodiment, the compound is deposited onto a foil substrate and an alternating magnetic field is swept along a foil substrate heating the substrate such that the compound is vaporized sequentially over no more than about a one second period of time. Because of the sweeping action of the magnetic field, each segment of the compound has a heat-up time that is much less than one second. Additionally a reduced cross section of the airway is established in the heating zone thereby increasing the speed of the mixing air in that section and that section alone. In this preferred embodiment, the compound is laid down on a thin metallic foil. In the example set forth below, stainless steel (alloy of 302, 304, or 316) was used in which the surface was treated to produce a rough texture. Other foil materials can be used but it is important that the surface and texture of the material is such that it is “wetted” by the compound, when the compound is in its liquid phase. When the compound is in the liquid phase, it is possible for it to “ball” up if the surface of the substrate is not of this type. If this happens, the compound can be blown by and picked up into the airflow without ever vaporizing. This leads to a particle size that is uncontrolled and undesirable.

Stainless steel has advantages over materials like aluminum by having a lower thermal conductivity value, while not having an appreciable increase in thermal mass. A low thermal conductivity is helpful because the heat generated should stay in the immediate area of interest.

In one example, the compound was deposited onto the stainless steel foil so that the thickness of the compound was less than 10 microns. The foil 6 was held in a frame 4 shown in FIGS. 31-35. The frame 4 should be made so that the trailing edge of the foil 6 has no lip on it so that the compound 5, once mixed with the air is free to travel downstream without causing turbulence. The foil 6 needs to have a constant cross section, because without it the electrical currents induced in the heating zone 3 will not be uniform. The frame 4 should be non-conductive, composed of a material that can withstand moderate heat (200° C.) and be non-chemically reactive with the compound. For this specific example, Ultem (PEI) was chosen as the material for frame 4.

The foil 6 was heated by placing it in an alternating magnetic field. It is preferable for the magnetic field to be confined in heating zone 3, the area that is being heated. In order to do this, a ferrite core 1 was used. When using a ferrite core 1, the alternating frequency of the field is limited to below 1 MHz. In this preferred embodiment a frequency between 100 and 300 kHz was used. For a given frequency and material, the skin depth of a magnetic field can be determined using Formula #3 below

$$σ = \sqrt{2} {ε}_{o} c$$

$$σ \dot{ω}$$

Where:
εₒ=8.85×10⁻¹²
c=speed of light in meters/second
σ=1.38×10⁶ for stainless steel (1/Ohm-meters)
\acute{ω}=frequency in radians.

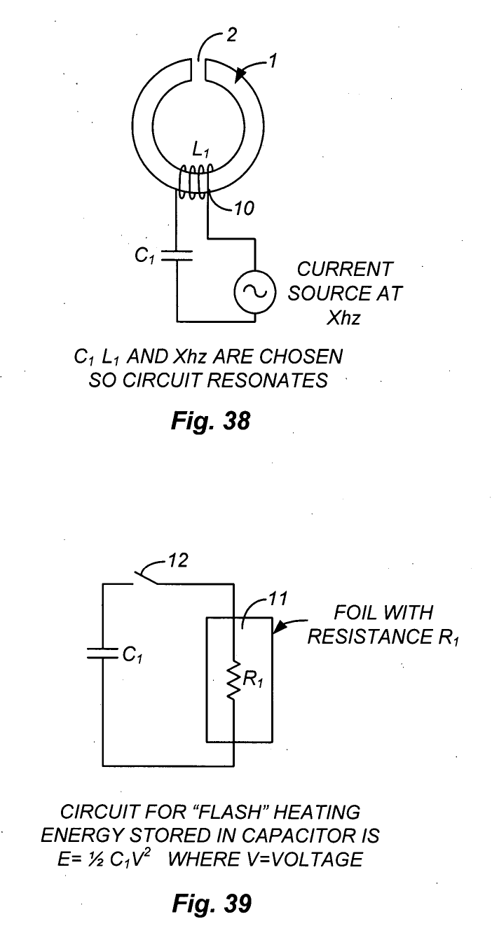

It is important to consider the skin depth because if the skin depth is much greater that the thickness of the foil, the magnetic field will pass through the foil and not induce any heating. The thicker the stainless steel foil that is used, the better the coupling of the magnetic field into the foil, but the more energy is needed to achieve a given temperature rise. A thickness for the foil 6 of 0.002 inches was chosen. The foil 6 in the frame 4 may be placed into a movable slide controlled by a motor, not shown. The slide allows the foil 6 to be moved through the magnetic field and thereby heated sequentially. In order to minimize the temperature to which the compound was exposed at the time of vaporization, a rapid movement of mixing air across the compound surface was utilized. This was best accomplished by making the cross section of the airflow small, thereby raising the speed of the air. This can cause the mixing air to be heated. Since the air is to be delivered to the lung, excessively heated air is not desirable. Restriction of the airflow, by decreasing the cross sectional area, also results in increasing the pressure drop through the device. For a device designed for human use, an upper reasonable limit to the pressure drop is 10 inches of water. To optimize these three considerations, increased air speed, minimizing temperature rise and minimizing pressure drop through the device, a narrowing section 9 of the cross section of tube 7 directly over the heating/vaporization zone 3 was used. The balance of the cross section of tube 7 was left large as this decreases to pressure drop. The narrow cross-section 9 is 0.05 inch resulting in an airflow speed of between about 10 to 50 meters per second. In this example, the airflow created an acceptable 8 to 12° C. temperature rise of the air. In order to have the magnetic field result in a narrow heating zone 3 on the foil 6 a ferrite toroid 1 with a narrow slit or air gap in it was employed to form the ferrite toroid 1. One of the advantages of this configuration, by laying the foil on its side, was that the effective thickness of the foil 6 relative to the skin depth of the magnetic field was increased. For this preferred embodiment, a ferrite toroid manufactured by the Fair Right Company was used. The slit 2 was 0.100 inch wide. FIG. 38 shows a typical circuit for ferrite toroid 1. Control and monitoring of the heat-up of the foil 6 was accomplished with a number of temperature measurement techniques including thermocouples and RTD's, not shown. This was accomplished in the present example by direct measurement of the magnetic field. Correlation back to the temperature is stored in a calibration table.

In this example, energy is stored in a capacitor in the form of electrical potential to result in flash vaporization of the compound in from about 0.001 to about 0.1 seconds. The energy stored in a capacitor is: ε=½ cv², where: c is capacitance in farads, V=voltage. This energy can be discharged into a resistive element through a switch. That switch can be in the form of a solid-state relay, or a contact closure. FIG. 39 shows the circuit. A thin layer of a drug was laid down on a thin foil of a conductive metal. It is preferable that the foil is of a material and size so that the internal resistance leads to a capacitive discharge rate resulting in a heat up rate that is desirable. The discharge rate of a capacitor is governed by the “RC” time constant. This states that the voltage in a capacitor will discharge 66% in a time that is the multiple of the resistance that the capacitor is discharged through (in Ohms) and the capacitance of the capacitor (in Farads). If too thick of a layer of compound is laid down for too fast a rate of heat up the compound will not be entirely vaporized but rather thrown off of the surface by the vaporized layer of compound lying directly adjacent to the foil. In other words if the layer is too thick or the heat-up rate too fast then the compound that is in direct contact with the foil is heated to the point of vaporization before the balance of the compound is heated. This causes some of the compound to be thrown from the surface by a portion of the compound that is vaporized. This results in a very uneven particle size distribution. For compounds similar to vitamin E and THC the limit of the layer thickness is a ratio no greater than ratio greater or equal to:

$$\frac{0.1 mg - 5 m sec}{3 {cm}^{2} - 300 ° C .}$$

If the desired dose of drug is 2 mg and the surface area of the drug layer is 3 cm, then the maximum temperature rise is 60° C. per millisecond. In this example, a 1 cm wide by 5 cm long and 0.0025 mm thick foil of alloy 316 or 304 stainless steel was connected to a capacitor. This results in a heat up of approximately 350° C. in 0.005 seconds.

Different sizes and materials for the foil can be chosen along with the value of the capacitor and the voltage it is changed to. With these choices one can control not only the temperature reached but also the rate of heat up. These materials could include aluminum, copper, brass, and other metallic and conductive materials. Composite materials can also be chosen for the following three reasons.

By choosing materials with different coefficients of thermal expansion, one can minimize the deflection of the foil upon heat up. By choosing a layer of non-reactive material to be placed and or adhered to the base material, decomposition of the compound can be reduced or eliminated. A non-conductive upper layer of material can be chosen so that the compound will be electrically insulated from the current flowing through the base material.

In another example, air is passed into thin walled tube having a coating of drug on inside of tube while mixing air is run through tube. This is another example that allows for rapid heat up while controlling the direction of the vaporized compound. A capacitor is then discharged through the tube while a carrier gas, e.g., air, N₂ and the like, is passed down the tube. Another advantage of this example is that if material is “thrown” from the interior wall of the tube before it can be vaporized it will be thrown onto the other side of the tube and vaporized there upon adhesion. The energy calculations that apply to the above are applicable to this example.

In yet another example, the compound is placed into a small sealed container, possibly a foil pouch, or a thin walled tube with a sealed end, and is heated. The gas that is generated is forced to leave the container. While rapid heating will in some instances preclude or retard decomposition, additional steps may need to be taken to lower the decomposition to an acceptable level. One of these steps is to remove or reduce the presence of oxygen during the heat up period. This example accomplishes this is by having the compound in the small sealed container with either no atmosphere in the container or in an inert gas atmosphere. Once the compound has become a gas it can then be ejected into an air stream as outlined later.

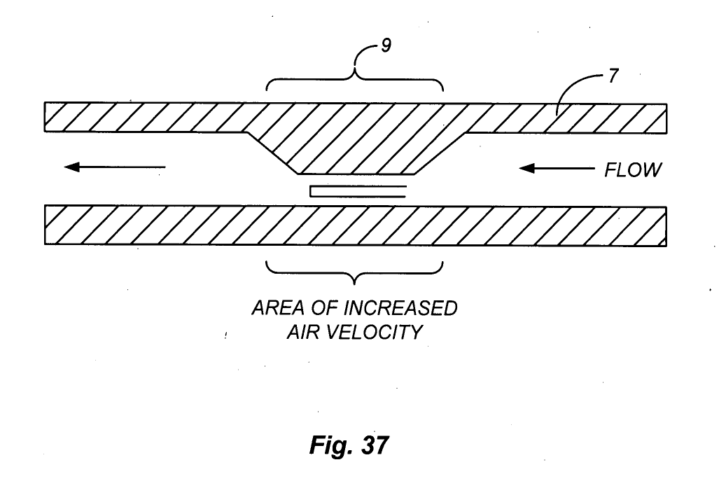

In this example, air is channeled though through a fine mesh screen that has had the drug deposited thereon as shown in FIG. 37 Rapid heating or rapid cooling, as stated above, can preclude decomposition. This example involves rapidly mixing the compound, once it has become and gas, into the air. A thin (0.01 to 10 micron) layer of compound can be deposited onto the fine meshed screen, e.g., 200 and 400 mesh screens have been used in this example.

Upon discharge of the capacitor, the screen is heated and the compound vaporized. Because there is air movement through the screen, once the compound becomes a gas it rapidly mixes with air and cools. This rapid cooling arrests decomposition. Stainless steel (304 alloy) has a desirable resistance when the dimensions are 2.54 cm by 2.54 cm. The current from the capacitor is passed between one edge and another. It is not necessary that the screen reach comparable temperatures as the thin foil because the compound becomes a gas at a lower temperature due to the rapid air movement. The rapid air movement allows for the compound to become a gas with a lower vapor pressure as the air is constantly removing the compound.

In yet another example, progressive heating is used in which multiple sections of substrate upon which is deposited the compound are heated in turn. In order to subject the compound to rapid heat up, while at the same time not vaporizing the compound all at once, a movable heating zone is used. In this example, a relatively small heating area, compared to the entire surface area that the compound is laid down on, was generated and moved, or “swept out” over compound deposition area. There are a number of specific means for accomplishing this as described below.

1) Moving Heater Relative to Substrate:

A variety of heating methods can be envisioned that would cause the heating of a zone in a substrate in which a compound has been laid down on, or directly heating a segment or portion of a compound. In the preferred embodiment described above, this heating method is an inductive heater, which heats a zone in a foil substrate. Regardless of the heating method, as long as only a zone of the compound and/or the substrate is heated it is possible to move the heater relative to the substrate/compound. In the preferred embodiment an inductive heating zone is induced in a conductive substrate that is in direct contact with the compound. The substrate is moved relative to this magnetic field, causing the compound to be locally vaporized.

2) Thermal Gradient:

An alternative method of producing a moving heating zone is to heat a thermally conductive substrate at one location and allow the thermal energy to travel across, or along the substrate. This produces, when looked at in a particular location, a heat up rate that is determined from the characteristics of the thermally conductive substrate. By varying the material and its cross sectional area it is possible to control the rate of heat up.

The source of the thermal energy can be from a variety of heating methods, including a simple resistive heater. This resistive heater can be held and/or imbedded in the substrate at an end, both ends, or in a variety of positions along the substrate, allowing the temperature gradient to move across the carrier and/or substrate.

3) Discrete Heating Zones:

Another method is to establish a set of heated zones, which are energized sequentially. These heating zones could be produced from any of the methods in the Rosen patent application including resistive heater. For example a substrate could have three (3) sections A, B, C where section A is first heated until the compound have been vaporized followed by the section B and so forth.

4) Inductive Heater, Vary Field to Heat Different Zones:

Another method is to heat a zone in a substrate with an inductive heater, and then by manipulating the magnetic field, cause the induced current in the substrate to move along the substrate. This can be accomplished by a number of methods, one of which is to use a ferrite that has a saturation value so that by increasing the electrical field internal to the ferrite the resultant magnetic field will leave the confines of the ferrite and enter a different area of the substrate. Another method is to construct a ferrite with a shape that can be changed, such as opening up an air gap, and by doing so changing the shape of the magnetic field.

5) The Use of Radiative Heating:

An additional method is to heat, incrementally a substrate through the focusing and/or de-focusing of photon energy. This would apply to all forms of photon, especially in the visible and IR spectrum.

Dosage of Drug Containing Aerosols:

The dose of a drug compound or compounds in aerosol form is generally no greater than twice the standard dose of the drug given orally. Typically, it will be equal to or less than 100% of the standard oral dose. Preferably, it will be less than 80%, and more preferably less than 40%, and most preferably less than 20% of the standard oral dose. For medications currently given intravenously, the drug dose in the aerosol will generally be similar to or less than the standard intravenous dose. Preferably it will be less than 200%, more preferably less than 100%, and most preferably less than 50% of the standard intravenous dose. Oral and/or intravenous doses for most drugs are readily available in the Physicians Desk Reference.

A dosage of a drug-containing aerosol may be administered in a single inhalation or may be administered in more than one inhalation, such as a series of inhalations. Where the drug is administered as a series of inhalations, the inhalations are typically taken within an hour or less (dosage equals sum of inhaled amounts). When the drug is administered as a series of inhalations, a different amount may be delivered in each inhalation.

The dose of a drug delivered in the aerosol refers to a unit dose amount that is generated by heating of the drug under defined conditions, cooling the ensuing vapor, and delivering the resultant aerosol. A “unit dose amount” is the total amount of drug in a given volume of inhaled aerosol. The unit dose amount may be determined by collecting the aerosol and analyzing its composition as described herein, and comparing the results of analysis of the aerosol to those of a series of reference standards containing known amounts of the drug. The amount of drug or drugs required in the starting composition for delivery as a aerosol depends on the amount of drug or drugs entering the thermal vapor phase when heated (i.e., the dose produced by the starting drug or drugs), the bioavailability of the aerosol drug or drugs, the volume of patient inhalation, and the potency of the aerosol drug or drugs as a function of plasma drug concentration.

One can determine the appropriate dose of a drug-containing aerosol to treat a particular condition using methods such as animal experiments and a dose-finding (Phase I/II) clinical trial. These experiments may also be used to evaluate possible pulmonary toxicity of the aerosol. One animal experiment involves measuring plasma concentrations of drug in an animal after its exposure to the aerosol. Mammals such as dogs or primates are typically used in such studies, since their respiratory systems are similar to that of a human and they typically provide accurate extrapolation of test results to humans. Initial dose levels for testing in humans are generally less than or equal to the dose in the mammal model that resulted in plasma drug levels associated with a therapeutic effect in humans. Dose escalation in humans is then performed, until either an optimal therapeutic response is obtained or a dose-limiting toxicity is encountered.

The actual effective amount of drug for a particular patient can vary according to the specific drug or combination thereof being utilized, the particular composition formulated, the mode of administration and the age, weight, and condition of the patient and severity of the episode being treated.

Particle Size:

Efficient aerosol delivery to the lungs requires that the particles have certain penetration and settling or diffusional characteristics. Deposition in the deep lungs occurs by gravitational settling and requires particles to have an effective settling size, defined as mass median aerodynamic diameter (MMAD), typically between 1-3.5 μm. For smaller particles, deposition to the deep lung occurs by a diffusional process that requires having a particle size in the 10-100 nm, typically 20-100 nm range. Particle sizes in the range between 0.1-1.0 μm, however, are generally too small to settle onto the lung wall and too massive to diffuse to the wall in a timely manner. These types of particles are typically removed from the lung by exhalation, and thus are generally not used to treat disease. Therefore, an inhalation drug-delivery device for deep lung delivery should produce an aerosol having particles in one of these two size ranges, preferably between about 1-3 μm MMAD. Typically, in order to produce particles having a desired MMAD, gas or air is passed over the solid support at a certain flow rate.

During the condensation stage the MMAD of the aerosol is increasing over time. Typically, in variations of the invention, the MMAD increases within the size range of 0.01-3 microns as the vapor condenses as it cools by contact with the carrier gas then further increases as the aerosol particles collide with each other and coagulate into larger particles. Most typically, the MMAD grows from <0.5 micron to >1 micron in less than 1 second. Thus typically, immediately after condensing into particles, the condensation aerosol MMAD doubles at least once per second, often at least 2, 4, 8, or 20 times per second. In other variations, the MMAD increases within the size range of 0.1-3 microns.

Typically, the higher the flow rate, the smaller the particles that are formed. Therefore, in order to achieve smaller or larger particles, the flow rate through the condensation region of the delivery device may be altered. A desired particle size is achieved by mixing a compound in its vapor-state into a volume of a carrier gas, in a ratio such that the desired particle size is achieved when the number concentration of the mixture reaches approximately 10⁹ particles/mL. The particle growth at this number concentration is then slow enough to consider the particle size to be “stable” in the context of a single deep inhalation. This may be done, for example, by modifying a gas-flow control valve to increase or decrease the volumetric airflow rate. To illustrate, condensation particles in the size range 1-3.5 μm MMAD may be produced by selecting the gas-flow rate to be in a range of 4-50 L/minute, preferably in the range of 5-30 L/min.

Additionally, as will be appreciated by one of skill in the art, particle size may also be altered by modifying the cross-section of the chamber condensation region to increase or decrease linear gas velocity for a given volumetric flow rate. In addition, particle size may also be altered by the presence or absence of structures that produce turbulence within the chamber. Thus, for example to produce condensation particles in the size range 10-100 nm MMAD, the chamber may provide gas-flow barriers for creating air turbulence within the condensation chamber. These barriers are typically placed within a few thousandths of an inch from the substrate surface.

Analysis of Drug Containing Aerosols:

Purity of a drug-containing aerosol may be determined using a number of different methods. Byproducts for example, are those unwanted products produced during vaporization. For example, byproducts include thermal degradation products as well as any unwanted metabolites of the active compound or compounds. Examples of suitable methods for determining aerosol purity are described in Sekine et al., Journal of Forensic Science 32:1271-1280 (1987) and in Martin et al., Journal of Analytic Toxicology 13:158-162 (1989).

One suitable method involves the use of a trap. In this method, the aerosol is collected in a trap in order to determine the percent or fraction of byproduct. Any suitable trap may be used. Suitable traps include filters, glass wool, impingers, solvent traps, cold traps, and the like. Filters are often most desirable. The trap is then typically extracted with a solvent, e.g. acetonitrile, and the extract subjected to analysis by any of a variety of analytical methods known in the art, for example, gas, liquid, and high performance liquid chromatography particularly useful.

The gas or liquid chromatography method typically includes a detector system, such as a mass spectrometry detector or an ultraviolet absorption detector. Ideally, the detector system allows determination of the quantity of the components of the drug composition and of the byproduct, by weight. This is achieved in practice by measuring the signal obtained upon analysis of one or more known mass(es) of components of the drug composition or byproduct (standards) and then comparing the signal obtained upon analysis of the aerosol to that obtained upon analysis of the standard(s), an approach well known in the art.

In many cases, the structure of a byproduct may not be known or a standard for it may not be available. In such cases, one may calculate the weight fraction of the byproduct by assuming it has an identical response coefficient (e.g. for ultraviolet absorption detection, identical extinction coefficient) to the drug component or components in the drug composition. When conducting such analysis, byproducts present in less than a very small fraction of the drug compound, e.g. less than 0.1% or 0.03% of the drug compound, are typically excluded. Because of the frequent necessity to assume an identical response coefficient between drug and byproduct in calculating a weight percentage of byproduct, it is often more desirable to use an analytical approach in which such an assumption has a high probability of validity. In this respect, high performance liquid chromatography with detection by absorption of ultraviolet light at 225 nm is typically desirable. UV absorption at 250 nm may be used for detection of compounds in cases where the compound absorbs more strongly at 250 nm or for other reasons one skilled in the art would consider detection at 250 nm the most appropriate means of estimating purity by weight using HPLC analysis. In certain cases where analysis of the drug by UV are not viable, other analytical tools such as GC/MS or LC/MS may be used to determine purity.

It is possible that modifying the form of the drug may impact the purity of the aerosol obtained. Although not always the case, the free base or free acid form of the drug as opposed to the salt, generally results in either a higher purity or yield of the resultant aerosol. Therefore, in certain circumstances, it may be more desirable to use the free base or free acid forms of the compounds used. Similarly, it is possible that changing the gas under which vaporization of the composition occurs may also impact the purity.

Other Analytical Methods:

Particle size distribution of a drug-containing aerosol may be determined using any suitable method in the art (e.g., cascade impaction). An Andersen Eight Stage Non-viable Cascade Impactor (Andersen Instruments, Smyrna, Ga.) linked to a furnace tube by a mock throat (USP throat, Andersen Instruments, Smyrna, Ga.) is one system used for cascade impaction studies.

Inhalable aerosol mass density may be determined, for example, by delivering a drug-containing aerosol into a confined chamber via an inhalation device and measuring the mass collected in the chamber. Typically, the aerosol is drawn into the chamber by having a pressure gradient between the device and the chamber, wherein the chamber is at lower pressure than the device. The volume of the chamber should approximate the inhalation volume of an inhaling patient, typically about 2 liters.

Inhalable aerosol drug mass density may be determined, for example, by delivering a drug-containing aerosol into a confined chamber via an inhalation device and measuring the amount of active drug compound collected in the chamber. Typically, the aerosol is drawn into the chamber by having a pressure gradient between the device and the chamber, wherein the chamber is at lower pressure than the device. The volume of the chamber should approximate the inhalation volume of an inhaling patient, typically about 2 liters. The amount of active drug compound collected in the chamber is determined by extracting the chamber, conducting chromatographic analysis of the extract and comparing the results of the chromatographic analysis to those of a standard containing known amounts of drug.

Inhalable aerosol particle density may be determined, for example, by delivering aerosol phase drug into a confined chamber via an inhalation device and measuring the number of particles of given size collected in the chamber. The number of particles of a given size may be directly measured based on the light-scattering properties of the particles. Alternatively, the number of particles of a given size may be determined by measuring the mass of particles within the given size range and calculating the number of particles based on the mass as follows: Total number of particles=Sum (from size range 1 to size range N) of number of particles in each size range. Number of particles in a given size range=Mass in the size range/Mass of a typical particle in the size range. Mass of a typical particle in a given size range=π*D3*φ/6, where D is a typical particle diameter in the size range (generally, the mean boundary MMADs defining the size range) in microns, φ is the particle density (in g/mL) and mass is given in units of picograms (g-12).

Rate of inhalable aerosol particle formation may be determined, for example, by delivering aerosol phase drug into a confined chamber via an inhalation device. The delivery is for a set period of time (e.g., 3 s), and the number of particles of a given size collected in the chamber is determined as outlined above. The rate of particle formation is equal to the number of 100 nm to 5 micron particles collected divided by the duration of the collection time.

Rate of aerosol formation may be determined, for example, by delivering aerosol phase drug into a confined chamber via an inhalation device. The delivery is for a set period of time (e.g., 3 s), and the mass of particulate matter collected is determined by weighing the confined chamber before and after the delivery of the particulate matter. The rate of aerosol formation is equal to the increase in mass in the chamber divided by the duration of the collection time. Alternatively, where a change in mass of the delivery device or component thereof can only occur through release of the aerosol phase particulate matter, the mass of particulate matter may be equated with the mass lost from the device or component during the delivery of the aerosol. In this case, the rate of aerosol formation is equal to the decrease in mass of the device or component during the delivery event divided by the duration of the delivery event.

Rate of drug aerosol formation may be determined, for example, by delivering a drug-containing aerosol into a confined chamber via an inhalation device over a set period of time (e.g., 3 s). Where the aerosol is a pure drug, the amount of drug collected in the chamber is measured as described above. The rate of drug aerosol formation is equal to the amount of drug collected in the chamber divided by the duration of the collection time. Where the drug-containing aerosol comprises a pharmaceutically acceptable excipient, multiplying the rate of aerosol formation by the percentage of drug in the aerosol provides the rate of drug aerosol formation.

Kits

In an embodiment of the invention, a kit is provided for use by a healthcare provider, or more preferably a patient. The kit for delivering a condensation aerosol typically comprises a composition comprising a drug, and a device for forming a condensation aerosol. The composition is typically void of solvents and excipients and generally comprises a heat stable drug. The device for forming a condensation aerosol typically comprises an element configured to heat the composition to form a vapor, an element allowing the vapor to condense to form a condensation aerosol, and an element permitting a user to inhale the condensation aerosol. The device in the kit may further comprise features such as breath-actuation or lockout elements. An exemplary kit will provide a hand-held aerosol delivery device and at least one dose.

In another embodiment, kits for delivering a drug aerosol comprising a thin film of a drug composition and a device for dispensing said film as a condensation aerosol are provided. The composition may contain pharmaceutical excipients. The device for dispensing said film of a drug composition as an aerosol comprises an element configured to heat the film to form a vapor, and an element allowing the vapor to condense to form a condensation aerosol.

In the kits of the invention, the composition is typically coated as a thin film, generally at a thickness between about 0.5-20 microns, on a substrate which is heated by a heat source. Heat sources typically supply heat to the substrate at a rate that achieves a substrate temperature of at least 200° C., preferably at least 250° C., or more preferably at least 300° C. or 350° C., and produces substantially complete volatilization of the drug composition from the substrate within a period of 2 seconds, preferably, within 1 second, or more preferably within 0.5 seconds. To prevent drug degradation, it is preferable that the heat source does not heat the substrate to temperature greater than 600° C. while the drug film is on the substrate to prevent. More preferably, the heat source does not heat the substrate in to temperatures in excess of 500° C.

The kit of the invention can be comprised of various combinations of drugs and drug delivery devices. In some embodiments the device may also be present with another drug. The other drug may be administered orally or topically. Generally, instructions for use are included in the kits.

Utility

As can be appreciated from the above examples showing generation of a pure drug condensation aerosol, from thin films (i.e. 0.05-20 μm) of the drug, the invention finds use in the medical field in compositions and kits for delivery of a drug. Thus, the invention includes, in one aspect, condensation aerosols.

These aerosols can be used for treating a variety of disease states and/or intermittent and acute conditions where rapid systemic absorption and therapeutic effect are highly desirable. Typically the methods of treatment comprise the step of administering a therapeutically effective amount of a drug condensation aerosol to a person with a condition or disease. Typically the step of administering the drug condensation aerosol comprises the step of administering an orally inhalable drug condensation aerosol to the person with the condition. The drug condensation aerosol may be administered in a single inhalation, or in more than one inhalation, as described above.

The drug condensation aerosol may comprise a drug composition as described above. The drug composition typically is a “heat stable drug”. In some variations, the condensation aerosol comprises at least one drug selected from the group consisting of acebutolol, acetaminophen, alprazolam, amantadine, amitriptyline, apomorphine diacetate, apomorphine hydrochloride, atropine, azatadine, betahistine, brompheniramine, bumetanide, buprenorphine, bupropion hydrochloride, butalbital, butorphanol, carbinoxamine maleate, celecoxib, chlordiazepoxide, chlorpheniramine, chlorzoxazone, ciclesonide, citalopram, clomipramine, clonazepam, clozapine, codeine, cyclobenzaprine, cyproheptadine, dapsone, diazepam, diclofenac ethyl ester, diflunisal, disopyramide, doxepin, estradiol, ephedrine, estazolam, ethacrynic acid, fenfluramine, fenoprofen, flecainide, flunitrazepam, galanthamine, granisetron, haloperidol, hydromorphone, hydroxychloroquine, ibuprofen, imipramine, indomethacin ethyl ester, indomethacin methyl ester, isocarboxazid, ketamine, ketoprofen, ketoprofen ethyl ester, ketoprofen methyl ester, ketorolac ethyl ester, ketorolac methyl ester, ketotifen, lamotrigine, lidocaine, loperamide, loratadine, loxapine, maprotiline, memantine, meperidine, metaproterenol, methoxsalen, metoprolol, mexiletine HCl, midazolam, mirtazapine, morphine, nalbuphine, naloxone, naproxen, naratriptan, nortriptyline, olanzapine, orphenadrine, oxycodone, paroxetine, pergolide, phenyloin, pindolol, piribedil, pramipexole, procainamide, prochloperazine, propafenone, propranolol, pyrilamine, quetiapine, quinidine, rizatriptan, ropinirole, sertraline, selegiline, sildenafit, spironolactone, tacrine, tadalafil, terbutaline, testosterone, thalidomide, theophylline, tocainide, toremifene, trazodone, triazolam, trifluoperazine, valproic acid, venlafaxine, vitamin E, zaleplon, zotepine, amoxapine, atenolol, benztropine, caffeine, doxylamine, estradiol 17-acetate, flurazepam, flurbiprofen, hydroxyzine, ibutilide, indomethacin norcholine ester, ketorolac norcholine ester, melatonin, metoclopramide, nabumetone, perphenazine, protriptyline HCl, quinine, triamterene, trimipramine, zonisamide, bergapten, chlorpromazine, colchicine, diltiazem, donepezil, eletriptan, estradiol-3,17-diacetate, efavirenz, esmolol, fentanyl, flunisolide, fluoxetine, hyoscyamine, indomethacin, isotretinoin, linezolid, meclizine, paracoxib, pioglitazone, rofecoxib, sumatriptan, tolterodine, tramadol, tranylcypromine, trimipramine maleate, valdecoxib, vardenafil, verapamil, zolmitriptan, zolpidem, zopiclone, bromazepam, buspirone, cinnarizine, dipyridamole, naltrexone, sotalol, telmisartan, temazepam, albuterol, apomorphine hydrochloride diacetate, carbinoxamine, clonidine, diphenhydramine, thambutol, fluticasone proprionate, fluconazole, lovastatin, lorazepam N,O-diacetyl, methadone, nefazodone, oxybutynin, promazine, promethazine, sibutramine, tamoxifen, tolfenamic acid, aripiprazole, astemizole, benazepril, clemastine, estradiol 17-heptanoate, fluphenazine, protriptyline, ethambutal, frovatriptan, pyrilamine maleate, scopolamine, and triamcinolone acetonide. In other variations, the drug is selected from the group consisting of alprazolam, amoxapine, apomorphine hydrochloride, atropine, bumetanide, buprenorphine, butorphanol, celecoxib, ciclesonide, clomipramine, donepezil, eletriptan, fentanyl, hydromorphone, loxapine, midazolam, morphine, nalbuphine, naratriptan, olanzapine, parecoxib, paroxetine, prochlorperazine, quetiapine, sertraline, sibutramine, sildenafil, sumatriptan, tadalafil, valdecoxib, vardenafil, venlafaxine, and zolpidem. In some variations, the drug condensation aerosol has a MMAD in the range of about 1-3 μm.

In another aspect of the invention, kits are provided that include a drug composition and a condensation aerosol delivery device for production of a thermal vapor that contains drug-aerosol particles. The drug delivery article in the device includes a substrate coated with a film of a drug composition to be delivered to a subject, preferably a human subject. The thickness of the drug composition film is selected such that upon vaporizing the film by heating the substrate to a temperature sufficient to vaporize at least 50% of the drug composition film, typically to a temperature of at least about 200° C., preferably at least about 250° C., more preferably at least about 300° C. or 350° C., a thermal vapor is generated that has 10% or less drug-degradation product. The area of the substrate is selected to provide a therapeutic dose, and is readily determined based on the equations discussed above.

### EXAMPLES

The following examples further illustrate the invention described herein and are in no way intended to limit the scope of the invention.

Materials

Solvents were of reagent grade or better and purchased commercially.

Unless stated otherwise, the drug free base or free acid form was used in the Examples.

Methods

A. Preparation of Drug-Coating Solution

Drug was dissolved in an appropriate solvent. Common solvent choices included methanol, dichloromethane, methyl ethyl ketone, diethyl ether, 3:1 chloroform:methanol mixture, 1:1 dichloromethane:methyl ethyl ketone mixture, dimethylformamide, and deionized water. Sonication and/or heat were used as necessary to dissolve the compound. The drug concentration was typically between 50-200 mg/mL.

B. Preparation of Drug-Coated Stainless Steel Foil Substrate

Strips of clean 304 stainless steel foil (0.0125 cm thick, Thin Metal Sales) having dimensions 1.3 cm by 7.0 cm were dip-coated with a drug solution. The foil was then partially dipped three times into solvent to rinse drug off of the last 2-3 cm of the dipped end of the foil. Alternatively, the drug coating from this area was carefully scraped off with a razor blade. The final coated area was between 2.0-2.5 cm by 1.3 cm on both sides of the foil, for a total area of between 5.2-6.5 cm2 Foils were prepared as stated above and then some were extracted with methanol or acetonitrile as standards. The amount of drug was determined from quantitative HPLC analysis. Using the known drug-coated surface area, the thickness was then obtained by:
film thickness (cm)=drug mass (g)/[drug density (g/cm³)×substrate area (cm²).
If the drug density is not known, a value of 1 g/cm³ is assumed. The film thickness in microns is obtained by multiplying the film thickness in cm by 10,000.

After drying, the drug-coated foil was placed into a volatilization chamber constructed of a Dehin® block (the airway) and brass bars, which served as electrodes. The dimensions of the airway were 1.3 cm high by 2.6 cm wide by 8.9 cm long. The drug-coated foil was placed into the volatilization chamber such that the drug-coated section was between the two sets of electrodes. After securing the top of the volatilization chamber, the electrodes were connected to a 1 Farad capacitor (Phoenix Gold). The back of the volatilization chamber was connected to a two micron Teflon® filter (Savillex) and filter housing, which were in turn connected to the house vacuum. Sufficient airflow was initiated (typically 30 L/min=1.5 m/sec), at which point the capacitor was charged with a power supply, typically to between 14-17 Volts. The circuit was closed with a switch, causing the drug-coated foil to resistively heat to temperatures of about 280-430° C. (as measured with an infrared camera (FLIR Thermacam SC3000)), in about 200 milliseconds. (For comparison purposes, see FIG. 4A, thermocouple measurement in still air.) After the drug had vaporized, airflow was stopped and the Teflon®V filter was extracted with acetonitrile. Drug extracted from the filter was analyzed generally by HPLC UV absorbance generally at 225 nm using a gradient method aimed at detection of impurities to determine percent purity. Also, the extracted drug was quantified to determine a percent yield, based on the mass of drug initially coated onto the substrate. A percent recovery was determined by quantifying any drug remaining on the substrate and chamber walls, adding this to the quantity of drug recovered in the filter and comparing it to the mass of drug initially coated onto the substrate.

C. Preparation of Drug-Coated Aluminum Foil Substrate

A substrate of aluminum foil (10 cm×5.5 cm; 0.0005 inches thick) was precleaned with acetone. A solution of drug in a minimal amount of solvent was coated onto the foil substrate to cover an area of approximately 7-8 cm×2.5 cm. The solvent was allowed to evaporate. The coated foil was wrapped around a 300 watt halogen tube (Feit Electric Company, Pico Rivera, Calif.), which was inserted into a glass tube sealed at one end with a rubber stopper. Sixty volts of alternating current (driven by line power controlled by a Variac) were run through the bulb for 5-15 seconds, or in some studies 90 V for 3.5-6 seconds, to generate a thermal vapor (including aerosol) which was collected on the glass tube walls. In some studies, the system was flushed through with argon prior to volatilization. The material collected on the glass tube walls was recovered and the following determinations were made: (1) the amount emitted, (2) the percent emitted, and (3) the purity of the aerosol by reverse-phase HPLC analysis with detection typically by absorption of 225 nm light. The initial drug mass was found by weighing the aluminum foil substrate prior to and after drug coating. The drug coating thickness was calculated in the same manner as described in Method B.

D. Preparation of Drug-Coated Stainless Steel Cylindrical Substrate

A hollow stainless steel cylinder with thin walls, typically 0.12 mm wall thickness, a diameter of 13 mm, and a length of 34 mm was cleaned in dichloromethane, methanol, and acetone, then dried, and fired at least once to remove any residual volatile material and to thermally passivate the stainless steel surface. The substrate was then dip-coated with a drug coating solution (prepared as disclosed in Method A). The dip-coating was done using a computerized dip-coating machine to produce a thin layer of drug on the outside of the substrate surface. The substrate was lowered into the drug solution and then removed from the solvent at a rate of typically 5-25 cm/sec. (To coat larger amounts of material on the substrate, the substrate was removed more rapidly from the solvent or the solution used was more concentrated.) The substrate was then allowed to dry for 30 minutes inside a fume hood. If either dimethylformamide (DMF) or a water mixture was used as a dip-coating solvent, the substrate was vacuum dried inside a desiccator for a minimum of one hour. The drug-coated portion of the cylinder generally has a surface area of 8 cm2. By assuming a unit density for the drug, the initial drug coating thickness was calculated. The amount of drug coated onto the substrates was determined in the same manner as that described in Method B: the substrates were coated, then extracted with methanol or acetonitrile and analyzed with quantitative HPLC methods, to determine the mass of drug coated onto the substrate.

The drug-coated substrate was placed in a surrounding glass tube connected at the exit end via Tygon® tubing to a filter holder fitted with a Teflon® filter (Savillex). The junction of the tubing and the filter was sealed with paraffin film. The substrate was placed in a fitting for connection to two 1 Farad capacitors wired in parallel and controlled by a high current relay. The capacitors were charged by a separate power source to about 18-22 Volts and most of the power was channeled to the substrate by closing a switch and allowing the capacitors to discharge into the substrate. The substrate was heated to a temperature of between about 300-500° C. (see FIGS. 5A & 5B) in about 100 milliseconds. The heating process was done under an airflow of 15 L/min, which swept the vaporized drug aerosol into a 2 micron Teflon® filter.

After volatilization, the aerosol captured on the filter was recovered for quantification and analysis. The quantity of material recovered in the filter was used to determine a percent yield, based on the mass of drug coated onto the substrate. The material recovered in the filter was also analyzed generally by HPLC UV absorbance at typically 225 nm using a gradient method aimed at detection of impurities, to determine purity of the thermal vapor. Any material deposited on the glass sleeve or remaining on the substrate was also recovered and quantified to determine a percent total recovery ((mass of drug in filter+mass of drug remaining on substrate and glass sleeve)/mass of drug coated onto substrate). For compounds without UV absorption GC/MS or LC/MS was used to determine purity and to quantify the recovery. Some samples were further analyzed by LC/MS to confirm the molecular weight of the drug and any degradants.

E. Preparation of Drug-Coated Stainless Steel Cylindrical Substrate

A hollow stainless steel cylinder like that described in Example D was prepared, except the cylinder diameter was 7.6 mm and the length was 51 mm. A film of a selected drug was applied as described in Example D.

Energy for substrate heating and drug vaporization was supplied by two capacitors (1 Farad and 0.5 Farad) connected in parallel, charged to 20.5 Volts. The airway, airflow, and other parts of the electrical set up were as described in Example D. The substrate was heated to a temperature of about 420° C. in about 50 milliseconds. After drug film vaporization, percent yield, percent recovery, and purity analysis were done as described in Example D.

F. Preparation of Drug-Coated Aluminum Foil Substrate

A solution of drug was coated onto a substrate of aluminum foil (5 cm²-150 cm²; 0.0005 inches thick). In some studies, the drug was in a minimal amount of solvent, which was allowed to evaporate. The coated foil was inserted into a glass tube in a furnace (tube furnace). A glass wool plug was placed in the tube adjacent to the foil sheet and an air flow of 2 L/min was applied. The furnace was heated to 200-550° C. for 30, 60, or 120 seconds. The material collected on the glass wool plug was recovered and analyzed by reverse-phase HPLC analysis with detection typically by absorption of 225 nm light or GC/MS to determine the purity of the aerosol.

G. Preparation of Drug-Coated Aluminum Foil Substrate

A substrate of aluminum foil (3.5 cm×7 cm; 0.0005 inches thick) was precleaned with acetone. A solution of drug in a minimal amount of solvent was coated onto the foil substrate. The solvent was allowed to evaporate. The coated foil was wrapped around a 300 watt halogen tube (Feit Electric Company, Pico Rivera, Calif.), which was inserted into a T-shaped glass tube sealed at two ends with parafilm. The parafilm was punctured with ten to fifteen needles for air flow. The third opening was connected to a 1 liter, 3-neck glass flask. The glass flask was further connected to a piston capable of drawing 1.1 liters of air through the flask. Ninety volts of alternating current (driven by line power controlled by a Variac) was run through the bulb for 6-7 seconds to generate a thermal vapor (including aerosol) which was drawn into the 1 liter flask. The aerosol was allowed to sediment onto the walls of the 1 liter flask for 30 minutes. The material collected on the flask walls was recovered and the following determinations were made: (1) the amount emitted, (2) the percent emitted, and (3) the purity of the aerosol by reverse-phase HPLC analysis with detection by typically by absorption of 225 nm light. Additionally, any material remaining on the substrate was collected and quantified.

### Example 1

Acebutolol (MW 336, melting point 123° C., oral dose 400 mg), a beta adrenergic blocker (cardiovascular agent), was coated on a stainless steel cylinder (8 cm2) according to Method D. 0.89 mg of drug was applied to the substrate, for a calculated drug film thickness of 1.1 μm. The substrate was heated as described in Method D at 20.5 V and purity of the drug-aerosol particles were determined to be 98.9%. 0.53 mg was recovered from the filter after vaporization, for a percent yield of 59.6%. A total mass of 0.81 mg was recovered from the test apparatus and substrate, for a total recovery of 91%.

High speed photographs were taken as the drug-coated substrate was heated to monitor visually formation of a thermal vapor. The photographs showed that a thermal vapor was initially visible 30 milliseconds after heating was initiated, with the majority of the thermal vapor formed by 130 milliseconds. Generation of the thermal vapor was complete by 500 milliseconds.

### Example 2

Acetaminophen (MW 151, melting point 171° C., oral dose 650 mg), an analgesic agent, was coated on an aluminum foil substrate (20 cm²) according to Method C. 2.90 mg of drug was applied to the substrate, for a calculated thickness of the drug film of 1.5 μm. The substrate was heated under argon as described in Method C at 60 V for 6 seconds. The purity of the drug-aerosol particles were determined to be >99.5%. 1.9 mg was recovered from the glass tube walls after vaporization, for a percent yield of 65.5%.

### Example 3

Albuterol (MW 239, melting point 158° C., oral dose 0.18 mg), a bronchodilator, was coated onto six stainless steel foil substrates (5 cm²) according to Method B. The calculated thickness of the drug film on each substrate ranged from about 1.5 μm to about 6.1 μm. The substrates were heated as described in Method B by charging the capacitors to 15 V. Purity of the drug-aerosol particles from each substrate was determined and the results are shown in FIG. 23.

Albuterol was also coated on a stainless steel cylinder (8 cm²) according to Method D. 1.20 mg of drug was applied to the substrate, for a calculated drug film thickness of 2.4 μm. The substrate was heated as described in Method D by charging the capacitors to 20.5 V. The purity of the drug-aerosol particles was determined to be 94.4%. 0.69 mg was recovered from the filter after vaporization, for a percent yield of 57.2%. A total mass of 0.9 mg was recovered from the test apparatus and substrate, for a total recovery of 73.5%.

### Example 4

Alprazolam (MW 309, melting point 229° C., oral dose 0.25 mg), an anti-anxiety agent (Xanax®), was coated onto 13 stainless steel cylinder substrates (8 cm²) according to Method D. The calculated thickness of the drug film on each substrate ranged from about 0.1 μm to about 1.4 μm. The substrates were heated as described in Method D by charging the capacitors to 20.5 V. Purity of the drug-aerosol particles from each substrate was determined and the results are shown in FIG. 21.

Another substrate (stainless steel cylinder, 8 cm²) was coated with 0.92 mg of drug, for a calculated drug film thickness of 1.2 μm. The substrate was heated as described in Method D by charging the capacitors to 22.5 V. Purity of the drug-aerosol particles was 99.8%. 0.61 mg was recovered from the filter after vaporization, for a percent yield of 66.2%. A total mass of 0.92 mg was recovered from the test apparatus and substrate, for a total recovery of 100%.

Alprazolam was also coated on an aluminum foil substrate (28.8 cm²) according to Method C. 2.6 mg of the drug was coated on the substrate for a calculated thickness of the drug film of 0.9 μm. The substrate was heated substantially as described in Method C at 75 V for 6 seconds. The purity of the drug-aerosol particles was determined to be 99.9%.

High speed photographs were taken as the drug-coated substrate according to Method D was heated to monitor visually formation of a thermal vapor. The photographs showed that a thermal vapor was initially visible 35 milliseconds after heating was initiated, with the majority of the thermal vapor formed by 100 milliseconds. Generation of the thermal vapor was complete by 400 milliseconds.

### Example 5

Amantadine (MW 151, melting point 192° C., oral dose 100 mg), a dopaminergic agent and an anti-infective agent, was coated on an aluminum foil substrate (20 cm²) according to Method C. A mass of 1.6 mg was coated onto the substrate and the calculated thickness of the drug film was 0.8 μm. The substrate was heated as described in Method C at 90 V for 4 seconds. The purity of the drug-aerosol particles was determined to be 100%. 1.5 mg was recovered from the glass tube walls after vaporization, for a percent yield of 93.8%.

### Example 6

Amitriptyline (MW 277, oral dose 50 mg), a tricyclic antidepressant, was coated on a piece of aluminum foil (20 cm²) according to Method C. The calculated thickness of the drug film was 5.2 μm. The substrate was heated as described in Method C at 90 V for 5 seconds. The purity of the drug-aerosol particles was determined to be 98.4%. 5.3 mg was recovered from the glass tube walls after vaporization, for a percent yield of 51.5%.

Amitriptyline was also coated on an identical substrate to a thickness of 1.1 μm. The substrate was heated as described in Method C under an argon atmosphere at 90 V for 3.5 seconds. The purity of the drug-aerosol particles was determined to be 99.3%. 1.4 mg was recovered from the glass tube walls after vaporization, for a percent yield of 63.6%.

Apomorphine diacetate (MW 351), a dopaminergic agent used as an anti-Parkinsonian drug, was coated on a piece of aluminum foil (20 cm²) according to Method C. The calculated thickness of the drug film was 1.1 μm. The substrate was heated as described in Method C at 90 V for 3 seconds. The purity of the drug-aerosol particles was determined to be 96.9%. 2 mg was recovered from the glass tube walls after vaporization, for a percent yield of 90.9%.

### Example 8

The hydrochloride salt form of apomorphine was also tested. Apomorphine hydrochloride (MW 304) was coated on a stainless steel foil (6 cm²) according to Method B. 0.68 mg of drug was applied to the substrate, for a calculated drug film thickness of 1.1 μm. The substrate was heated as described in Method B by charging the capacitor to 15 V. The purity of the drug-aerosol particles was determined to be 98.1%. 0.6 mg was recovered from the filter after vaporization, for a percent yield of 88.2%. A total mass of 0.68 mg was recovered from the test apparatus and substrate, for a total recovery of 100%.

### Example 9

The hydrochloride diacetate salt of apomorphine was also tested (MW 388). Apomorphine hydrochloride diacetate was coated on a piece of aluminum foil (20 cm²) according to Method C. The calculated thickness of the drug film was 1.0 μm. The substrate was heated as described in Method C at 90 V for 3 second. purity of the drug-aerosol particles was determined to be 94.0%. 1.65 mg was recovered from the glass tube walls after vaporization, for a percent yield of 86.8%.

### Example 10

Atropine (MW 289, melting point 116° C., oral dose 0.4 mg), an muscarinic antagonist, was coated on five stainless steel cylinder substrates (8 cm²) according to Method D. The calculated thickness of the drug films ranged from about 1.7 μm to 9.0 μm. The substrate was heated as described in Method D by charging the capacitors to 19 or 22 V. Purity of the drug-aerosol particles from each substrate was determined. The results are shown in FIG. 6. For the substrate having a drug film thickness of 1.7 μm, 1.43 mg of drug was applied to the substrate. After volatilization of drug from this substrate with a capacitor charged to 22 V, 0.95 mg was recovered from the filter, for a percent yield of 66.6%. The purity of the drug aerosol recovered from the filter was found to be 98.5%. A total mass of 1.4 mg was recovered from the test apparatus and substrate, for a total recovery of 98.2%.

High speed photographs were taken as the drug-coated substrate was heated to monitor visually formation of a thermal vapor. The photographs showed that a thermal vapor was initially visible 28 milliseconds after heating was initiated, with the majority of the thermal vapor formed by 90 milliseconds. Generation of the thermal vapor was complete by 140 milliseconds.

Azatadine (MW 290, melting point 126° C., oral dose 1 mg), an antihistamine, was coated on an aluminum foil substrate (20 cm²) according to Method C. 5.70 mg of drug was applied to the substrate, for a calculated thickness of the drug film of 2.9 μm. The substrate was heated as described in Method C at 60 V for 6 seconds. The purity of the drug-aerosol particles was determined to be 99.6%. 2.8 mg was recovered from the glass tube walls after vaporization, for a percent yield of 49.1%.

Another azatadine coated substrate was prepared according to Method G. The substrate was heated as described in Method G at 60 V for 6 seconds under an argon atmosphere. The purity of the drug-aerosol particles was determined to be 99.6%. The percent yield of the aerosol was 62%.

### Example 12

Bergapten (MW 216, melting point 188° C., oral dose 35 mg), an anti-psoriatic agent, was coated on a stainless steel cylinder (8 cm²) according to Method D. 1.06 mg of drug was applied to the substrate, for a calculated drug film thickness of 1.3 μm. The substrate was heated as described in Method D by charging the capacitors to 20.5 V. The purity of the drug-aerosol particles was determined to be 97.8%. 0.72 mg was recovered from the filter after vaporization, for a percent yield of 67.9%. A total mass of 1.0 mg was recovered from the test apparatus and substrate, for a total recovery of 98.1%.

High speed photographs were taken as the drug-coated substrate was heated to monitor visually formation of a thermal vapor. The photographs showed that a thermal vapor was initially visible 40 milliseconds after heating was initiated, with the majority of the thermal vapor formed by 85 milliseconds. Generation of the thermal vapor was complete by 140 milliseconds.

### Example 13

Betahistine (MW 136, melting point <25° C., oral dose 8 mg), a vertigo agent, was coated on a metal substrate according to Method F and heated to 300° C. to form drug-aerosol particles. Purity of the drug-aerosol particles was determined to be 99.3%. 17.54 mg was recovered from the glass wool after vaporization, for a percent yield of 58.5%.

### Example 14

Brompheniramine (MW 319, melting point <25° C., oral dose 4 mg), an anti-histamine agent, was coated on an aluminum foil substrate (20 cm²) according to Method C. 4.50 mg of drug was applied to the substrate, for a calculated thickness of the drug film of 2.3 μm. The substrate was heated as described in Method C at 60 V for 8 seconds. The purity of the drug-aerosol particles was determined to be 99.8%. 3.12 mg was recovered from the glass tube walls after vaporization, for a percent yield of 69.3%.

An identical substrate with the same thickness of brompheniramine (4.5 mg drug applied to substrate) was heated under an argon atmosphere at 60 V for 8 seconds. The purity of the drug-aerosol particles was determined to be 99.9%. 3.3 mg was recovered from the glass tube walls after vaporization, for a percent yield of 73.3%.

The maleate salt form of the drug was also tested. Brompheniramine maleate (MW 435, melting point 134° C., oral dose 2 mg) was coated onto an aluminum foil substrate (20 cm²) according to Method C. The calculated thickness of the drug film was 2.8 μm. The substrate was heated as described in Method C at 60 V for 7 seconds. The purity of the drug-aerosol particles was determined to be 99.6%. 3.4 mg was recovered from the glass tube walls after vaporization, for a percent yield of 60.7%.

An identical substrate with a 3.2 μm brompheniramine maleate film was heated under an argon atmosphere at 60 V for 7 seconds. The purity of the drug-aerosol particles was determined to be 100%. 3.2 mg was recovered from the glass tube walls after vaporization, for a percent yield of 50%.

### Example 15

Bumetanide (MW 364, melting point 231° C., oral dose 0.5 mg), a cardiovascular agent and diuretic, was coated on a stainless steel cylinder (8 cm²) according to Method D. 1.09 mg of drug was applied to the substrate, for a calculated drug film thickness of 1.3 μm. The substrate was heated as described in Method D by charging the capacitors to 20.5 V. The purity of the drug-aerosol particles was determined to be 98.4%. 0.56 mg was recovered from the filter after vaporization, for a percent yield of 51.4%. A total mass of 0.9 mg was recovered from the test apparatus and substrate, for a total recovery of 82.6%.

High speed photographs were taken as the drug-coated substrate was heated to monitor visually formation of a thermal vapor. The photographs showed that a thermal vapor was initially visible 40 milliseconds after heating was initiated, with the majority of the thermal vapor formed by 300 milliseconds. Generation of the thermal vapor was complete by 1200 milliseconds.

### Example 16

Buprenorphine (MW 468, melting point 209° C., oral dose 0.3 mg), an analgesic narcotic, was coated on a piece of aluminum foil (20 cm²) according to Method C. The calculated thickness of the drug film was 0.7 μm. The substrate was heated as described in Method C at 60 V for 5 seconds. The purity of the drug-aerosol particles was determined to be 98%. 1.34 mg was recovered from the glass tube walls after vaporization, for a percent yield of 95.7%.

Buprenorphine was also coated onto five stainless steel cylinder substrates (8 cm2) according to Method D except that a 1.5 Farad capacitor was used as opposed to a 2.0 Farad capacitor. The calculated thickness of the drug film on each substrate ranged from about 0.3 μm to about 1.5 μm. The substrates were heated as described in Method D (with the single exception that the circuit capacitance was 1.5 Farad, not 2.0 Farad) and purity of the drug-aerosol particles determined. The results are shown in FIG. 9. For the substrate having a 1.5 μm drug film, 1.24 mg of drug was applied to the substrate. After volatilization of drug from this substrate by charging the capacitors to 20.5 V, 0.865 mg was recovered from the filter, for a percent yield of 69.5%. A total mass of 1.2 mg was recovered from the test apparatus and substrate, for a total recovery of 92.9%. The purity of the drug aerosol recovered from the filter was determined to be 97.1%.

High speed photographs were taken as one of the drug-coated substrates was heated, to monitor visually formation of a thermal vapor. The photographs, shown in FIGS. 26A-26E, showed that a thermal vapor was initially visible 30 milliseconds after heating was initiated, with the majority of the thermal vapor formed by 120 milliseconds. Generation of the thermal vapor was complete by 300 milliseconds.

The salt form of the drug, buprenorphine hydrochloride (MW 504), was also tested. The drug was coated on a piece of aluminum foil (20 cm2) according to Method C. 2.10 mg of drug was applied to the substrate, for a calculated thickness of the drug film of 1.1 μm. The substrate was heated as described in Method C at 60 V for 15 seconds. The purity of the drug-aerosol particles was determined to be 91.4%. 1.37 mg was recovered from the glass tube walls after vaporization, for a percent yield of 65.2%.

Buprenorphine was further coated on an aluminum foil substrate (24.5 cm2) according to Method G. 1.2 mg of the drug was applied to the substrate, for a calculated thickness of the drug film of 0.49 μm. The substrate was heated substantially as described in Method G at 90 V for 6 seconds, except that two of the openings of the T-shaped tube were left open and the third connected to the 1 L flask. The purity of the drug-aerosol particles was determined to be >99%. 0.7 mg of the drug was found to have aerosolized, for a percent yield of 58%.

### Example 17

Bupropion hydrochloride (MW 276, melting point 234° C., oral dose 100 mg), an antidepressant psychotherapeutic agent, was coated on a piece of aluminum foil (20 cm2) according to Method C. The calculated thickness of the drug film was 1.2 μm. The substrate was heated as described in Method C at 90 V for 3.5 seconds. The purity of the drug-aerosol particles was determined to be 98.5%. 2.1 mg was recovered from the glass tube walls after vaporization, for a percent yield of 91.3%. An identical substrate having the same drug film thickness was heated under an argon atmosphere according to Method C at 90 V for 3.5 seconds. 1.8 mg was recovered from the glass tube walls after vaporization, for a percent yield of 78.3%. The recovered vapor had a purity of 99.1%.

### Example 18

Butalbital (MW 224, melting point 139° C., oral dose 50 mg), a sedative and hypnotic barbituate, was coated on a piece of aluminum foil (20 cm2) according to Method C. 2.3 mg were coated on the foil, for a calculated thickness of the drug film of 1.2 μm. The substrate was heated as described in Method C at 90 V for 3.5 seconds. The purity of the drug-aerosol particles was determined to be >99.5%. 1.69 mg were collected for a percent yield of 73%.

### Example 19

Butorphanol (MW 327, melting point 217° C., oral dose 1 mg), an analgesic narcotic agent, was coated on a piece of aluminum foil (20 cm2) according to Method C. The calculated thickness of the drug film was 1.0 μm. The substrate was heated as described in Method C at 90 V for 3.5 seconds. The purity of the drug-aerosol particles was determined to be 98.7%.

Butorphanol was also coated on a stainless steel cylinder (6 cm2) according to Method E. 1.24 mg of drug was applied to the substrate, for a calculated drug film thickness of 2.1 μm. The substrate was heated as described in Method E and purity of the drug-aerosol particles was determined to be 99.4%. 0.802 mg was recovered from the filter after vaporization, for a percent yield of 64.7%. A total mass of 1.065 mg was recovered from the test apparatus and substrate, for a total recovery of 85.9%.

High speed photographs were taken as the drug-coated substrate was heated to monitor visually formation of a thermal vapor. The photographs showed that a thermal vapor was initially visible 35 milliseconds after heating was initiated, with the majority of the thermal vapor formed by 60 milliseconds. Generation of the thermal vapor was complete by 90 milliseconds.

### Example 20

Carbinoxamine (MW 291, melting point <25° C., oral dose 2 mg), an antihistamine, was coated on a piece of aluminum foil (20 cm2) according to Method C. 5.30 mg of drug was applied to the substrate, for a calculated thickness of the drug film of 2.7 μm. The substrate was heated as described in Method C at 60 V for 6 seconds. The purity of the drug-aerosol particles was determined to be 92.5%. 2.8 mg was recovered from the glass tube walls after vaporization, for a percent yield of 52.8%.

A second substrate was coated with carbinoxamine (6.5 mg drug) to a thickness of 3.3 μm. The substrate was heated as described in Method C at 90 V for 6 seconds under an argon atmosphere. The purity of the drug-aerosol particles determined was to be 94.8%. 3.1 mg was recovered from the glass tube walls after vaporization, for a percent yield of 47.7%.

The maleate salt form of the drug was also tested. Carbinoxamine maleate (MW 407, melting point 119° C., oral dose 4 mg) was coated on a piece of aluminum foil (20 cm2) according to Method C. The calculated thickness of the drug film was 3.9 μm. The substrate was heated as described in Method C at 90 V for 6 seconds. The purity of the drug-aerosol particles was determined to be 99%. 4.8 mg was recovered from the glass tube walls after vaporization, for a percent yield of 62.3%.

### Example 21

Celecoxib (MW 381, melting point 159° C., oral dose 100 mg), an analgesic non-steroidal anti-inflammatory agent, was coated on a piece of stainless steel foil (5 cm2) according to Method B. 4.6 mg of drug was applied to the substrate, for a calculated drug film thickness of 8.7 μm. The substrate was heated as described in Method B by charging the capacitors to 16 V. The purity of the drug-aerosol particles was determined to be >99.5%. 4.5 mg was recovered from the filter after vaporization, for a percent yield of 97.8%. A total mass of 4.6 mg was recovered from the test apparatus and substrate, for a total recovery of 100%.

Celecoxib was also coated on a piece of aluminum foil (100 cm2) according to Method G. The calculated thickness of the drug film was 3.1 μm. The substrate was heated as described in Method G at 60 V for 15 seconds. The purity of the drug-aerosol particles was determined to be 99%. 24.5 mg was recovered from the glass tube walls after vaporization, for a percent yield of 79%.

### Example 22

Chlordiazepoxide (MW 300, melting point 237° C., oral dose 5 mg), a sedative and hypnotic agent, was coated on a piece of aluminum foil (20 cm2) according to Method C. The calculated thickness of the drug film was 2.3 μm. The substrate was heated as described in Method C at 45 V for 15 seconds. The purity of the drug-aerosol particles was determined to be 98.2%. 2.5 mg was recovered from the glass tube walls after vaporization, for a percent yield of 54.3%.

### Example 23

Chlorpheniramine (MW 275, melting point <25° C., oral dose 4 mg), an antihistamine, was coated onto an aluminum foil substrate (20 cm2) according to Method C. 5.90 mg of drug was applied to the substrate, for a calculated thickness of the drug film of 3 μm. The substrate was heated as described in Method C at 60 V for 10 seconds. The purity of the drug-aerosol particles was determined to be 99.8%. 4.14 mg was recovered from the glass tube walls after vaporization, for a percent yield of 70.2%.

The maleate salt form (MW 391, melting point 135° C., oral dose 8 mg) was coated on an identical substrate to a thickness of 1.6 μm. The substrate was heated as described in Method C at 60 V for 7 seconds. The purity of the drug-aerosol particles was determined to be 99.6%. 2.1 mg was recovered from the glass tube walls after vaporization, for a percent yield of 65.6%.

### Example 24

Chlorpromazine (MW 319, melting point <25° C., oral dose 300 mg), an antipsychotic, psychotherapeutic agent, was coated on an aluminum foil substrate (20 cm2) according to Method C. 9.60 mg of drug was applied to the substrate, for a calculated thickness of the drug film of 4.8 μm. The substrate was heated as described in Method C at 90 V for 5 seconds. The purity of the drug-aerosol particles was determined to be 96.5%. 8.6 mg was recovered from the glass tube walls after vaporization, for a percent yield of 89.6%.

### Example 25

Chlorzoxazone (MW 170, melting point 192° C., oral dose 250 mg), a muscle relaxant, was coated on a piece of aluminum foil (20 cm2) according to Method C. The calculated thickness of the drug film was 1.3 μm. The substrate was heated as described in Method C at 90 V for 3.5 seconds. The purity of the drug-aerosol particles was determined to be 99.7%. 1.55 mg was recovered from the glass tube walls after vaporization, for a percent yield of 59.6%.

### Example 26

Ciclesonide free base (MW 541, melting point 206.5-207° C., oral dose 0.2 mg) a glucocorticoid, was coated on stainless steel foil substrates (6 cm2) according to Method B. Eight substrates were prepared, with the drug film thickness ranging from about 0.4 μm to about 2.4 μm. The substrates were heated as described in Method B, with the capacitors charged with 15.0 or 15.5 V. Purity of the drug-aerosol particles from each substrate was determined and the results are shown in FIG. 11. The substrate having a thickness of 0.4 μm was prepared by depositing 0.204 mg drug on the substrate surface. After volatilization of drug from this substrate using capacitors charged to 15.0 V, 0.201 mg was recovered from the filter, for a percent yield of 98.5%. The purity of the drug aerosol particles was determined to be 99%. A total mass of 0.204 mg was recovered from the test apparatus and substrate, for a total recovery of 100%.

### Example 27

Citalopram (MW 324, melting point <25° C., oral dose 20 mg), a psychotherapeutic agent, was coated onto an aluminum foil substrate (20 cm2) according to Method C. 8.80 mg of drug was applied to the substrate, for a calculated thickness of the drug film of 4.4 μm. The substrate was heated as described in Method C at 90 V for 4 seconds. The purity of the drug-aerosol particles was determined to be 92.3%. 5.5 mg was recovered from the glass tube walls after vaporization, for a percent yield of 62.5%.

Another substrate containing citalopram coated (10.10 mg drug) to a film thickness of 5 μm was prepared by the same method and heated under an argon atmosphere. The purity of the drug-aerosol particles was determined to be 98%. 7.2 mg was recovered from the glass tube walls after vaporization, for a percent yield of 71.3%.

### Example 28

Clomipramine (MW 315, melting point <25° C., oral dose 150 mg), a psychotherapeutic agent, was coated onto eight stainless steel cylindrical substrates according to Method E. The calculated thickness of the drug film on each substrate ranged from about 0.8 μm to about 3.9 μm. The substrates were heated as described in Method E and purity of the drug-aerosol particles determined. The results are shown in FIG. 10. For the substrate having a drug film thickness of 0.8 μm, 0.46 mg of drug was applied to the substrate. After volatilization of drug from this substrate, 0.33 mg was recovered from the filter, for a percent yield of 71.7%. Purity of the drug-aerosol particles was determined to be 99.4%. A total mass of 0.406 mg was recovered from the test apparatus and substrate, for a total recovery of 88.3%.

High speed photographs were taken as the drug-coated substrate was heated to monitor visually formation of a thermal vapor. The photographs showed that a thermal vapor was initially visible 40 milliseconds after heating was initiated, with the majority of the thermal vapor formed by 75 milliseconds. Generation of the thermal vapor was complete by 115 milliseconds.

### Example 29

Clonazepam (MW 316, melting point 239° C., oral dose 1 mg), an anticonvulsant, was coated on an aluminum foil substrate (50 cm2) and heated according to Method F to a temperature of 350° C. to form drug-aerosol particles. 46.4 mg of the drug was applied to the substrate, for a calculated thickness of the drug film of 9.3 μm. Purity of the drug-aerosol particles was determined to be 14%.

Clonazepam was further coated on an aluminum foil substrate (24 cm2) according to Method C. 5 mg of the drug was applied to the substrate, for a calculated thickness of the drug film of 2.1 μm. The substrate was heated substantially as described in Method C at 60 V for 8 seconds. The purity of the drug-aerosol particles was determined to be 99.9%.

### Example 30

Clonidine (MW 230, melting point 130° C., oral dose 0.1 mg), a cardiovascular agent, was coated on an aluminum foil substrate (50 cm2) and heated according to Method F at 300° C. to form drug-aerosol particles. Purity of the drug-aerosol particles was determined to be 94.9%. The yield of aerosol particles was 90.9%.

### Example 31

Clozapine (MW 327, melting point 184° C., oral dose 150 mg), a psychotherapeutic agent, was coated on an aluminum foil substrate (20 cm2) according to Method C. 14.30 mg of drug was applied to the substrate, for a calculated thickness of the drug film of 7.2 μm. The substrate was heated as described in Method C at 90 V for 5 seconds. The purity of the drug-aerosol particles was determined to be 99.1%. 2.7 mg was recovered from the glass tube walls after vaporization, for a percent yield of 18.9%.

Another substrate containing clozapine coated (2.50 mg drug) to a film thickness of 1.3 μm was prepared by the same method and heated under an argon atmosphere at 90 V for 3.5 seconds. The purity of the drug-aerosol particles was determined to be 99.5%. 1.57 mg was recovered from the glass tube walls after vaporization, for a percent yield of 62.8%.

### Example 32

Codeine (MW 299, melting point 156° C., oral dose 15 mg), an analgesic, was coated on an aluminum foil substrate (20 cm2) according to Method C. 8.90 mg of drug was applied to the substrate, for a calculated thickness of the drug film of 4.5 μm. The substrate was heated as described in Method C at 90 V for 5 seconds. The purity of the drug-aerosol particles was determined to be 98.1%. 3.46 mg was recovered from the glass tube walls after vaporization, for a percent yield of 38.9%.

Another substrate containing codeine coated (2.0 mg drug) to a film thickness of 1 μm was prepared by the same method and heated under an argon atmosphere at 90 V for 3.5 seconds. The purity of the drug-aerosol particles was determined to be >99.5%. 1 mg was recovered from the glass tube walls after vaporization, for a percent yield of 50%.

### Example 33

Colchicine (MW 399, melting point 157° C., oral dose 0.6 mg), a gout preparation, was coated on a stainless steel cylinder (8 cm2) according to Method D. 1.12 mg of drug was applied to the substrate, for a calculated drug film thickness of 1.3 μm. The substrate was heated as described in Method D by charging the capacitors to 20.5 V. The purity of the drug-aerosol particles was determined to be 97.7%. 0.56 mg was recovered from the filter after vaporization, for a percent yield of 50%. A total mass of 1.12 mg was recovered from the test apparatus and substrate, for a total recovery of 100%.

High speed photographs were taken as the drug-coated substrate was heated to monitor visually formation of a thermal vapor. The photographs showed that a thermal vapor was initially visible 30 milliseconds after heating was initiated, with the majority of the thermal vapor formed by 140 milliseconds. Generation of the thermal vapor was complete by 700 milliseconds.

### Example 34

Cyclobenzaprine (MW 275, melting point <25° C., oral dose 10 mg), a muscle relaxant, was coated on an aluminum foil substrate (20 cm2) according to Method C. 9.0 mg of drug was applied to the substrate, for a calculated thickness of the drug film of 4.5 μm. The substrate was heated as described in Method C at 90 V for 5 seconds. The purity of the drug-aerosol particles was determined to be 99%. 6.33 mg was recovered from the glass tube walls after vaporization, for a percent yield of 70.3%.

### Example 35

Cyproheptadine (MW 287, melting point 113° C., oral dose 4 mg), an antihistamine, was coated on an aluminum foil substrate (20 cm2) according to Method C. 4.5 mg of drug was applied to the substrate, for a calculated thickness of the drug film of 2.3 μm. The substrate was heated as described in Method C at 60 V for 8 seconds. The purity of the drug-aerosol particles was determined to be >99.5%. 3.7 mg was recovered from the glass tube walls after vaporization, for a percent yield of 82.2%.

Cyproheptadine HCl salt (MW 324, melting point 216° C., oral dose 4 mg) was coated on an identical substrate to a thickness of 2.2 μm. The substrate was heated at 60V for 8 seconds. The purity of the drug-aerosol particles was determined to be 99.6%. 2.6 mg was recovered from the glass tube walls after vaporization, for a percent yield of 60.5%.

### Example 36

Dapsone (MW 248, melting point 176° C., oral dose 50 mg), an anti-infective agent, was coated on a stainless steel cylinder (8 cm2) according to Method D. 0.92 mg of drug was applied to the substrate, for a calculated drug film thickness of 1.1 μm. The substrate was heated as described in Method D by charging the capacitors to 20.5 V. The purity of the drug-aerosol particles was determined to be >99.5%. 0.92 mg was recovered from the filter after vaporization, for a percent yield of 100%. The total mass was recovered from the test apparatus and substrate, for a total recovery of about 100%.

### Example 37

Diazepam (MW 285, melting point 126° C., oral dose 2 mg), a sedative and hypnotic, was coated on an aluminum foil substrate (20 cm2) according to Method C. 5.30 mg of drug was applied to the substrate, for a calculated thickness of the drug film of 2.7 μm. The substrate was heated as described in Method C at 40 V for 17 seconds. The purity of the drug-aerosol particles were determined to be 99.9%. 4.2 mg was recovered from the glass tube walls after vaporization, for a percent yield of 79.2%.

Diazepam was also coated on a circular aluminum foil substrate (78.5 cm2). 10.0 mg of drug was applied to the substrate, for a calculated film thickness of the drug of 1.27 μm. The substrate was secured to the open side of a petri dish (100 mm diameter×50 mm height) using parafilm. The glass bottom of the petri dish was cooled with dry ice, and the aluminum side of the apparatus was placed on a hot plate at 240° C. for 10 seconds. The material collected on the beaker walls was recovered and analyzed by HPLC analysis with detection by absorption of 225 nm light used to determine the purity of the aerosol. Purity of the drug-aerosol particles was determined to be 99.9%.

Diazepam was also coated on an aluminum foil substrate (36 cm2) according to Method G. 5.1 mg of drug was applied to the substrate, for a calculated thickness of the drug film of 1.4 μm. The substrate was heated substantially as described in Method G, except that 90 V for 6 seconds was used, and purity of the drug-aerosol particles was determined to be 99%. 3.8 mg was recovered from the glass tube walls after vaporization, for a percent yield of 74.5%.

### Example 38

Diclofenac ethyl ester (MW 324, oral dose 50 mg), an antirheumatic agent, was coated on a metal substrate (50 cm2) and heated according to Method F at 300° C. to form drug-aerosol particles. 50 mg of drug was applied to the substrate, for a calculated thickness of the drug film of 10 μm. Purity of the drug-aerosol particles was determined to be 100% by GC analysis. The yield of aerosol particles was 80%.

### Example 39

Diflunisal (MW 250, melting point 211° C., oral dose 250 mg), an analgesic, was coated on a piece of aluminum foil (20 cm2) according to Method C. The calculated thickness of the drug film was 5.3 μm. The substrate was heated as described in Method C at 60 V for 6 seconds. The purity of the drug-aerosol particles was determined to be >99.5%. 5.47 mg was recovered from the glass tube walls after vaporization, for a percent yield of 51.6%.

### Example 40

Diltiazem (MW 415, oral dose 30 mg), a calcium channel blocker used as a cardiovascular agent, was coated on a stainless steel cylinder (8 cm2) according to Method D. 0.8 mg of drug was applied to the substrate, for a calculated drug film thickness of 1 μm. The substrate was heated as described in Method D by charging the capacitors to 20.5V. The purity of the drug-aerosol particles was determined to be 94.2%: 0.53 mg was recovered from the filter after vaporization, for a percent yield of 66.3%. A total mass of 0.8 mg was recovered from the test apparatus and substrate, for a total recovery of 100%.

The drug was also coated on a piece of aluminum foil (20 cm2) according to Method C. The calculated thickness of the drug film was 1.0 μm. The substrate was heated as described in Method C at 90 V for 3.5 seconds. The purity of the drug-aerosol particles was determined to be 85.5%. 1.91 mg was recovered from the glass tube walls after vaporization, for a percent yield of 95.5%.

Diltiazam was also coated on a piece of aluminum foil (20 cm2) according to Method C. The calculated thickness of the drug film was 1.1 μm. The substrate was heated as described in Method C at 90 V for 3.5 seconds under an argon atmosphere. The purity of the drug-aerosol particles was determined to be 97.1%. 1.08 mg was recovered from the glass tube walls after vaporization, for a percent yield of 49.1%.

### Example 41

Diphenhydramine (MW 255, melting point <25° C., oral dose 25 mg), an antihistamine, was coated on an aluminum foil substrate (20 cm2) according to Method C. 5.50 mg of drug was applied to the substrate, for a calculated thickness of the drug film of 2.8 μm. The substrate was heated as described in Method C at 108 V for 2.25 seconds. The purity of the drug-aerosol particles was determined to be 93.8%. 3.97 mg was recovered from the glass tube walls after vaporization, for a percent yield of 72.2%.

The hydrochloride salt was also tested. 4.90 mg of drug was coated onto an aluminum substrate, for a calculated thickness of the drug film of 2.5 μm. The substrate was heated under an argon atmosphere as described in Method C at 60 V for 10 seconds. The purity of the drug-aerosol particles was determined to be 90.3%. 3.70 mg was recovered from the glass tube walls after vaporization, for a percent yield of 75.5%. Another experiment with the hydrochloride salt was done under an argon atmosphere. 5.20 mg of drug was coated onto an aluminum substrate, for a calculated thickness of the drug film of 2.6 μm. The substrate was heated as described in Method C at 60 V for 10 seconds. The purity of the drug-aerosol particles was determined to be 93.3%. 3.90 mg was recovered from the glass tube walls after vaporization, for a percent yield of 75.0%.

### Example 42

Disopyramide (MW 339, melting point 95° C., oral dose 100 mg), a cardiovascular agent, was coated on a stainless steel cylinder (8 cm2) according to Method D. 1.07 mg of drug was applied to the substrate, for a calculated drug film thickness of 1.3 μm. The substrate was heated as described in Method D by charging the capacitors to 20.5 V. The purity of the drug-aerosol particles was determined to be 99%. 0.63 mg was recovered from the filter after vaporization, for a percent yield of 58.9%. A total mass of 0.9 mg was recovered from the test apparatus and substrate, for a total recovery of 84.1%.

High speed photographs were taken as the drug-coated substrate was heated to monitor visually formation of a thermal vapor. The photographs, shown in FIGS. 25A-25D, showed that a thermal vapor was initially visible 50 milliseconds after heating was initiated, with the majority of the thermal vapor formed by 100 milliseconds. Generation of the thermal vapor was complete by 200 milliseconds.

### Example 43

Doxepin (MW 279, melting point <25° C. oral dose 75 mg), a psychotherapeutic agent, was coated on an aluminum foil substrate (20 cm2) according to Method C. 2.0 mg of drug was applied to the substrate, for a calculated thickness of the drug film of 1.0 μm. The substrate was heated as described in Method C at 90 V for 3.5 seconds. The purity of the drug-aerosol particles was determined to be 99%. The total mass recovered from the glass tube walls after vaporization ˜100%.

Another substrate containing doxepin was also prepared. On an aluminum foil substrate (20 cm2) 8.6 mg of drug was applied to the substrate, for a calculated thickness of the drug film of 4.5 μm. The substrate was heated as described in Method C at 90 V for 5 seconds. The purity of the drug-aerosol particles was determined to be 81.1%. 6.4 mg was recovered from the glass tube walls after vaporization, for a percent yield of 74.4%.

Another substrate containing doxepin was also prepared for testing under argon. On an aluminum foil substrate (20 cm2) 1.8 mg of drug was applied to the substrate, for a calculated thickness of the drug film of 0.9 μm. The substrate was heated as described in Method C at 90 V for 3.5 seconds. The purity of the drug-aerosol particles was determined to be 99.1%. The total mass recovered from the glass tube walls after vaporization ˜100%.

### Example 44

Donepezil (MW 379, oral dose 5 mg), a drug used in management of Alzheimer's, was coated on a stainless steel cylinder (8 cm2) according to Method D. 5.73 mg of drug was applied to the substrate, for a calculated drug film thickness of 6.9 μm. The substrate was heated as described in Method D by charging the capacitors to 20.5 V. The purity of the drug-aerosol particles was determined to be 96.9%. 3 mg was recovered from the filter after vaporization, for a percent yield of 52.4%. A total mass of 3 mg was recovered from the test apparatus and substrate, for a total recovery of 52.4%.

Donepezil was also tested according to Method B, by coating a solution of the drug onto a piece of stainless steel foil (5 cm2). Six substrates were prepared, with film thicknesses ranging from about 0.5 μm to about 3.2 μm. The substrates were heated as described in Method B by charging the capacitors to 14.5 or 15.5 V. Purity of the drug aerosol particles from each substrate was determined. The results are shown in FIG. 7.

Donepezil was also tested by coating a solution of the drug onto a piece of stainless steel foil (5 cm2). The substrate having a drug film thickness of 2.8 μm was prepared by depositing 1.51 mg of drug. After volatilization of drug from the substrate by charging the capacitors to 14.5 V. 1.37 mg of aerosol particles were recovered from the filter, for a percent yield of 90.9%. The purity of drug compound recovered from the filter was 96.5%. A total mass of 1.51 mg was recovered from the test apparatus and substrate, for a total recovery of 100%.

### Example 45

Eletriptan (MW 383, oral dose 3 mg), a serotonin 5-HT receptor agonist used as a migraine preparation, was coated on a piece of stainless steel foil (6 cm2) according to Method B. 1.38 mg of drug was applied to the substrate, for a calculated drug film thickness of 2.2 μm. The substrate was heated as described in Method B by charging the capacitors to 16 V. The purity of the drug-aerosol particles was determined to be 97.8%. 1.28 mg was recovered from the filter after vaporization, for a percent yield of 93%. The total mass was recovered from the test apparatus and substrate, for a total recovery of 100%.

### Example 46

Estradiol (MW 272, melting point 179° C., oral dose 2 mg), a hormonal agent, was coated on a piece of aluminum foil (20 cm2) according to Method C. The calculated thickness of the drug film was 1.3 μm. The substrate was heated as described in Method C at 60 V for 9 seconds. The purity of the drug-aerosol particles was determined to be 98.5%. 1.13 mg was recovered from the glass tube walls after vaporization, for a percent yield of 45.2%.

Another substrate containing estradiol was also prepared for testing under argon. On an aluminum foil substrate (20 cm2) 2.6 mg of drug was applied to the substrate, for a calculated thickness of the drug film of 1.3 μm. The substrate was heated as described in Method C at 60 V for 9 seconds. The purity of the drug-aerosol particles was determined to be 98.7%. 1.68 mg was recovered from the glass tube walls after vaporization, for a percent yield of 64.6%.

### Example 47

Estradiol-3,17-diacetate (MW 357, oral dose 2 mg), a hormonal prodrug, was coated on a piece of aluminum foil (20 cm2) according to Method C. The calculated thickness of the drug film was 0.9 μm. The substrate was heated as described in Method C at 60 V for 7 seconds. The purity of the drug-aerosol particles was determined to be 96.9%. 1.07 mg was recovered from the glass tube walls after vaporization, for a percent yield of 62.9%.

### Example 48

Efavirenz (MW 316, melting point 141° C., oral dose 600 mg), an anti-infective agent, was coated on a stainless steel cylinder (8 cm2) according to Method D. 0.82 mg of drug was applied to the substrate, for a calculated drug film thickness of 1 μm. The substrate was heated as described in Method D by charging the capacitors to 20.5 V. The purity of the drug-aerosol particles was determined to be 97.9%. 0.52 mg was recovered from the filter after vaporization, for a percent yield of 63.4%. A total mass of 0.6 mg was recovered from the test apparatus and substrate, for a total recovery of 73.2%.

### Example 49

Ephedrine (MW 165, melting point 40° C., oral dose 10 mg), a respiratory agent, was coated on an aluminum foil substrate (20 cm2) according to Method C. 8.0 mg of drug was applied to the substrate, for a calculated thickness of the drug film of 4.0 μm. The substrate was heated as described in Method C at 90 V for 5 seconds. The purity of the drug-aerosol particles was determined to be 99%. 7.26 mg was recovered from the glass tube walls after vaporization, for a percent yield of 90.8%.

### Example 50

Esmolol (MW 295, melting point 50° C., oral dose 35 mg), a cardiovascular agent, was coated on a piece of aluminum foil (20 cm2) according to Method C. The calculated thickness of the drug film was 4.9 μm. The substrate was heated as described in Method C at 90 V for 5 seconds. The purity of the drug-aerosol particles was determined to be 95.8%. 6.4 mg was recovered from the glass tube walls after vaporization, for a percent yield of 65.3%.

Esmolol was coated on a stainless steel cylinder (8 cm2) according to Method D. 0 83 mg of drug was applied to the substrate, for a calculated drug film thickness of 1.4 μm. The substrate was heated as described in Method D by charging the capacitors to 20.5 V. The purity of the drug-aerosol particles was determined to be 93%. 0.63 mg was recovered from the filter after vaporization, for a percent yield of 75.9%. A total mass of 0.81 mg was recovered from the test apparatus and substrate, for a total recovery of 97.6%.

High speed photographs were taken as the drug-coated substrate was heated to monitor visually formation of a thermal vapor. The photographs showed that a thermal vapor was initially visible 25 milliseconds after heating was initiated, with the majority of the thermal vapor formed by 60 milliseconds. Generation of the thermal vapor was complete by 75 milliseconds.

### Example 51

Estazolam (MW 295, melting point 229° C., oral dose 2 mg), a sedative and hypnotic, was coated on an aluminum foil substrate (20 cm2) according to Method C. 2.0 mg of drug was applied to the substrate, for a calculated thickness of the drug film of 1.0 μm. The substrate was heated basically as described in Method C at 60 V for 3 seconds then 45 V for 11 seconds. The purity of the drug-aerosol particles was determined to be 99.9%. 1.4 mg was recovered from the glass tube walls after vaporization, for a percent yield of 70%.

### Example 52

Ethacrynic acid (MW 303, melting point 122° C., oral dose 25.0 mg), a cardiovascular agent, was coated on a stainless steel cylinder (8 cm2) according to Method E. 1.10 mg of drug was applied to the substrate, for a calculated drug film thickness of 1.3 μm. The substrate was heated as described in Method E and purity of the drug-aerosol particles was determined to be 99.8%. 0.85 mg was recovered from the filter after vaporization, for a percent yield of 77.3%. A total mass of 1.1 mg was recovered from the test apparatus and substrate, for a total recovery of 100%.

### Example 53

Ethambutol (MW 204, melting point 89° C., oral dose 1000 mg), a anti-infective agent, was coated on a stainless steel cylinder (8 cm2) according to Method D. 0.85 mg of drug was applied to the substrate, for a calculated drug film thickness of 1 μm. The substrate was heated as described in Method D by charging the capacitors to 20.5 V. The purity of the drug-aerosol particles was determined to be 90%. 0.50 mg was recovered from the filter after vaporization, for a percent yield of 58.8%. A total mass of 0.85 mg was recovered from the test apparatus and substrate, for a total recovery of 100%.

High speed photographs were taken as the drug-coated substrate was heated to monitor visually formation of a thermal vapor. The photographs showed that a thermal vapor was initially visible 25 milliseconds after heating was initiated, with the majority of the thermal vapor formed by 50 milliseconds. Generation of the thermal vapor was complete by 90 milliseconds.

### Example 54

Fluticasone propionate (MW 501, melting point 272° C., oral dose 0.04 mg), a respiratory agent, was coated on a piece of stainless steel foil (5 cm2) according to Method B. The calculated thickness of the drug film was 0.6 μm. The substrate was heated as described in Method B by charging the capacitors to 15.5 V. The purity of the drug-aerosol particles was determined to be 91.6%. 0.211 mg was recovered from the filter after vaporization, for a percent yield of 70.1%. A total mass of 0.215 mg was recovered from the test apparatus and substrate, for a total recovery of 71.4%.

### Example 55

Fenfluramine (MW 231, melting point 112° C., oral dose 20 mg), an obesity management, was coated on a piece of aluminum foil (20 cm2) according to Method C. 9.2 mg were coated. The calculated thickness of the drug film was 4.6 μm. The substrate was heated as described in Method C at 90 V for 5 seconds. The purity of the drug-aerosol particles was determined to be >99.5%. The total mass was recovered from the glass tube walls after vaporization, for a percent yield of ˜100%.

### Example 56

Fenoprofen (MW 242, melting point <25° C., oral dose 200 mg), an analgesic, was coated on a piece of aluminum foil (20 cm2) according to Method C. The calculated thickness of the drug film was 3.7 μm. The substrate was heated as described in Method C at 60 V for 5 seconds. The purity of the drug-aerosol particles was determined to be 98.7%. 4.98 mg was recovered from the glass tube walls after vaporization, for a percent yield of 67.3%.

### Example 57

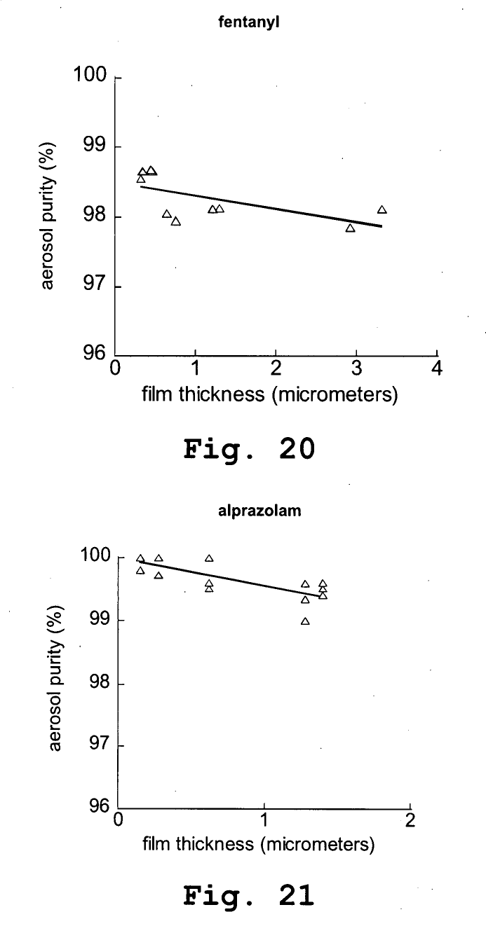

Fentanyl (MW 336, melting point 84° C., oral dose 0.2 mg), an analgesic, was coated onto ten stainless steel foil substrates (5 cm2) according to Method B. The calculated thickness of the drug film on each substrate ranged from about 0.2 μm to about 3.3 μm. The substrates were heated as described in Method B by charging the capacitors to 14 V. Purity of the drug-aerosol particles from each substrate was determined and the results are shown in FIG. 20.

Fentanyl was also coated on a stainless steel cylinder (8 cm2) according to Method D. 0.29 mg of drug was applied to the substrate, for a calculated drug film thickness of 0.4 μm. The substrate was heated as described in Method D by charging the capacitors to 18 V. The purity of the drug-aerosol particles was determined to be 97.9%. 0.19 mg was recovered from the filter after vaporization, for a percent yield of 64%. A total mass of 0.26 mg was recovered from the test apparatus and substrate, for a total recovery of 89%.

High speed photographs were taken as the drug-coated substrate was heated to monitor visually formation of a thermal vapor. The photographs showed that a thermal vapor was initially visible 30 milliseconds after heating was initiated, with the majority of the thermal vapor formed by 100 milliseconds. Generation of the thermal vapor was complete by 250 milliseconds.

### Example 58

Flecainide (MW 414, oral dose 50 mg), a cardiovascular agent, was coated on a stainless steel cylinder (8 cm2) according to Method D. 0.80 mg of drug was applied to the substrate, for a calculated drug film thickness of 1 μm. The substrate was heated as described in Method D by charging the capacitors to 20.5 V. The purity of the drug-aerosol particles was determined to be 99.6%. 0.54 mg was recovered from the filter after vaporization, for a percent yield of 67.5%. A total mass of 0.7 mg was recovered from the test apparatus and substrate, for a total recovery of 90%.

High speed photographs were taken as the drug-coated substrate was heated to monitor visually formation of a thermal vapor. The photographs showed that a thermal vapor was initially visible 25 milliseconds after heating was initiated, with the majority of the thermal vapor formed by 65 milliseconds. Generation of the thermal vapor was complete by 110 milliseconds.

### Example 59

Fluconazole (MW 306, melting point 140° C., oral dose 200 mg), an anti-infective agent, was coated on a piece of stainless steel foil (5 cm2) according to Method B. 0.737 mg of drug was applied to the substrate, for a calculated drug film thickness of 1.4 μm. The substrate was heated as described in Method B by charging the capacitors to 15.5 V. The purity of the drug-aerosol particles was determined to be 94.3%. 0.736 mg was recovered from the filter after vaporization, for a percent yield of 99.9%. A total mass of 0.737 mg was recovered from the test apparatus and substrate, for a total recovery of 100%.

### Example 60

Flunisolide (MW 435, oral dose 0.25 mg), a respiratory agent, was coated was coated on a stainless steel cylinder (8 cm2) according to Method E. 0.49 mg of drug was applied to the substrate, for a calculated drug film thickness of 0.6 μm. The substrate was heated as described in Method E and purity of the drug-aerosol particles was determined to be 97.6%. 0.3 mg was recovered from the filter after vaporization, for a percent yield of 61.2%. A total mass of 0.49 mg was recovered from the test apparatus and substrate, for a total recovery of 100%.

Another substrate (stainless steel foil, 5 cm2) was prepared by applying 0.302 mg drug to form a film having a thickness of 0.6 μm. The substrate was heated as described in Method B by charging the capacitor to 15.0 V. The purity of the drug-aerosol particles was determined to be 94.9%. 0.296 mg was recovered from the filter after vaporization, for a percent yield of 98%. A total mass of 0.302 mg was recovered from the test apparatus and substrate, for a total recovery of 100%.

### Example 61

Flunitrazepam (MW 313, melting point 167° C., oral dose 0.5 mg), a sedative and hypnotic, was coated on a piece of aluminum foil (24.5 cm2) according to Method G. The calculated thickness of the drug film was 0.6 μm. The substrate was heated as described in Method G at 90 V for 6 seconds. The purity of the drug-aerosol particles was determined to be 99.8%. 0.73 mg was recovered from the glass tube walls after vaporization, for a percent yield of 60.8%.

Flunitrazepam was further coated on an aluminum foil substrate (24 cm2) according to Method C. 5 mg of the drug was applied to the substrate, for a calculated thickness of the drug film of 2.08 μm. The substrate was heated substantially as described in Method C at 60 V for 7 seconds. The purity of the drug-aerosol particles was determined to be at least 99.9%.

### Example 62

Fluoxetine (MW 309, oral dose 20 mg), a psychotherapeutic agent, was coated on an aluminum foil substrate (20 cm2) according to Method C. 1.90 mg of drug was applied to the substrate, for a calculated thickness of the drug film of 1.0 μm. The substrate was heated as described in Method C at 90 V for 3.5 seconds. The purity of the drug-aerosol particles was determined to be 97.4%. 1.4 mg was recovered from the glass tube walls after vaporization, for a percent yield of 73.7%.

Another substrate containing fluoxetine coated (2.0 mg drug) to a film thickness of 1.0 μm was prepared by the same method and heated under an argon atmosphere at 90 V for 3.5 seconds. The purity of the drug-aerosol particles was determined to be 96.8%. 1.7 mg was recovered from the glass tube walls after vaporization, for a percent yield of 85.0%.

### Example 63

Galanthamine (MW 287, oral dose 4 mg) was coated on a stainless steel cylinder (8 cm2) according to Method D. 1.4 mg of drug was applied to the substrate, for a calculated drug film thickness of 1.7 μm. The substrate was heated as described in Method D by charging the capacitors to 20.5 V. The purity of the drug-aerosol particles was determined to be >99.5%. 1.16 mg was recovered from the filter after vaporization, for a percent yield of 82.6%. A total mass of 1.39 mg was recovered from the test apparatus and substrate, for a total recovery of 99.1%.

### Example 64

Granisetron (MW 312, oral dose 1 mg), a gastrointestinal agent, was coated on an aluminum foil substrate (20 cm2) according to Method C. 1.50 mg of drug was applied to the substrate, for a calculated thickness of the drug film of 0.8 μm. The substrate was heated as described in Method C at 30 V for 45 seconds. The purity of the drug-aerosol particles was determined to be 99%. 1.3 mg was recovered from the glass tube walls after vaporization, for a percent yield of 86.7%.

mg of granisetron was also coated on an aluminum foil substrate (24.5 cm2) to a calculated drug film thickness of 0.45 μm. The substrate was heated substantially as described in Method G at 90 V for 6 seconds. The purity of the drug-aerosol particles was determined to be 93%. 0.4 mg was recovered from the glass tube walls, for a percent yield of 36%.

### Example 65

Haloperidol (MW 376, melting point 149° C., oral dose 2 mg), a psychotherapeutic agent, was coated on an aluminum foil substrate (20 cm2) according to Method C. 2.20 mg of drug was applied to the substrate, for a calculated thickness of the drug film of 1.1 μm. The substrate was heated as described in Method C at 108 V for 2.25 seconds. The purity of the drug-aerosol particles was determined to be 99.8%. 0.6 mg was recovered from the glass tube walls after vaporization, for a percent yield of 27.3%.

Haloperidol was further coated on an aluminum foil substrate according to Method C. The substrate was heated as described in Method C. When 2.1 mg of the drug was heated at 90 V for 3.5 seconds, the purity of the resultant drug-aerosol particles was determined to be 96%. 1.69 mg of aerosol particles were collected for a percent yield of the aerosol of 60%. When 2.1 mg of drug was used and the system was flushed with argon prior to volatilization, the purity of the drug-aerosol particles was determined to be 97%. The percent yield of the aerosol was 29%.

### Example 66

Hydromorphone (MW 285, melting point 267° C., oral dose 2 mg), an analgesic, was coated on a stainless steel cylinder (9 cm2) according to Method D. 5.62 mg of drug was applied to the substrate, for a calculated drug film thickness of 6.4 μm. The substrate was heated as described in Method D by charging the capacitors to 19 V. The purity of the drug-aerosol particles was determined to be 99.4%. 2.34 mg was recovered from the filter after vaporization, for a percent yield of 41.6%. A total mass of 5.186 mg was recovered from the test apparatus and substrate, for a total recovery of 92.3%.

Hydromorphone was also coated on a piece of aluminum foil (20 cm2) according to Method C. The calculated thickness of the drug film was 1.1 μm. The substrate was heated as described in Method C at 90 V for 3.5 seconds. The purity of the drug-aerosol particles was determined to be 98.3%. 0.85 mg was recovered from the glass tube walls after vaporization, for a percent yield of 40.5%.

Hydromorphone was also coated onto eight stainless steel cylinder substrates (8 cm2) according to Method D. The calculated thickness of the drug film on each substrate ranged from about 0.7 μm to about 2.8 μm. The substrates were heated as described in Method D by charging the capacitors to 20.5 V. The purity of the drug-aerosol particles determined. The results are shown in FIG. 8. For the substrate having a drug film thickness of 1.4 μm, 1.22 mg of drug was applied to the substrate. After vaporization of this substrate, 0.77 mg was recovered from the filter, for a percent yield of 63.21%. The purity of the drug-aerosol particles was determined to be 99.6%. A total mass of 1.05 mg was recovered from the test apparatus and substrate, for a total recovery of 86.1%.

### Example 67

Hydroxychloroquine (MW 336, melting point 91° C., oral dose 400 mg), an antirheumatic agent, was coated on a stainless steel cylinder (8 cm2) according to Method D. 6.58 mg of drug was applied to the substrate, for a calculated drug film thickness of 11 μm. The substrate was heated as described in Method D by charging the capacitors to 20.5 V. The purity of the drug-aerosol particles was determined to be 98.9%. 3.48 mg was recovered from the filter after vaporization, for a percent yield of 52.9%. A total mass of 5.1 mg was recovered from the test apparatus and substrate, for a total recovery of 77.8%.

Hyoscyamine (MW 289, melting point 109° C., oral dose 0.38 mg), a gastrointestinal agent, was coated on a piece of aluminum foil (20 cm2) according to Method C. The calculated thickness of the drug film was 0.9 μm. The substrate was heated as described in Method C at 60 V for 8 seconds. The purity of the drug-aerosol particles was determined to be 95.9%. 0.86 mg was recovered from the glass tube walls after vaporization, for a percent yield of 50.6%.

### Example 69

Ibuprofen (MW 206, melting point 77° C., oral dose 200 mg), an analgesic, was coated on an aluminum foil substrate (20 cm2) according to Method C. 10.20 mg of drug was applied to the substrate, for a calculated thickness of the drug film of 5.1 μm. The substrate was heated as described in Method C at 60 V for 5 seconds. The purity of the drug-aerosol particles was determined to be 99.7%. 5.45 mg was recovered from the glass tube walls after vaporization, for a percent yield of 53.4%.

### Example 70

Imipramine (MW 280, melting point <25° C., oral dose 50 mg), a psycho-therapeutic agent, was coated on a piece of aluminum foil (20 cm2) according to Method C. 1.8 mg was coated on the aluminum foil. The calculated thickness of the drug film was 0.9 μm. The substrate was heated as described in Method C at 90 V for 3.5 seconds. The purity of the drug-aerosol particles was determined to be 98.3%. The total mass recovered from the glass tube walls after vaporization was ˜100%.

Another substrate containing imipramine coated to a film thickness of 0.9 μm was prepared by the same method and heated under an argon atmosphere at 90 V for 3.5 seconds. The purity of the drug-aerosol particles was determined to be 99.1%. 1.5 mg was recovered from the glass tube walls after vaporization, for a percent yield of 83.3%.

### Example 71

Indomethacin (MW 358, melting point 155° C., oral dose 25 mg), an analgesic, was coated on a piece of aluminum foil (20 cm2) according to Method C. The calculated thickness of the drug film was 1.2 μm. The substrate was heated as described in Method C at 60 V for 6 seconds. The purity of the drug-aerosol particles was determined to be 96.8%. 1.39 mg was recovered from the glass tube walls after vaporization, for a percent yield of 60.4%.

Another substrate containing indomethacin coated to a film thickness of 1.5 μm was prepared by the same method and heated under an argon atmosphere at 60 V for 6 seconds. The purity of the drug-aerosol particles was determined to be 99%. 0.61 mg was recovered from the glass tube walls after vaporization, for a percent yield of 20.3%.

### Example 72

Indomethacin ethyl ester (MW 386, oral dose 25 mg), an analgesic, was coated on a piece of aluminum foil (20 cm2) according to Method C. The calculated thickness of the drug film was 2.6 μm. The substrate was heated as described in Method C at 60 V for 9 seconds. The purity of the drug-aerosol particles was determined to be 99%. 2.23 mg was recovered from the glass tube walls after vaporization, for a percent yield of 42.9%.

Another substrate containing indomethacin ethyl ester coated to a film thickness of 2.6 μm was prepared by the same method and heated under an argon atmosphere at 60 V for 9 seconds. The purity of the drug-aerosol particles was determined to be 99%. 3.09 mg was recovered from the glass tube walls after vaporization, for a percent yield of 59.4%.

### Example 73

Indomethacin methyl ester (MW 372, oral dose 25 mg), an analgesic, was coated on a piece of aluminum foil (20 cm2) according to Method C. The calculated thickness of the drug film was 2.1 μm. The substrate was heated as described in Method C at 60 V for 6 seconds. The purity of the drug-aerosol particles was determined to be 99%. 1.14 mg was recovered from the glass tube walls after vaporization, for a percent yield of 27.1%.

Another substrate containing indomethacin methyl ester coated to a film thickness of 1.2 μm was prepared by the same method and heated under an argon atmosphere at 60 V for 6 seconds. The purity of the drug-aerosol particles was determined to be 99%. 1.44 mg was recovered from the glass tube walls after vaporization, for a percent yield of 60%.

### Example 74

Isocarboxazid (MW 231, melting point 106° C., oral dose 10 mg), a psychotherapeutic agent, was coated on a stainless steel cylinder (8 cm2) according to Method D. 0.97 mg of drug was applied to the substrate, for a calculated drug film thickness of 1.2 μm. The substrate was heated as described in Method D by charging the capacitors to 20.5 V. The purity of the drug-aerosol particles was determined to be 99.6%. 0.52 mg was recovered from the filter after vaporization, for a percent yield of 53%. A total mass of 0.85 mg was recovered from the test apparatus and substrate, for a total recovery of 87.7%.

High speed photographs were taken as the drug-coated substrate was heated to monitor visually formation of a thermal vapor. The photographs showed that a thermal vapor was initially visible 30 milliseconds after heating was initiated, with the majority of the thermal vapor formed by 70 milliseconds. Generation of the thermal vapor was complete by 200 milliseconds.

### Example 75

Isotretinoin (MW 300, melting point 175° C., oral dose 35 mg), a skin and mucous membrane agent, was coated on a stainless steel cylinder (8 cm2) according to Method D. 1.11 mg of drug was applied to the substrate, for a calculated drug film thickness of 1.4 μm. The substrate was heated as described in Method D by charging the capacitors to 20.5 V. The purity of the drug-aerosol particles was determined to be 96.6%. 0.66 mg was recovered from the filter after vaporization, for a percent yield of 59.5%. A total mass of 0.86 mg was recovered from the test apparatus and substrate, for a total recovery of 77.5%.

High speed photographs were taken as the drug-coated substrate was heated to monitor visually formation of a thermal vapor. The photographs showed that a thermal vapor was initially visible 30 milliseconds after heating was initiated, with the majority of the thermal vapor formed by 65 milliseconds. Generation of the thermal vapor was complete by 110 milliseconds.

### Example 76

Ketamine (MW 238, melting point 93° C., IV dose 100 mg), an anesthetic, was coated on a stainless steel cylinder (8 cm2) according to Method D. 0.836 mg of drug was applied to the substrate, for a calculated drug film thickness of 1.0 μm. The substrate was heated as described in Method D by charging the capacitors to 20.5 V. The purity of the drug-aerosol particles was determined to be 99.9%. 0.457 mg was recovered from the filter after vaporization, for a percent yield of 54.7%. A total mass of 0.712 mg was recovered from the test apparatus and substrate, for a total recovery of 85.2%.

High speed photographs were taken as the drug-coated substrate was heated to monitor visually formation of a thermal vapor. The photographs showed that a thermal vapor was initially visible 30 milliseconds after heating was initiated, with the majority of the thermal vapor formed by 75 milliseconds. Generation of the thermal vapor was complete by 100 milliseconds.

### Example 77

Ketoprofen (MW 254, melting point 94° C., oral dose 25 mg), an analgesic, was coated on an aluminum foil substrate (20 cm2) according to Method C. 10.20 mg of drug was applied to the substrate, for a calculated thickness of the drug film of 5.1 μm. The substrate was heated as described in Method C at 60 V for 16 seconds. The purity of the drug-aerosol particles was determined to be 98%. 7.24 mg was recovered from the glass tube walls after vaporization, for a percent yield of 71%.

### Example 78

Ketoprofen ethyl ester (MW 282, oral dose 25 mg), an analgesic, was coated on a piece of aluminum foil (20 cm2) according to Method C. The calculated thickness of the drug film was 2.0 μm. The substrate was heated as described in Method C at 60 V for 8 seconds. The purity of the drug-aerosol particles was determined to be 99%. 3.52 mg was recovered from the glass tube walls after vaporization, for a percent yield of 88%.

Another substrate containing ketroprofen ethyl ester coated to a film thickness of 2.7 μm was prepared by the same method and heated under an argon atmosphere at 60 V for 8 seconds. The purity of the drug-aerosol particles was determined to be 99.6%. 4.1 mg was recovered from the glass tube walls after vaporization, for a percent yield of 77.4%.

### Example 79

Ketoprofen Methyl Ester (MW 268, oral dose 25 mg), an analgesic, was coated on a piece of aluminum foil (20 cm2) according to Method C. The calculated thickness of the drug film was 2.0 μm. The substrate was heated as described in Method C at 60 V for 8 seconds purity of the drug-aerosol particles was determined to be 99%. 2.25 mg was recovered from the glass tube walls after vaporization, for a percent yield of 56.3%.

Another substrate containing ketoprofen methyl ester coated to a film thickness of 3.0 μm was prepared by the same method and heated under an argon atmosphere at 60 V for 8 seconds. The purity of the drug-aerosol particles was determined to be 99%. 4.4 mg was recovered from the glass tube walls after vaporization, for a percent yield of 73.3%.

### Example 80

Ketorolac ethyl ester (MW 283, oral dose 10 mg), an analgesic, was coated on an aluminum foil substrate (20 cm2) according to Method C. 9.20 mg of drug was applied to the substrate, for a calculated thickness of the drug film of 4.6 μm. The substrate was heated as described in Method C at 60 V for 12 seconds. The purity of the drug-aerosol particles was determined to be 99%. 5.19 mg was recovered from the glass tube walls after vaporization, for a percent yield of 56.4%.

### Example 81

Ketorolac methyl ester (MW 269, oral dose 10 mg) was also coated on an aluminum foil substrate (20 cm2) to a drug film thickness of 2.4 μm (4.8 mg drug applied). The substrate was heated as described in Method C at 60 V for 6 seconds. The purity of the drug-aerosol particles was determined to be 98.8%. 3.17 mg was recovered from the glass tube walls after vaporization, for a percent yield of 66.0%.

### Example 82

Ketotifen (MW 309, melting point 152° C., used as 0.025% solution in the eye) was coated on a stainless steel cylinder (8 cm2) according to Method D. 0.544 mg of drug was applied to the substrate, for a calculated drug film thickness of 0.7 μm. The substrate was heated as described in Method D by charging the capacitors to 20.5 V. The purity of the drug-aerosol particles was determined to be 99.9%. 0.435 mg was recovered from the filter after vaporization, for a percent yield of 80%. A total mass of 0.544 mg was recovered from the test apparatus and substrate, for a total recovery of 100%.

### Example 83

Lamotrigine (MW 256, melting point 218° C., oral dose 150 mg), an anticonvulsant, was coated on a stainless steel cylinder (8 cm2) according to Method D. 0.93 mg of drug was applied to the substrate, for a calculated drug film thickness of 1.1 μm. The substrate was heated as described in Method D by charging the capacitors to 20.5 V. The purity of the drug-aerosol particles was determined to be 99.1%. 0.58 mg was recovered from the filter after vaporization, for a percent yield of 62.4%. A total mass of 0.93 mg was recovered from the test apparatus and substrate, for a total recovery of 100%.

### Example 84

Lidocaine (MW 234, melting point 69° C., oral dose 30 mg), an anesthetic, was coated on an aluminum foil substrate (20 cm2) according to Method C. 9.50 mg of drug was applied to the substrate, for a calculated thickness of the drug film of 4.8 μm. The substrate was heated as described in Method C at 90 V for 5 seconds. The purity of the drug-aerosol particles was determined to be 99.8%. 7.3 mg was recovered from the glass tube walls after vaporization, for a percent yield of 76.8%.

Lidocaine was further coated on an aluminum foil substrate (24.5 cm2) according to Method G. 10.4 mg of the drug was applied to the substrate, for a calculated thickness of the drug film of 4.24 μm. The substrate was heated as described in Method G at 90 V for 6 seconds. The purity of the drug-aerosol particles was determined to be >99%. 10.2 mg of the drug was found to have aerosolized, for a percent yield of 98%.

### Example 85

Linezolid (MW 337, melting point 183° C., oral dose 600 mg), an anti-infective agent, was coated on a stainless steel cylinder (8 cm2) according to Method D. 1.09 mg of drug was applied to the substrate, for a calculated drug film thickness of 1.3 μm. The substrate was heated as described in Method D by charging the capacitors to 20.5 V. The purity of the drug-aerosol particles was determined to be 95%. 0.70 mg was recovered from the filter after vaporization, for a percent yield of 64.2%. A total mass of 1.09 mg was recovered from the test apparatus and substrate, for a total recovery of 100%.

### Example 86

Loperamide (MW 477, oral dose 4 mg), a gastrointestinal agent, was coated on a stainless steel cylinder (9 cm2) according to Method D. 1.57 mg of drug was applied to the substrate, for a calculated drug film thickness of 1.8 μm. The substrate was heated as described in Method D by charging the capacitors to 20.5 V. The purity of the drug-aerosol particles was determined to be 99.4%. 0.871 mg was recovered from the filter after vaporization, for a percent yield of 55.5%. A total mass of 1.57 mg was recovered from the test apparatus and substrate, for a total recovery of 100%.

High speed photographs were taken as the drug-coated substrate was heated to monitor visually formation of a thermal vapor. The photographs showed that a thermal vapor was initially visible 30 milliseconds after heating was initiated, with the majority of the thermal vapor formed by 80 milliseconds. Generation of the thermal vapor was complete by 165 milliseconds.

### Example 87

Loratadine (MW 383, melting point 136° C., oral dose 10 mg), an antihistamine, was coated on an aluminum foil substrate (20 cm2) according to Method C. 5.80 mg of drug was applied to the substrate, for a calculated thickness of the drug film of 2.9 μm. The substrate was heated as described in Method C at 60 V for 9 seconds. The purity of the drug-aerosol particles was determined to be 99%. 3.5 mg was recovered from the glass tube walls after vaporization, for a percent yield of 60.3%.

Another substrate containing loratadine coated (6.60 mg drug) to a film thickness of 3.3 μm was prepared by the same method and heated under an argon atmosphere at 60 V for 9 seconds. The purity of the drug-aerosol particles was determined to be 99.6%. 4.5 mg was recovered from the glass tube walls after vaporization, for a percent yield of 68.2%.

Loratadine was further coated on an aluminum foil substrate (24.5 cm2) according to Method G. 10.4 mg of the drug was applied to the substrate, for a calculated thickness of the drug film of 4.24 μm. The substrate was heated substantially as described in Method G at 90 V for 6 seconds, except that two of the openings of the T-shaped tube were left open and the third connected to the 1 L flask. The purity of the drug-aerosol particles was determined to be >99%. 3.8 mg of the drug was found to have aerosolized, for a percent yield of 36.5%.

### Example 88

Lovastatin (MW 405, melting point 175° C., oral dose 20 mg), a cardiovascular agent, was coated on a stainless steel cylinder (8 cm2) according to Method D. 0.71 mg of drug was applied to the substrate, for a calculated drug film thickness of 0.9 μm. The substrate was heated as described in Method D by charging the capacitors to 20.5 V. The purity of the drug-aerosol particles was determined to be 94.1%. 0.43 mg was recovered from the filter after vaporization, for a percent yield of 60.6%. A total mass of 0.63 mg was recovered from the test apparatus and substrate, for a total recovery of 88.7%.

### Example 89

Lorazepam N,O-diacetyl (typical inhalation dose 0.5 mg), was coated on a piece of aluminum foil (20 cm2) according to Method C. The calculated thickness of the drug film was 0.5 μm. The substrate was heated as described in Method C at 60 V for 7 seconds. The purity of the drug-aerosol particles was determined to be 90%. 0.87 mg was recovered from the glass tube walls after vaporization, for a percent yield of 87%.

### Example 90

Loxapine (MW 328, melting point 110° C., oral dose 30 mg), a psychotherapeutic agent, was coated on a stainless steel cylinder (8 cm2) according to Method D. 7.69 mg of drug was applied to the substrate, for a calculated drug film thickness of 9.2 μm. The substrate was heated as described in Method D by charging the capacitors to 20.5 V. The purity of the drug-aerosol particles was determined to be 99.7%. 3.82 mg was recovered from the filter after vaporization, for a percent yield of 50%. A total mass of 6.89 mg was recovered from the test apparatus and substrate, for a total recovery of 89.6%.

### Example 91

Maprotiline (MW 277, melting point 94° C., oral dose 25 mg), a psychotherapeutic agent, was coated on an aluminum foil substrate (20 cm2) according to Method C. 2.0 mg of drug was applied to the substrate, for a calculated thickness of the drug film of 1.0 μm. The substrate was heated as described in Method C at 90 V for 3.5 seconds. The purity of the drug-aerosol particles was determined to be 99.7%. 1.3 mg was recovered from the glass tube walls after vaporization, for a percent yield of 65.0%.

Another substrate containing maprotiline coated to a film thickness of 1.0 μm was prepared by the same method and heated under an argon atmosphere at 90 V for 3.5 seconds. The purity of the drug-aerosol particles was determined to be 99.6%. 1.5 mg was recovered from the glass tube walls after vaporization, for a percent yield of 75%.

### Example 92

Meclizine (MW 391, melting point <25° C., oral dose 25 mg), a vertigo agent, was coated on an aluminum foil substrate (20 cm2) according to Method C. 5.20 mg of drug was applied to the substrate, for a calculated thickness of the drug film of 2.6 μm. The substrate was heated as described in Method C at 60 V for 7 seconds. The purity of the drug-aerosol particles was determined to be 90.1%. 3.1 mg was recovered from the glass tube walls after vaporization, for a percent yield of 59.6%.

The same drug coated on an identical substrate (aluminum foil (20 cm2)) to a calculated drug film thickness of 12.5 μm was heated under an argon atmosphere as described in Method C at 60 V for 10 seconds. The purity of the drug-aerosol particles was determined to be 97.3%. 4.81 mg was recovered from the glass tube walls after vaporization, for a percent yield of 19.2%.

The dihydrochloride salt form of the drug was also tested. Meclizine dihydrochloride (MW 464, oral dose 25 mg) was coated on a piece of aluminum foil (20 cm2) according to Method C. 19.4 mg of drug was applied to the substrate, for a calculated thickness of the drug film of 9.7 μm. The substrate was heated as described in Method C at 60 V for 6 seconds. The purity of the drug-aerosol particles was determined to be 75.3%. 0.5 mg was recovered from the glass tube walls after vaporization, for a percent yield of 2.6%.

An identical substrate having a calculated drug film thickness of 11.7 μm was heated under an argon atmosphere at 60 V for 6 seconds. Purity of the drug-aerosol particles was determined to be 70.9%. 0.4 mg was recovered from the glass tube walls after vaporization, for a percent yield of 1.7%.

### Example 93

Memantine (MW 179, melting point <25° C., oral dose 20 mg), an antiparkinsonian agent, was coated on a stainless steel cylinder (8 cm2) according to Method D. The calculated thickness of the drug film was 1.7 μm. The substrate was heated as described in Method D by charging the capacitors to 20.5 V. The purity of the drug-aerosol particles determined by LC/MS was >99.5%. 0.008 mg was recovered from the glass tube walls after vaporization, for a percent yield of 0.6%. The total mass recovered was 0.06 mg, for a total recovery yield of 4.5%. The amount of drug trapped on the filter was low, most of the aerosol particles escaped into the vacuum line.

### Example 94

Meperidine (MW 247, oral dose 50 mg), an analgesic, was coated on an aluminum foil substrate (20 cm2) according to Method C. 1.8 mg of drug was applied to the substrate, for a calculated thickness of the drug film of 0.9 μm. The substrate was heated as described in Method C at 90 V for 3.5 seconds. The purity of the drug-aerosol particles was determined to be 98.8%. 0.95 mg was recovered from the glass tube walls after vaporization, for a percent yield of 52.8%.

Another substrate containing meperidine coated to a film thickness of 1.1 μm was prepared by the same method and heated under an argon atmosphere at 90 V for 3.5 seconds. The purity of the drug-aerosol particles was determined to be 99.9%. 1.02 mg was recovered from the glass tube walls after vaporization, for a percent yield of 48.6%.

### Example 95

Metaproterenol (MW 211, melting point 100° C., oral dose 1.3 mg), a respiratory agent, was coated on a stainless steel cylinder (8 cm2) according to Method D. 1.35 mg of drug was applied to the substrate, for a calculated drug film thickness of 1.6 μm. The substrate was heated as described in Method D by charging the capacitors to 20.5 V. The purity of the drug-aerosol particles was determined to be 99.1%. 0.81 mg was recovered from the filter after vaporization, for a percent yield of 60%. A total mass of 1.2 mg was recovered from the test apparatus and substrate, for a total recovery of 88.9%.

High speed photographs were taken as the drug-coated substrate was heated to monitor visually formation of a thermal vapor. The photographs showed that a thermal vapor was initially visible 30 milliseconds after heating was initiated, with the majority of the thermal vapor formed by 150 milliseconds. Generation of the thermal vapor was complete by 300 milliseconds.

### Example 96

Methadone (MW 309, melting point 78° C., oral dose 2.5 mg), an analgesic, was coated on an aluminum foil substrate (20 cm2) according to Method C. 1.80 mg of drug was applied to the substrate, for a calculated thickness of the drug film of 0.9 μm. The substrate was heated as described in Method C at 90 V for 3.5 seconds. The purity of the drug-aerosol particles was determined to be 92.3%. 1.53 mg was recovered from the glass tube walls after vaporization, for a percent yield of 85%.

### Example 97

Methoxsalen (MW 216, melting point 148° C., oral dose 35 mg), a skin and mucous membrane agent, was coated on a stainless steel cylinder (8 cm2) according to Method D. 1.03 mg of drug was applied to the substrate, for a calculated drug film thickness of 1.2 μm. The substrate was heated as described in Method D by charging the capacitors to 20.5 V. The purity of the drug-aerosol particles was determined to be 99.6%. 0.77 mg was recovered from the filter after vaporization, for a percent yield of 74.8%. A total mass of 1.03 mg was recovered from the test apparatus and substrate, for a total recovery of 100%.

High speed photographs were taken as the drug-coated substrate was heated to monitor visually formation of a thermal vapor. The photographs showed that a thermal vapor was initially visible 35 milliseconds after heating was initiated, with the majority of the thermal vapor formed by 80 milliseconds. Generation of the thermal vapor was complete by 135 milliseconds.

### Example 98

Metoprolol (MW 267, oral dose 15 mg), a cardiovascular agent, was coated on an aluminum foil substrate (20 cm2) according to Method C. 10.8 mg of drug was applied to the substrate, for a calculated thickness of the drug film of 5.4 μm. The substrate was heated as described in Method C at 90 V for 5 seconds. The purity of the drug-aerosol particles was determined to be 99.2%. 6.7 mg was recovered from the glass tube walls after vaporization, for a percent yield of 62.0%.

Metoprolol was further coated on an aluminum foil substrate (24.5 cm2) according to Method G. 12.7 mg of the drug was applied to the substrate, for a calculated thickness of the drug film of 5.18 μm. The substrate was heated as described in Method G at 90 V for 6 seconds. The purity of the drug-aerosol particles was determined to be >99%. All of the drug was found to have aerosolized, for a percent yield of 100%.

### Example 99

Mexiletine HCl (MW 216, melting point 205° C., oral dose 200 mg), a cardiovascular agent, was coated on a stainless steel cylinder (8 cm2) according to Method D. 0.75 mg of drug was applied to the substrate, for a calculated drug film thickness of 0.9 μm. The substrate was heated as described in Method D by charging the capacitors to 20.5 V. The purity of the drug-aerosol particles was determined to be 99.4%. 0.44 mg was recovered from the filter after vaporization, for a percent yield of 58.7%. A total mass of 0.75 mg was recovered from the test apparatus and substrate, for a total recovery of 100%.

High speed photographs were taken as the drug-coated substrate was heated to monitor visually formation of a thermal vapor. The photographs showed that a thermal vapor was initially visible 25 milliseconds after heating was initiated, with the majority of the thermal vapor formed by 75 milliseconds. Generation of the thermal vapor was complete by 200 milliseconds.

### Example 100

Midazolam (MW 326, melting point 160° C., oral dose 2.5 mg), a sedative and hypnotic, was coated onto five stainless steel cylindrical substrates according to Method E. The calculated thickness of the drug film on each substrate ranged from about 1.1 μm to about 5.8 μm. The substrates were heated as described in Method E and purity of the drug-aerosol particles determined. The results are shown in FIG. 12.

Another substrate (stainless steel cylindrical, 6 cm2) was prepared by depositing 5.37 mg drug to obtain a drug film thickness of 9 μm. After volatilization of drug from this substrate according to Method E, 3.11 mg was recovered from the filter, for a percent yield of 57.9%. A total mass of 5.06 mg was recovered from the test apparatus and substrate, for a total recovery of 94.2%. Purity of the drug aerosol particles was 99.5%. The yield of aerosol particles was 57.9%.

High speed photographs were taken as the drug-coated substrate was heated to monitor visually formation of a thermal vapor. The photographs showed that a thermal vapor was initially visible 35 milliseconds after heating was initiated, with the majority of the thermal vapor formed by 130 milliseconds. Generation of the thermal vapor was complete by 240 milliseconds.

Midazolam was also coated on an aluminum foil substrate (28.8 cm2) according to Method C. 5.0 mg of the drug was applied to the substrate, for a calculated thickness of the drug film of 1.74 μm. The substrate was heated substantially as described in Method C at 60 V for 6 seconds. The purity of the drug-aerosol particles was determined to be 99.9%.

Another aluminum foil substrate (36 cm2) was prepared essentially according to Method G. 16.7 mg of midazolam was applied to the substrate, for a calculated thickness of the drug film of 4.64 μm. The substrate was heated substantially as described in Method G at 90 V for 6 seconds, except that one of the openings of the T-shaped tube was sealed with a rubber stopper, one was loosely covered with the end of the halogen tube, and the third connected to the 1 L flask. The purity of the drug-aerosol particles was determined to be >99%. All of the drug was found to have aerosolized, for a percent yield of 100%.

### Example 101

Mirtazapine (MW 265, melting point 116° C., oral dose 10 mg), a psychotherapeutic agent used as an antidepressant, was coated on an aluminum foil substrate (24.5 cm2) according to Method G. 20.7 mg of drug was applied to the substrate, for a calculated thickness of the drug film of 8.4 μm. The substrate was heated as described in Method G at 90 V for 6 seconds. The purity of the drug-aerosol particles was determined to be 99%. 10.65 mg was recovered from the glass tube walls after vaporization, for a percent yield of 51.4%.

### Example 102

Morphine (MW 285, melting point 197° C., oral dose 15 mg), an analgesic, was coated on a stainless steel cylinder (8 cm2) according to Method D. 2.33 mg of drug was applied to the substrate, for a calculated drug film thickness of 2.8 μm. The substrate was heated as described in Method D by charging the capacitors to 20.5 V. The purity of the drug-aerosol particles was determined to be 99.1%. 1.44 mg was recovered from the filter after vaporization, for a percent yield of 61.8%. A total mass of 2.2 mg was recovered from the test apparatus and substrate, for a total recovery of 94.2%.

Morphine (MW 285, melting point 197° C., oral dose 15 mg), an analgesic, was coated on a piece of aluminum foil (20 cm2) according to Method C. The calculated thickness of the drug film was 4.8 μm. The substrate was heated as described in Method C at 90 V for 5 seconds. The purity of the drug-aerosol particles was determined to be 92.5%. 3.1 mg was recovered from the glass tube walls after vaporization, for a percent yield of 32.3%.

### Example 103

Nalbuphine (MW 357, melting point 231° C., oral dose 10 mg), an analgesic, was coated onto four stainless steel cylinder substrates (8 cm2) according to Method D. The calculated thickness of the drug film on each substrate ranged from about 0.7 μm to about 2.5 μm. The substrates were heated as described in Method D by charging the capacitors to 20.5 V. The purity of the drug-aerosol particles from each substrate was determined and the results are shown in FIG. 13. For the substrate having a drug film thickness of 0.7 μm, 0.715 mg of drug was applied to the substrate. After volatilization of this substrate, 0.455 mg was recovered from the filter, for a percent yield of 63.6%. A total mass of 0.715 mg was recovered from the test apparatus and substrate, for a total recovery of 100%.

### Example 104

Naloxone (MW 327, melting point 184° C., oral dose 0.4 mg), an antidote, was coated on an aluminum foil (20 cm2) according to Method C. 2.10 mg of drug was applied to the substrate, for a calculated thickness of the drug film of 1.1 μm. The substrate was heated as described in Method C at 90 V for 3.5 seconds. The purity of the drug-aerosol particles was determined to be 78.4%. 1.02 mg was recovered from the glass tube walls after vaporization, for a percent yield of 48.6%.

Another substrate containing naloxone coated to a film thickness of 1.0 μm was prepared by the same method and heated under an argon atmosphere at 90 V for 3.5 seconds. The purity of the drug-aerosol particles was determined to be 99.2%. 1.07 mg was recovered from the glass tube walls after vaporization, for a percent yield of 53.5%.

### Example 105

Naproxen (MW 230, melting point 154° C., oral dose 200 mg), an analgesic, was coated on a piece of aluminum foil (20 cm2) according to Method C. 8.7 mg were coated on the foil for a calculated thickness of the drug film of 4.4 μm. The substrate was heated as described in Method C at 60 V for 7 seconds. The purity of the drug-aerosol particles was determined to be >99.5%. 4.4 mg was recovered from the glass tube walls after vaporization, for a percent yield of 50.5%.

### Example 106

Naratriptan (MW 335, melting point 171° C., oral dose 1 mg), a migraine preparation, was coated onto seven stainless steel cylinder substrates (8 cm2) according to Method D. The calculated thickness of the drug film on each substrate ranged from about 0.5 μm to about 2.5 μm. The substrates were heated as described in Method D by charging the capacitors to 20.5 V. Purity of the drug-aerosol particles from each substrate was determined and the results are shown in FIG. 14. For the substrate having a drug film thickness of 0.6 μm, 0.464 mg of drug was applied to the substrate. After vaporization of this substrate by charging the capacitors to 20.5 V. 0.268 mg was recovered from the filter, for a percent yield of 57.8%. The purity was determined to be 98.7%. A total mass of 0.464 mg was recovered from the test apparatus and substrate, for a total recovery of 100%.

High speed photographs were taken as the drug-coated substrate was heated to monitor visually formation of a thermal vapor. The photographs showed that a thermal vapor was initially visible 35 milliseconds after heating was initiated, with the majority of the thermal vapor formed by 100 milliseconds. Generation of the thermal vapor was complete by 250 milliseconds.

### Example 107

Nefazodone (MW 470, melting point 84° C., oral dose 75 mg), a psychotherapeutic agent, was coated on a piece of aluminum foil (20 cm2) according to Method C. The calculated thickness of the drug film was 4.6 μm. The substrate was heated as described in Method C at 60 V for 15 seconds. The purity of the drug-aerosol particles was determined to be 91%. 4.4 mg was recovered from the glass tube walls after vaporization, for a percent yield of 47.8%.

Another substrate containing nefazodone coated to a film thickness of 3.2 μm was prepared by the same method and heated under an argon atmosphere at 60 V for 15 seconds. The purity of the drug-aerosol particles was determined to be 97.5%. 4.3 mg was recovered from the glass tube walls after vaporization, for a percent yield of 68.3%.

### Example 108

Nortriptyline (MW 263, oral dose 15 mg), a psychotherapeutic agent, was coated on an aluminum foil substrate (20 cm2) according to Method C. The calculated thickness of the drug film was 1.0 μm. The substrate was heated as described in Method C at 90 V for 3.5 seconds. The purity of the drug-aerosol particles was determined to be 99.1%. 1.4 mg was recovered from the glass tube walls after vaporization, for a percent yield of 70.0%.

Another substrate containing nortriptyline was prepared for testing under an argon atmosphere. 1.90 mg of drug was applied to the substrate, for a calculated thickness of the drug film of 1.0 μm. The substrate was heated as described in Method C at 90 V for 3.5 seconds. The purity of the drug-aerosol particles was determined to be 97.8%. 1.6 mg was recovered from the glass tube walls after vaporization, for a percent yield of 84.2%.

### Example 109

Olanzapine (MW 312, melting point 195° C., oral dose 10 mg), a psychotherapeutic agent, was coated onto eight stainless steel cylinder substrates (8-9 cm2) according to Method D. The calculated thickness of the drug film on each substrate ranged from about 1.2 μm to about 7.1 μm. The substrates were heated as described in Method D by charging the capacitors to 20.5 V. Purity of the drug-aerosol particles from each substrate was determined and the results are shown in FIG. 15. The substrate having a thickness of 3.4 μm was prepared by depositing 2.9 mg of drug. After volatilization of drug from this substrate by charging the capacitors to 20.5 V. 1.633 mg was recovered from the filter, for a percent yield of 54.6%. The purity of the drug aerosol recovered from the filter was found to be 99.8%. The total mass was recovered from the test apparatus and substrate, for a total recovery of 100%.

High speed photographs were taken as the drug-coated substrate was heated to monitor visually formation of a thermal vapor. The photographs showed that a thermal vapor was initially visible 30 milliseconds after heating was initiated, with the majority of the thermal vapor formed by 80 milliseconds. Generation of the thermal vapor was complete by 130 milliseconds.

Olanzapine was also coated on an aluminum foil substrate (24.5 cm2) according to Method G. 11.3 mg of drug was applied to the substrate, for a calculated thickness of the drug film of 4.61 μm. The substrate was heated as described in Method G at 90 V for 6 seconds. The purity of the drug-aerosol particles was determined to be >99%. 7.1 mg was collected for a percent yield of 62.8%.

### Example 110

Orphenadrine (MW 269, melting point <25° C., oral dose 60 mg), a muscle relaxant, was coated on a piece of aluminum foil (20 cm2) according to Method C. The calculated thickness of the drug film was 1.0 μm. The substrate was heated as described in Method C at 90 V for 3.5 seconds. The purity of the drug-aerosol particles was determined to be >99.5%. 1.35 mg was recovered from the glass tube walls after vaporization, for a percent yield of 71.1%.

### Example 111

Oxycodone (MW 315, melting point 220° C., oral dose 5 mg), an analgesic, was coated on an aluminum foil substrate (20 cm2) according to Method C. 2.4 mg of drug was applied to the substrate, for a calculated thickness of the drug film of 1.2 μm. The substrate was heated as described in Method C at 90 V for 3.5 seconds. The purity of the drug-aerosol particles was determined to be 99.9%. 1.27 mg was recovered from the glass tube walls after vaporization, for a percent yield of 52.9%.

### Example 112

Oxybutynin (MW 358, oral dose 5 mg), a urinary tract agent, was coated on a piece of aluminum foil (20 cm2) according to Method C. The calculated thickness of the drug film was 2.8 μm. The substrate was heated as described in Method C at 60 V for 6 seconds. The purity of the drug-aerosol particles was determined to be 90.6%. 3.01 mg was recovered from the glass tube walls after vaporization, for a percent yield of 54.7%.

### Example 113

Parecoxib (MW 370, oral dose 10 mg), a non-steroidal anti-inflammatory analgesic, was coated on a piece of stainless steel foil (5 cm2) according to Method B. The calculated thickness of the drug film was 6.0 μm. The substrate was heated as described in Method B by charging the capacitors to 15.5 V. The purity of the drug-aerosol particles was determined to be 80%. 1.264 mg was recovered from the filter after vaporization, for a percent yield of 39.5%.

Another substrate (stainless steel foil, 5 cm2) was prepared by applying 0.399 mg drug to form a film having a thickness of 0.8 μm. The substrate was heated as described in Method B by charging the capacitors to 15 V. The purity of the drug-aerosol particles was determined to be 97.2%. 0.323 mg was recovered from the filter after vaporization, for a percent yield of 81.0%. A total mass of 0.324 mg was recovered from the test apparatus and substrate, for a total recovery of 81.3%.

### Example 114

Paroxetine (MW 329, oral dose 20 mg), a psychotherapeutic agent, was coated on a stainless steel cylinder (8 cm2) according to Method D. 2.02 mg of drug was applied to the substrate, for a calculated drug film thickness of 2.4 μm. The substrate was heated as described in Method D (with the single exception that the circuit capacitance was 1.5 Farad, not 2.0 Farad), and purity of the drug-aerosol particles was determined to be 99.5%. 1.18 mg was recovered from the filter after vaporization, for a percent yield of 58.4%. A total mass of 1.872 mg was recovered from the test apparatus and substrate, for a total recovery of 92.7%.

Paroxetine was also coated on an aluminum foil substrate (24.5 cm2) as described in Method G. 19.6 mg of drug was applied to the substrate, for a calculated drug film thickness of 8 μm. The substrate was heated as described in Method G at 90 V for 6 seconds purity of the drug-aerosol particles was determined to be 88%. 7.4 mg were lost from the substrate after vaporization, for a percent yield of 37.8%.

### Example 115

Pergolide (MW 314, melting point 209° C., oral dose 1 mg), an antiparkinsonian agent, was coated on a stainless steel cylinder (8 cm2) according to Method D. 1.43 mg of drug was applied to the substrate, for a calculated drug film thickness of 1.9 μm. The substrate was heated as described in Method D by charging the capacitors to 20.5 V. The purity of the drug-aerosol particles was determined to be 99.7%. 1.18 mg was recovered from the filter after vaporization, for a percent yield of 82.5%. A total mass of 1.428 mg was recovered from the test apparatus and substrate, for a total recovery of 99.9%.

Pergolide was also coated on a piece of aluminum foil (20 cm2) according to Method C. The calculated thickness of the drug film was 1.2 μm. The substrate was heated as described in Method C at 90 V for 3.5 seconds. The purity of the drug-aerosol particles was determined to be 98%. 0.52 mg was recovered from the glass tube walls after vaporization, for a percent yield of 22.6%.

High speed photographs were taken as the drug-coated substrate according to Method D was heated to monitor visually formation of a thermal vapor. The photographs showed that a thermal vapor was initially visible 30 milliseconds after heating was initiated, with the majority of the thermal vapor formed by 225 milliseconds. Generation of the thermal vapor was complete by 800 milliseconds.

Pergolide was further coated on an aluminum foil substrate (24.5 cm2) according to Method G. 1.0 mg of the drug was applied to the substrate, for a calculated thickness of the drug film of 0.4 μm. The substrate was heated substantially as described in Method G at 90 V for 6 seconds, except that two of the openings of the T-shaped tube were left open and the third connected to the 1 L flask. The purity of the drug-aerosol particles was determined to be >99%. All of the drug was found to have aerosolized via weight loss from the substrate, for a percent yield of 100%.

### Example 116

Phenyloin (MW 252, melting point 298° C., oral dose 300 mg), an anti-convulsant, was coated on a stainless steel cylinder (8 cm2) according to Method D. 0.9 mg of drug was applied to the substrate, for a calculated drug film thickness of 1.1 μm. The substrate was heated as described in Method D by charging the capacitors to 20.5 V. The purity of the drug-aerosol particles was determined to be >99.5%. 0.6 mg was recovered from the filter after vaporization, for a percent yield of 66.7%. A total mass of 0.84 mg was recovered from the test apparatus and substrate, for a total recovery of 93.3%.

High speed photographs were taken as the drug-coated substrate was heated to monitor visually formation of a thermal vapor. The photographs, shown in FIGS. 24A-24D, showed that a thermal vapor was initially visible 25 milliseconds after heating was initiated, with the majority of the thermal vapor formed by 90 milliseconds. Generation of the thermal vapor was complete by 225 milliseconds.

### Example 117

Pindolol (MW 248, melting point 173° C., oral dose 5 mg), a cardiovascular agent, was coated on an aluminum foil substrate (20 cm2) according to Method C. 4.7 mg of drug was applied to the substrate, for a calculated thickness of the drug film of 2.4 μm. The substrate was heated as described in Method C at 60 V for 7 seconds. The purity of the drug-aerosol particles was determined to be >99.5%. 2.77 mg was recovered from the glass tube walls after vaporization, for a percent yield of 58.9%.

Another substrate containing pindolol coated to a film thickness of 3.3 μm was prepared by the same method and heated under an argon atmosphere at 60 V for 7 seconds. The purity of the drug-aerosol particles was determined to be >99.5%. 3.35 mg was recovered from the glass tube walls after vaporization, for a percent yield of 50.8%.

### Example 118

Pioglitazone (MW 356, melting point 184° C., oral dose 15 mg), an antidiabetic agent, was coated on a stainless steel cylinder (8 cm2) according to Method D. 0.48 mg of drug was applied to the substrate, for a calculated drug film thickness of 0.6 μm. The substrate was heated as described in Method D by charging the capacitors to 20.5 V. The purity of the drug-aerosol particles was determined to be 95.6%. 0.30 mg was recovered from the filter after vaporization, for a percent yield of 62.5%. A total mass of 0.37 mg was recovered from the test apparatus and substrate, for a total recovery of 77.1%.

High speed photographs were taken as the drug-coated substrate was heated to monitor visually formation of a thermal vapor. The photographs showed that a thermal vapor was initially visible 35 milliseconds after heating was initiated, with the majority of the thermal vapor formed by 100 milliseconds. Generation of the thermal vapor was complete by 125 milliseconds.

### Example 119

Piribedil (MW 298, melting point 98° C., IV dose 3 mg), an antiparkinsonian agent, was coated on a stainless steel cylinder (8 cm2) according to Method D. 1.1 mg of drug was applied to the substrate, for a calculated drug film thickness of 1.5 μm. The substrate was heated as described in Method D by charging the capacitors to 20.5 V. The purity of the drug-aerosol particles was determined to be 99.7%. 1.01 mg was recovered from the filter after vaporization, for a percent yield of 91.8%. A total mass of 1.1 mg was recovered from the test apparatus and substrate, for a total recovery of 100%.

### Example 120

Pramipexole (MW 211, oral dose 0.5 mg), an antiparkinsonian agent, was coated on a stainless steel cylinder (8 cm2) according to Method D. 1.05 mg of drug was applied to the substrate, for a calculated drug film thickness of 1.4 μm. The substrate was heated as described in Method D by charging the capacitors to 20.5 V. The purity of the drug-aerosol particles was determined to be 99.3%. 0.949 mg was recovered from the filter after vaporization, for a percent yield of 90.4%. A total mass of 1.05 mg was recovered from the test apparatus and substrate, for a total recovery of 100%.

Pramipexole was also coated on a piece of stainless steel foil (5 cm2) according to Method B. 0.42 mg of drug was applied to the substrate, for a calculated drug film thickness of 0.9 μm. The substrate was heated as described in Method B by charging the capacitors to 14 V. The purity of the drug-aerosol particles was determined to be 98.9%. 0.419 mg was recovered from the filter after vaporization, for a percent yield of 99.8%. A total mass of 0.42 mg was recovered from the test apparatus and substrate, for a total recovery of 100%.

High speed photographs were taken as the drug-coated substrate was heated to monitor visually formation of a thermal vapor. The photographs showed that a thermal vapor was initially visible 25 milliseconds after heating was initiated, with the majority of the thermal vapor formed by 80 milliseconds. Generation of the thermal vapor was complete by 140 milliseconds.

### Example 121

Procainamide (MW 236, oral dose 125 mg), a cardiovascular agent, was coated on a stainless steel cylinder (8 cm2) according to Method D. 0.95 mg of drug was applied to the substrate, for a calculated drug film thickness of 1.1 μm. The substrate was heated as described in Method D by charging the capacitors to 20.5 V. The purity of the drug-aerosol particles was determined to be >99.5%. 0.56 mg was recovered from the filter after vaporization, for a percent yield of 58.9%. A total mass of 0.77 mg was recovered from the test apparatus and substrate, for a total recovery of 81.1%.

High speed photographs were taken as the drug-coated substrate was heated to monitor visually formation of a thermal vapor. The photographs showed that a thermal vapor was initially visible 25 milliseconds after heating was initiated, with the majority of the thermal vapor formed by 90 milliseconds. Generation of the thermal vapor was complete by 250 milliseconds.

### Example 122

Prochlorperazine free base (MW 374, melting point 60 oC, oral dose 5 mg), a psychotherapeutic agent, was coated onto four stainless steel foil substrates (5 cm2) according to Method B. The calculated thickness of the drug film on each substrate ranged from about 2.3 μm to about 10.1 μm. The substrates were heated as described in Method B by charging the capacitors to 15 V. Purity of the drug-aerosol particles from each substrate was determined and the results are shown in FIG. 18.

Prochlorperazine, a psychotherapeutic agent, was also coated on a stainless steel cylinder (8 cm2) according to Method D. 1.031 mg of drug was applied to the substrate, for a calculated drug film thickness of 1.0 μm. The substrate was heated as described in Method D by charging the capacitors to 19 V. The purity of the drug-aerosol particles was determined to be 98.7%. 0.592 mg was recovered from the filter after vaporization, for a percent yield of 57.4%. A total mass of 1.031 mg was recovered from the test apparatus and substrate, for a total recovery of 100%.

### Example 123

Promazine (MW 284, melting point <25° C., oral dose 25 mg), a psychotherapeutic agent, was coated on a piece of aluminum foil (20 cm2) according to Method C. The calculated thickness of the drug film was 5.3 μm. The substrate was heated as described in Method C at 90 V for 5 seconds. The purity of the drug-aerosol particles was determined to be 94%. 10.45 mg was recovered from the glass tube walls after vaporization, for a percent yield of 99.5%.

### Example 124

Promethazine (MW 284, melting point 60° C., oral dose 12.5 mg), a gastrointestinal agent, was coated on an aluminum foil substrate (20 cm2) according to Method C. 5.10 mg of drug was applied to the substrate, for a calculated thickness of the drug film of 2.6 μm. The substrate was heated as described in Method C at 60 V for 10 seconds. The purity of the drug-aerosol particles was determined to be 94.5%. 4.7 mg was recovered from the glass tube walls after vaporization, for a percent yield of 92.2%.

### Example 125

Propafenone (MW 341, oral dose 150 mg), a cardiovascular agent, was coated on a stainless steel cylinder (8 cm2) according to Method D. 0.77 mg of drug was applied to the substrate, for a calculated drug film thickness of 0.9 μm. The substrate was heated as described in Method D by charging the capacitors to 20.5 V. The purity of the drug-aerosol particles was determined to be >99.5%. 0.51 mg was recovered from the filter after vaporization, for a percent yield of 66.2%. A total mass of 0.77 mg was recovered from the test apparatus and substrate, for a total recovery of 100%.

High speed photographs were taken as the drug-coated substrate was heated to monitor visually formation of a thermal vapor. The photographs showed that a thermal vapor was initially visible 20 milliseconds after heating was initiated, with the majority of the thermal vapor formed by 60 milliseconds. Generation of the thermal vapor was complete by 110 milliseconds.

### Example 126

Propranolol (MW 259, melting point 96° C., oral dose 40 mg), a cardiovascular agent, was coated on an aluminum foil substrate (20 cm2) according to Method C. 10.30 mg of drug was applied to the substrate, for a calculated thickness of the drug film of 5.2 μm. The substrate was heated as described in Method C at 90 V for 5 seconds. The purity of the drug-aerosol particles was determined to be 99.6%. 8.93 mg was recovered from the glass tube walls after vaporization, for a percent yield of 86.7%.

### Example 127

Quetiapine (MW 384, oral dose 75 mg), a psychotherapeutic agent, was coated onto eight stainless steel cylinder substrates (8 cm2) according to Method D. The calculated thickness of the drug film on each substrate ranged from about 0.1 μm to about 7.1 μm. The substrates were heated as described in Method D by charging the capacitors to 20.5 V. Purity of the drug-aerosol particles from each substrate was determined and the results are shown in FIG. 16. The substrate having a drug film thickness of 1.8 μm was prepared by depositing 1.46 mg drug. After volatilization of drug this substrate by charging the capacitors to 20.5 V. 0.81 mg was recovered from the filter, for a percent yield of 55.5%. The purity of the drug aerosol recovered from the filter was found to be 99.1%. A total mass of 1.24 mg was recovered from the test apparatus and substrate, for a total recovery of 84.9%.

### Example 128

Quinidine (MW 324, melting point 175° C., oral dose 100 mg), a cardiovascular agent, was coated on a stainless steel cylinder (8 cm2) according to Method D. 1.51 mg of drug was applied to the substrate, for a calculated drug film thickness of 1.8 μm. The substrate was heated as described in Method D by charging the capacitors to 20.5 V. The purity of the drug-aerosol particles was determined to be >99.5%. 0.88 mg was recovered from the filter after vaporization, for a percent yield of 58.3%. A total mass of 1.24 mg was recovered from the test apparatus and substrate, for a total recovery of 82.1%.

### Example 129

Rizatriptan (MW 269, melting point 121° C., oral dose 5 mg), a migraine preparation, was coated on a stainless steel cylinder (6 cm2) according to Method E. 2.1 mg of drug was applied to the substrate, for a calculated drug film thickness of 3.5 μm. The substrate was heated as described in Method E and purity of the drug-aerosol particles was determined to be 99.2%. 1.66 mg was recovered from the filter after vaporization, for a percent yield of 79%. A total mass of 2.1 mg was recovered from the test apparatus and substrate, for a total recovery of 100%.

Rizatriptan was further coated on an aluminum foil substrate (150 cm2) according to Method F. 10.4 mg of the drug was applied to the substrate, for a calculated thickness of the drug film of 0.7 μm. The substrate was heated as described in Method F at 250° C. and the purity of the drug-aerosol particles was determined to be 99%. 1.9 mg was collected in glass wool for a percent yield of 18.3%.

Another aluminum foil substrate (36 cm2) was prepared according to Method G. 11.6 mg of rizatriptan was applied to the substrate, for a calculated thickness of the drug film of 3.2 μm. The substrate was heated substantially as described in Method G at 90 V for 7 seconds, except that one of the openings of the T-shaped tube was sealed with a rubber stopper, one was loosely covered with the end of the halogen tube, and the third connected to the 1 L flask. The purity of the drug-aerosol particles was determined to be >99%. All of the drug was found to have aerosolized, for a percent yield of 100%.

### Example 130

Rofecoxib (MW 314, oral dose 50 mg), an analgesic, was coated on an aluminum foil substrate (20 cm2) according to Method C. 6.5 mg of drug was applied to the substrate, for a calculated thickness of the drug film of 3.3 μm. The substrate was heated as described in Method C at 60 V for 17 seconds. The purity of the drug-aerosol particles was determined to be 97.5%. 4.1 mg was recovered from the glass tube walls after vaporization, for a percent yield of 63.1%.

### Example 131

Ropinirole (MW 260, oral dose 0.25 mg), an antiparkinsonian agent, was coated on a stainless steel cylinder (8 cm2) according to Method D. 0.754 mg of drug was applied to the substrate, for a calculated drug film thickness of 1.0 μm. The substrate was heated as described in Method D by charging the capacitors to 20.5 V. The purity of the drug-aerosol particles was determined to be 99%. 0.654 mg was recovered from the filter after vaporization, for a percent yield of 86.7%. A total mass of 0.728 mg was recovered from the test apparatus and substrate, for a total recovery of 96.6%.

### Example 132

Sertraline (MW 306, oral dose 25 mg), a psychotherapeutic agent used as an antidepressant (Zoloft®), was coated on a stainless steel cylinder (6 cm2) according to Method E. 3.85 mg of drug was applied to the substrate, for a calculated drug film thickness of 6.4 μm. The substrate was heated as described in Method E and purity of the drug-aerosol particles was determined to be 99.5%. 2.74 mg was recovered from the filter after vaporization, for a percent yield of 71.2%.

Sertraline was also coated on a piece of aluminum foil (20 cm2) according to Method C. The calculated thickness of the drug film was 3.3 μm. The substrate was heated as described in Method C at 60 V for 10 seconds. The purity of the drug-aerosol particles was determined to be 98.0%. 5.35 mg was recovered from the glass tube walls after vaporization, for a percent yield of 81.1%.

Another sertraline coated substrate (aluminum foil, 20 cm2) having a drug film thickness of 0.9 μm was heated as described in Method C under a pure argon atmosphere at 90 V for 3.5 seconds. The purity of the drug-aerosol particles was determined to be 98.7%. 1.29 mg was recovered from the glass tube walls after vaporization, for a percent yield of 75.9%.

High speed photographs were taken as the drug-coated substrate from Method D was heated to monitor visually formation of a thermal vapor. The photographs showed that a thermal vapor was initially visible 30 milliseconds after heating was initiated, with the majority of the thermal vapor formed by 135 milliseconds. Generation of the thermal vapor was complete by 250 milliseconds.

### Example 133

Selegiline (MW 187, melting point <25° C., oral dose 5 mg), an antiparkinsonian agent, was coated on an aluminum foil substrate (20 cm2) according to Method C. 3.7 mg of drug was applied to the substrate, for a calculated thickness of the drug film of 1.9 μm. The substrate was heated as described in Method C at 60 V for 8 seconds. The purity of the drug-aerosol particles was determined to be 99.2%. 2.41 mg was recovered from the glass tube walls after vaporization, for a percent yield of 65.1%.

### Example 134

Sildenafil (MW 475, melting point 189° C., oral dose 25 mg), an agent used for erectile dysfunction (Viagra®), was coated onto six stainless steel foil substrates (5 cm2) according to Method B. The calculated thickness of the drug film on each substrate ranged from about 0.5 μm to about 1.6 μm. The substrates were heated as described in Method B by charging the capacitors to 16 V. Purity of the drug-aerosol particles from each substrate was determined and the results are shown in FIG. 22.

Sildenafil was also coated on a stainless steel cylinder (6 cm2) according to Method E. 1.9 mg of drug was applied to the substrate, for a calculated drug film thickness of 3.2 μm. The substrate was heated as described in Method E and purity of the drug-aerosol particles was determined to be 81%. 1.22 mg was recovered from the filter after vaporization, for a percent yield of 64.2%. A total mass of 1.5 mg was recovered from the test apparatus and substrate, for a total recovery of 78.6%.

Sildenafil was also coated on a piece of aluminum foil (20 cm2) according to Method C. The calculated thickness of the drug film was 2.5 μm. The substrate was heated as described in Method C at 90 V for 4 seconds. The purity of the drug-aerosol particles was determined to be 66.3%. 1.05 mg was recovered from the glass tube walls after vaporization, for a percent yield of 21%.

Sildenafil was also coated on a piece of stainless steel foil (6 cm2) according to Method B. 0.227 mg of drug was applied to the substrate, for a calculated drug film thickness of 0.4 μm. The substrate was heated as described in Method B by charging the capacitors to 16 V. The purity of the drug-aerosol particles was determined to be 99.3%. 0.224 mg was recovered from the filter after vaporization, for a percent yield of 98.7%. A total mass of 0.227 mg was recovered from the test apparatus and substrate, for a total recovery of 100%.

High speed photographs were taken as the drug-coated substrate was heated to monitor visually formation of a thermal vapor. The photographs showed that a thermal vapor was initially visible 45 milliseconds after heating was initiated, with the majority of the thermal vapor formed by 250 milliseconds. Generation of the thermal vapor was complete by 400 milliseconds.

Sildenafil was also coated on a piece of aluminum foil at a calculated film thickness of 3.4 μm, 3.3 μm, 1.6 μm, 0.8 μm, 0.78 μm, 0.36 μm, 0.34 μm, 0.29 μm, and 0.1 μm. The coated substrate was placed on an aluminum block that was preheated to 275° C. using a hot plate. A Pyrex© beaker was synchronously placed over the foil and the substrate was heated for 1 minute. The material collected on the beaker walls was recovered and analyzed by reverse-phase HPLC analysis with detection by absorption of 250 nm light to determine the purity of the aerosol. The purity of the drug-aerosol particles was determined to be 84.8% purity at 3.4 μm thickness; 80.1% purity at 3.3 μm thickness; 89.8% purity at 1.6 μm thickness; 93.8% purity at 0.8 μm thickness; 91.6% purity at 0.78 μm thickness; 98.0% purity at 0.36 μm thickness; 98.6% purity at 0.34 μm thickness; 97.6% purity at 0.29 μm thickness; and 100% purity at 0.1 μm thickness.

### Example 135

Spironolactone (MW 417, melting point 135° C., oral dose 25 mg), a cardiovascular agent, was coated on a stainless steel cylinder (8 cm2) according to Method D. 0.71 mg of drug was applied to the substrate, for a calculated drug film thickness of 0.9 μm. The substrate was heated as described in Method D by charging the capacitors to 20.5 V. The purity of the drug-aerosol particles was determined to be >99.5%. 0.41 mg was recovered from the filter after vaporization, for a percent yield of 57.7%. A total mass of 0.7 mg was recovered from the test apparatus and substrate, for a total recovery of 98.6%.

### Example 136

Sumatriptan (MW 295, melting point 171° C., oral dose 6 mg), a migraine preparation, was coated on a stainless steel cylinder (8 cm2) according to Method E. 1.22 mg of drug was applied to the substrate, for a calculated drug film thickness of 1.5 μm. The substrate was heated as described in Method E and purity of the drug-aerosol particles was determined to be 97.9%. 0.613 mg was recovered from the filter after vaporization, for a percent yield of 50.2%. A total mass of 1.03 mg was recovered from the test apparatus and substrate, for a total recovery of 84.4%.

High speed photographs were taken as the drug-coated substrate was heated to monitor visually formation of a thermal vapor. The photographs showed that a thermal vapor was initially visible 35 milliseconds after heating was initiated, with the majority of the thermal vapor formed by 175 milliseconds. Generation of the thermal vapor was complete by 600 milliseconds.

### Example 137

Sibutramine (MW 280, oral dose 10 mg), an obesity management appetite suppressant, was coated on a stainless steel cylinder (8 cm2) according to Method D. 1.667 mg of drug was applied to the substrate, for a calculated drug film thickness of 2 μm. The substrate was heated as described in Method D (with the single exception that the circuit capacitance was 1.5 Farad, not 2.0 Farad), and purity of the drug-aerosol particles was determined to be 94%. 0.861 mg was recovered from the filter after vaporization, for a percent yield of 51.6%. A total mass of 1.35 mg was recovered from the test apparatus and substrate, for a total recovery of 81%.

High speed photographs were taken as the drug-coated substrate was heated to monitor visually formation of a thermal vapor. The photographs showed that a thermal vapor was initially visible 25 milliseconds after heating was initiated, with the majority of the thermal vapor formed by 55 milliseconds. Generation of the thermal vapor was complete by 150 milliseconds.

### Example 138

Tamoxifen (MW 372, melting point 98° C., oral dose 10 mg), an antineoplastic, was coated on a stainless steel cylinder (8 cm2) according to Method D. 0.46 mg of drug was applied to the substrate, for a calculated drug film thickness of 0.6 μm. The substrate was heated as described in Method D by charging the capacitors to 20.5 V. The purity of the drug-aerosol particles was determined to be 91.4%. 0.27 mg was recovered from the filter after vaporization, for a percent yield of 58.7%. A total mass of 0.39 mg was recovered from the test apparatus and substrate, for a total recovery of 84.8%.

High speed photographs were taken as the drug-coated substrate was heated to monitor visually formation of a thermal vapor. The photographs showed that a thermal vapor was initially visible 30 milliseconds after heating was initiated, with the majority of the thermal vapor formed by 70 milliseconds. Generation of the thermal vapor was complete by 250 milliseconds.

### Example 139

Tacrine (MW 198, melting point 184° C.), an Alzheimer's disease manager, was coated on a stainless steel cylinder (8 cm2) according to Method D. 0.978 mg of drug was applied to the substrate, for a calculated drug film thickness of 1.2 μm. The substrate was heated as described in Method D by charging the capacitors to 20.5 V. The purity of the drug-aerosol particles was determined to be 99.8%. 0.502 mg was recovered from the filter after vaporization, for a percent yield of 51.3%. A total mass of 0.841 mg was recovered from the test apparatus and substrate, for a total recovery of 86%.

### Example 140

Tadalafil (MW 389, oral dose 5 mg), an erectile dysfunction therapeutic agent, was coated onto eight stainless steel foil substrates (5 cm2) according to Method B. The calculated thickness of the drug film on each substrate ranged from about 0.5 μm to about 2.9 μm. The substrates were heated as described in Method B by charging the capacitors to 16 V. Purity of the drug-aerosol particles from each substrate was determined and the results are shown in FIG. 17.

Tadalafil was also coated on a stainless steel cylinder (8 cm2). The calculated thickness of the drug film was 4.5 μm. The substrate was heated as described by the flashbulb and the purity of the drug-aerosol particles was determined to be 94.9%. 0.67 mg was recovered from the filter after vaporization, for a percent yield of 18.1%. A total mass of 1.38 mg was recovered from the test apparatus and substrate, for a total recovery of 37.3%.

Tadalafil was also coated on a piece of aluminum foil (20 cm2) according to Method C. The calculated thickness of the drug film was 0.5 μm. The substrate was heated as described in Method C at 60 V for 13 seconds. The purity of the drug-aerosol particles was determined to be 91.2%. 0.45 mg was recovered from the glass tube walls after vaporization, for a percent yield of 45%.

Tadalafil was also coated on a piece of stainless steel foil (5 cm2) according to Method B. 1.559 mg of drug was applied to the substrate, for a calculated drug film thickness of 2.9 μm. The substrate was heated as described in Method B by charging the capacitors to 16 V. The purity of the drug-aerosol particles was determined to be 95.8%. 1.42 mg was recovered from the filter after vaporization, for a percent yield of 91.1%. A total mass of 1.559 mg was recovered from the test apparatus and substrate, for a total recovery of 100%.

The drug was also coated (1.653 mg) to a thickness of 3.1 μm on a piece of stainless steel foil (5 cm2) according to Method B. The substrate was heated under an N2 atmosphere by charging the capacitors to 16 V. The purity of the drug-aerosol particles was determined to be 99.2%. 1.473 mg was recovered from the filter after vaporization, for a percent yield of 89.1%. A total mass of 1.653 mg was recovered from the test apparatus and substrate, for a total recovery of 100%.

### Example 141

Terbutaline (MW 225, melting point 122° C., oral dose 0.2 mg), a respiratory agent, was coated on a stainless steel cylinder (9 cm2) according to Method D. 2.32 mg of drug was applied to the substrate, for a calculated drug film thickness of 2.7 μm. The substrate was heated as described in Method D by charging the capacitors to 20.5 V. The purity of the drug-aerosol particles was determined to be 99.3%. 1.54 mg was recovered from the filter after vaporization, for a percent yield of 66.4%. A total mass of 1.938 mg was recovered from the test apparatus and substrate, for a total recovery of 83.5%.

### Example 142

Testosterone (MW 288, melting point 155° C., oral dose 3 mg), a hormone, was coated on a stainless steel cylinder (8 cm2) according to Method D. 0.96 mg of drug was applied to the substrate, for a calculated drug film thickness of 1.2 μm. The substrate was heated as described in Method D by charging the capacitors to 20.5 V. The purity of the drug-aerosol particles was determined to be 99.6%. 0.62 mg was recovered from the filter after vaporization, for a percent yield of 64.6%. A total mass of 0.96 mg was recovered from the test apparatus and substrate, for a total recovery of 100%.

### Example 143

Thalidomide (MW 258, melting point 271° C., oral dose 100 mg), an immunomodulator, was coated on a stainless steel cylinder (8 cm2) according to Method D. 0.57 mg of drug was applied to the substrate, for a calculated drug film thickness of 0.7 μm. The substrate was heated as described in Method D by charging the capacitors to 20.5 V. The purity of the drug-aerosol particles was determined to be >99.5%. 0.43 mg was recovered from the filter after vaporization, for a percent yield of 75.4%. A total mass of 0.54 mg was recovered from the test apparatus and substrate, for a total recovery of 94.7%.

### Example 144

Theophylline (MW 180, melting point 274° C., oral dose 200 mg), a respiratory agent, was coated on a stainless steel cylinder (8 cm2) according to Method D. 0.859 mg of drug was applied to the substrate, for a calculated drug film thickness of 1.0 μm. The substrate was heated as described in Method D by charging the capacitors to 20.5 V. The purity of the drug-aerosol particles was determined to be 100.0%. 0.528 mg was recovered from the filter after vaporization, for a percent yield of 61.5%. A total mass of 0.859 mg was recovered from the test apparatus and substrate, for a total recovery of 100%.

High speed photographs were taken as the drug-coated substrate was heated to monitor visually formation of a thermal vapor. The photographs showed that a thermal vapor was initially visible 40 milliseconds after heating was initiated, with the majority of the thermal vapor formed by 160 milliseconds. Generation of the thermal vapor was complete by 350 milliseconds.

### Example 145

Tocainide (MW 192, melting point 247° C., oral dose 400 mg), a cardiovascular agent, was coated on a stainless steel cylinder (8 cm2) according to Method D. 0.86 mg of drug was applied to the substrate, for a calculated drug film thickness of 1 μm. The substrate was heated as described in Method D by charging the capacitors to 20.5 V. The purity of the drug-aerosol particles was determined to be 99.7%. 0.65 mg was recovered from the filter after vaporization, for a percent yield of 75.6%. A total mass of 0.86 mg was recovered from the test apparatus and substrate, for a total recovery of 100%.

High speed photographs were taken as the drug-coated substrate was heated to monitor visually formation of a thermal vapor. The photographs showed that a thermal vapor was initially visible 25 milliseconds after heating was initiated, with the majority of the thermal vapor formed by 75 milliseconds. Generation of the thermal vapor was complete by 130 milliseconds.

### Example 146

Tolfenamic Acid (MW 262, melting point 208° C., oral dose 200 mg), an analgesic, was coated on a piece of aluminum foil (20 cm2) according to Method C. The calculated thickness of the drug film was 5.0 μm. The substrate was heated as described in Method C at 60 V for 6 seconds. The purity of the drug-aerosol particles was determined to be 94.2%. 6.49 mg was recovered from the glass tube walls after vaporization, for a percent yield of 65.6%.

### Example 147

Tolterodine (MW 325, oral dose 2 mg), an urinary tract agent, was coated on a stainless steel cylinder (8 cm2) according to Method D. 1.39 mg of drug was applied to the substrate, for a calculated drug film thickness of 1.7 μm. The substrate was heated as described in Method D by charging the capacitors to 20.5 V. The purity of the drug-aerosol particles was determined to be 96.9%. 1.03 mg was recovered from the filter after vaporization, for a percent yield of 74.1%. A total mass of 1.39 mg was recovered from the test apparatus and substrate, for a total recovery of 100%.

High speed photographs were taken as the drug-coated substrate was heated to monitor visually formation of a thermal vapor. The photographs showed that a thermal vapor was initially visible 30 milliseconds after heating was initiated, with the majority of the thermal vapor formed by 80 milliseconds. Generation of the thermal vapor was complete by 100 milliseconds.

### Example 148

Toremifene (MW 406, melting point 110° C., oral dose 60 mg), an antineoplastic, was coated on a stainless steel cylinder (8 cm2). 1.20 mg of drug was applied to the substrate, for a calculated thickness of the drug film of 1.4 μm, and heated to form drug-aerosol particles according to Method D by charging the capacitors to 20.5 V. The purity of the drug-aerosol particles was determined to be 98.7%. The yield of aerosol particles was 50%. 1.09 mg of total mass was recovered for a total recovery yield of 90.8%.

### Example 149

Tramadol (MW 263, oral dose 50 mg), an analgesic, was coated on an aluminum foil substrate (20 cm2) according to Method C. 4.90 mg of drug was applied to the substrate, for a calculated thickness of the drug film of 2.5 μm. The substrate was heated as described in Method C at 108 V for 2.25 seconds. The purity of the drug-aerosol particles was determined to be 96.9%. 3.39 mg was recovered from the glass tube walls after vaporization, for a percent yield of 69.2%.

Tramadol (2.6 mg) was also coated on a piece of aluminum foil (20 cm2) according to Method C to a film thickness (calculated) of 1.3 μm. The substrate was heated as described in Method C under an argon atmosphere at 90 V for 3.5 seconds. The purity of the drug-aerosol particles was determined to be 96.1%. 1.79 mg was recovered from the glass tube walls after vaporization, for a percent yield of 68.8%.

Tramadol (2.1 mg) was also coated on a piece of aluminum foil (20 cm2) according to Method C to a film thickness (calculated) of 1.1 μm. The substrate was heated as described in Method C under air at 90 V for 3.5 seconds. The purity of the drug-aerosol particles was determined to be 96.6%. 1.33 mg was recovered from the glass tube walls after vaporization, for a percent yield of 63.8%.

The hydrochloride salt form was also tested. 2.6 mg of drug was coated onto an aluminum foil substrate (20 cm2) according to Method C to a film thickness (calculated) of 1.3 μm. The substrate was heated as described in Method C and purity of the drug-aerosol particles was determined to be 97.6%. 1.67 mg was recovered from the glass tube walls after vaporization, for a percent yield of 64.2%. An identical substrate having an identical drug film thickness was tested under an argon atmosphere at 90 V for 3.5 seconds. The purity of the drug-aerosol particles was determined to be 89%. 1.58 mg was recovered from the glass tube walls after vaporization, for a percent yield of 60.8%

Tramadol (17.5 mg) was also coated on a piece of aluminum foil (40 cm2) according to Method F to a film thickness (calculated) of 4.38 μm. The substrate was heated as described in Method F and purity of the drug-aerosol particles was determined to be 97.3%.

### Example 150

Tranylcypromine (MW 133, melting point <25° C., oral dose 30 mg), a psychotherapeutic agent, was coated on a piece of aluminum foil (20 cm2) according to Method C. The calculated thickness of the drug film was 5.4 μm. The substrate was heated as described in Method C at 90 V for 5 seconds. The purity of the drug-aerosol particles was determined to be 93.7%. 7.4 mg was recovered from the glass tube walls after vaporization, for a percent yield of 68.5%.

Another substrate containing tranylcypromine coated to a film thickness of 2.7 μm was prepared by the same method and heated under an argon atmosphere at 90 V for 3.5 seconds. The purity of the drug-aerosol particles was determined to be 95.9%. 3 mg was recovered from the glass tube walls after vaporization, for a percent yield of 56.6%.

Tranylcypromine HCl (MW 169, melting point 166° C., oral dose 30 mg), a psychotherapeutic agent, was coated on a piece of aluminum foil (20 cm2) according to Method C. The calculated thickness of the drug film was 1.2 μm. The substrate was heated as described in Method C at 90 V for 3.5 seconds. The purity of the drug-aerosol particles was determined to be 97.5%. 1.3 mg was recovered from the glass tube walls after vaporization, for a percent yield of 56.5%.

### Example 151

Trazodone (MW 372, melting point 87° C., oral dose 400 mg), a psychotherapeutic agent, was coated on an aluminum foil substrate (20 cm2) according to Method C. 10.0 mg of drug was applied to the substrate, for a calculated thickness of the drug film of 5.0 μm. The substrate was heated as described in Method C at 60 V for 15 seconds. The purity of the drug-aerosol particles was determined to be 98.9%. 8.5 mg was recovered from the glass tube walls after vaporization, for a percent yield of 85%.

Trazodone was further coated on an aluminum foil substrate according to Method G. The substrate was heated as described in Method G at 90 V for 3.5 seconds. The purity of the drug-aerosol particles was determined to be 97.9%. The percent yield of the aerosol was 29.1%. The purity of the drug-aerosol particles was determined to be 98.5% when the system was flushed through with argon prior to volatilization. The percent yield of the aerosol was 25.5%.

### Example 152

Triazolam (MW 343, melting point 235° C., oral dose 0.13 mg), a sedative and hypnotic, was coated on an aluminum foil substrate (20 cm2) according to Method C. 1.7 mg of drug was applied to the substrate, for a calculated thickness of the drug film of 0.9 μm. The substrate was heated as described in Method C at 45 V for 18 seconds. The purity of the drug-aerosol particles was determined to be 99.2%. 1.6 mg was recovered from the glass tube walls after vaporization, for a percent yield of 94.1%.

Another aluminum foil substrate (28.8 cm2) was prepared according to Method C. 1.7 mg of triazolam was applied to the substrate, for a calculated thickness of the drug film of 0.69 μm. The substrate was heated substantially as described in Method C at 75 V for 2 seconds and then at 45 V for 8 seconds. The purity of the drug-aerosol particles was determined to be 99.3%. 1.7 mg of aerosol particles were collected for a percent yield of 100%

Triazolam was also applied to an aluminum foil substrate (36 cm2) according to Method G. 0.6 mg of the drug was applied to the substrate, for a calculated thickness of the drug film of 0.17 μm. The substrate was heated substantially as described in Method G at 90 V for 6 seconds, except that one of the openings of the T-shaped tube was sealed with a rubber stopper, one was loosely covered with the end of the halogen tube, and the third connected to the 1 L flask. The purity of the drug-aerosol particles was determined to be >99%. All of the drug was found to have aerosolized, for a percent yield of 100%.

### Example 153

Trifluoperazine (MW 407, melting point <25° C., oral dose 7.5 mg), a psychotherapeutic agent, was coated on a stainless steel cylinder (9 cm2) according to Method D. 1.034 mg of drug was applied to the substrate, for a calculated drug film thickness of 1.1 μm. The substrate was heated as described in Method D by charging the capacitors to 19 V. The purity of the drug-aerosol particles was determined to be 99.8%. 0.669 mg was recovered from the filter after vaporization, for a percent yield of 64.7%. A total mass of 1.034 mg was recovered from the test apparatus and substrate, for a total recovery of 100%.

Trifluoperazine 2HCl salt (MW 480, melting point 243° C., oral dose 7.5 mg) was coated on an identical substrate. Specifically, 0.967 mg of drug was applied to the substrate, for a calculated drug film thickness of 1.1 μm. The substrate was heated as described in Method D by charging the capacitors to 20.5 V. The purity of the drug-aerosol particles was determined to be 87.5%. 0.519 mg was recovered from the filter after vaporization, for a percent yield of 53.7%. A total mass of 0.935 mg was recovered from the test apparatus and substrate, for a total recovery of 96.7%.

High speed photographs of trifluoperazine 2HCl were taken as the drug-coated substrate was heated to monitor visually formation of a thermal vapor. The photographs showed that a thermal vapor was initially visible 25 milliseconds after heating was initiated, with the majority of the thermal vapor formed by 120 milliseconds. Generation of the thermal vapor was complete by 250 milliseconds.

### Example 154

Trimipramine maleate (MW 411, melting point 142° C., oral dose 50 mg), a psychotherapeutic agent, was coated on a piece of aluminum foil (20 cm2) according to Method C. The calculated thickness of the drug film was 1.2 μm. The substrate was heated as described in Method C at 90 V for 3.5 seconds. The purity of the drug-aerosol particles was determined to be 95.9%. 1.6 mg was recovered from the glass tube walls after vaporization, for a percent yield of 66.7%.

Another substrate containing trimipramine maleate coated to a film thickness of 1.1 μm was prepared by the same method and heated under an argon atmosphere at 90 V for 3.5 seconds. The purity of the drug-aerosol particles was determined to be 97.4%. 2.1 mg was recovered from the glass tube walls after vaporization, for a percent yield of 95.5%.

### Example 155

Valdecoxib (MW 314, melting point 155° C., oral dose 10 mg), an anti-rheumatic agent, was coated on a piece of stainless steel foil (5 cm2) according to Method B. The calculated thickness of the drug film was 8.0 μm. The substrate was heated as described in Method B by charging the capacitors to 15.5 V. The purity of the drug-aerosol particles was determined to be 96.9%. 1.235 mg was recovered from the filter after vaporization, for a percent yield of 28.9%. A total mass of 3.758 mg was recovered from the test apparatus and substrate, for a total recovery of 87.9%.

Valdecoxib was also coated on a piece of stainless steel foil (6 cm2) according to Method B. 0.716 mg of drug was applied to the substrate, for a calculated drug film thickness of 1.3 μm. The substrate was heated as described in Method B by charging the capacitors to 15 V. The purity of the drug-aerosol particles was determined to be 98.6%. 0.466 mg was recovered from the filter after vaporization, for a percent yield of 65.1%. A total mass of 0.49 mg was recovered from the test apparatus and substrate, for a total recovery of 68.4%.

### Example 156

Valproic Acid (MW 144, melting point <25° C., oral dose 60 mg), an anticonvulsant, was coated on a metal substrate (50 cm2) according to Method F. 82.4 mg of drug was applied to the substrate, for a calculated drug film thickness of 16.5 μm. The substrate was heated according to Method F at 300° C. to form drag-aerosol particles. Purity of the drug-aerosol particles was determined to be 99.7% by GC analysis. 60 mg of the drug were collected for a percent yield of 72.8%.

### Example 157

Vardenafil (MW 489, oral dose 5 mg), an erectile dysfunction therapy agent, was coated on a stainless steel cylinder (6 cm2) according to Method E. The calculated thickness of the drug film was 2.7 μm. The substrate was heated as described in Method E and purity of the drug-aerosol particles was determined to be 79%. 0.723 mg was recovered from the filter after vaporization, for a percent yield of 44.4%.

Another substrate (stainless steel cylinder (6 cm2)) was prepared by applying 0.18 mg drug to form a film 0.3 μm in thickness. The substrate was heated as described in Method E and purity of the drug-aerosol particles was determined to be 96.8%. 0.11 mg was recovered from the filter after vaporization, for a percent yield of 63.1%. A total mass of 0.14 mg was recovered from the test apparatus and substrate, for a total recovery of 81.8%.

High speed photographs were taken as the drug-coated substrate was heated to monitor visually formation of a thermal vapor. The photographs showed that a thermal vapor was initially visible 30 milliseconds after heating was initiated, with the majority of the thermal vapor formed by 90 milliseconds. Generation of the thermal vapor was complete by 110 milliseconds.

### Example 158

Venlafaxine (MW 277, oral dose 50 mg), a psychotherapeutic agent, was coated on a stainless steel cylinder (6 cm2) according to Method E. 5.85 mg of drug was applied to the substrate, for a calculated drug film thickness of 9.8 μm. The substrate was heated as described in Method E and purity of the drug-aerosol particles was determined to be 99.4%. 3.402 mg was recovered from the filter after vaporization, for a percent yield of 58.1%. A total mass of 5.85 mg was recovered from the test apparatus and substrate, for a total recovery of 100%.

High speed photographs were taken as the drug-coated substrate was heated to monitor visually formation of a thermal vapor. The photographs showed that a thermal vapor was initially visible 30 milliseconds after heating was initiated, with the majority of the thermal vapor formed by 100 milliseconds. Generation of the thermal vapor was complete by 400 milliseconds.

### Example 159

Verapamil (MW 455, melting point <25° C., oral dose 40 mg), a cardiovascular agent, was coated on a piece of aluminum foil (20 cm2) according to Method C. The calculated thickness of the drug film was 1.1 μm. The substrate was heated under an argon atmosphere at 90 V for 3.5 seconds. The purity of the drug-aerosol particles was determined to be 96.2%. 1.41 mg was recovered from the glass tube walls after vaporization, for a percent yield of 64.1%.

Verapamil was also coated on a stainless steel cylinder (8 cm2) according to Method D. 0.75 mg of drug was applied to the substrate, for a calculated drug film thickness of 0.9 μm. The substrate was heated as described in Method D by charging the capacitors to 20.5 V. The purity of the drug-aerosol particles was determined to be 89.6%. 0.32 mg was recovered from the filter after vaporization, for a percent yield of 42.7%. A total mass of 0.6 mg was recovered from the test apparatus and substrate, for a total recovery of 80%.

### Example 160

Vitamin E (MW 430, melting point 4° C.), a dietary supplement, was coated on a stainless steel cylinder (8 cm2) according to Method D. 0.78 mg of drug was applied to the substrate, for a calculated drug film thickness of 0.9 μm. The substrate was heated as described in Method D by charging the capacitors to 20.5 V. The purity of the drug-aerosol particles was determined to be 99.3%. 0.48 mg was recovered from the filter after vaporization, for a percent yield of 61.8%. A total mass of 0.6 mg was recovered from the test apparatus and substrate, for a total recovery of 81.4%.

### Example 161

Zaleplon (MW 305, melting point 159° C., oral dose 5 mg), a sedative and hypnotic, was coated on a piece of aluminum foil (20 cm2) according to Method C. The calculated thickness of the drug film was 2.3 μm. The substrate was heated as described in Method C at 60 V for 12 seconds. The purity of the drug-aerosol particles was determined to be 99.5%. 4.07 mg was recovered from the glass tube walls after vaporization, for a percent yield of 90.4%.

### Example 162

Zolmitriptan (MW 287, melting point 141° C., oral dose 1.25 mg), a migraine preparation, was coated on a piece of aluminum foil (20 cm2) according to Method C. The calculated thickness of the drug film was 1.6 μm. The substrate was heated as described in Method C at 60 V for 11 seconds. The purity of the drug-aerosol particles was determined to be 93%. 1.1 mg was recovered from the glass tube walls after vaporization, for a percent yield of 35.5%.

Another substrate containing zolmitriptan coated to a film thickness of 2.0 μm was prepared by the same method and heated under an argon atmosphere at 90 V for 4 seconds. The purity of the drug-aerosol particles was determined to be 98.4%. 0.6 mg was recovered from the glass tube walls after vaporization, for a percent yield of 15%.

Another substrate (36 cm2) containing zolmitriptan was prepared according to Method C. 9.8 mg of the drug was applied to the substrate, for a calculated thickness of the drug film of 2.7 μm. The substrate was heated substantially as described in Method C at 60 V for 15 seconds. The purity of the drug-aerosol particles was determined to be 98%. The aerosol percent yield was 38%.

Zolmitriptan was further coated on an aluminum foil substrate (24.5 cm2) according to Method G. 2.6 mg of the drug was applied to the substrate, for a calculated thickness of the drug film of 1.1 μm. The substrate was heated as described in Method G at 90 V for 6 seconds. The purity of the drug-aerosol particles was determined to be >96%. 1.5 mg of the drug was found to have aerosolized, for a percent yield of 57.7%.

### Example 163

Zolpidem (MW 307, melting point 196° C., oral dose 5 mg), a sedative and hypnotic, was coated onto six stainless steel cylindrical substrates according to Method E. The calculated thickness of the drug film on each substrate ranged from about 0.1 μm to about 4.2 μm. The substrates were heated as described in Method E and purity of the drug-aerosol particles generated from each substrate determined. The results are shown in FIG. 19.

Zolpidem was also coated on a stainless steel cylinder (6 cm2) according to Method E. 4.13 mg of drug was applied to the substrate, for a calculated drug film thickness of 6.9 μm. The substrate was heated as described in Method E and purity of the drug-aerosol particles was determined to be 96.6%. 2.6 mg was recovered from the filter after vaporization, for a percent yield of 63%. A total mass of 3.18 mg was recovered from the test apparatus and substrate, for a total recovery of 77%.

High speed photographs were taken as the drug-coated substrate was heated to monitor visually formation of a thermal vapor. The photographs showed that a thermal vapor was initially visible 35 milliseconds after heating was initiated, with the majority of the thermal vapor formed by 120 milliseconds. Generation of the thermal vapor was complete by 225 milliseconds.

Zolpidem was also coated on an aluminum substrate (24.5 cm2) according to Method G. 8.3 mg of drug was applied to the substrate, for a calculated drug film thickness of 3.4 μm. The substrate was heated as described in Method G at 90 V for 6 seconds. The purity of the drug-aerosol particles was determined to be >97%. 7.4 mg of the drug was found to have aerosolized by weight loss from substrate mass, for a percent yield of 89.2%.

### Example 164

Zopiclone (MW 388, melting point 178° C., oral dose 7.50 mg), a sedative and hypnotic, was coated on an aluminum foil substrate (20 cm2) according to Method C. 3.7 mg of drug was applied to the substrate, for a calculated thickness of the drug film of 1.9 μm. The substrate was heated as described in Method C at 60 V for 9 seconds. The purity of the drug-aerosol particles was determined to be 97.9%. 2.5 mg was recovered from the glass tube walls after vaporization, for a percent yield of 67.6%.

Zopiclone was further coated on an aluminum foil substrate (24 cm2) according to Method C. 3.5 mg of drug was applied to the substrate, for a calculated thickness of the drug film of 1.5 μm. The substrate was heated substantially as described in Method C at 60 V for 6 seconds. The purity of the drug-aerosol particles was determined to be >99%.

### Example 165

Zotepine (MW 332, melting point 91° C., oral dose 25 mg), a psychotherapeutic agent, was coated on a stainless steel cylinder (8 cm2) according to Method D. 0.82 mg of drug was applied to the substrate, for a calculated drug film thickness of 1 μm. The substrate was heated as described in Method D by charging the capacitors to 20.5 V. The purity of the drug-aerosol particles was determined to be 98.3%. 0.72 mg was recovered from the filter after vaporization, for a percent yield of 87.8%. A total mass of 0.82 mg was recovered from the test apparatus and substrate, for a total recovery of 100%.

High speed photographs were taken as the drug-coated substrate was heated to monitor visually formation of a thermal vapor. The photographs showed that a thermal vapor was initially visible 30 milliseconds after heating was initiated, with the majority of the thermal vapor formed by 60 milliseconds. Generation of the thermal vapor was complete by 110 milliseconds.

### Example 166

Adenosine (MW 267, melting point 235° C., oral dose 6 mg), an anti-arrhythmic cardiovascular agent, was coated on a stainless steel cylinder (8 cm2) according to Method D. 1.23 mg of drug was applied to the substrate, for a calculated drug film thickness of 1.5 μm. The substrate was heated as described in Method D by charging the capacitors to 20.5 V. The purity of the drug-aerosol particles was determined to be 70.6%. 0.34 mg was recovered from the filter after vaporization, for a percent yield of 27.6%. A total mass of 0.68 mg was recovered from the test apparatus and substrate, for a total recovery of 55.3%.

High speed photographs were taken as the drug-coated substrate was heated to monitor visually formation of a thermal vapor. The photographs showed that a thermal vapor was initially visible 40 milliseconds after heating was initiated, with the majority of the thermal vapor formed by 250 milliseconds. Generation of the thermal vapor was complete by 535 milliseconds.

### Example 167

Amoxapine (MW 314, melting point 176° C., oral dose 25 mg), an anti-psychotic agent, was coated on a stainless steel cylinder (8 cm2) according to Method D. 6.61 mg of drug was applied to the substrate, for a calculated drug film thickness of 7.9 μm. The substrate was heated as described in Method D by charging the capacitors to 20.5 V. The purity of the drug-aerosol particles was determined to be 99.7%. 3.13 mg was recovered from the filter after vaporization, for a percent yield of 47.4%. A total mass of 6.61 mg was recovered from the test apparatus and substrate, for a total recovery of 100%.

### Example 168

Apomorphine 10,11 cyclocarbonate (MW 293, typical aerosol dose 1 mg), a dopaminergic agent used in Parkinson's patients, was coated on a piece of aluminum foil (20 cm2) according to Method C. The calculated thickness of the drug film was 1.2 μm. The substrate was heated as described in Method C at 90 V for 3 seconds. The purity of the drug-aerosol particles was determined to be 78.4%. 1.46 mg was recovered from the glass tube walls after vaporization, for a percent yield of 60.8%.

### Example 169

Aripiprazole (MW 448, melting point 140° C., oral dose 5 mg), an anti-psychotic agent, was coated on a stainless steel cylinder (8 cm2) according to Method D. 1.139 mg of drug was applied to the substrate, for a calculated drug film thickness of 1.4 μm. The substrate was heated as described in Method D by charging the capacitors to 20.5 V. The purity of the drug-aerosol particles was determined to be 91.1%. 0.251 mg was recovered from the filter after vaporization, for a percent yield of 22%. A total mass of 1.12 mg was recovered from the test apparatus and substrate, for a total recovery of 98%.

High speed photographs were taken as the drug-coated substrate was heated to monitor visually formation of a thermal vapor. The photographs showed that a thermal vapor was initially visible 55 milliseconds after heating was initiated, with the majority of the thermal vapor formed by 300 milliseconds. Generation of the thermal vapor was complete by 1250 milliseconds.

A second substrate coated with arirpirazole was prepared for testing. 1.139 mg was coated on a stainless steel cylinder (8 cm2) according to Method D, for a calculated drug film thickness of 1.4 μm. The substrate was heated as described in Method D by charging the capacitors to 20.5 V. The purity of the drug-aerosol particles was determined to be 86.9%. 0.635 mg was recovered from the filter after vaporization, for a percent yield of 55.8%. A total mass of 1.092 mg was recovered from the test apparatus and substrate, for a total recovery of 95.8%.

High speed photographs were taken as the drug-coated substrate was heated to monitor visually formation of a thermal vapor. The photographs showed that a thermal vapor was initially visible 30 milliseconds after heating was initiated, with the majority of the thermal vapor formed by 200 milliseconds. Generation of the thermal vapor was complete by 425 milliseconds.

### Example 170

Aspirin (MW 180, melting point 135° C., oral dose 325 mg), an analgesic agent, was coated on a piece of aluminum foil (20 cm2) according to Method C. The calculated thickness of the drug film was 1.2 μm. The substrate was heated as described in Method C at 60 V for 5 seconds. The purity of the drug-aerosol particles was determined to be 82.1%. 1.23 mg was recovered from the glass tube walls after vaporization, for a percent yield of 53.5%.

### Example 171

Astemizole (MW 459, melting point 173° C., oral dose 10 mg), an antihistamine, was coated on an aluminum foil substrate (20 cm2) according to Method C. 5.0 mg of drug was applied to the substrate, for a calculated thickness of the drug film of 2.5 μm. The substrate was heated as described in Method C at 60 V for 11 seconds. The purity of the drug-aerosol particles was determined to be 88%. 1.6 mg was recovered from the glass tube walls after vaporization, for a percent yield of 32.0%.

A similarly prepared substrate having the same film thickness was heated at 60 V for 11 seconds under a pure argon atmosphere. The purity of the drug-aerosol particles was determined to be 93.9%. 1.7 mg was recovered from the glass tube walls after vaporization, for a percent yield of 34.0%.

### Example 172

Atenolol (MW 266, melting point 152° C., oral dose 25 mg), a beta adrenergic blocking agent, was coated on a piece of aluminum foil (20 cm2) according to Method C. 22.6 mg was applied to the substrate, for a calculated thickness of the drug film of 11.3 μm. The substrate was heated as described in Method C at 60 V for 11 seconds. The purity of the drug-aerosol particles was determined to be 94%. 1.0 mg was recovered from the glass tube walls after vaporization, for a percent yield of 4.4%.

Another atenolol-coated substrate was prepared by the same method, with 17.9 mg of drug applied to the substrate, for a calculated film thickness of 9.0 μm. The substrate was heated under an argon atmosphere according to Method C at 60 V for 3.5 seconds. The purity of the drug-aerosol particles was determined to be >99.5%. 2.0 mg was recovered from the glass tube walls after vaporization, for a percent yield of 11%.

Atenolol was further coated on an aluminum foil substrate according to Method G. The substrate was heated as described in Method G, and the purity of the drug-aerosol particles was determined to be 100%. The percent yield of the aerosol was 10%.

### Example 173

Benazepril (MW 424, melting point 149° C., oral dose 10 mg), an ACE inhibitor, cardiovascular agent, was coated on a stainless steel cylinder (8 cm2) according to Method D. The calculated thickness of the drug film was 0.9 μm. The substrate was heated as described in Method D by charging the capacitors to 20.5 V. The purity of the drug-aerosol particles was determined to be 90%. 0.34 mg was recovered from the filter after vaporization, for a percent yield of 45.3%. A total mass of 0.6 mg was recovered from the test apparatus and substrate, for a total recovery of 77.3%.

### Example 174

Benztropine (MW 307, melting point 143° C., oral dose 1 mg), an anti-cholinergic, antiparkinsonian agent, was coated onto an aluminum foil substrate (20 cm2) according to Method C. 2.10 mg of drug was applied to the substrate, for a calculated thickness of the drug film of 1.1 μm. The substrate was heated as described in Method C at 90 V for 3.5 seconds. The purity of the drug-aerosol particles was determined to be 98.3%. 0.83 mg was recovered from the glass tube walls after vaporization, for a percent yield of 39.5%.

Another benztropine-coated substrate was prepared by the same method, with 2.0 mg of drug was applied to the substrate, for a calculated film thickness of 1.0 μm. The substrate was heated under an argon atmosphere at 90 V for 3.5 seconds. The purity of the drug-aerosol particles was determined to be 99.5%. 0.96 mg was recovered from the glass tube walls after vaporization, for a percent yield of 48%.

### Example 175

Bromazepam (MW 316, melting point 239° C., oral dose 2 mg), a psychotherapeutic agent used as an anti-anxiety drug, was coated on a piece of aluminum foil (20 cm2) according to Method C. The calculated thickness of the drug film was 5.2 μm. The substrate was heated as described in Method C at 30 V for 45 seconds. The purity of the drug-aerosol particles was determined to be 96.9%. 2.2 mg was recovered from the glass tube walls after vaporization, for a percent yield of 21.2%.

### Example 176

Budesonide (MW 431, melting point 232° C., oral dose 0.2 mg), an anti-inflammatory steroid used as a respiratory agent, was coated on a stainless steel cylinder (9 cm2) according to Method D. 1.46 mg of drug was applied to the substrate, for a calculated drug film thickness of 1.7 μm. The substrate was heated as described in Method D by charging the capacitors to 20.5 V. The purity of the drug-aerosol particles was determined to be 70.5%. 0.37 mg was recovered from the filter after vaporization, for a percent yield of 25.3%. A total mass of 0.602 mg was recovered from the test apparatus and substrate, for a total recovery of 41.2%.

### Example 177

Buspirone (MW 386, oral dose 15 mg), a psychotherapeutic agent, was coated on an aluminum foil substrate (20 cm2) according to Method C. 7.60 mg of drug was applied to the substrate, for a calculated thickness of the drug film of 3.8 μm. The substrate was heated as described in Method C at 60 V for 7 seconds. The purity of the drug-aerosol particles was determined to be 96.5%. 1.75 mg was recovered from the glass tube walls after vaporization, for a percent yield of 23%.

Another substrate containing buspirone coated to a film thickness of 4.6 μm was prepared by the same method and heated under an argon atmosphere at 60 V for 7 seconds. The purity of the drug-aerosol particles was determined to be 96.1%. 2.7 mg was recovered from the glass tube walls after vaporization, for a percent yield of 29.7%.

The hydrochloride salt (MW 422) was also tested. Buspirone hydrochloride was coated on a piece of aluminum foil (20 cm2) according to Method C. 8.30 mg of drug was applied to the substrate, for a calculated thickness of the drug film of 4.2 μm. The substrate was heated as described in Method C at 90 V for 5 seconds. The purity of the drug-aerosol particles was determined to be 97.8%. 2.42 mg was recovered from the glass tube walls after vaporization, for a percent yield of 29.2%.

### Example 178

Caffeine (MW 194, melting point 238° C., oral dose 100 mg), a central nervous system stimulant, was coated on a metal substrate (50 cm2). 100 mg of drug was applied to the substrate, for a calculated drug film thickness of 14 μm and heated to 300° C. according to Method F to form drug-aerosol particles. Purity of the drug-aerosol particles was determined to be >99.5%. 40 mg was recovered from the glass wool after vaporization, for a percent yield of 40%.

### Example 179

Captopril (MW 217, melting point 104° C., oral dose 25 mg), an ACE inhibitor, cardiovascular agent, was coated on a stainless steel cylinder (8 cm2) according to Method D. 0.88 mg of drug was applied to the substrate, for a calculated drug film thickness of 1.1 μm. The substrate was heated as described in Method D by charging the capacitors to 20.5 V. The purity of the drug-aerosol particles was determined to be 87.5%. 0.54 mg was recovered from the filter after vaporization, for a percent yield of 61.4%. A total mass of 0.8 mg was recovered from the test apparatus and substrate, for a total recovery of 90.9%.

High speed photographs were taken as the drug-coated substrate was heated to monitor visually formation of a thermal vapor. The photographs showed that a thermal vapor was initially visible 20 milliseconds after heating was initiated, with the majority of the thermal vapor formed by 100 milliseconds. Generation of the thermal vapor was complete by 170 milliseconds.

### Example 180

Carbamazepine (MW 236, melting point 193° C., oral dose 200 mg), an anticonvulsant agent, was coated on a stainless steel cylinder (8 cm2) according to Method D. 0.73 mg of drug was applied to the substrate, for a calculated drug film thickness of 0.9 μm. The substrate was heated as described in Method D by charging the capacitors to 20.5 V. The purity of the drug-aerosol particles was determined to be 88.9%. 0.43 mg was recovered from the filter after vaporization, for a percent yield of 58.9%. A total mass of 0.6 mg was recovered from the test apparatus and substrate, for a total recovery of 78.1%.

### Example 181

Cinnarizine (MW 369, oral dose 15 mg), an antihistamine, was coated on an aluminum foil substrate (20 cm2) according to Method C. 18.0 mg of drug was applied to the substrate, for a calculated thickness of the drug film of 9 μm. The substrate was heated as described in Method C at 60 V for 8 seconds. The purity of the drug-aerosol particles was determined to be 96.7%. 3.15 mg was recovered from the glass tube walls after vaporization, for a percent yield of 17.5%.

Another substrate containing cinnarizine coated (5.20 mg drug) to a film thickness of 2.6 μm was prepared by the same method and heated under an argon atmosphere at 60 V for 8 seconds. The purity of the drug-aerosol particles was determined to be 91.8%. 2.3 mg was recovered from the glass tube walls after vaporization, for a percent yield of 44.2%.

### Example 182

Clemastine (MW 344, melting point <25° C., oral dose 1 mg), a antihistamine, was coated on a piece of aluminum foil (20 cm2) according to Method C. The calculated thickness of the drug film was 3.2 μm. The substrate was heated as described in Method C at 60 V for 7 seconds. The purity of the drug-aerosol particles was determined to be 94.3%. 3 mg was recovered from the glass tube walls after vaporization, for a percent yield of 46.9%.

Clemastine fumarate (MW 460, melting point 178° C., oral dose 1.34 mg) was coated on an identical substrate to a thickness of 2.9 μm. The substrate was heated at 60 V for 8 seconds. The purity of the drug-aerosol particles was determined to be 76.6%. 1.8 mg was recovered from the glass tube walls after vaporization, for a percent yield of 31.6%.

### Example 183

Clofazimine (MW 473, melting point 212° C., oral dose 100 mg), an anti-infective agent, was coated on a stainless steel cylinder (6 cm2) according to Method D. 0.48 mg of drug was applied to the substrate, for a calculated drug film thickness of 0.8 μm. The substrate was heated as described in Method D by charging the capacitors to 20.5 V. The purity of the drug-aerosol particles was determined to be 84.4%. 0.06 mg was recovered from the filter after vaporization, for a percent yield of 12.5%. A total mass of 0.48 mg was recovered from the test apparatus and substrate, for a total recovery of 100%.

High speed photographs were taken as the drug-coated substrate was heated to monitor visually formation of a thermal vapor. The photographs showed that a thermal vapor was initially visible 45 milliseconds after heating was initiated, with the majority of the thermal vapor formed by 300 milliseconds. Generation of the thermal vapor was complete by 1200 milliseconds.

### Example 184

Desipramine (MW 266, melting point <25° C., oral dose 25 mg), a psychotherapeutic agent, was coated on a piece of aluminum foil (20 cm2) according to Method C. The calculated thickness of the drug film was 5.2 μm. The substrate was heated as described in Method C at 90 V for 5 seconds. The purity of the drug-aerosol particles was determined to be 82.2%. 7.2 mg was recovered from the glass tube walls after vaporization, for a percent yield of 69.9%.

### Example 185

Dipyridamole (MW 505, melting point 163° C., oral dose 75 mg), a blood modifier, was coated on a stainless steel cylinder (6 cm2) according to Method D. 1.15 mg of drug was applied to the substrate, for a calculated drug film thickness of 1.9 μm. The substrate was heated as described in Method D by charging the capacitors to 20.5 V. The purity of the drug-aerosol particles was determined to be 95.3%. 0.22 mg was recovered from the filter after vaporization, for a percent yield of 19.1%. A total mass of 1.1 mg was recovered from the test apparatus and substrate, for a total recovery of 94.8%.

### Example 186

Dolasetron (MW 324, oral dose 100 mg), a gastrointestinal agent, was coated on a piece of aluminum foil (20 cm2) according to Method C. The calculated thickness of the drug film was 5 μm. The substrate was heated as described in Method C at 30 V for 45 seconds. The purity of the drug-aerosol particles was determined to be 83%. 6 mg was recovered from the glass tube walls after vaporization, for a percent yield of 60%.

Dolasetron was further coated on an aluminum foil substrate according to Method C. The substrate was heated substantially as described in Method C, and the purity of the drug-aerosol particles was determined to be 99%.

### Example 187

Doxylamine (MW 270, melting point <25° C., oral dose 12.5 mg), an antihistamine, was coated on a stainless steel cylinder (8 cm2) according to Method D. The calculated thickness of the drug film was 7.8 μm. The substrate was heated as described in Method D by charging the capacitors to 20.5 V. The purity of the drug-aerosol particles was determined to be 99.8%. 2.96 mg was recovered from the filter after vaporization, for a percent yield of 45.6%. A total mass of 6.49 mg was recovered from the test apparatus and substrate, for a total recovery of 100%.

### Example 188

Droperidol (MW 379, melting point 147° C., oral dose 1 mg), a psychotherapeutic agent, was coated on a piece of aluminum foil (20 cm2) according to Method C. The calculated thickness of the drug film was 1.1 μm. The substrate was heated as described in Method C at 90 V for 3.5 seconds. The purity of the drug-aerosol particles was determined to be 51%. 0.27 mg was recovered from the glass tube walls after vaporization, for a percent yield of 12.9%.

Another substrate containing droperidol coated to a film thickness of 1.0 μm was prepared by the same method and heated under an argon atmosphere at 90 V for 3.5 seconds. The purity of the drug-aerosol particles was determined to be 65%. 0.24 mg was recovered from the glass tube walls after vaporization, for a percent yield of 12.6%.

### Example 189

Enalapril maleate (MW 493, melting point 145° C., oral dose 5 mg), a cardiovascular agent, was coated on a stainless steel cylinder (8 cm2) according to Method D. The calculated thickness of the drug film was 1.1 μm. The substrate was heated as described in Method D by charging the capacitors to 20.5 V. The purity of the drug-aerosol particles was determined to be 61%. 0.29 mg was recovered from the filter after vaporization, for a percent yield of 34.1%. A total mass of 0.71 mg was recovered from the test apparatus and substrate, for a total recovery of 83.5%.

### Example 190

Estradiol-17-acetate (MW 314, oral dose 2 mg), a hormonal pro-drug, was coated on a piece of aluminum foil (20 cm2) according to Method C. The calculated thickness of the drug film was 0.9 μm. The substrate was heated as described in Method C at 60 V for 6 seconds. The purity of the drug-aerosol particles was determined to be 98.6%. 0.59 mg was recovered from the glass tube walls after vaporization, for a percent yield of 34.7%.

### Example 191

Estradiol 17-heptanoate (MW 384 melting point 94° C., oral dose 1 mg), a hormone, was coated on a metal substrate (50 cm2). 42 mg was applied to the substrate, for a calculated drug film thickness of 8.4 μm and heated according to Method F at 300° C. to form drug-aerosol particles. Purity of the drug-aerosol particles was determined to be 90% by GC analysis. The total mass recovered was 11.9%.

### Example 192

Fluphenazine (MW 438, melting point <25° C., oral dose 1 mg), a psychotherapeutic agent, was coated on a piece of aluminum foil (20 cm2) according to Method C. The calculated thickness of the drug film was 1.1 μm. The substrate was heated as described in Method C at 90 V for 3.5 seconds. The purity of the drug-aerosol particles was determined to be 93%. 0.7 mg was recovered from the glass tube walls after vaporization, for a percent yield of 33.3%.

The fluphenazine 2HCl salt form of the drug (MW 510, melting point 237° C.) was also tested. The drug was coated on a metal substrate (10 cm2) according to Method D. The calculated thickness of the drug film was 0.8 μm. The substrate was heated as described in Method D by charging the capacitors to 20.5 V. The purity of the drug-aerosol particles was determined to be 80.7%. 0.333 mg was recovered from the filter after vaporization, for a percent yield of 42.6%. A total mass of 0.521 mg was recovered from the test apparatus and substrate, for a total recovery of 66.7%.

### Example 193

Flurazepam (MW 388, melting point 82° C., oral dose 15 mg), sedative and hypnotic, was coated on a piece of aluminum foil (20 cm2) according to Method C. The calculated thickness of the drug film was 2.5 μm. The substrate was heated as described in Method C at 60 V for 6 seconds. The purity of the drug-aerosol particles was determined to be 99.2%. 1.8 mg was recovered from the glass tube walls after vaporization, for a percent yield of 36%.

Flurazepam was further coated on an aluminum foil substrate (24 cm2) according to Method C. 5 mg of the drug was applied to the substrate, for a calculated thickness of the drug film of 2.08 μm. The substrate was heated substantially as described in Method C at 60 V for 5 seconds. The purity of the drug-aerosol particles was determined to be 99.6%. The percent yield of the aerosol was 36%.

### Example 194

Flurbiprofen (MW 244, melting point 111° C., oral dose 50 mg), an analgesic, was coated on a piece of aluminum foil (20 cm2) according to Method C. The calculated thickness of the drug film was 4.7 μm. The substrate was heated as described in Method C at 60 V for 5 seconds. The purity of the drug-aerosol particles was determined to be >99.5%. 4.1 mg was recovered from the glass tube walls after vaporization, for a percent yield of 43.6%.

### Example 195

Fluvoxamine (MW 318, oral dose 50 mg), a psychotherapeutic agent, was coated on a piece of aluminum foil (20 cm2) according to Method C. The calculated thickness of the drug film was 4.4 μm. The substrate was heated as described in Method C at 90 V for 5 seconds. The purity of the drug-aerosol particles was determined to be 65%. 6.5 mg was recovered from the glass tube walls after vaporization, for a percent yield of 77.8%.

Another substrate containing fluvoxamine coated to a film thickness of 4.4 μm was prepared by the same method and heated under an argon atmosphere at 60 V for 8 seconds. The purity of the drug-aerosol particles was determined to be 88%. 6.9 mg was recovered from the glass tube walls after vaporization, for a percent yield of 78.4%.

### Example 196

Frovatriptan (MW 379, melting point 102° C., oral dose 2.5 mg), a migraine preparation, was coated on a piece of aluminum foil (20 cm2) according to Method C. The calculated thickness of the drug film was 3.3 μm. The substrate was heated as described in Method C at 60 V for 12 seconds. The purity of the drug-aerosol particles was determined to be 73%. 1.4 mg was recovered from the glass tube walls after vaporization, for a percent yield of 21.2%.

Frovatriptan was further coated on an aluminum foil substrate (24.5 cm2) according to Method G. 5.0 mg of the drug was applied to the substrate, for a calculated thickness of the drug film of 2.0 μm. The substrate was heated substantially as described in Method G at 90 V for 6 seconds, except that two of the openings of the T-shaped tube were left open and the third connected to the 1 L flask. The purity of the drug-aerosol particles was determined to be >91%. 2.8 mg of the drug was found to have aerosolized by mass lost from substrate, for a percent yield of 56%.

### Example 197

Hydroxyzine (MW 375, oral dose 50 mg), an antihistamine, was coated on a piece of aluminum foil (20 cm2) according to Method C. The calculated thickness of the drug film was 14 μm. The substrate was heated as described in Method C at 60 V for 9 seconds. The purity of the drug-aerosol particles was determined to be 93%. 5.54 mg was recovered from the glass tube walls after vaporization, for a percent yield of 19.9%.

The same drug coated on an identical substrate (aluminum foil, 20 cm2) to a calculated drug film thickness of 7.6 μm was heated under an argon atmosphere as described in Method C at 60 V for 9 seconds. Purity of the drug-aerosol particles was determined to be 98.6%. 4.31 mg was recovered from the glass tube walls after vaporization, for a percent yield of 28.5%.

The dihydrochloride salt form of the drug was also tested. Hydroxyzine dihydrochloride (MW 448, melting point 193° C., oral dose 50 mg) was coated on a piece of aluminum foil (20 cm2) according to Method C. The calculated thickness of the drug film was 13.7 μm. The substrate was heated as described in Method C at 60 V for 7 seconds. The purity of the drug-aerosol particles was determined to be 41.2%. 0.25 mg was recovered from the glass tube walls after vaporization, for a percent yield of 0.9%.

The salt form of the drug coated on an identical substrate (aluminum foil, 20 cm2) to a calculated drug film thickness of 12.8 μm was heated under an argon atmosphere as described in Method C at 60 V for 7 seconds. Purity of the drug-aerosol particles was determined to be 70.8%. 1.4 mg was recovered from the glass tube walls after vaporization, for a percent yield of 5.5%.

### Example 198

Ibutilide was coated on a stainless steel cylinder (8 cm2) according to Method D. 1.436 mg of drug was applied to the substrate, for a calculated drug film thickness of 1.7 μm. The substrate was heated as described in Method D by charging the capacitors to 20.5 V. The purity of the drug-aerosol particles was determined to be 98.4%. 0.555 mg was recovered from the filter after vaporization, for a percent yield of 38.6%. A total mass of 1.374 mg was recovered from the test apparatus and substrate, for a total recovery of 95.7%.

High speed photographs were taken as the drug-coated substrate was heated to monitor visually formation of a thermal vapor. The photographs showed that a thermal vapor was initially visible 25 milliseconds after heating was initiated, with the majority of the thermal vapor formed by 300 milliseconds. Generation of the thermal vapor was complete by 1200 milliseconds.

### Example 199

Indomethacin norcholine ester (MW 429, oral dose 25 mg), an analgesic, was coated on a piece of aluminum foil (20 cm2) according to Method C. The calculated thickness of the drug film was 5.1 μm. The substrate was heated as described in Method C at 60 V for 7 seconds. The purity of the drug-aerosol particles was determined to be >99.5%. 2.94 mg was recovered from the glass tube walls after vaporization, for a percent yield of 29.1%.

### Example 200

Ketorolac (MW 254, melting point 161° C., oral dose 10 mg), an analgesic, was coated on a piece of aluminum foil (20 cm2) according to Method C. The calculated thickness of the drug film was 1.1 μm. The substrate was heated as described in Method C at 60 V for 6 seconds. The purity of the drug-aerosol particles was determined to be 65.7%. 0.73 mg was recovered from the glass tube walls after vaporization, for a percent yield of 33.2%.

### Example 201

Ketorolac norcholine ester (MW 326, oral dose 10 mg), was coated on an aluminum foil substrate (20 cm2) according to Method C. 2.70 mg of drug was applied to the substrate, for a calculated thickness of the drug film of 1.4 μm. The substrate was heated as described in Method C at 60 V for 5 seconds. The purity of the drug-aerosol particles was determined to be 98.5%. 1.1 mg was recovered from the glass tube walls after vaporization, for a percent yield of 40.7%.

### Example 202

Levodopa (MW 197, melting point 278° C., oral dose 500 mg), an antiparkinsonian agent, was coated on a piece of aluminum foil (20 cm2) according to Method C. The calculated thickness of the drug film was 3.7 μm. The substrate was heated as described in Method C at 45 V for 15 seconds, then at 30 V for 10 seconds. The purity of the drug-aerosol particles was determined to be 60.6%. The percent yield of the aerosol was 7.2%.

### Example 203

Melatonin (MW 232, melting point 118° C., oral dose 3 mg), a dietary supplement, was coated on an aluminum foil substrate (20 cm2) according to Method C. 2.0 mg of drug was applied to the substrate, for a calculated thickness of the drug film of 1.0 μm. The substrate was heated as described in Method C at 90 V for 3.5 seconds. The purity of the drug-aerosol particles was determined to be >99.5%. 0.43 mg was recovered from the glass tube walls after vaporization, for a percent yield of 21.5%.

Another substrate containing melatonin coated to a film thickness of 1.1 μm was prepared by the same method and heated under an argon atmosphere at 90 V for 3.5 seconds. The purity of the drug-aerosol particles was determined to be >99.5%. 1.02 mg was recovered from the glass tube walls after vaporization, for a percent yield of 46.4%.

### Example 204

Methotrexate (oral dose 2.5 mg) was coated on a stainless steel cylinder (8 cm2) according to Method D. The calculated thickness of the drug film was 1.3 μm. The substrate was heated as described in Method D by charging the capacitors to 20.5 V. The purity of the drug-aerosol particles was determined to be 66.3%. The percent yield of the aerosol was 2.4%.

### Example 205

Methysergide (MW 353, melting point 196° C., oral dose 2 mg), a migraine preparation, was coated on a piece of aluminum foil (20 cm2) according to Method C. The calculated thickness of the drug film was 1.0 μm. The substrate was heated as described in Method C at 90 V for 3.5 seconds. The purity of the drug-aerosol particles was determined to be 67.5%. 0.21 mg was recovered from the glass tube walls after vaporization, for a percent yield of 10.5%.

### Example 206

Metoclopramide (MW 300, melting point 148° C., oral dose 10 mg), a gastrointestinal agent, was coated on an aluminum foil substrate (20 cm2) according to Method C. 2.0 mg of drug was applied to the substrate, for a calculated thickness of the drug film of 1.0 μm. The substrate was heated as under an argon atmosphere at 90 V for 3.5 seconds. The purity of the drug-aerosol particles was determined to be 99.1%. 0.43 mg was recovered from the glass tube walls after vaporization, for a percent yield of 21.7%.

### Example 207

Nabumetone (MW 228, melting point 80° C., oral dose 1000 mg), an analgesic, was coated on a piece of aluminum foil (20 cm2) according to Method C. The calculated thickness of the drug film was 4.9 μm. The substrate was heated as described in Method C at 60 V for 6 seconds. The purity of the drug-aerosol particles was determined to be >99.5%. 4.8 mg was recovered from the glass tube walls after vaporization, for a percent yield of 49%.

### Example 208

Naltrexone (MW 341, melting point 170° C., oral dose 25 mg), an antidote, was coated on an aluminum foil substrate (20 cm2) according to Method C. 10.3 mg of drug was applied to the substrate, for a calculated thickness of the drug film of 5.2 μm. The substrate was heated as described in Method C at 90 V for 5 seconds. The purity of the drug-aerosol particles was determined to be 96%. 3.3 mg was recovered from the glass tube walls after vaporization, for a percent yield of 32%.

Naltrexone was coated on an aluminum foil substrate (20 cm2) according to Method C. 1.8 mg of drug was applied to the substrate, for a calculated thickness of the drug film of 0.9 μm. The substrate was heated as described in Method C at 90 V for 3.5 seconds under an argon atmosphere. The purity of the drug-aerosol particles was determined to be 97.4%. 1.0 mg was recovered from the glass tube walls after vaporization, for a percent yield of 55.6%.

### Example 209

Nalmefene (MW 339, melting point 190° C., IV dose 0.5 mg), an antidote, was coated on a metal substrate (50 cm2). 7.90 mg of drug was coated on the substrate, to form a calculated film thickness of 1.6 μm, and heated according to Method F to form drug-aerosol particles. Purity of the drug-aerosol particles was determined to be 80%. 2.7 mg was recovered from the glass wool after vaporization, for a percent yield of 34%.

### Example 210

Perphenazine (MW 404, melting point 100° C., oral dose 2 mg), a psychotherapeutic agent, was coated on an aluminum foil substrate (20 cm2) according to Method C. 2.1 mg of drug was applied to the substrate, for a calculated thickness of the drug film of 1.1 μm. The substrate was heated as described in Method C at 90 V for 3.5 seconds. The purity of the drug-aerosol particles was determined to be 99.1%. 0.37 mg was recovered from the glass tube walls after vaporization, for a percent yield of 17.6%.

### Example 211

Pimozide (MW 462, melting point 218° C., oral dose 10 mg), a psychotherapeutic agent, was coated on a piece of aluminum foil (20 cm2) according to Method C. The calculated thickness of the drug film was 4.9 μm. The substrate was heated as described in Method C at 90 V for 5 seconds. The purity of the drug-aerosol particles was determined to be 79%. The percent yield of the aerosol was 6.5%.

### Example 212

Piroxicam (MW 248, melting point 200° C., oral dose 20 mg), a CNS-active steroid was coated on a piece of aluminum foil (20 cm2) according to Method C. The calculated thickness of the drug film was 5.0 μm. The substrate was heated as described in Method C at 60 V for 7 seconds. The purity of the drug-aerosol particles was determined to be 87.7%. 2.74 mg was recovered from the glass tube walls after vaporization, for a percent yield of 27.7%.

### Example 213

Pregnanolone (MW 318, melting point 150° C., typical inhalation dose 2 mg), an anesthetic, was coated on a metal substrate (50 cm2). 20.75 mg was coated on the substrate, for a calculated film thickness of 4.2 μm, and heated according to Method F at 300° C. to form drug-aerosol particles. Purity of the drug-aerosol particles was determined to be 87%. 9.96 mg of aerosol particles were collected for a percent yield of 48%).

### Example 214

Prochlorperazine 2HCl (MW 446, oral dose 5 mg), a psychotherapeutic agent, was coated on a stainless steel cylinder (8 cm2) according to Method D. 0.653 mg of drug was applied to the substrate, for a calculated drug film thickness of 0.8 μm. The substrate was heated as described in Method D by charging the capacitors to 20.5 V. The purity of the drug-aerosol particles was determined to be 72.4%. 0.24 mg was recovered from the filter after vaporization, for a percent yield of 36.8%. A total mass of 0.457 mg was recovered from the test apparatus and substrate, for a total recovery of 70%.

### Example 215

Protriptyline HCl (MW 299, melting point 171° C., oral dose 15 mg), a psychotherapeutic agent, was coated on an aluminum foil substrate (20 cm2) according to Method C. 2.20 mg of drug was applied to the substrate, for a calculated thickness of the drug film of 1.1 μm. The substrate was heated as described in Method C at 90 V for 3.5 seconds. The purity of the drug-aerosol particles was determined to be 99.7%. 0.99 mg was recovered from the glass tube walls after vaporization, for a percent yield of 45.0%.

### Example 216

Protriptyline (MW 263, oral dose 15 mg) was coated on an aluminum foil substrate (20 cm2) according to Method C. 5.6 mg of drug was applied to the substrate, for a calculated thickness of the drug film of 2.8 μm. The substrate was heated as described in Method C at 90 V for 3.5 seconds. The purity of the drug-aerosol particles was determined to be 89.8%. 1.4 mg was recovered from the glass tube walls after vaporization, for a percent yield of 25%.

Another substrate containing protriptyline coated to a film thickness of 2.7 μm was prepared by the same method and heated under an argon atmosphere at 90 V for 3.5 seconds. The purity of the drug-aerosol particles was determined to be 90.8%. 1.4 mg was recovered from the glass tube walls after vaporization, for a percent yield of 26.4%.

### Example 217

Pyrilamine (MW 285, melting point <25° C., oral dose 25 mg), an antihistamine, was coated on a piece of aluminum foil (20 cm2) according to Method C. The calculated thickness of the drug film was 5.2 μm. The substrate was heated as described in Method C at 60 V for 6 seconds. The purity of the drug-aerosol particles was determined to be 98.4%. 4.3 mg was recovered from the glass tube walls after vaporization, for a percent yield of 41.7%.

Pyrilamine maleate (MW 401, melting point 101° C., oral dose 25 mg), an antihistamine, was coated on a piece of aluminum foil (20 cm2) according to Method C. The calculated thickness of the drug film was 10.8 μm. The substrate was heated as described in Method C at 60 V for 7 seconds. The purity of the drug-aerosol particles was determined to be 93.7%. 10.5 mg was recovered from the glass tube walls after vaporization, for a percent yield of 48.8%.

### Example 218

Quinine (MW 324, melting point 177° C., oral dose 260 mg), an anti-infective agent, was coated on a piece of aluminum foil (20 cm2) according to Method C. The calculated thickness of the drug film was 1.1 μm. The substrate was heated as described in Method C at 60 V for 6 seconds. The purity of the drug-aerosol particles was determined to be >99.5%. 0.9 mg was recovered from the glass tube walls after vaporization, for a percent yield of 40.9%.

### Example 219

Ramipril (MW 417, melting point 109° C., oral dose 1.25 mg), a cardiovascular agent, was coated on a stainless steel cylinder (8 cm2) and heated to form drug-aerosol particles according to Method D by charging the capacitors to 20.5 V. The. purity of the drug-aerosol particles was determined to be 61.5%. 0.27 mg was recovered from the filter after vaporization, for a percent yield of 30%. A total mass of 0.56 mg was recovered from the test apparatus and substrate, for a total recovery of 62.2%.

### Example 220

Risperidone (MW 410, melting point 170° C., oral dose 2 mg), a psychotherapeutic agent, was coated on a piece of aluminum foil (20 cm2) according to Method C. The calculated thickness of the drug film was 1.4 μm. The substrate was heated as described in Method C at 90 V for 3.5 seconds. The purity of the drug-aerosol particles was determined to be 79%. The percent yield of the aerosol was 7.9%.

Risperidone was also coated on a stainless steel cylinder (8 cm2). 0.75 mg of drug was manually applied to the substrate, for a calculated drug film thickness of 0.9 μm. The substrate was heated as described in Method D by charging the capacitors to 20.5 V. The purity of the drug-aerosol particles was determined to be 87.3%. The percent yield of aerosol particles was 36.7%. A total mass of 0.44 mg was recovered from the test apparatus and substrate, for a total recovery of 59.5%.

### Example 221

Scopolamine (MW 303, melting point <25° C., oral dose 1.5 mg), a gastrointestinal agent, was coated on a metal substrate (50 cm2) according to Method F at 200° C. 37.5 mg of drug was applied to the substrate, for a calculated drug film thickness of 7.5 μm. The substrate was heated according to Method F to form drug-aerosol particles. Purity of the drug-aerosol particles was determined to be 90% by GC analysis. 1.2 mg were recovered for a percent yield of 3.2%.

### Example 222

Sotalol (MW 272, oral dose 80 mg), a cardiovascular agent, was coated on a stainless steel cylinder (8 cm2) according to Method D. 1.8 mg of drug was applied to the substrate, for a calculated drug film thickness of 2.3 μm. The substrate was heated as described in Method D by charging the capacitors to 20.5 V. The purity of the drug-aerosol particles was determined to be 96.9%. 0.66 mg was recovered from the filter after vaporization, for a percent yield of 36.7%. A total mass of 1.06 mg was recovered from the test apparatus and substrate, for a total recovery of 58.9%.

High speed photographs were taken as the drug-coated substrate was heated to monitor visually formation of a thermal vapor. The photographs showed that a thermal vapor was initially visible 30 milliseconds after heating was initiated, with the majority of the thermal vapor formed by 90 milliseconds. Generation of the thermal vapor was complete by 500 milliseconds.

### Example 223

Sulindac (MW 356, melting point 185° C., oral dose 150 mg), an analgesic, was coated on a piece of aluminum foil (20 cm2) according to Method C. The calculated thickness of the drug film was 4.3 μm. The substrate was heated as described in Method C at 60 V for 8 seconds. The purity of the drug-aerosol particles was determined to be 80.4%. 1.19 mg was recovered from the glass tube walls after vaporization, for a percent yield of 14%.

### Example 224

Terfenadine (MW 472, melting point 149° C., oral dose 60 mg), an antihistamine, was coated on a piece of aluminum foil (20 cm2) according to Method C. The calculated thickness of the drug film was 2.5 μm. The substrate was heated as described in Method C at 60 V for 8 seconds. The purity of the drug-aerosol particles was determined to be 75.4%. 0.178 mg was recovered from the glass tube walls after vaporization, for a percent yield of 3.6%.

An identical substrate coated with terfenadine (2.8 μm thick) was heated under an argon atmosphere at 60 V for 8 seconds. The purity of the drug-aerosol particles was determined to be 74.7%. 0.56 mg was recovered from the glass tube walls after vaporization, for a percent yield of 10.2%.

### Example 225

Triamcinolone acetonide (MW 434, melting point 294° C., oral dose 0.2 mg), a respiratory agent, was coated on a stainless steel cylinder (6 cm2) according to Method D. 0.2 mg of drug was applied to the substrate, for a calculated drug film thickness of 0.3 μm. The substrate was heated as described in Method D by charging the capacitors to 20.5 V. The purity of the drug-aerosol particles was determined to be 92%. 0.02 mg was recovered from the filter after vaporization, for a percent yield of 10%. A total mass of 0.09 mg was recovered from the test apparatus and substrate, for a total recovery of 45%.

### Example 226

Trihexyphenidyl (MW 302, melting point 115° C., oral dose 2 mg), an antiparkinsonian agent, was coated on a piece of aluminum foil (20 cm2) according to Method C. The calculated thickness of the drug film was 1.4 μm. The substrate was heated as described in Method C at 90 V for 3.5 seconds. The purity of the drug-aerosol particles was determined to be 77%. 1.91 mg was recovered from the glass tube walls after vaporization, for a percent yield of 68.2%.

### Example 227

Thiothixene (MW 444, melting point 149° C., oral dose 10 mg), a psychotherapeutic agent used as an anti-psychotic, was coated on a piece of aluminum foil (20 cm2) according to Method C. The calculated thickness of the drug film was 1.3 μm. The substrate was heated as described in Method C at 90 V for 3.5 seconds. The purity of the drug-aerosol particles was determined to be 74.0%. 1.25 mg was recovered from the glass tube walls after vaporization, for a percent yield of 48.1%.

### Example 228

Telmisartan (MW 515, melting point 263° C., oral dose 40 mg), a cardiovascular agent, was coated on a stainless steel cylinder (8 cm2) according to Method D. 2.73 mg of drug was applied to the substrate, for a calculated drug film thickness of 3.3 μm. The substrate was heated as described in Method D by charging the capacitors to 20.5 V. The purity of the drug-aerosol particles was determined to be 96%. 0.64 mg was recovered from the filter after vaporization, for a percent yield of 23.4%. A total mass of 2.73 mg was recovered from the test apparatus and substrate, for a total recovery of 100%.

High speed photographs were taken as the drug-coated substrate was heated to monitor visually formation of a thermal vapor. The photographs showed that a thermal vapor was initially visible 50 milliseconds after heating was initiated, with the majority of the thermal vapor formed by 400 milliseconds. Generation of the thermal vapor was complete by 1100 milliseconds.

### Example 229

Temazepam (MW 301, melting point 121° C., oral dose 7.5 mg), a sedative and hypnotic, was coated on an aluminum foil substrate (20 cm2) according to Method C. 4.50 mg of drug was applied to the substrate, for a calculated thickness of the drug film of 2.3 μm. The substrate was heated as described in Method C at 60 V for 7 seconds. The purity of the drug-aerosol particles was determined to be 97.1%. 1.9 mg was recovered from the glass tube walls after vaporization, for a percent yield of 42.2%.

### Example 230

Triamterene (MW 253, melting point 316° C., oral dose 100 mg), a cardiovascular agent, was coated on a stainless steel cylinder (8 cm2) according to Method D. 0.733 mg of drug was applied to the substrate, for a calculated drug film thickness of was 0.9 μm. The substrate was heated as described in Method D by charging the capacitors to 20.5 V. The purity of the drug-aerosol particles was determined to be >99.5%. 0.233 mg was recovered from the filter after vaporization, for a percent yield of 31.8%.

### Example 231

Trimipramine (MW 294, melting point 45° C., oral dose 50 mg), a psychotherapeutic agent, was coated on a piece of aluminum foil (20 cm2) according to Method C. The calculated thickness of the drug film was 2.8 μm. The substrate was heated as described in Method C at 90 V for 3.5 seconds. The purity of the drug-aerosol particles was determined to be 99.2%. 2.6 mg was recovered from the glass tube walls after vaporization, for a percent yield of 46.4%.

### Example 232

Ziprasidone (MW 413, oral dose 20 mg), an anti-psychotic agent, was coated on a stainless steel cylinder (8 cm2) according to Method D. 0.74 mg of drug was applied to the substrate, for a calculated drug film thickness of 0.9 μm. The substrate was heated as described in Method D by charging the capacitors to 20.5 V. The purity of the drug-aerosol particles was determined to be 87.3%. 0.28 mg was recovered from the filter after vaporization, for a percent yield of 37.8%. A total mass of 0.44 mg was recovered from the test apparatus and substrate, for a total recovery of 59.5%.

### Example 233

Zonisamide (MW 212, melting point 163° C., oral dose 75 mg), an anticonvulsant, was coated on a metal substrate and heated to form drug-aerosol particles. The substrate was heated as described in Method C and the purity of the drug-aerosol particles was determined to be 99.7%. The percent yield of the aerosol was 38.3%.

### Example 234

A. Preparation of Drug-Coated Stainless Steel Foil Substrate

Strips of clean 302/304 stainless-steel foil (0.0025 cm thick, Thin Metal Sales) having dimensions 1.5 cm by 7.0 cm were dip-coated with a drug solution. The final coated area was 5.1 cm by 1.5 cm on both sides of the foil, for a total area of 15 cm2. Foils were prepared as stated above and then extracted with acetonitrile. The amount of drug was determined from quantitative HPLC analysis. Using the known drug-coated surface area, the thickness was then obtained by:
film thickness (cm)=drug mass (g)/[drug density (g/cm³)×substrate area (cm²)]

If the drug density is not known, a value of 1 g/cm³ is assumed. The film thickness in microns is obtained by multiplying the film thickness in cm by 10,000.

After drying, the drug-coated foil was placed into a volatilization chamber constructed of a Delrin® block (the airway) and brass bars, which served as electrodes. The dimensions of the airway were 1.0 high by 5.1 wide by 15.2 cm long. The drug-coated foil was placed into the volatilization chamber such that the drug-coated section was between the two sets of electrodes. After securing the top of the volatilization chamber, the electrodes were connected to three 12V batteries wired in series with a switch controlled by circuit. The circuit was designed to close the switch in pulses so as to resistively heat the foil to a temperature within 50 milliseconds (typically between 320° and 470° C.) and maintain that temperature for up to 3 seconds. The back of the volatilization chamber was connected to a two micron Teflon® filter (Savillex) and filter housing, which were in turn connected to the house vacuum. Sufficient airflow was initiated (typically 30.5 L/min=1.0 m/sec). After the drug had vaporized, airflow was stopped and the Teflon® filter was extracted with acetonitrile. Drug extracted from the filter was analyzed by HPLC UV absorbance at 225 nm using a gradient method aimed at detection of impurities to determine percent purity. Also, the extracted drug was quantified to determine a percent yield, based on the mass of drug initially coated onto the substrate. A percent recovery was determined by quantifying any drug remaining on the substrate, adding this to the quantity of drug recovered in the filter and comparing it to the mass of drug initially coated onto the substrate.

Celecoxib and rizatriptan were tested together according to the method above, by coating a solution of the drug onto a piece of stainless steel foil (15 cm2). Twelve substrates were prepared, with film thicknesses ranging from about 4.4 μm to about 11.4 μm. The substrates were heated as described in the method above to 350° C. Purity of the drug aerosol particles from each substrate was determined. The substrate having a thickness of 4.4 μm was prepared by depositing 0.98 mg of rizatriptan and 5.82 mg of celecoxib. After volatilization of drug this substrate, 0.59 mg of rizatriptan and 4.40 mg of celecoxib were recovered from the filter, for a percent yield of 73.6%. The purity of the aerosol particles was 96.5%.

### Example 235

Using a solution of 50 mg sildenafil+10 mg caffeine per mL of solvent (2:1 chloroform:methanol), 0.0025 cm thick stainless steel foils (dimensions of 5.0×6.9 cm) were coated with 4.1 mg of sildenafil and 0.5 mg of caffeine on 45 cm2 of surface area. After drying, a variation of Method B was used. However, instead of a capacitive discharge, a feedback circuit, powered by three 12 V sealed lead acid batteries in series, was used to heat the foil to 425° C. and maintain the temperature for 500 milliseconds. Also, the 1.3×2.6×8.9 cm airway/vaporization chamber of Method B was replaced with a 5.1 by 1.0 by 15.3 cm airway to accommodate the larger foils. The airflow rate was set at 30.5 L/m (1.0 m/s). The generated aerosol was captured in a single Teflon filter, which was extracted with acetonitrile and analyzed on HPLC for purity and mass recovery. The purity of the aerosol was 91.9% by peak area under the curve at 225 nm. The mass recovery in the extracted filter was 2.9 mg sildenafil and 0.5 mg caffeine.

### Example 236

A number of other drugs were tested according to one of the above methods (A-G) or a similar method, but exhibited purity less than about 60%. These drugs were not further tested for optimization: amiloride, amiodarone, amoxicillin, beclomethasone, bromocriptine, bufexamac, candesartan, candesartan cilexetil, cetirizine, cortisone, cromolyn, cyclosporin A, dexamethasone, diclofenac, dihydroergotamine, disulfiram, dofetilide, edrophonium chloride, famotidine, fexofenadine, formoterol, furosemide, heparin, ipratropium bromide, irbesartan, labetalol, lansoprazole, lisuride, lorazepam, losartan, methocarbamol, metolazone, modafinil, montelukast, myricetin, nadolol, omeprazole, ondansetron, oxazepam, phenelzine, phentermine, propantheline bromide, quinapril hydrochloride, rabeprazole, raloxifene, rosiglitazone, tolmetin, torsemide, valsartan, and zafirlukast.

Although the invention has been described with respect to particular embodiments, it will be apparent to those skilled in the art that various changes and modifications can be made without departing from the invention.

### Example 237

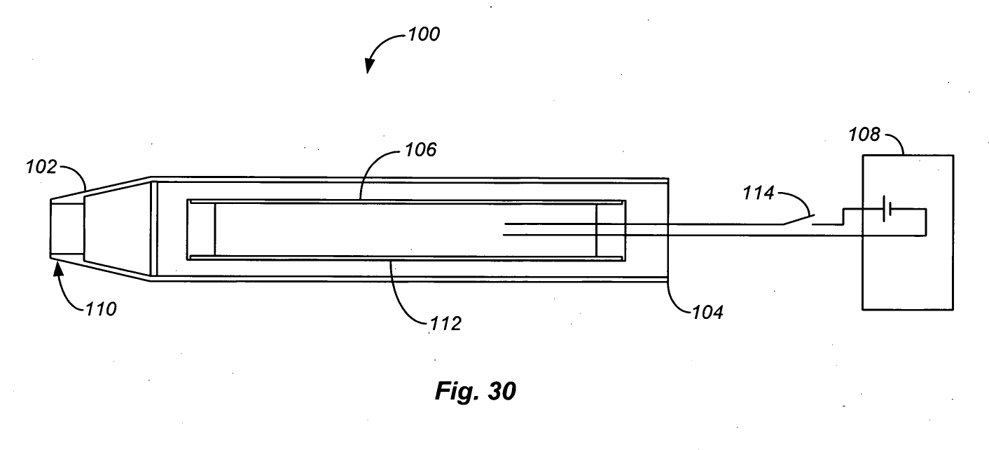

Another device used to deliver alprazolam, estazolam, midazolam or triazolam containing aerosol is described in reference to FIG. 30. Delivery device 100 has a proximal end 102 and a distal end 104, a heating module 106, a power source 108, and a mouthpiece 110. An alprazolam, estazolam, midazolam or triazolam composition is deposited on a surface 112 of heating module 106. Upon activation of a user activated switch 114, power source 108 initiates heating of heating module 106 (e.g, through ignition of combustible fuel or passage of current through a resistive heating element). The alprazolam, estazolam, midazolam or triazolam composition volatilizes due to the heating of heating module 106 and condenses to form a condensation aerosol prior to reaching the mouthpiece 110 at the proximal end of the device 102. Air flow traveling from the device distal end 104 to the mouthpiece 110 carries the condensation aerosol to the mouthpiece 110, where it is inhaled by the mammal.

Devices, if desired, contain a variety of components to facilitate the delivery of alprazolam, estazolam, midazolam or triazolam containing aerosols. For instance, the device may include any component known in the art to control the timing of drug aerosolization relative to inhalation (e.g., breath-actuation), to provide feedback to patients on the rate and/or volume of inhalation, to prevent excessive use (i.e., “lock-out” feature), to prevent use by unauthorized individuals, and/or to record dosing histories.

Purity of an alprazolam, estazolam, midazolam or triazolam containing aerosol is determined using a number of methods, examples of which are described in Sekine et al., Journal of Forensic Science 32:1271-1280 (1987) and Martin et al., Journal of Analytic Toxicology 13:158-162 (1989). One method involves forming the aerosol in a device through which a gas flow (e.g., air flow) is maintained, generally at a rate between 0.4 and 60 L/min. The gas flow carries the aerosol into one or more traps. After isolation from the trap, the aerosol is subjected to an analytical technique, such as gas or liquid chromatography, that permits a determination of composition purity.

A variety of different traps are used for aerosol collection. The following list contains examples of such traps: filters; glass wool; impingers; solvent traps, such as dry ice-cooled ethanol, methanol, acetone and dichloromethane traps at various pH values; syringes that sample the aerosol; empty, low-pressure (e.g., vacuum) containers into which the aerosol is drawn; and, empty containers that fully surround and enclose the aerosol generating device. Where a solid such as glass wool is used, it is typically extracted with a solvent such as ethanol. The solvent extract is subjected to analysis rather than the solid (i.e., glass wool) itself. Where a syringe or container is used, the container is similarly extracted with a solvent.

The gas or liquid chromatograph discussed above contains a detection system (i.e., detector). Such detection systems are well known in the art and include, for example, flame ionization, photon absorption and mass spectrometry detectors. An advantage of a mass spectrometry detector is that it can be used to determine the structure of alprazolam, estazolam, midazolam or triazolam degradation products.

Particle size distribution of an alprazolam, estazolam, midazolam or triazolam containing aerosol is determined using any suitable method in the art (e.g., cascade impaction). An Andersen Eight Stage Non-viable Cascade Impactor (Andersen Instruments, Smyrna, Ga.) linked to a furnace tube by a mock throat (USP throat, Andersen Instruments, Smyrna, Ga.) is one system used for cascade impaction studies.

Inhalable aerosol mass density is determined, for example, by delivering a drug-containing aerosol into a confined chamber via an inhalation device and measuring the mass collected in the chamber. Typically, the aerosol is drawn into the chamber by having a pressure gradient between the device and the chamber, wherein the chamber is at lower pressure than the device. The volume of the chamber should approximate the tidal volume of an inhaling patient.

Inhalable aerosol drug mass density is determined, for example, by delivering a drug-containing aerosol into a confined chamber via an inhalation device and measuring the amount of active drug compound collected in the chamber. Typically, the aerosol is drawn into the chamber by having a pressure gradient between the device and the chamber, wherein the chamber is at lower pressure than the device. The volume of the chamber should approximate the tidal volume of an inhaling patient. The amount of active drug compound collected in the chamber is determined by extracting the chamber, conducting chromatographic analysis of the extract and comparing the results of the chromatographic analysis to those of a standard containing known amounts of drug.

Inhalable aerosol particle density is determined, for example, by delivering aerosol phase drug into a confined chamber via an inhalation device and measuring the number of particles of given size collected in the chamber. The number of particles of a given size may be directly measured based on the light-scattering properties of the particles. Alternatively, the number of particles of a given size may be determined by measuring the mass of particles within the given size range and calculating the number of particles based on the mass as follows: Total number of particles=Sum (from size range 1 to size range N) of number of particles in each size range. Number of particles in a given size range=Mass in the size range/Mass of a typical particle in the size range. Mass of a typical particle in a given size range=π*D3*φ/6, where D is a typical particle diameter in the size range (generally, the mean boundary of the MMADs defining the size range) in microns, φ is the particle density (in g/mL) and mass is given in units of picograms (g-12).

Rate of inhalable aerosol particle formation is determined, for example, by delivering aerosol phase drug into a confined chamber via an inhalation device. The delivery is for a set period of time (e.g., 3 s), and the number of particles of a given size collected in the chamber is determined as outlined above. The rate of particle formation is equal to the number of 100 nm to 5 micron particles collected divided by the duration of the collection time.

Rate of aerosol formation is determined, for example, by delivering aerosol phase drug into a confined chamber via an inhalation device. The delivery is for a set period of time (e.g., 3 s), and the mass of particulate matter collected is determined by weighing the confined chamber before and after the delivery of the particulate matter. The rate of aerosol formation is equal to the increase in mass in the chamber divided by the duration of the collection time. Alternatively, where a change in mass of the delivery device or component thereof can only occur through release of the aerosol phase particulate matter, the mass of particulate matter may be equated with the mass lost from the device or component during the delivery of the aerosol. In this case, the rate of aerosol formation is equal to the decrease in mass of the device or component during the delivery event divided by the duration of the delivery event.

Rate of drug aerosol formation is determined, for example, by delivering an alprazolam, estazolam, midazolam or triazolam containing aerosol into a confined chamber via an inhalation device over a set period of time (e.g., 3 s). Where the aerosol is pure alprazolam, estazolam, midazolam or triazolam, the amount of drug collected in the chamber is measured as described above. The rate of drug aerosol formation is equal to the amount of alprazolam, estazolam, midazolam or triazolam collected in the chamber divided by the duration of the collection time. Where the alprazolam, estazolam, midazolam or triazolam containing aerosol comprises a pharmaceutically acceptable excipient, multiplying the rate of aerosol formation by the percentage of alprazolam, estazolam, midazolam or triazolam in the aerosol provides the rate of drug aerosol formation.

Typical uses for alprazolam, estazolam, midazolam, and triazolam-containing aerosols include without limitation the following: relief of the symptoms of situational anxiety, relief of acute panic attacks, relaxation of skeletal muscle, treatment of nausea and vomiting, induction of sleep, and sedation for medical or dental procedures. Alprazolam and estazolam containing-aerosols are distinguished from midazolam and triazolam-containing aerosols primarily by their durations of action, with alprazolam and estazolam having half-lives of approximately 12 hours and midazolam and triazolam having half-lives of approximately 3 hours. Thus triazolam or midazolam-containing aerosols are typically used in instances where a rapid offset of action is desired (e.g. in sedation for medical or dental procedures). In contrast, alprazolam or estazolam-containing aerosols are typically used in instances where a sustained action is desired (e.g. in the case of a panic attack, where a rapid offset of action might predispose to another episode of panic).

Alprazolam, estazolam and triazolam were purchased from Sigma (www.sigma-aldrich.com). Midazolam was obtained from Gyma Laboratories of America, Inc. (Westbury, N.Y.).s

Alprazolam can be volatized by the following procedures. A solution of 2.6 mg alprazolam in 120 μL dichloromethane was coated on a 3.6 cm×8 cm piece of aluminum foil. The dichloromethane was allowed to evaporate. The coated foil was wrapped around a 300 watt halogen tube (Feit Electric Company, Pico Rivera, Calif.), which was inserted into a glass tube sealed at one end with a rubber stopper. Running 75 V of alternating current (driven by line power controlled by a variac) through the bulb for 6 s afforded alprazolam thermal vapor (including alprazolam aerosol), which collected on the glass tube walls. Reverse-phase HPLC analysis with detection by absorption of 225 nm light showed the collected material to be at least 99.9% pure alprazolam. To obtain higher purity aerosols, one can coat a lesser amount of drug, yielding a thinner film to heat. A linear decrease in film thickness is associated with a linear decrease in impurities.

### Example 238

Volatilization of Ketoprofen Free Acid: Ketoprofen is a nonsteroidal anti-inflammatory drug with analgesic and antipyretic properties. It is a white or off-white, odorless, non-hygroscopic, fine to granular powder with a melting point of 94° C. Ketoprofen free acid (Sigma, St. Louis, Mo.) was heated to a temperature of 200° C., 300° C., or 400° C. for 60-120 seconds using a tube furnace. The evolved thermal vapor was trapped in glass wool (approximately 1.0 g), using a 2 L/min flow of air through the tube furnace to draw the evolved vapor into the glass wool trap. Extraction of the glass wool trap with acetone and methylene chloride, followed by analysis of the extract by GC/MS revealed that upon heating of 50 mg of ketoprofen free acid to 300-400° C., volatilization of approximately 30 mg of the drug occurred with the formation of 1.3% degradation products at 300° C., and 15% degradation products at 400° C.

Synthesis and Volatilization of Ketoprofen Ethyl Ester: The ketoprofen free acid, which contains a carboxylic acid group, was esterified in the following manner:

a) Four grams of ketoprofen free-acid were dissolved in 80 ml of anhydrous ethanol;

b) 0.8 ml of concentrated sulfuric acid was added and allowed to react under reflux for approximately 5 hours;

c) The ethanol was then reduced in volume to approximately 10 ml by rotary evaporation, to precipitate the product;

d) 100 mL of water was added;

e) Ketoprofen ethyl ester was extracted from the aqueous phase using 10 mL of diethyl ether (this step was repeated 3 times);

f) The organic phase was then extracted with 10 mL of saturated sodium bicarbonate solution to remove any residual acid from the organic phase (repeated 3 times);

g) Pure ketoprofen ethyl ester was then obtained in greater than 75% yield by rotary evaporation of the organic solvent from the organic phase.

Ketoprofen ethyl ester is a clear liquid at room temperature. Heating of 50 mg of ketoprofen ethyl ester to 300° C. for 120 seconds resulted in volatilization of 40 mg of drug with no formation of degradation products as detected by the method described in Example 238.

### Example 239

Volatilization of Cyclobenzaprine HCl: Cyclobenzaprine HCl (Sigma, St. Louis, Mo.) is a white, crystalline tricyclic amine salt with a melting point of 217° C. The heating of 50 mg of cyclobenzaprine HCl for 90 seconds at 300° C. resulted in the volatilization of 16.5 mg of the drug and the formation of 50% degradation products as detected by the method described in Example 238.

### Example 240

Synthesis and Volatilization of Cyclobenzaprine Free Base: Cyclobenzaprine HCl, which contains an amino group, was free-based in the following manner:

a) One gram of cyclobenzaprine HCl was dissolved in 5 ml of deionized water;

b) To this was added 4 ml of 1 N sodium hydroxide;

c) Cyclobenzaprine free base was then extracted from the aqueous solution with 6 ml of diethyl ether (repeated 3 times);

d) The diethyl ether was then evaporated to obtain greater than 75% yield of cyclobenzaprine free base.

Cyclobenzaprine free base is a translucent yellow oil. The heating of 50 mg of cyclobenzaprine free base for 120 seconds at 200° C. or for 30 seconds at 300° C. resulted in the volatilization of 10 mg of drug and no formation of degradation products as detected by the method described in Example 238.

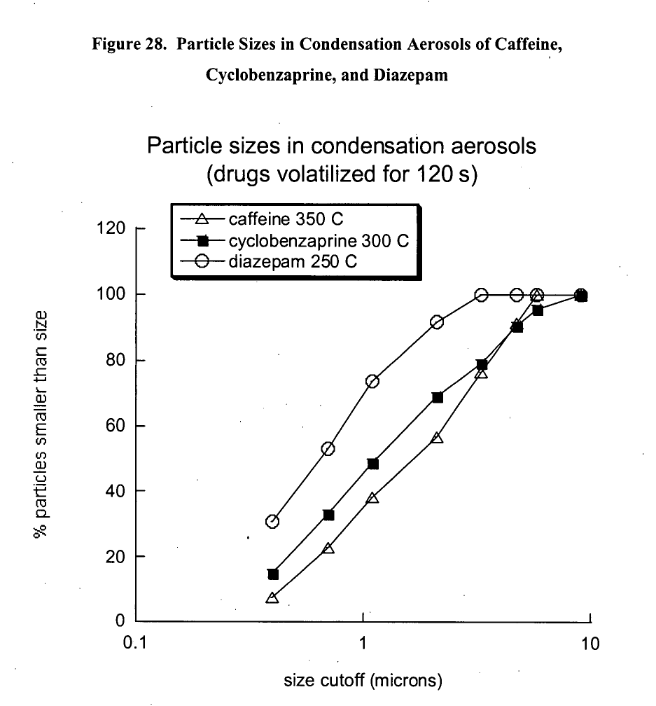

Cyclobenzaprine free base was aerosolized as follows: 50 mg of cyclobenzaprine free base was placed in a preheated 300° C. furnace tube, through which air was flowed at a rate comparable to normal inhalation (28 L/minute). Heating of the cyclobenzaprine free base followed by cooling and condensation of the volatilized free base drug in the flowing air resulted in formation of 10 mg of cyclobenzaprine aerosol in 30 s. The particle size distribution in the aerosol was analyzed by cascade impaction using an Andersen Eight Stage Non-viable Cascade Impactor (Andersen Instruments, Smyrna, Ga.) linked to the furnace tube by a mock throat (USP throat, Andersen Instruments, Smyrna, Ga.). As shown in FIG. 28, the mass median aerodynamic diameter of the aerosol was 0.8 micron, with a geometric standard deviation of 3. In an otherwise identical experiment, the air flow rate was reduced to 2 L/minute to facilitate trapping of the aerosol. The yield of volatilized cyclobenzaprine was similar to above. The purity of the aerosol was analyzed by collecting the thermal vapor in a glass wool trap. The trap was extracted multiple times with acetone, followed by methylene chloride. Analysis of the trap extract by tandem gas chromatography-mass spectrometry (GC-MS) revealed that the aerosol contained pure cyclobenzaprine free base, and no detectable contaminants or other compounds

### Example 241

Volatilization of Valproate Free Acid: Valproate free acid (valproic acid) is a colorless liquid with anticonvulsant, mood-stabilizing, and analgesic properties. It boils at 130° C. at 120 mmHg pressure. The heating of 80 mg of valproic acid (Sigma, St. Louis, Mo.) to 150° C.-300° C. for 120 seconds resulted in the volatilization of 50 mg of drug, with no formation of degradation products at 150° C. and 0.5% formation of degradation products at 300° C. as detected by the method described in Example 238.

### Example 242

Condensation Aerosol of Caffeine: Caffeine is a mild stimulant that tends to improve attentiveness, decrease sleepiness, and reduce pain, especially headache pain. Caffeine free base (Sigma, St. Louis, Mo.) was aerosolized in the following manner: 100 mg of caffeine free base powder was placed in a preheated 350° C. furnace tube, through which air was flowed at a rate comparable to normal inhalation (28 L/minute). Heating of the caffeine followed by cooling and condensation of the volatilized caffeine in the flowing air resulted in formation of 35 mg of caffeine aerosol in 2 minutes. Visually, the aerosol comprised dense white wisps of material, with the individual particles too small to be differentiated by the human eye. The aerosol was odorless. The particle size distribution in the aerosol was analyzed by cascade impaction using an Andersen Eight Stage Non-viable Cascade Impactor (Andersen Instruments, Smyrna, Ga.) linked to the furnace tube by a mock throat (USP throat, Andersen Instruments, Smyrna, Ga.). As shown in FIG. 28, the mass median aerodynamic diameter of the aerosol was 1.1 micron, with a geometric standard deviation of 3. In an otherwise identical experiment, the air flow rate was reduced to 2 L/minute to facilitate trapping of the aerosol. The yield of volatilized caffeine was similar to above. The purity of the aerosol was analyzed by collecting the thermal vapor in a glass wool trap. The trap was extracted multiple times with acetone, followed by methylene chloride. Analysis of the trap extract by tandem gas chromatography-mass spectrometry (GC-MS) revealed that the aerosol contained only caffeine free base. No contaminants or other compounds were detected.

Caffeine free base was alternatively aerosolized as follows: 10 mg of caffeine free base powder was placed on a thin glass slide. The glass slide was placed inside a solenoid composed of approximately 50 cm of heating wire (Nichrome wire CH15-500, Omega Engineering, Stamford, Conn.) wound into 20 coils of approximately 0.7 cm diameter spread over a linear distance of approximately 2 cm. Nine AC volts were applied to the wire for 60 s. During this time, greater than 95% of the added caffeine volatilized, forming a thermal vapor. The thermal vapor was either allowed to condense into a dense, white, odorless aerosol, or alternatively was collected in a sealed 40 mL glass vial in which the solenoid was contained. The purity of the aerosol was analyzed by extraction of the glass vial. Analysis of the vial extract by GC-MS revealed the presence of approximately 10 mg of pure free base caffeine and no other compounds (limit of detection approximately 0.02 mg), implying a purity of greater than 99.9%. Greater than 99% of the volatilized material could be accounted for by mass balance, verifying that greater than 99% of the aerosol consists of pure caffeine free base.

### Example 243

Condensation Aerosol of Diazepam: Diazepam is a benzodiazepam sedative. Diazepam free base (Sigma, St. Louis, Mo.) is a fine white powder with a melting point of 125° C. Diazepam free base was aerosolized in the following manner: 20 mg of diazepam free base powder was placed in a preheated 250° C. furnace tube, through which air was flowed at a rate comparable to normal inhalation (28 L/min). Heating of the diazepam free base followed by cooling and condensation of the volatilized freebase drug in the flowing air resulted in formation of a therapeutic quantity of diazepam aerosol, 5 mg in 120 s. The particle size distribution in the aerosol was analyzed by cascade impaction using an Andersen Eight Stage Non-viable Cascade Impactor (Andersen Instruments, Smyrna, Ga.) linked to the furnace tube by a mock throat (USP throat, Andersen Instruments, Smyrna, Ga.). As shown in FIG. 28, the mass median aerodynamic diameter of the aerosol was 0.7 micron, with a geometric standard deviation of 2. In an otherwise identical experiment, the air flow rate was reduced to 2 L/minute to facilitate trapping of the aerosol. The yield of volatilized diazepam was similar to above. The purity of the aerosol was analyzed by collecting the thermal vapor in a glass wool trap. The trap was extracted multiple times with acetone, followed by methylene chloride. Analysis of the trap extract by tandem gas chromatography-mass spectrometry (GC-MS) revealed that the aerosol contained pure diazepam freebase, with no degradation products found.

Diazepam was further aerosolized by placing 10 mg of free base powder onto a 5×7 cm piece of aluminum foil, which was then placed into a preheated 300° C. furnace tube, through which air was flowed slowly (2 L/min). Heating of the diazepam free base followed by cooling and condensation of the volatilized freebase drug in the flowing air resulted in formation of a therapeutic quantity of diazepam aerosol, 6 mg in 15 s. The purity of the aerosol was analyzed by collecting the thermal vapor in a glass wool trap. The trap was extracted multiple times with acetonitrile containing 0.1% trifluoroacetic acid. Analysis of the trap extract by high performance liquid chromatography with detection by ultraviolet and visible light absorption using a photodiode array detector revealed that the aerosol contained >99% pure diazepam freebase, with only trace degradation products found.

Diazepam was further aerosolized by first coating it onto a 10×15 cm piece of aluminum foil as follows:

a) 10 mg of diazepam was dissolved in 1.5 mL of diethyl ether;

b) The ether solution was slowly and evenly poured over the 10×15 cm piece of aluminum foil;

c) The ether was allowed to evaporate in a fume hood at room temperature for 15 minutes.

The foil increased in weight by 10 mg, corresponding to the weight of the added diazepam. The coated foil was then placed into a preheated 300° C. furnace tube, through which air was flowed slowly (2 L/min). Heating of the thin layer of diazepam free base followed by cooling and condensation of the volatilized freebase drug in the flowing air resulted in formation of a therapeutic quantity of diazepam aerosol, 10 mg in less than 15 s (all of the coated diazepam was volatilized). The purity of the aerosol was analyzed by collecting the thermal vapor in a glass wool trap. The trap was extracted multiple times with acetonitrile containing 0.1% trifluoroacetic acid. Analysis of the trap extract by high performance liquid chromatography with detection by ultraviolet and visible light absorption using a photodiode array detector revealed that the aerosol contained pure diazepam freebase with no detectable degradation products. Based on previous particle size analysis of diazepam free base aerosol (see above), it is estimated that at least 1.5×1010 diazepam particles were generated in less than 15 s, implying a rate of particle generation of at least 10⁹ particles per second.

### Example 244

Liquid Aerosolization of Diazepam: Diazepam is a solid at room temperature, and therefore cannot be aerosolized by standard liquid aerosolization methods. Furthermore, diazepam is poorly soluble in water, thus aqueous solutions of diazepam contain only small (e.g. <1%) amounts of diazepam by weight. Heating of diazepam free base to 150□C results in melting without any thermal decomposition (as measured by GC-MS). A pure, inhalable aerosol of diazepam free base is produced by pushing the warm free base through micron-sized holes using pressure applied by a plunger.

### Example 245

Synthesis of Ketoprofen Ester: Ketoprofen is a nonsteroidal anti-inflammatory drug with analgesic and antipyretic properties. It is a white or off-white, odorless, non-hygroscopic, fine to granular powder with a melting point of 94° C. Ketoprofen free acid (Sigma, St. Louis, Mo.), which contains a carboxylic acid group, was esterified in the following manner:

a) Four grams of ketoprofen free-acid were dissolved in 80 ml of anhydrous ethanol;

b) 0.8 ml of concentrated sulfuric acid was added and allowed to react under reflux for approximately 5 hours;

c) The ethanol was then reduced in volume to approximately 10 ml by rotary evaporation, to precipitate the product;

d) 100 mL of water was added;

e) Ketoprofen ethyl ester was extracted from the aqueous phase using 10 mL of diethyl ether (this step was repeated 3 times);

f) The organic phase was then extracted with 10 mL of saturated sodium bicarbonate solution to remove any residual acid from the organic phase (repeated 3 times);

g) The organic phase was dried with anhydrous sodium sulfate and then filtered to remove the sodium sulfate particles;

h) Pure ketoprofen ethyl ester was then obtained in greater than 75% yield by rotary evaporation of the organic solvent from the organic phase.

Ketoprofen ethyl ester is a clear liquid with a faint citrus odor at room temperature.

### Example 246

Condensation Aerosol of Ketoprofen Ester: Ketoprofen ethyl ester was vapor coated onto a 5×7 cm piece of aluminum foil as follows: 50 mg of ketoprofen ethyl ester was placed on a piece of aluminum foil in the center of a 250□C tube furnace. The piece of aluminum foil to be coated was placed approximately 6 cm away from the ketoprofen ethyl ester, also inside the tube furnace. Air was flowed at 2 L/min from the added compound towards the foil to be coated. Over 10 minutes, a thin coating of approximately 15 mg of ketoprofen ethyl ester was obtained on the foil to be coated. The vapor-coated foil was then introduced into a separate, pre-heated, 300° C. oven under a steady airflow. Within 20 seconds, the thin coat of ketoprofen ethyl ester was fully volatilized and condensed into an aerosol in the flowing air. Visually, the aerosol comprised dense clear particles, similar in appearance to fog. The aerosol had a faint citrus odor. The purity of the aerosol was analyzed by collecting the thermal vapor in a glass wool trap. The trap was extracted multiple times with acetone, followed by methylene chloride. Analysis of the trap extract by tandem gas chromatography-mass spectrometry (GC-MS) revealed that the aerosol contained pure ketoprofen ethyl ester.

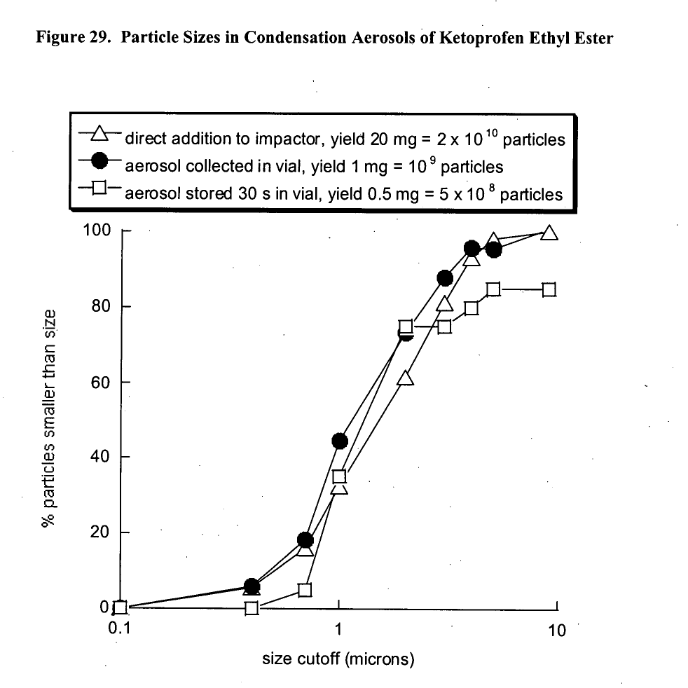

Ketoprofen ethyl ester was alternatively aerosolized as follows: 25 mg of ketoprofen ethyl ester was placed on a thin glass slide. The glass slide was placed inside a solenoid composed of approximately 50 cm of heating wire (Nichrome wire CH15-500, Omega Engineering, Stamford, Conn.) wound into 20 coils of approximately 0.7 cm diameter spread over a linear distance of approximately 2 cm. Twelve AC volts were applied to the wire for 10 s, followed by 9 AC volts for 20 s. During this time, greater than 90% of the added ketoprofen ethyl ester volatilized, forming a thermal vapor. When cool air was run over the coil at a flow rate mimicking inhalation, the volatilized ketoprofen ethyl ester rapidly condensed into an aerosol. The particle size distribution in the aerosol was analyzed by cascade impaction using an Andersen Eight Stage Non-viable Cascade Impactor (Andersen Instruments, Smyrna, Ga.) with a mock throat (USP throat, Andersen Instruments, Smyrna, Ga.). As shown in FIG. 29, the mass median aerodynamic diameter of the aerosol was approximately 1 micron, with a standard deviation of approximately 3 microns. The aerosol consisted of approximately 2×1010 particles, or approximately 10⁹ particles produced per second of active compound volatilization (during the first 10 seconds, the wire is heated but the compound does not volatilize substantially).

Ketoprofen ethyl ester was alternatively aerosolized using the above heating wire approach, but with the evolved vapors confined to a 40 mL vial. After the 30 s application of voltage, the aerosol content of the vial was analyzed. Approximately 1 mg of ketoprofen ethyl ester aerosol was found in the vial and approximately 19 mg of thermal vapor was condensed on the sides of the vial. As shown in FIG. 29, the particle size of the aerosol found in the vial was indistinguishable from that of aerosol formed by flowing cool air directly over the heated wire. The aerosol consisted of approximately 1×10⁹ particles in 40 mL, or 2.5×107 particles/mL.

The stability of ketoprofen ethyl ester aerosol was investigated by allowing the aerosol trapped in the vial to remain in the vial for 30 s (in the absence of application of voltage or any other form of heating). During these 30 s, approximately 60% of the aerosol collided with the sides of the vial and was thus lost. As shown in FIG. 2, the particle size of the aerosol did not change substantially during the 30 s time interval.

The purity of the ketoprofen ethyl ester aerosol was analyzed by allowing the entirety of the aerosol to condense on the sides of a glass vial. The purity of the aerosol was analyzed by extraction of the glass vial. Analysis of the vial extract by GC-MS revealed the presence of approximately 20 mg of ketoprofen ethyl ester and less than 1% degradation products. Greater than 99% of the volatilized material could be accounted for by mass balance, verifying that greater than 99% of the aerosol consists of pure ketoprofen ethyl ester.

### Example 247

Liquid Aerosolization of Ketoprofen Ester: Ketoprofen ethyl ester is substantially more viscous than water at room temperature, and thus cannot readily be aerosolized by standard liquid aerosolization methods. Heating of ketoprofen ethyl ester to 175° C. results in marked reduction in viscosity without any thermal decomposition (as measured by GC-MS). A pure, inhalable aerosol ketoprofen ethyl ester is produced by pushing warm ketoprofen ethyl ester through micron-sized holes using pressure applied by a plunger.

### Example 248

### General Procedure for Determining Whether a Drug is a “Heat Stable Drug”

Drug is dissolved or suspended in a solvent (e.g., dichloromethane or methanol). The solution or suspension is coated to about a 4 micron thickness on a stainless steel substrate of about 8 cm2 surface area. The substrate may either be a standard stainless steel foil or a heat-passivated stainless steel foil. The substrate is heated to a temperature sufficient to generate a thermal vapor (generally ˜350° C.) but at least to a temperature of 200° C. with an air flow typically of 20 L/min (1 m/s) passing over the film during heating. The heating is done in a volatilization chamber fitted with a trap (such as described in the Examples above). After vaporization is complete, airflow is discontinued and the resultant aerosol is analyzed for purity using the methods disclosed herein. If the resultant aerosol contains less than 10% drug degradation product, i.e., the TSR≧9, then the drug is a heat stable drug. If, however, at about 4 micron thickness, greater than 10% degradation is determined, the experiment is repeated at the same conditions, except that film thicknesses of about 1.5 microns, and of about 0.5 micron, respectively, are used. If a decrease in degradation products relative to the 4 micron thickness is seen at either of these thinner film thicknesses, a plot of film thickness versus purity is graphed and extrapolated out to a film thickness of 0.05 microns. The graph is used to determine if there exists a film thickness where the purity of the aerosol would be such that it contains less than 10% drug degradation products. If such a point exists on the graph, then the drug is defined as a heat stable drug

### Example 238

### General Procedure for Screening Drugs to Determine Aerosolization Preferability

Drug (1 mg) is dissolved or suspended in a minimal amount of solvent (e.g., dichloromethane or methanol). The solution or suspension is pipeted onto the middle portion of a 3 cm by 3 cm piece of aluminum foil. The coated foil is wrapped around the end of a 1½ cm diameter vial and secured with parafilm. A hot plate is preheated to approximately 300° C., and the vial is placed on it foil side down. The vial is left on the hotplate for 10 s after volatilization or decomposition has begun. After removal from the hotplate, the vial is allowed to cool to room temperature. The foil is removed, and the vial is extracted with dichloromethane followed by saturated aqueous NaHCO3. The organic and aqueous extracts are shaken together, separated, and the organic extract is dried over Na2SO4. An aliquot of the organic solution is removed and injected into a reverse-phase HPLC with detection by absorption of 225 nm light. A drug is preferred for aerosolization where the purity of the drug isolated by this method is greater than 85%. Such a drug has a decomposition index less than 0.15. The decomposition index is arrived at by subtracting the drug purity fraction (i.e., 0.85) from 1.

TABLE 2
Phase Transition Temperatures of Various
Pharmaceutical Formulations

| | | Melting Point |
|---|---|---|
| Compound | Form | (° C.) |
| Alizapide | free base | 139 |
| Alizapride | HCl | 206 |
| Aspirin | free acid | 135 |
| Aspirin | methyl ester | 51 |
| Aspirin | phenyl ester | 97 |
| Azacyclonol | free base | 160 |
| Azacyclonol | HCl | 283 |
| Benactyzine | free base | 51 |
| Benactyzine | HCl | 177 |
| Benactyzine | methobromide | 169 |
| Biperiden | free base | 114 |
| Biperiden | HCl | 238 |
| Buclizine | free base | bp_{0.001} = 218 |
| Buclizine | dihydrochloride | 235 |
| Bupropion | free base | bp_{0.005} = 52 |
| Bupropion | HCl | 233 |
| Clomethiazole | free base | bp₇ = 92 |
| Clomethiazole | HCl | 130 |
| Clomethiazole | methanedisulfonate | 120 |
| Clomethiazole | ethanedisulfonate | 124 |
| Clonidine | free base | 130 |
| Clonidine | HCl | 305 |
| Desipramine | free base | bp_{0.02} = 172 |
| Desipramine | HCl | 215 |
| Dihydrocodeine | free base | 112 |
| Dihydrocodeine | Bitartrate | 192 |
| Diphenhydramine | free base | bp_{2.0} = 150 |
| Diphenhydramine | HCl | 166 |
| Doxepin | free base | bp_{0.03} = 154 |
| Doxepin | HCl | 184 |
| Doxepin | Maleate | 161 |
| Eptastigmine | free base | 60 |
| Eptastigmine | tartrate | 122 |
| Flupirtine | free base | 115 |
| Flupirtine | HCl | 214 |
| Flupirtine | maleate | 175 |
| Flurazepam | free base | 77 |
| Flurazepam | dihydrochloride | 190 |
| Galanthamine | free base | 126 |
| Galanthamine | HCl | 246 (dec) |
| Galanthamine | HBr | 256 (dec) |
| Haloperidol | free base | 148 |
| Haloperidol | HCl | 226 |
| Hydromorphone | free base | 266 |
| Hydromorphone | HCl | 310 (dec) |
| Ketorolac | free acid | 160 |
| Ketorolac | tromethamine salt | 174 |
| Lofexidine | free base | 126 |
| Lofexidine | HCl | 221 |
| Maprotiline | free base | 92 |
| Maprotiline | HCl | 230 |
| Meclofenamic acid | free acid | 258 |
| Meclofenamic acid | sodium salt | 290 |
| | monohydrate | |
| Melperone | free base | Bp_{0.1} = 120 |
| Melperone | HCl | 210 |
| Mephenesin | free base | 70 |
| Mephenesin | carbamate | 93 |
| Methadone | free base | 78 |
| Methadone | HCl | 235 |
| Minaprine | free base | 122 |
| Minaprine | dihydrochloride | 182 |
| Morphine | free base | 200 (sublimes) |
| Morphine | HCl | 200 |
| Morphine | sulfate | 250 |
| Nalorphine | free base | 208 |
| Nalorphine | HCl | 260 |
| Nalorphine | HBr | 258 (dec) |
| Naloxone | free base | 184 |
| Naloxone | HCl | 205 |
| Naltrexone | free base | 168 |
| Naltrexone | HCl | 274 |
| Naproxen | free acid | 152 |
| Naproxen | sodium salt | 244 |
| Nefazodone | free base | 83 |
| Nefazodone | HCl | 180 |
| Perphenazine | free base | 95 |
| Perphenazine | dihydrochloride | 225 |
| Phenelzine | free base | Bp_{0.1} = 74 |
| Phenelzine | HCl | 174 |
| Promazine | free base | bp_{0.3} = 203 |
| Promazine | HCl | 181 (dec) |
| Ritanserin | free base | 145 |
| Ritanserin | tartrate | 198 |
| Selegiline | free base | bp_{0.8} = 92 |
| Selegiline | HCl | 141 |
| Sumatriptan | free base | 169 |
| Sumatriptan | succinate | 165 |
| Tandospirone | free base | 112 |
| Tandospirone | HCl | 227 |
| Tandospirone | citrate | 169 |
| Thioridazine | free base | 72 |
| Thioridazine | HCl | 155 |
| Thiothixene | free base | 116 |
| Thiothixene | dimaleate | 158 |
| Thiothixene | dioxalate | 229 |
| Tranylcypromine | free base | Bp_{1.5} = 79 |
| Tranylcypromine | HCl | 164 |
| Trazodone | free base | 86 |
| Trazodone | HCl | 223 |
| Trimipramine | free base | 45 |
| Trimipramine | maleate | 142 |
| Tropisetron | free base | 201 |
| Tropisetron | HCl | 283 (dec) |
| Valproic Acid | free acid | bp₂₀ 128 |
| Valproic Acid | sodium salt | solid at RT |
| Valeric Acid | free acid | bp₇₄₆ 186 |
| Valeric Acid | ethyl ester | bp₇₄₆ 145 |
| Yohimbine | free base | 234 |
| Yohimbine | HCl | 302 (dec) |

1-28. (canceled)

29. A method for treating a disorder of the central nervous system comprising administering to the respiratory tract of a patient in need of treatment benzodiazepine for treating said disorder, wherein the benzodiazepine is administered in a dose that is at least about two times less than that required by oral administration and wherein delivery is to the pulmonary system.

30. The method of claim 29 wherein delivery is to the alveoli region of the pulmonary system.

31. The method of claim 29 wherein administering is for rescue therapy.

32. The method of claim 29 wherein the benzodiazepine is present in dry powder particles.

33. The method of claim 32 wherein the benzodiazepine is present in the dry powder particles in an amount of at least 20 weight percent.

34. The method of claim 32 wherein the benzodiazepine is present in the dry powder particles in an amount of at least 40 weight percent.

35. The method of claim 32 wherein the benzodiazepine is present in the dry powder particles in an amount of at least 50 weight percent.

36. The method of claim 32 wherein the particles have a tap density of less than about 0.4 g/cm.sup.3.

37. The method of claim 32 wherein the particles have a mass median aerodynamic diameter of less than about 5 microns.

38. The method of claim 32 wherein the particles have a mass median geometric diameter greater than about 5 microns.

39. The method of claim 32 wherein the particles have a mass median aerodynamic diameter of less than about 3 microns.

40. The method of claim 32 wherein the particles include a phospholipid.

41. The method of claim 32 wherein the particles include citrate and a multivalent salt.

42. The method of claim 32 wherein the particles include an amino acid.

43. The method of claim 42 wherein the amino acid is leucine.

44. The method of claim 32 wherein the particles are administered via a dry powder inhaler.

45. The method of any one of claims 29-44, wherein the benzodiazepine is alprazolam.
